(19) United States
(12) Patent Application Publication
(10) Pub. No.: US 2026/0048216 A1
(43) Pub. Date: Feb. 19, 2026

(54) RESPIRATORY VENTILATION APPARATUS

(71) Applicants: BMC MEDICAL CO., LTD., Beijing (CN); BMC (TIANJIN) MEDICAL CO., LTD., Tianjin (CN)

(72) Inventors: Jianxin ZHI, Beijing (CN); Lijun LIU, Beijing (CN); Jie ZHANG, Beijing (CN); Jiayin ZHANG, Beijing (CN); Xiaoyang LIU, Tianjin (CN); Mingzhao ZHOU, Tianjin (CN); Yajie WANG, Tianjin (CN); Libao LIN, Tianjin (CN); Yu WANG, Tianjin (CN); Xiaoshuai GUO, Tianjin (CN); Wei HE, Tianjin (CN); Zhi ZHUANG, Beijing (CN)

(73) Assignees: BMC MEDICAL CO., LTD., Beijing (CN); BMC (TIANJIN) MEDICAL CO., LTD., Tianjin (CN)

(21) Appl. No.: 19/367,848

(22) Filed: Oct. 24, 2025

Related U.S. Application Data
(63) Continuation of application No. PCT/CN2024/086531, filed on Apr. 8, 2024.

(30) Foreign Application Priority Data
Apr. 25, 2023 (CN) 202310456467.6
Apr. 25, 2023 (CN) 202310456483.5
Apr. 25, 2023 (CN) 202310456713.8
Apr. 25, 2023 (CN) 202310457610.3
Apr. 25, 2023 (CN) 202310457714.4
Apr. 25, 2023 (CN) 202320959625.5
Apr. 25, 2023 (CN) 202320964150.9
Apr. 25, 2023 (CN) 202320965161.9
Apr. 25, 2023 (CN) 202320965277.2
Apr. 25, 2023 (CN) 202320965661.2
Apr. 25, 2023 (WO) PCT/CN2023/090623

(51) Int. Cl. A61M 16/00

(52) U.S. Cl. CPC A61M 16/0066; A61M 16/0003; A61M 2202/0007; A61M 2202/02; A61M 2205/3606; A61M 2205/42

(57) ABSTRACT

Embodiments of the present disclosure provides a respiratory ventilation apparatus including: a main body configured to generate a high pressure gas above atmospheric pressure. The main body may include a noise reduction device, the noise reduction device being configured to reduce a noise generated during an operation of the main body. The noise reduction device may include a gas inlet structure, a noise reduction housing, a blower cavity, and a gas channel. A gas may enter the gas channel through the gas inlet structure. The gas channel may be formed between a sidewall of the noise reduction housing and a sidewall of the blower cavity, and the gas channel may be configured to transfer the gas. The blower cavity may be disposed with at least one cavity gas inlet along a gasflow direction. The gas in the gas channel may enters the blower cavity through the cavity gas inlet.

### CROSS-REFERENCE TO RELATED APPLICATIONS

This application is a continuation of International Application No. PCT/CN2024/086531, filed on Apr. 8, 2024, which claims priority of Chinese Patent Application No. 202310456483.5 filled on Apr. 25, 2023, Chinese Patent Application No. 202310456713.8 filed on Apr. 25, 2023, Chinese Patent Application No. 202320965661.2 filed on Apr. 25, 2023, Chinese Patent Application No. 202310457610.3 filed on Apr. 25, 2023, Chinese Patent Application No. 202320965161.9 filed on Apr. 25, 2023, Chinese Patent Application No. 202320964150.9 filed on Apr. 25, 2023, Chinese Patent Application No. 202310456467.6 filed on Apr. 25, 2023, Chinese Patent Application No. 202320965277.2 filed on Apr. 25, 2023, Chinese Patent Application No. 202310457714.4 filed on Apr. 25, 2023, Chinese Patent Application No. 202320959625.5 filed on Apr. 25, 2023, and International Application No. PCT/CN2023/090623 filed on Apr. 25, 2023, the contents of each of which are entirely incorporated herein by reference.

### TECHNICAL FIELD

The present disclosure relates to the technical field of medical equipment, and in particular, to a respiratory ventilation apparatus.

### BACKGROUND

A respiratory ventilation apparatus, as a device with a function of artificial ventilation, has been commonly used in a treatment of respiratory failure, sleep apnea hypoventilation syndrome and other diseases due to various causes. The respiratory ventilation apparatus holds a very important position in the field of modern medicine. When the user uses the respiratory ventilation apparatus to treat respiratory diseases, the respiratory ventilation apparatus needs to inhale gas from the outside environment, carry out a series of processes on the gas before supply the user with breathing. The current respiratory ventilation apparatus often has more problems, for example, a noise generated by a blower will seriously affect the patient's rest, and even interfere with the patient's treatment, and for example, water in the reservoir refluxes into a gas channel and lead to choking.

In order to solve the above problem, the present disclosure aims to provide a respiratory ventilation apparatus.

### SUMMARY

One embodiment of the present disclosure provides a respiratory ventilation apparatus including: a main body configured to generate a high pressure gas above atmospheric pressure. The main body may include a noise reduction device, the noise reduction device being configured to reduce a noise generated during an operation of the main body. The noise reduction device may include a gas inlet structure, a noise reduction housing, a blower cavity, and a gas channel. A gas may enter the gas channel through the gas inlet structure. The gas channel may be formed between a sidewall of the noise reduction housing and a sidewall of the blower cavity, and the gas channel may be configured to transfer the gas. The blower cavity may be disposed with at least one cavity gas inlet along a gasflow direction.

The gas in the gas channel may enters the blower cavity through the cavity gas inlet.

In some embodiments, the gas channel may be disposed around an outer wall of the blower cavity.

In some embodiments, the blower cavity may be disposed with at least two cavity gas inlets in the gasflow direction. By adopting the at least two cavity gas inlets to divert the gas in the gas channel into the blower cavity, a turbulence of gas may be reduced. In this way, the noise of the gasflow entering the blower cavity may be effectively attenuated.

In some embodiments, the gas inlet structure may be located at a beginning of an extension direction of the gas channel, and the cavity gas inlet may be located at an end of the extension direction of the gas channel; an angle between a first connecting line connecting a gas outlet of the gas inlet structure and a center of the gas channel and a second connecting line connecting the cavity gas inlet and the center of the gas channel may be greater than 180°. An annular gas channel with a turning angle greater that 180° may be disposed around an outside of the blower cavity, and the gas inlet and the cavity gas inlet may be respective located at the beginning and the end of the extension direction of the gas channel. In this way, the gasflow may flow for a longer path in the annular gas channel, and the noise may be better reduced. Moreover, as the annular gas channel is reducing the noise generated by the gasflow inside, the annular gas channel may further reduce the noise generated by an operation of the blower in the blower cavity.

In some embodiments, the main body may include the blower. The blower may be fixedly mounted inside the noise reduction device, and the blower may be fixedly mounted on a bottom, a top or a sidewall of the noise reduction device by a blower mounting structure.

In some embodiments, there may be a gap between an outer wall of the blower and an inner wall of the noise reduction device. No matter how the blower is mounted in the noise reduction device, the gap between the outer wall of the blower and the inner wall of the noise reduction device may effectively prevent the noise generated when the blower operates from transmitting to the outside, which further reduces the noise of the main body as a whole.

In some embodiments, the respiratory ventilation apparatus may further include a connecting device including a breather pipe receiving the high pressure gas.

In some embodiments, the respiratory ventilation apparatus may further include a sealing structure configured for a sealing connection between the main body and the connecting device.

In some embodiments, the sealing structure may further include an elastic pipe, the elastic pipe or the breather pipe being disposed with an annular protrusion, the annular protrusion being used for a sealing connection between the elastic pipe and the breather pipe.

The respiratory ventilation apparatus may be provided with the annular protrusion on the breather pipe or the elastic pipe of the sealing structure, so that during a process of mating the breather pipe of the connecting device and the elastic pipe of the sealing structure, the annular protrusion is allowed to be deformed by squeezing. After deformation, the annular protrusion may produce a large friction force between the breather pipe and the elastic pipe to limit separation of the breather pipe and the elastic pipe, which not only ensures sealing performance and firmness of connecting the breather pipe and the elastic pipe, but also simplifies assembly operations, thereby effectively improving assembly efficiency.

In some embodiments, the connecting device may include a reservoir or a cover plate without a reservoir.

In some embodiments, the reservoir may include: a reservoir housing including a reservoir upper housing and a reservoir lower housing, the reservoir housing including a reservoir cavity for holding a liquid; a heat transfer component disposed on the reservoir lower housing for a heat transfer, so that the liquid in the reservoir cavity vaporizes; a gas inlet channel disposed on the reservoir upper housing for introducing the gas from outside of the reservoir into the reservoir cavity, the gas inlet channel including a reservoir first gas inlet for the gas from outside of the reservoir to enter the gas inlet channel, and a reservoir first gas outlet channel for the gas inside the gas inlet channel to enter the reservoir cavity; and a gas outlet channel disposed on the reservoir upper housing for conducting the gas out of the reservoir cavity, the gas outlet channel including a reservoir second gas inlet for the gas within the reservoir cavity to enter the gas outlet channel and a reservoir second gas outlet for conducting gas out of the gas outlet channel.

In some embodiments, the reservoir may further include a reflux prevention component configured to prevent a reflux of liquid within the reservoir cavity to at least one of the gas inlet channel or the gas outlet channel.

By disposing the gas channel in the reservoir, overcome a problem of a liquid in the reservoir refluxing into the gas inlet or the gas outlet to damage the respiratory ventilation apparatus, or cause the patient to choke, thereby ensuring a normal use of the respiratory ventilation apparatus and improving the patient's experience.

In some embodiments, the reservoir may further include a pressing button disposed on an outer surface of the reservoir housing, the pressing button being configured to be able to press in a direction close to an interior of the reservoir housing under an external force to enable the reservoir to be assembled to or disassembled from the main body.

In some embodiments, the pressing button may further include an elastic structure configured to: produce an elastic deformation when the pressing button is pressed in a direction close to the interior of the reservoir housing; and recover, when the external force is released, when the external force is released, deformation to drive the pressing button to rebound to reset in a direction away from the interior of the reservoir housing, so that the reservoir is fixed to the main body of the respiratory ventilation apparatus or is reset after being disassembled.

Disposing the pressing button may realize a detachable connection between the reservoir and the main body of the respiratory ventilation apparatus, reduce an operation difficulty of disassembling the reservoir, and make the connection between the reservoir and the main body of the respiratory ventilation apparatus flexible to be set. Disposing the elastic structure on the pressing button may make it possible for the pressing button to be automatically reset after being pressed, with no additional operation required, and the operations may be simple. Through a movable setup of the pressing button relative to the reservoir housing, the reservoir may be kept stable during disassembly and is not easy to shake relative to the main body of the respiratory ventilation apparatus. In such cases, not only the connection between the reservoir and the main body of the respiratory ventilation apparatus main body is improved, but also the difficulty of the reservoir to enter and exit the main body of the respiratory ventilation apparatus is reduced, ensuring a simpler disassembly and installation of the reservoir.

In some embodiments, the reservoir may further include a heating device configured to heat the liquid in the reservoir.

In some embodiments, the heating device may be detachably mounted at a lower end portion of the reservoir. By detachably connecting the separated reservoir and the heating device, the reservoir may be taken as a disposable reservoir, which solves a problem of the reservoir being difficult to clean. The heating device may be removed for cleaning, and then replaced with a new reservoir for recycling.

In some embodiments, the noise reduction device may include a porous sound-absorbing plate configured to improve a noise reduction effect of the noise reduction device; the porous sound-absorbing plate being disposed with through holes along a thickness direction, including: a first surface in contact with the gasflow; and a second surface away from the gasflow. The through holes of the porous sound-absorbing plate may communicate the first surface and the second surface, the through holes may at least including through hole of a first type and through hole of a second type, an aperture of through hole of the first type being smaller than an aperture of through hole of the second type.

By disposing the porous sound-absorbing plate in the noise reduction device, the noise reduction may be performed through the through holes on the porous sound-absorbing plate, and the through holes with different apertures may be adopted to improve a noise reduction range of the porous sound-absorbing plate.

In some embodiments, the respiratory ventilation apparatus may further include a liquid level detection device configured to detect a liquid level of a humidifier in the respiratory ventilation apparatus.

In the liquid level detection device of some embodiments, the master control chip may be connected to the circuit of the capacitance sensor, and based on the capacitance value, whether the liquid level is lower than the height of the position of the sensor in the vertical direction may be determined. Capacitance sensing of the liquid level detection device of some embodiments may be a kind of remote sensing effect, in which a direct contact is not required between the sensor and a measured object (such as water, etc.). With simple structures, the sidewall of the main body and the sidewall of the reservoir may not need to be punched and a style of the reservoir may not be limited, making a circuit detection simpler, the detection more accurate, an anti-interference ability higher, and a cost lower.

In some embodiments, the respiratory ventilation apparatus may further include a flow detection device configured to detect a flow of the gasflow output from the respiratory ventilation apparatus. The flow detection device may be mounted within the noise reduction device, so that the gasflow forms a pressure difference on two sides of the flow detection device along the gasflow direction, thereby realizing the flow detection using a pressure difference principle.

In some embodiments, the respiratory ventilation apparatus may include a pipeline interface communicated with an external device. An external gas may be pressurized by the main body and then transported to the external device through the pipeline interface. The pipeline interface may be disposed with a temperature detection device inside the pipeline interface for detecting the temperature of the gas at the pipeline interface. A heating temperature of the heating device in the humidifier may be further adjusted according to the temperature detected by the temperature detection device.

In some embodiments, the respiratory ventilation apparatus may further include a main body button disposed on a main body housing of the respiratory ventilation apparatus, the main body button including a function button configured for a user to manipulate an operation of the respiratory ventilation apparatus. By disposing the function button, the user may be convenient to manipulate the operation of the respiratory ventilation apparatus, thereby realizing operations like power on and off control and parameter setting, etc.

One of the embodiments of the present disclosure provide a noise reduction device. The noise reduction device may include a gas inlet structure, a noise reduction housing, a blower cavity, and a gas channel. The gas channel may be located between an inner wall of the noise reduction housing and an outer wall of the blower cavity. The gas channel may be configured for transmitting a gasflow. The blower cavity may be disposed with at least two cavity gas inlets along a gasflow direction. The gas may enter the gas channel through the gas inlet structure, and then enter the blower cavity through the cavity gas inlet.

In some embodiments, the gas channel may include three spaces in the direction along the gasflow direction. The at least three spaces may be arranged in a sequence of large cavities and small cavities at intervals, and the cavity gas inlet may be disposed in one of the at least three spaces with large cavity.

In some embodiments, the gas channel may include a first space, a second space and a third space in the gasflow direction. The first space may be greater than the second space and the second space may be smaller than the third space. The blower cavity may include a first gas inlet and a second gas inlet, the first gas inlet being located in the first space, and the second gas inlet being located in the third space.

In some embodiments, the gas channel may further include a fourth space and a fifth space in the gasflow direction. The fourth space may be smaller than the third space and the fifth space may be greater than the fourth space. The blower cavity may further include a third gas inlet, the third inlet being located in the fifth space.

In some embodiments, the gas inlet structure may include a gas inlet pipe. The gas inlet pipe may be disposed with one or more separating components inside the gas inlet pipe so as to divide the gas inlet pipe into two or more gas inlet sub-pipes.

In some embodiments, a porous sound-absorbing plate and/or a sound-absorbing cotton may be disposed in the gas channel. The porous sound-absorbing plate may be disposed with a plurality of sound-absorbing holes in a thickness direction.

In some embodiments, a height of a position of a gas outlet of the gas inlet structure in a vertical direction may be smaller than a height of a position of a gas inlet in the vertical direction.

One of the embodiments of the present disclosure may provide a respiratory ventilation apparatus, including the noise reduction device of any one of the above embodiments.

One of the embodiments of the present disclosure may provide another noise reduction device, including: a noise reduction housing, a gas inlet structure, a blower cavity, and a gas channel. The gas may enter the gas channel through a gas outlet of the gas inlet structure. The gas channel may be disposed between an inner wall of the noise reduction housing and an outer wall of the blower cavity, and the gas channel may be disposed along the outer wall of the blower cavity. The blower cavity may be disposed with a cavity gas inlet. The gas in the gas channel may enter the blower cavity through the cavity gas inlet. The gas inlet structure may be located at the beginning of an extension direction of the gas channel, and the cavity gas inlet may be located at an end of the extension direction of the gas channel. An angle between a first connecting line connecting a gas outlet of the gas inlet structure and a center of the gas channel and a second connecting line connecting the cavity gas inlet and the center of the gas channel may be greater than 180°.

In some embodiments, the gas inlet structure may include a gas inlet pipe with a bending structure, so that the gas may flow into the gas channel after turning in the gas inlet pipe.

In some embodiments, a first gasflow direction of a gas inlet of the gas inlet pipe may be different from a second gasflow direction of a gas outlet of the gas inlet pipe.

In some embodiments, the bending structure may make the gas inlet of the gas inlet pipe and a gas outlet of the gas inlet pipe located at different heights.

In some embodiments, the noise reduction housing may further include a water collection cavity. The blower cavity may further include a cavity gas outlet, the cavity gas outlet being communicated with the water collection cavity.

In some embodiments, the noise reduction housing may be disposed with a main outlet, the gas may flow out from the cavity gas outlet into the water collection cavity, and flow out of the main outlet after being lifted.

In some embodiments, the cavity gas outlet may be located at a height higher than a bottom surface of the water collection cavity in the vertical direction.

In some embodiments, the porous sound-absorbing plate and/or a sound-absorbing cotton may be placed in the gas channel. The porous sound-absorbing plate may be disposed with a number of sound-absorbing holes in a thickness direction.

One of the embodiments of the present disclosure provide another respiratory ventilation apparatus, including the noise reduction device of any of the above embodiments.

One of the embodiments of the present disclosure may provide a porous sound-absorbing plate, including a first surface in contact with the gasflow; and a second surface away from the gasflow. The through holes of the porous sound-absorbing plate may communicate the first surface and the second surface. The through holes may at least include through hole of a first type and through hole of a second type, an aperture of through hole of the first type being smaller than an aperture of through hole of the second type.

In some embodiments, the through hole may include a first opening on the first surface; a second opening on the second surface; and a sidewall of the through hole communicating the first opening with the second opening. An aperture of the first opening may be less than or equal to an aperture of the second opening.

In some embodiments, the porous sound-absorbing plate may include: a first porous sound-absorbing plate disposed along a first plane; and a second porous sound-absorbing plate disposed at a preset angle with the first plane. The through hole in the first porous sound-absorbing plate may be through hole of the first type and the through hole in the second porous sound-absorbing plate may be through hole of the second type.

In some embodiments, the porous sound-absorbing plate may include: a splitting component, when the gasflow flows through the splitting component, the gasflow may flow through both separating component surfaces of the splitting component; and/or a guiding component, when the gasflow flows through the guiding component, the gasflow may flow sequentially through both separating component surfaces of the guiding component.

In some embodiments, the second porous sound-absorbing plate may further include: at least one porous sound-absorbing plate bending; and a fourth separating component group disposed at the porous sound-absorbing plate bending, an angle between a disposing direction and the gasflow direction satisfying a second threshold condition. The fourth separating component group may include at least one first side separating component and at least one second side separating component, the first side separating component being disposed on one side of the porous sound-absorbing plate bending and the second side separating component being disposed on the other side of the porous sound-absorbing plate bending, the first side separating component being adjacent to the second side separating component and being staggered in the disposing direction of the second porous sound-absorbing plate, the adjacent first side separating component and the second side separating component having a staggered portion in the disposing direction of the second porous sound-absorbing plate. When the gasflow flows sequentially through the first side separating component and the second side separating component adjacent to each other, the gasflow may flow along a first surface of the first side separating component and turn and enter the gasflow channel formed by the staggered portion upon reaching the end of the first side separating component to reach the first surface of the second side separating component and flow out.

One of the embodiments of the present disclosure provide another respiratory ventilation apparatus, including the porous sound-absorbing plate of any of the above embodiments.

One of the embodiments of the present disclosure provide another respiratory ventilation apparatus, including a main body configured to generate a high pressure gas above the atmospheric pressure; a connecting device including a breather pipe for receiving the high pressure gas; and a sealing structure configured for a sealing connection between the main body and the connecting device; the sealing structure including an elastic pipe, the elastic pipe or the breather pipe being disposed with an annular protrusion, the annular protrusion being configured for the sealing connection between the elastic pipe and the breather pipe.

In some embodiments, the annular protrusion may be disposed on an inner wall of the elastic pipe or on an outer wall of the breather pipe such that the breather pipe is able to extend into the elastic pipe; or the annular protrusion may be disposed on an inner wall of the breather pipe or on an outer wall of the elastic pipe such that the elastic pipe is able to extend into the breather pipe.

In some embodiments, the outer wall of the elastic pipe or the outer wall of the breather pipe may be disposed with a limiting groove. The main body may include a limiting structure. The limiting structure may be coupled with the limiting groove, and the limiting structure matches the limiting groove for limiting a movement range of the elastic pipe or the breather pipe in a radial direction.

In some embodiments, the limiting groove may include one or more limiting protrusions, the one or more limiting protrusions being circumferentially distributed along an outer wall of the limiting groove, a distance between an outer wall of the one or more limiting protrusions and a central axis of the limiting groove being greater than or equal to an inner diameter of the limiting structure.

One of the embodiments of the present disclosure provides a reservoir including: a reservoir housing including a reservoir upper housing and a reservoir lower housing, the reservoir housing further including a reservoir cavity for holding a liquid; a heat transfer component disposed in the reservoir lower housing for conducting heat to the liquid in the reservoir cavity to vaporize the liquid in the reservoir cavity; a gas inlet channel disposed on the reservoir upper housing for introducing the gas into the reservoir cavity, the gas inlet channel including a reservoir first gas inlet for the gas outside of the reservoir to enter the gas inlet channel, and a reservoir first gas outlet for the gas in the gas inlet channel to enter the reservoir cavity. A gas outlet channel may be disposed in the reservoir upper housing for conducting the gas out of the reservoir cavity, the gas outlet channel including a reservoir second gas inlet for the gas within the reservoir cavity to enter the gas outlet channel and a reservoir second gas outlet for conducting the gas out of the outlet channel. A reflux prevention component may be disposed for preventing the liquid in the reservoir cavity from refluxing into the gas inlet channel and/or the gas outlet channel.

In some embodiments, the reflux prevention component may include one or more of a first bending portion on the gas inlet channel and a second bending portion on the gas outlet channel and/or a flow guide rib. The first bending portion and the second bending portion may bend towards a center of the reservoir cavity, the first gas outlet of the reservoir may be disposed on the first bending portion, the second gas inlet of the reservoir may be disposed on the second bending portion. The flow guide rib may be disposed between the reservoir first gas outlet and an upper housing sidewall adjacent to the reservoir first gas outlet and/or between the reservoir second gas inlet and an upper housing sidewall adjacent to the reservoir second gas inlet.

In some embodiments, a distance between the reservoir first gas outlet and/or the reservoir second gas inlet and the center of the reservoir cavity may be less than a preset distance threshold.

In some embodiments, the reservoir first gas outlet may be a gas outlet in a first opening direction, the reservoir second gas outlet may be a gas outlet in a second opening direction. The first opening direction may be opposite or perpendicular to the second opening direction.

In some embodiments, at least one side of the gas inlet channel and/or the gas outlet channel may be a detachable structure.

One of the embodiments of the present disclosure provides another respiratory ventilation apparatus including the main body and the reservoir of any of the above embodiments.

One of the embodiments of the present disclosure provides another reservoir, including a reservoir housing and a pressing button disposed on an outer surface of the reservoir housing, the pressing button being configured to be capable of being pressed in a direction proximate to an interior of the reservoir housing under an external force to enable the reservoir to be assembled to or disassembled from the main body. The pressing button may include an elastic structure, the elastic structure being configured to: produce an elastic deformation when the pressing button is pressed in a direction close to the interior of the reservoir housing; and recover, when the external force is released, deformation to drive the pressing button to rebound to reset in a direction away from the interior of the reservoir housing, so that the reservoir is fixed to the main body of the respiratory ventilation apparatus or is reset after disassembly.

In some embodiments, the pressing button may further include a connection structure, the connection structure being disposed at one end of the pressing button near the main body, the connection structure being detachably connected to the main body, when the pressing button is pressed in a direction proximate the interior of the reservoir housing, the connecting structure may separate from the main body.

In some embodiments, the elastic structure may include at least one of an elastic arm and an elastic component; the elastic arm including a free end and a fixed end, the elastic arm being configured such that when the pressing button is pressed in the direction proximate to the interior of the reservoir housing, the free end may slide in a direction away from the fixed end and the elastic arm may undergo an elastic deformation. When the external force is released, the free end may slide in a direction proximate to the fixed end and the elastic arm recovers its deformation. The elastic component may be configured such that when the pressing button is pressed in a direction proximate to the interior of the reservoir housing, the height of the elastic component may decrease and undergo elastic deformation; and when the external force is released, the height of the elastic component may increase and recover deformation.

In some embodiments, one end of the elastic component may be fixedly connected to one of the side of the pressing button proximate to the interior of the reservoir housing or the outer surface of the reservoir housing, and the other end of the elastic component may abut against the other of the side of the pressing button proximate to the interior of the reservoir housing or the outer surface of the reservoir housing.

In some embodiments, at least one of a limiting structure and a guiding structure may further be disposed between the pressing button and the outer surface of the reservoir housing. The limiting structure may be configured to limit a range of movement of the pressing button in the direction away from the interior of the reservoir housing; and the guiding structure may be configured to guide the pressing button to move according to a preset path.

In some embodiments, the outer surface of the reservoir housing may include a recessed structure, the pressing button being accommodated within the recessed structure, the elastic structure being disposed between the side of the pressing button proximate to the interior of the reservoir housing and a bottom of the recessed structure.

One of the embodiments of the present disclosure provides another respiratory ventilation apparatus, including the reservoir of any one of the above embodiments.

One of the embodiments of the present disclosure provides a liquid level detection device configured for a liquid level detection of a humidifier of a respiratory ventilation apparatus, including: at least one sensor configured for detecting the liquid level of the humidifier; the sensor being disposed on one end of a main body sidewall of the respiratory ventilation apparatus away from or facing a humidifier sidewall, the main body sidewall being adjacent to the humidifier sidewall.

In some embodiments, a difference between a height of a first position of the sensor and a height of a second position of a bottom plate of the humidifier may be in a range of 2 mm to 50 mm.

In some embodiments, there may be a plurality of sensors, each of the sensors being disposed at a preset positional height in a vertical direction.

In some embodiments, when the sensor is disposed on one side of the main body sidewall away from the humidifier sidewall, a distance between the sensor and an inner wall of the humidifier sidewall in a horizontal direction may range from 1 mm to 8 mm.

In some embodiments, the sensor may include a capacitive sensor, the liquid level detection device may further include: a main control chip connected to a circuit of the sensor, the main control chip being configured to receive a capacitance value collected by the sensor and determine, based on the capacitance value, whether the liquid level is below a height of a position of the sensor in a vertical direction.

One of the embodiments of the present disclosure provides another respiratory ventilation apparatus, including the liquid level detection device of any one of the above embodiments.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which the same reference numbers represent the same structures, and wherein:

FIG. 1A is a schematic diagram illustrating an exemplary system for a respiratory ventilation apparatus according to some embodiments of the present disclosure;

FIG. 1B is a schematic diagram illustrating the respiratory ventilation apparatus according to some embodiments of the present disclosure;

FIG. 1C is a schematic diagram illustrating a structure of a noise reduction device in the respiratory ventilation apparatus according to some embodiments of the present disclosure;

FIG. 1D is a schematic diagram illustrating atop hoisting mounting of a blower in the noise reduction device according to some embodiments of the present disclosure;

FIG. 1E is a schematic diagram illustrating a bottom supporting mounting of the blower in the noise reduction device according to some embodiments of the present disclosure;

FIG. 1F is a schematic diagram illustrating a sidewall suspending mounting of the blower in the noise reduction device according to some embodiments of the present disclosure;

FIG. 1G is a schematic diagram illustrating a three-dimensional (3D) structure of a sectional view along BB direction in FIG. 1F;

FIG. 1H is a dissembled schematic diagram of a reservoir and a heating device of the respiratory ventilation apparatus according to some embodiments of the present disclosure;

FIG. 11 is a dissembled schematic diagram of the reservoir and the heating device of the respiratory ventilation apparatus according to some other embodiments of the present disclosure;

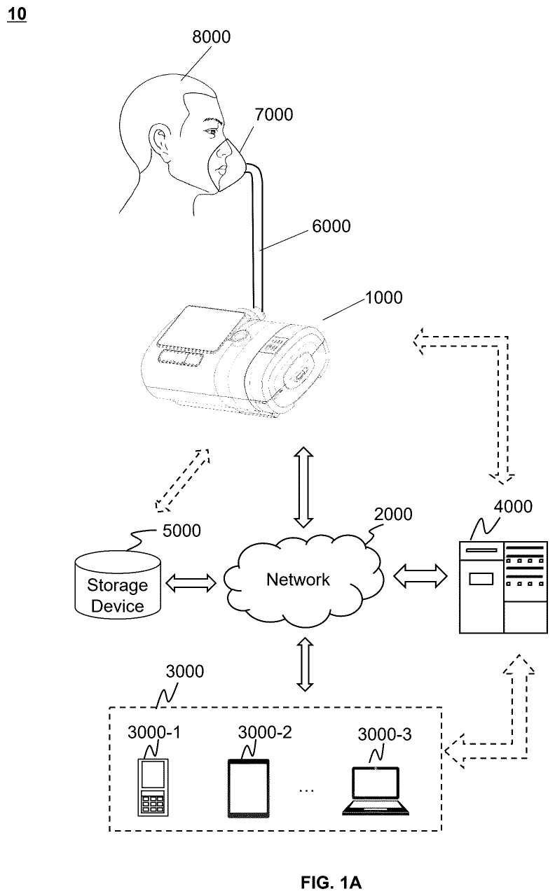

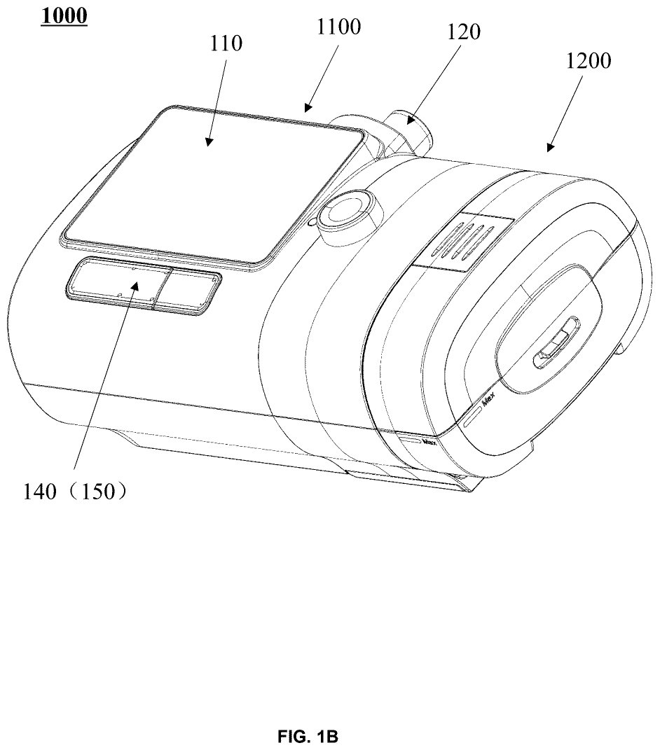

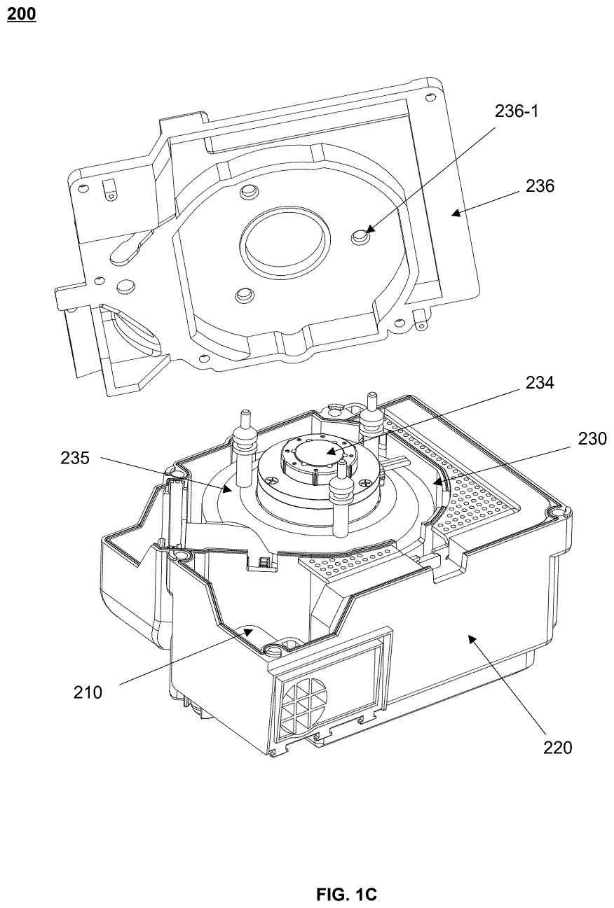

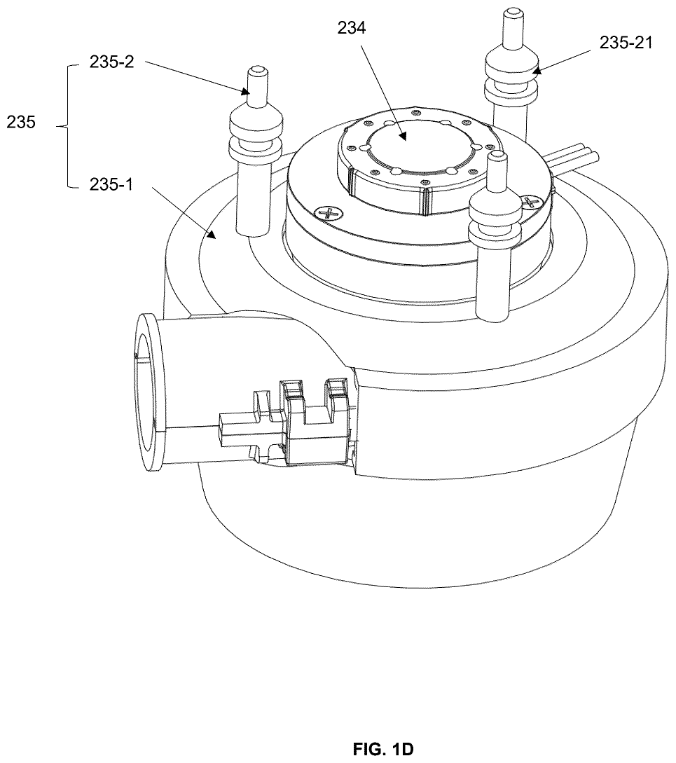

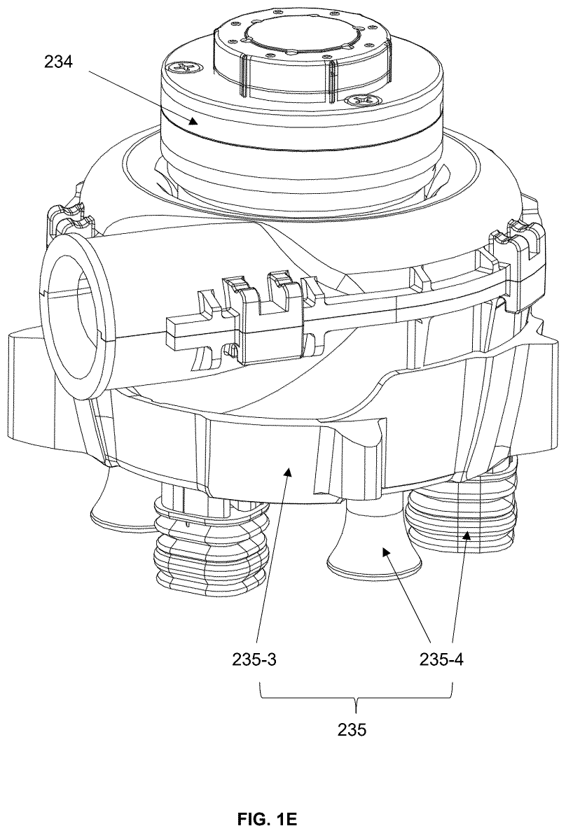

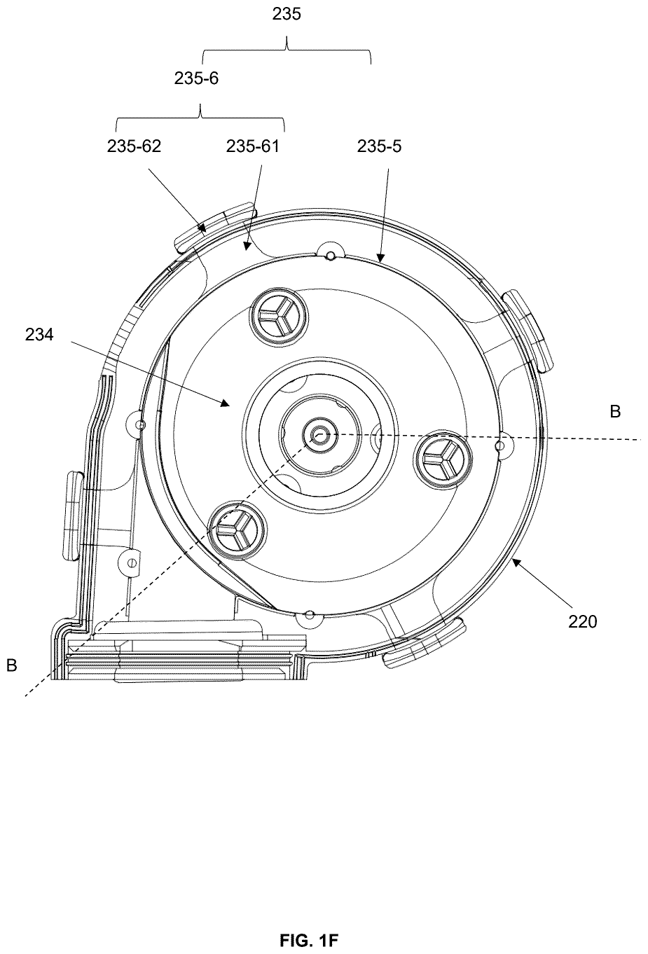

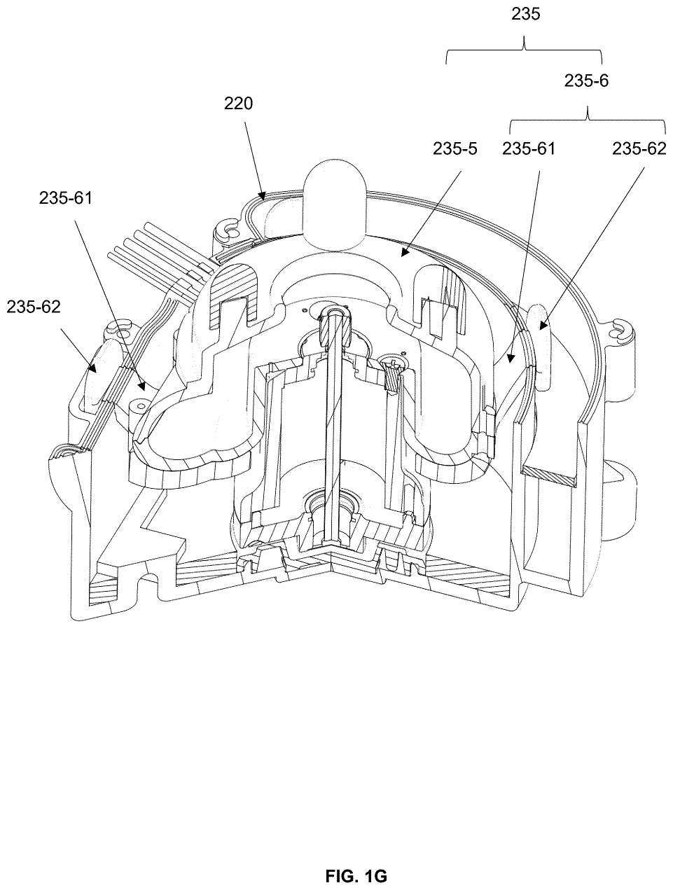

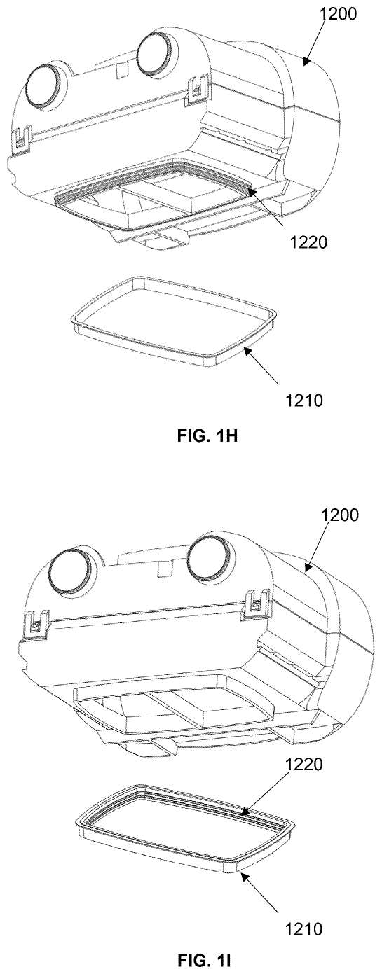

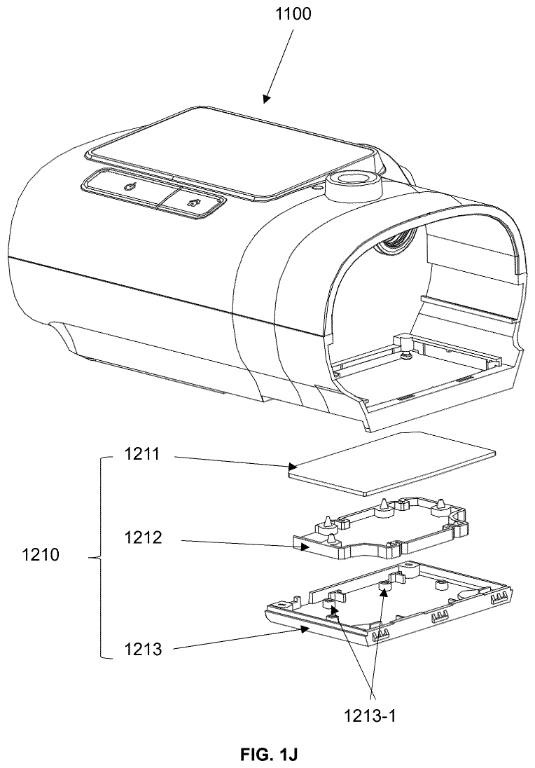

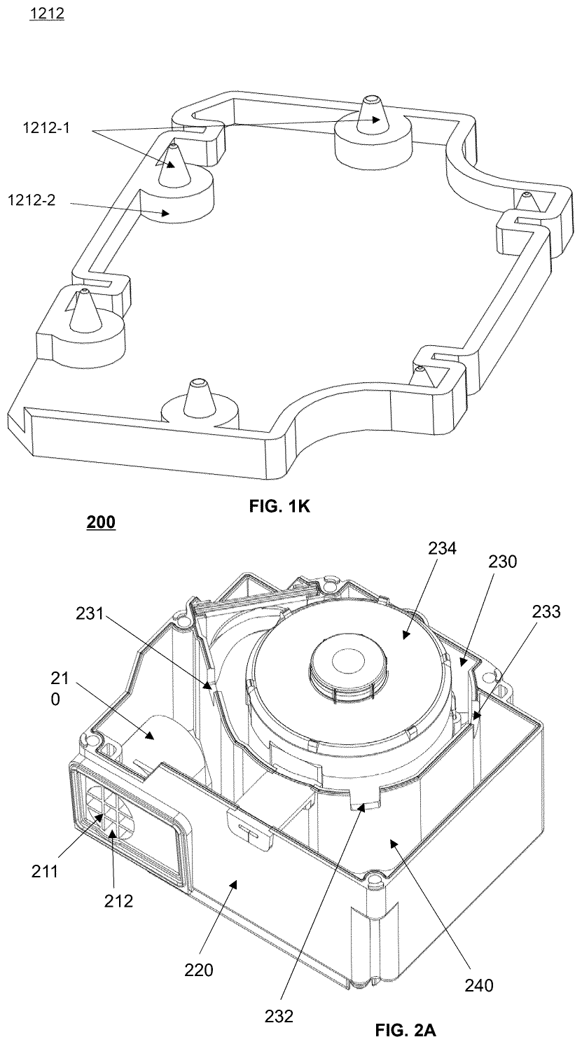

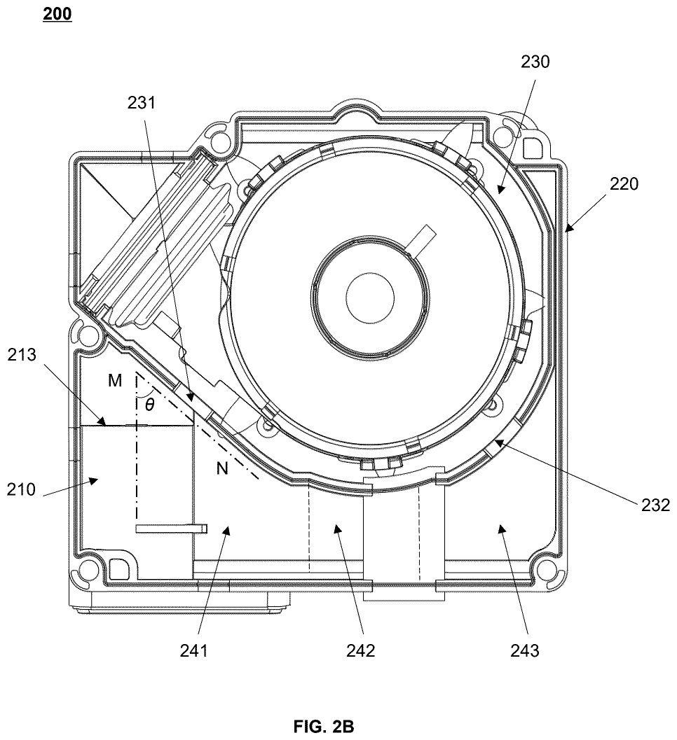

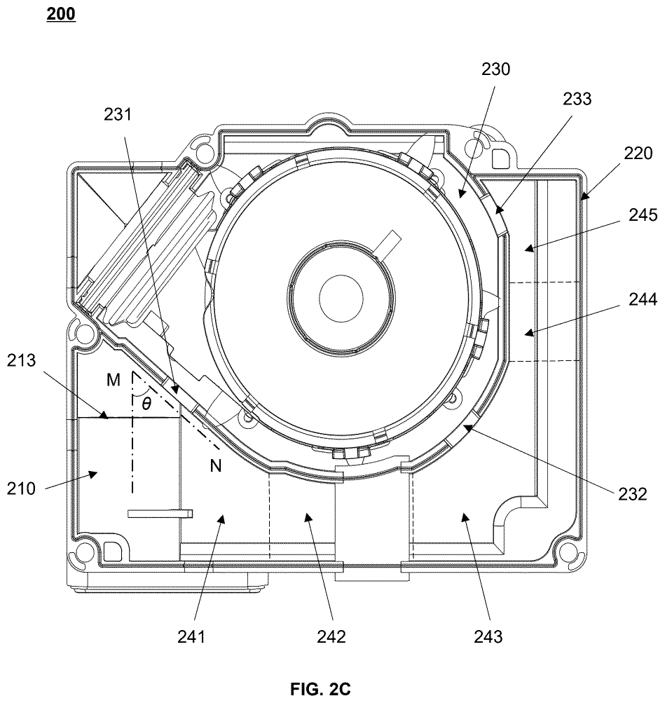

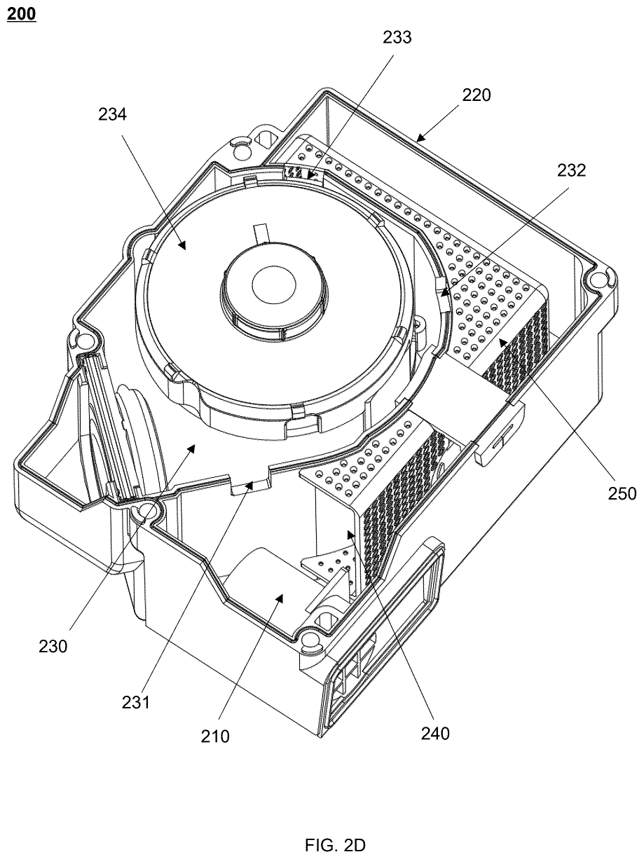

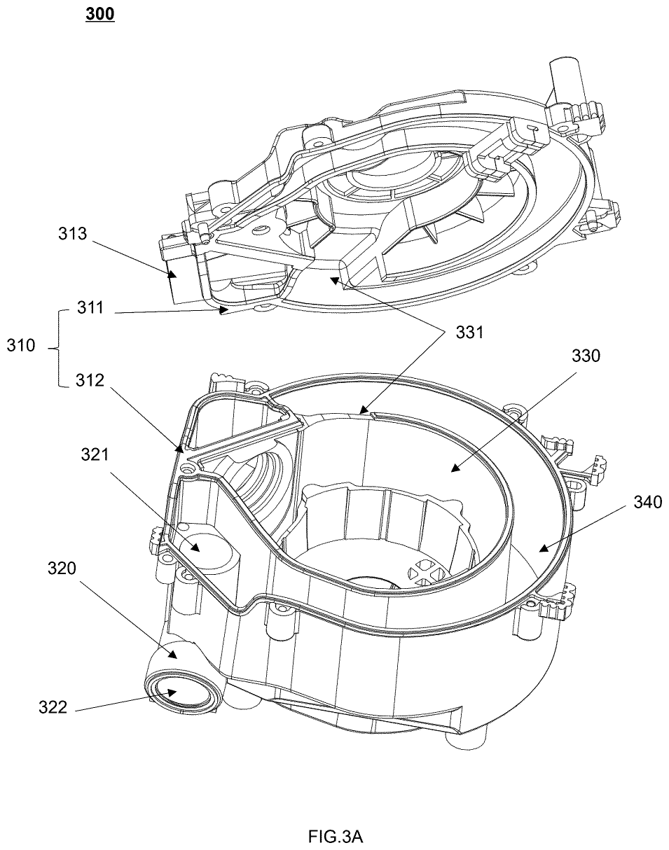

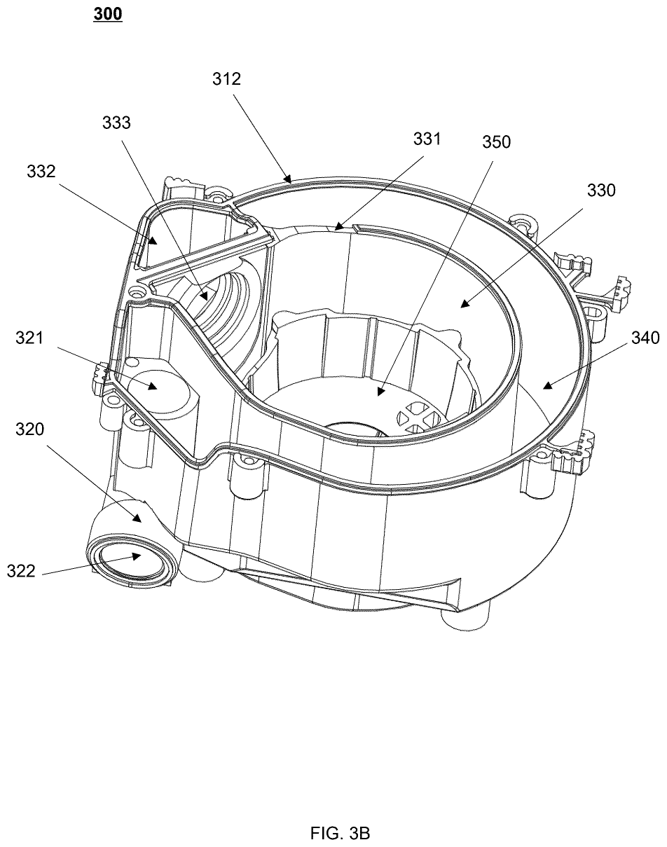

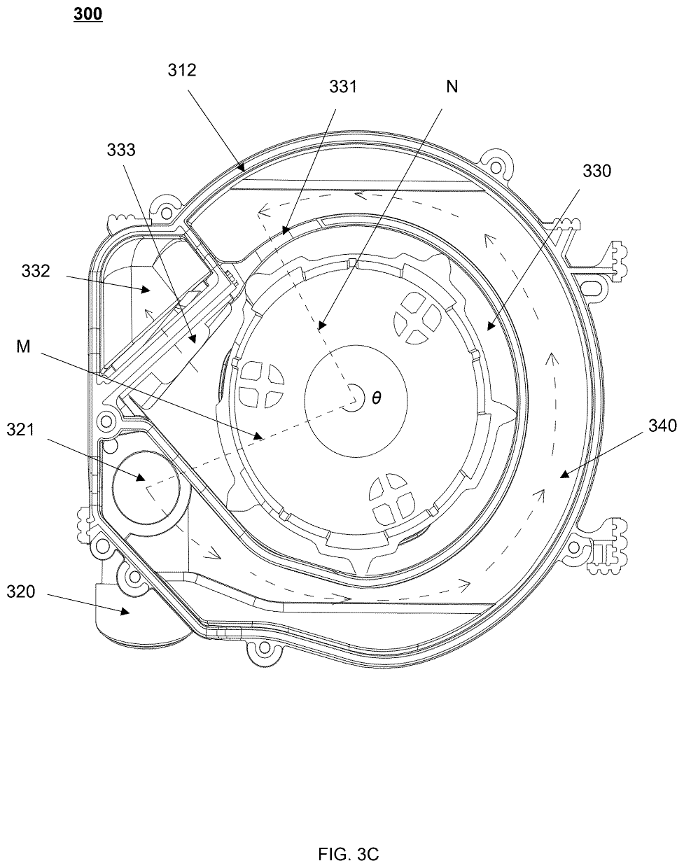

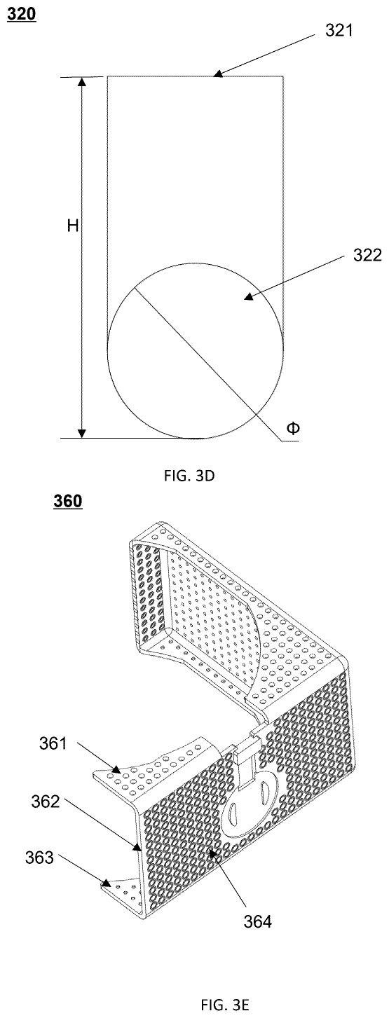

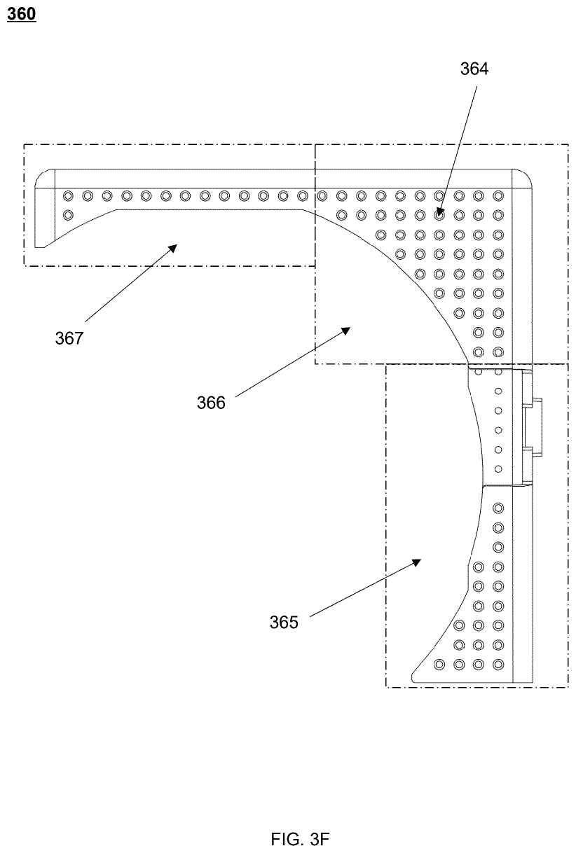

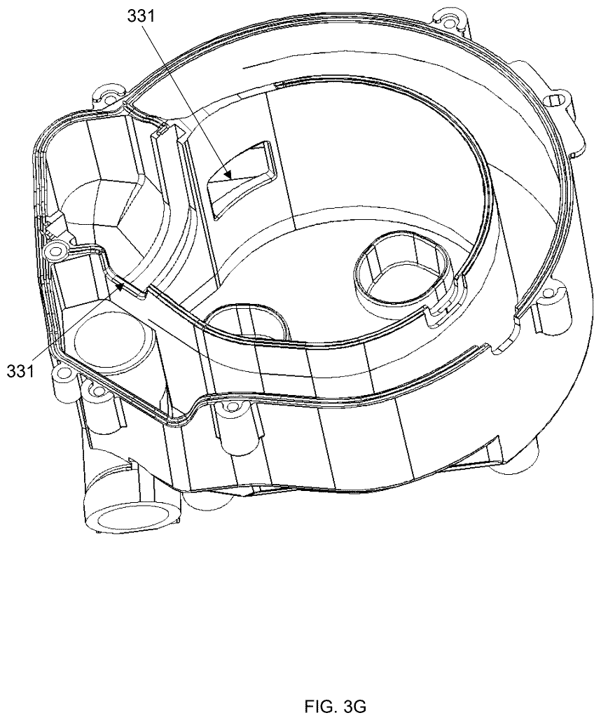

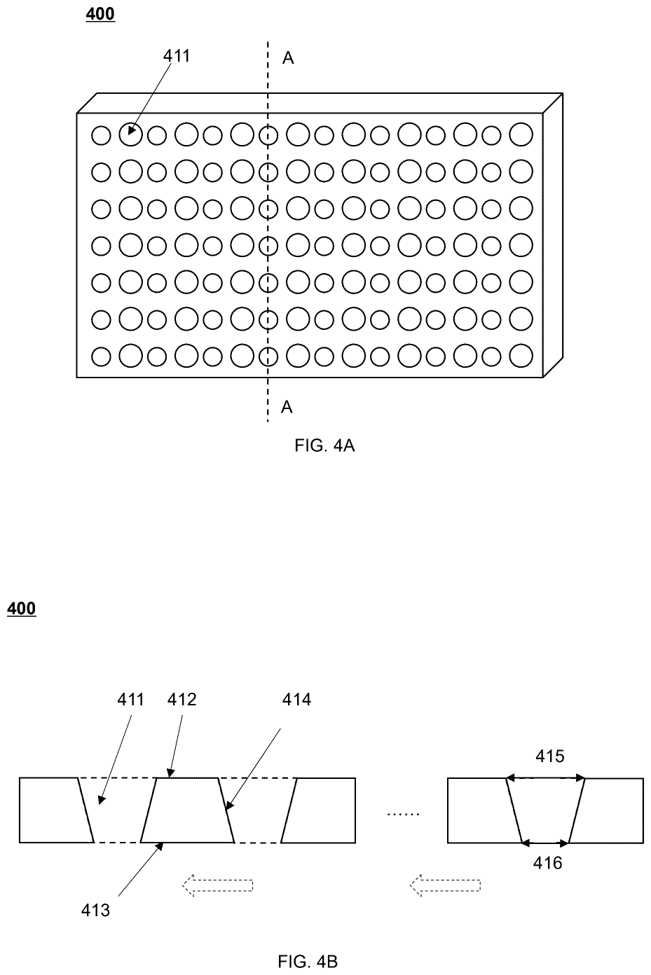

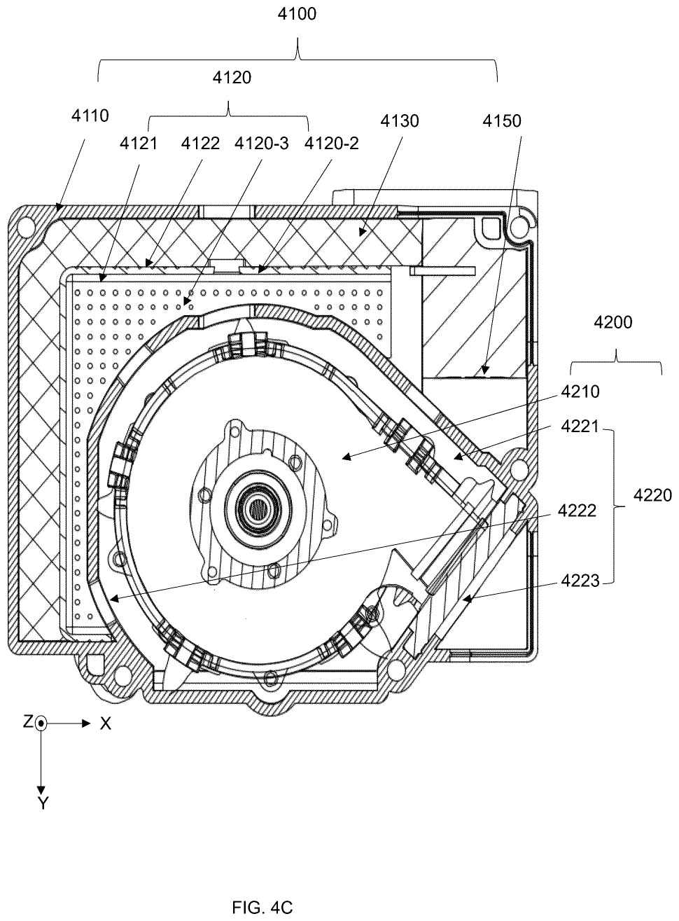

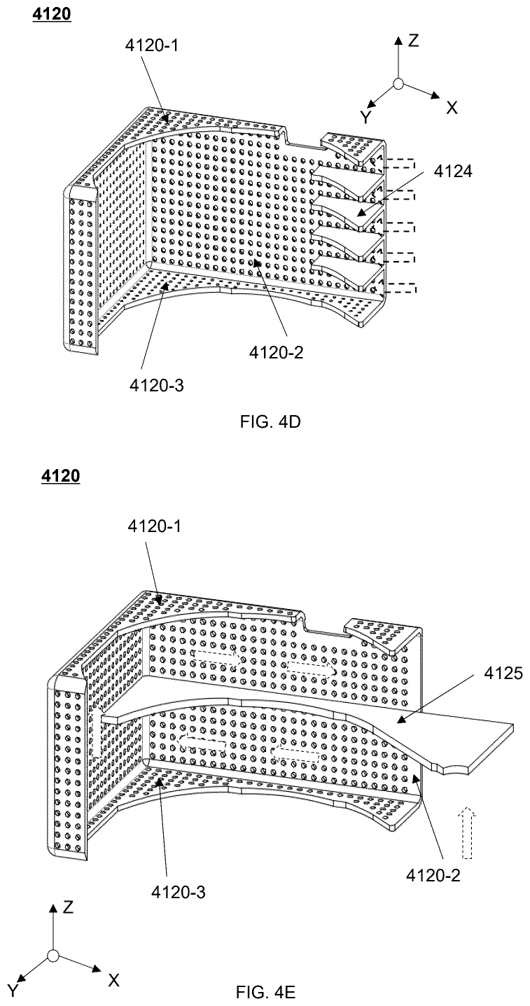

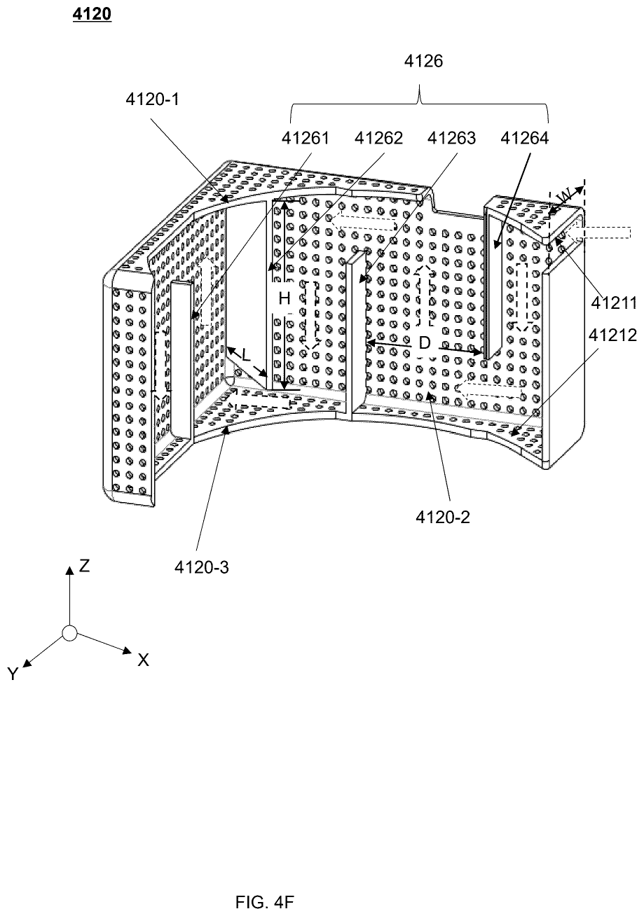

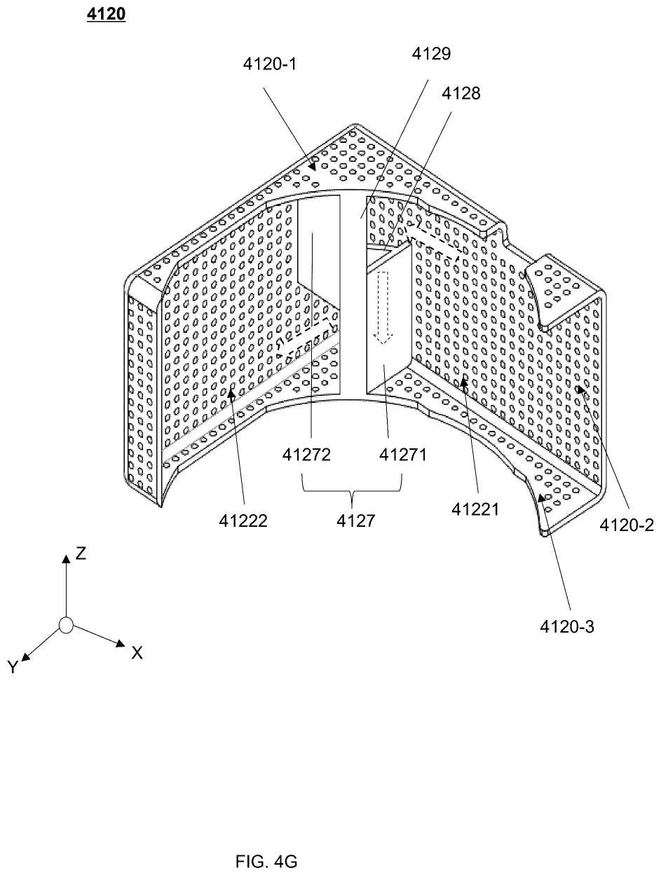

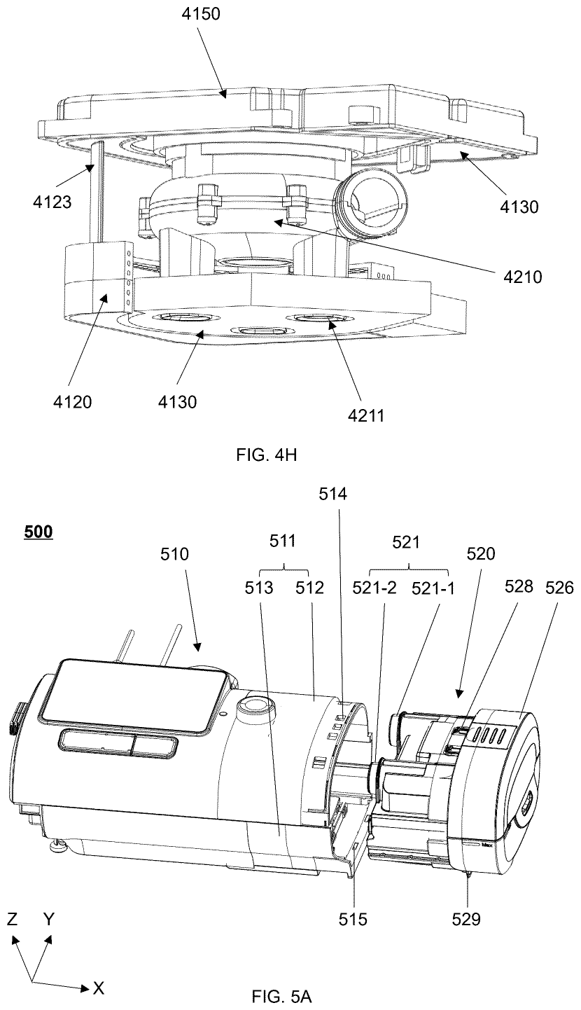

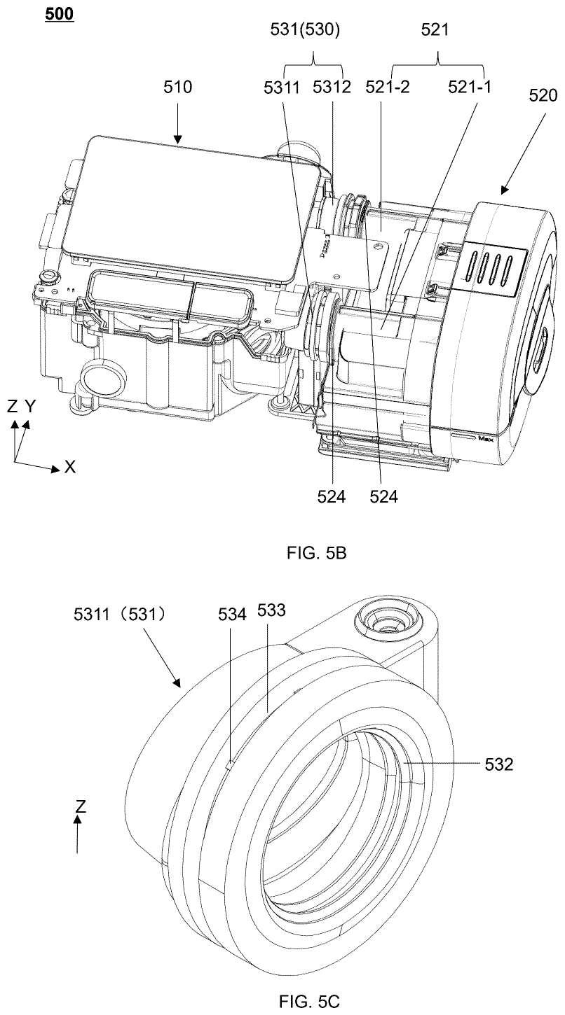

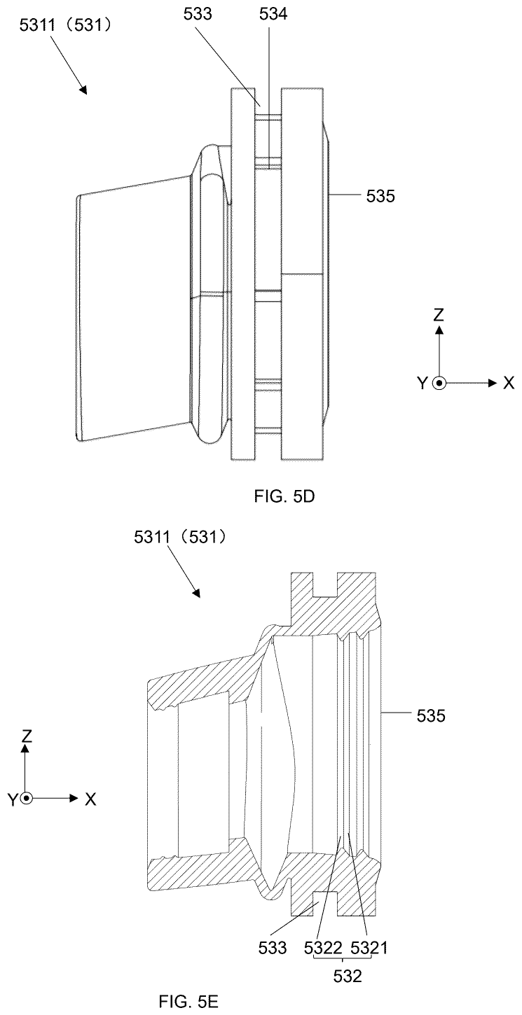

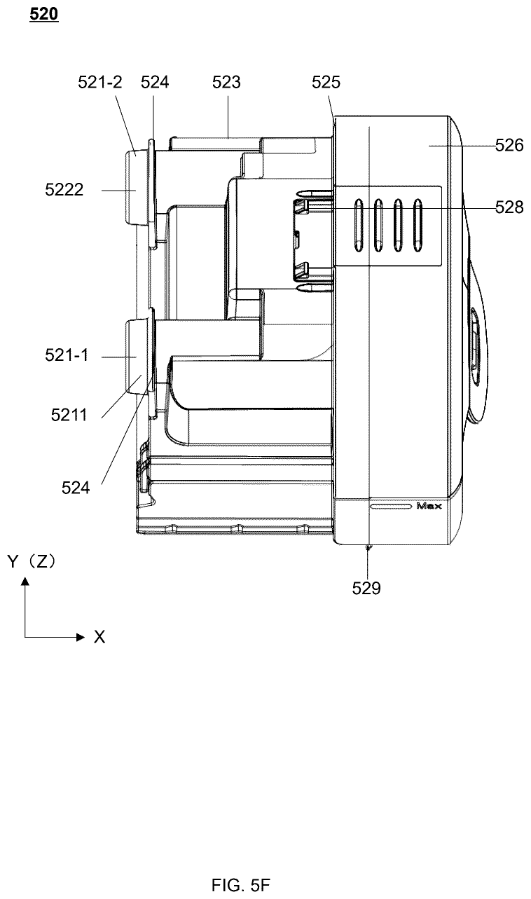

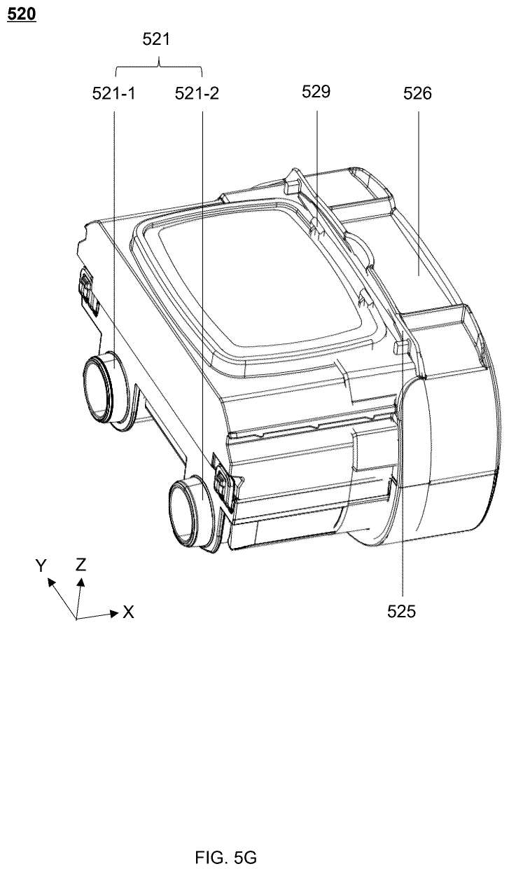

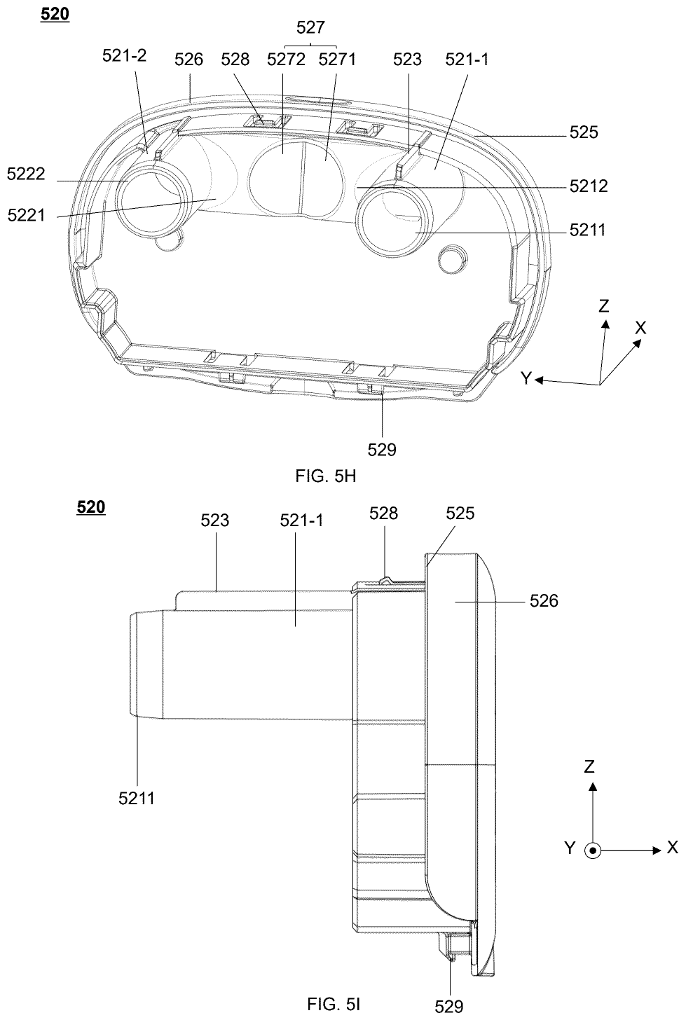

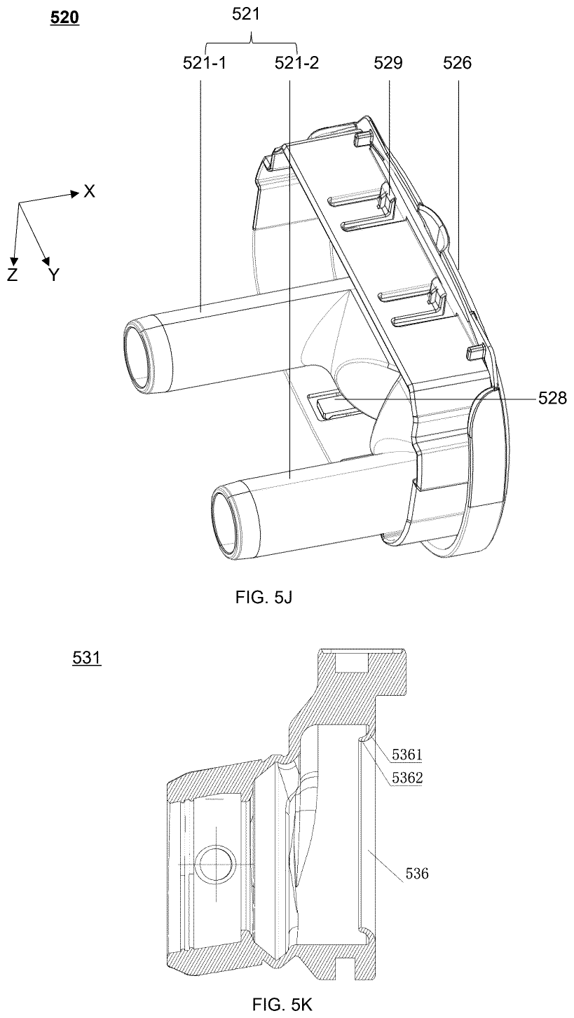

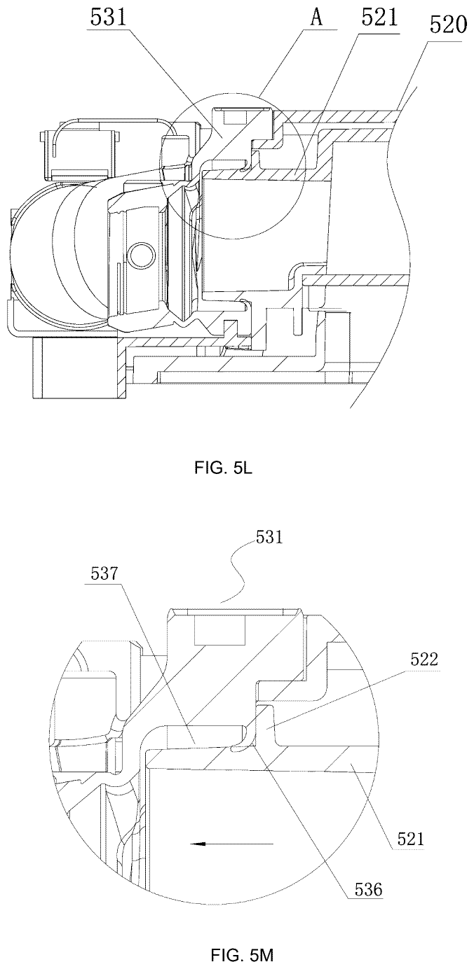

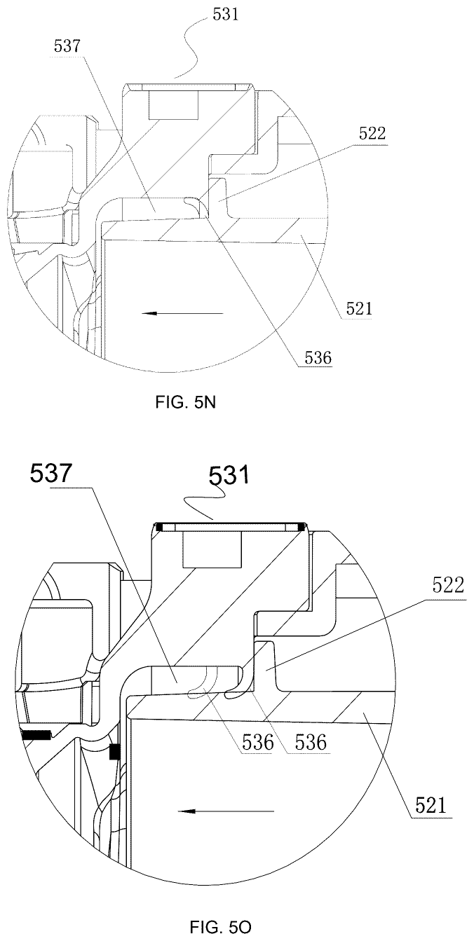

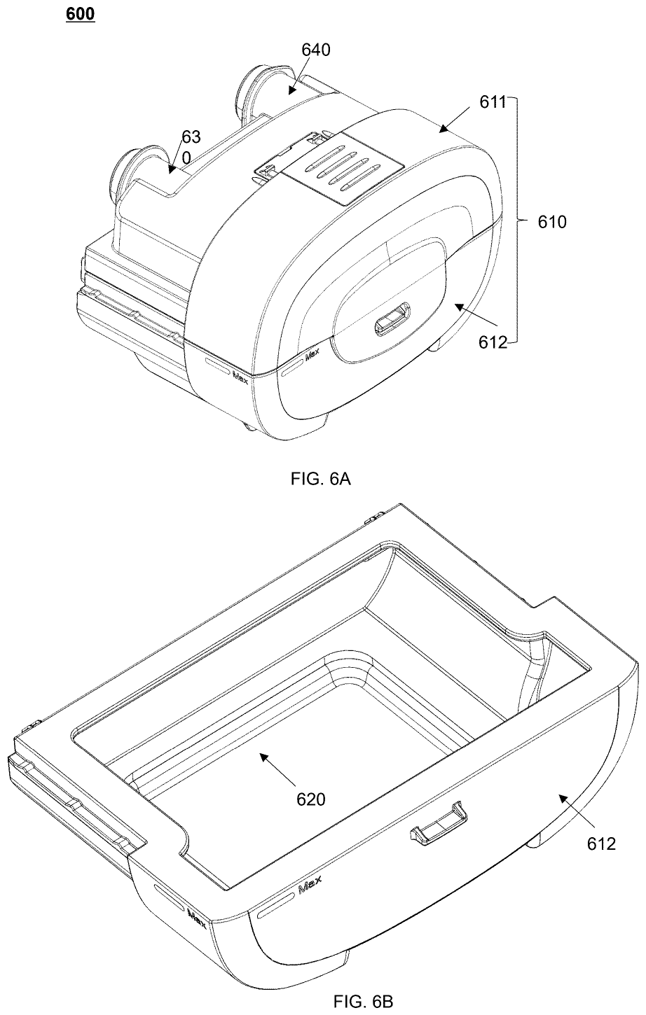

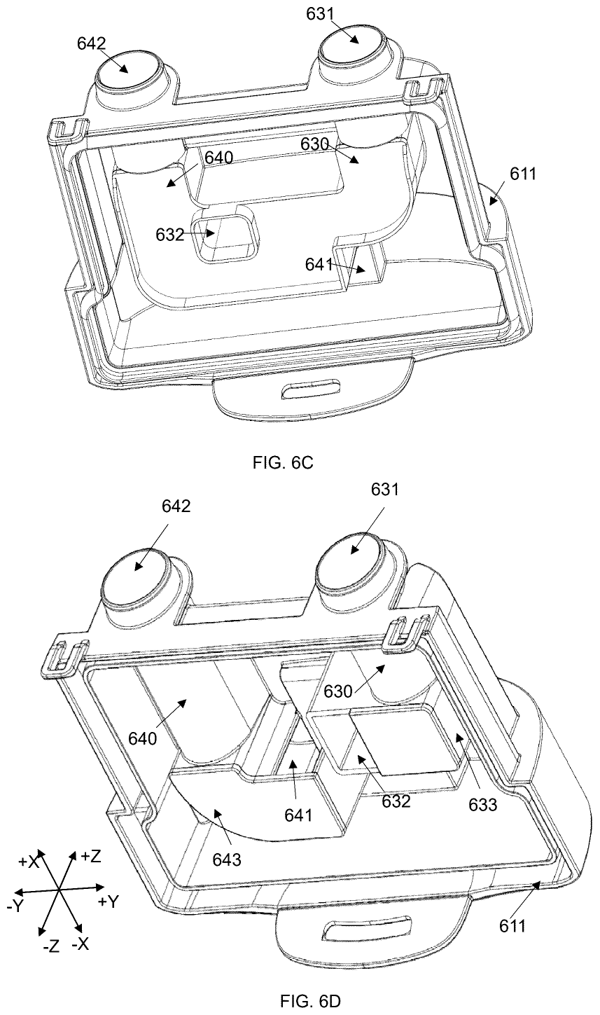

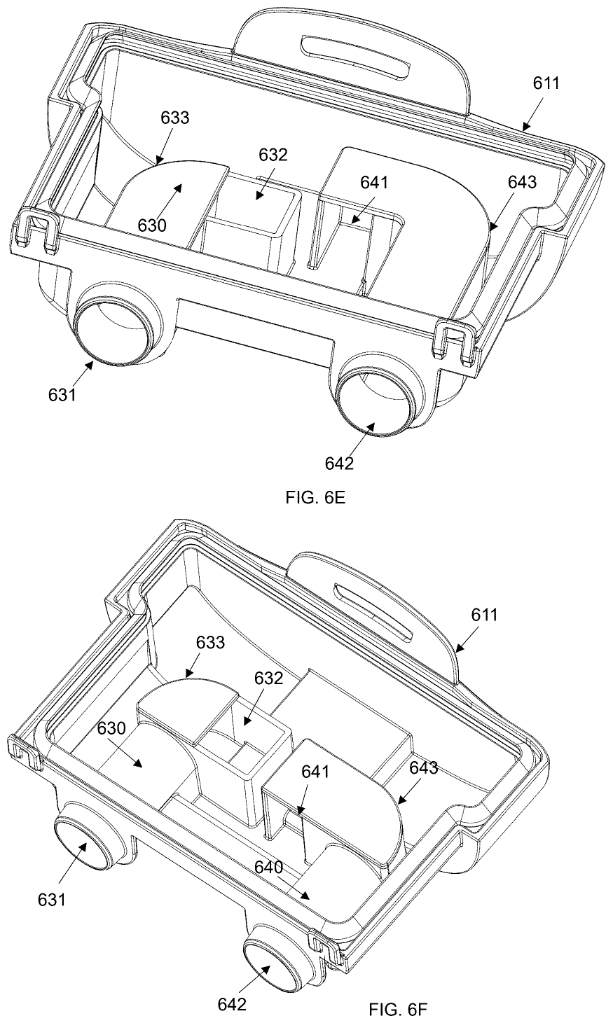

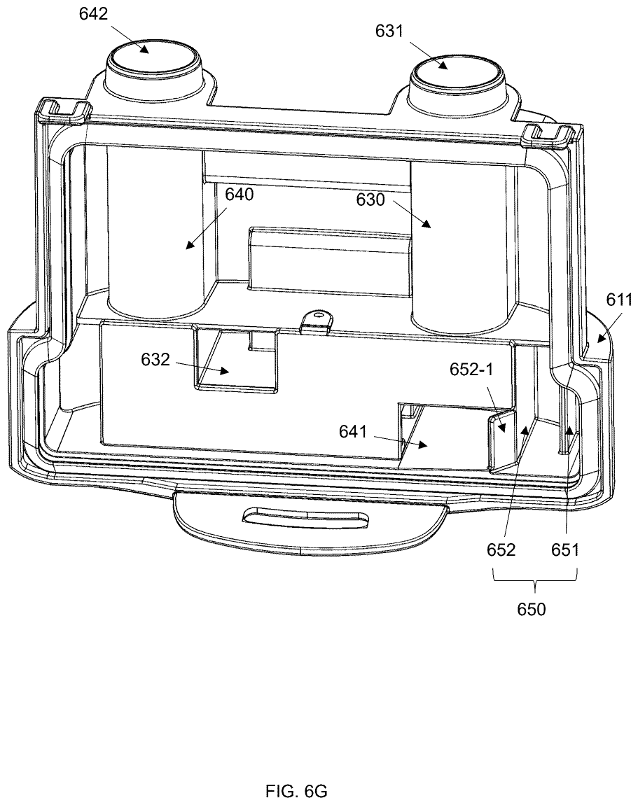

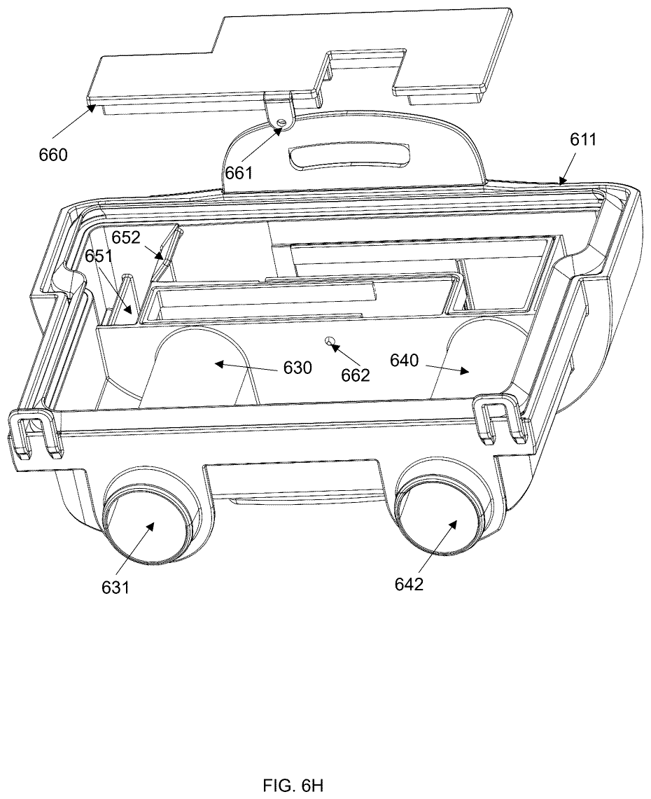

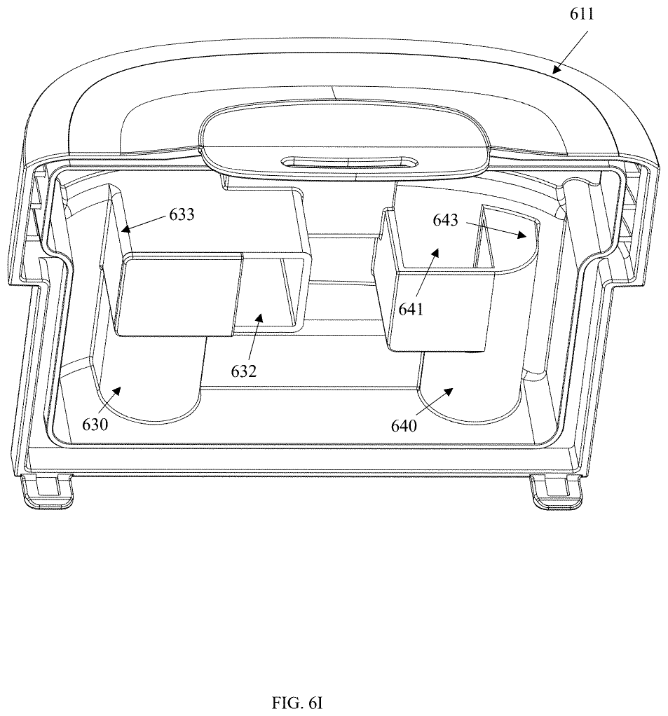

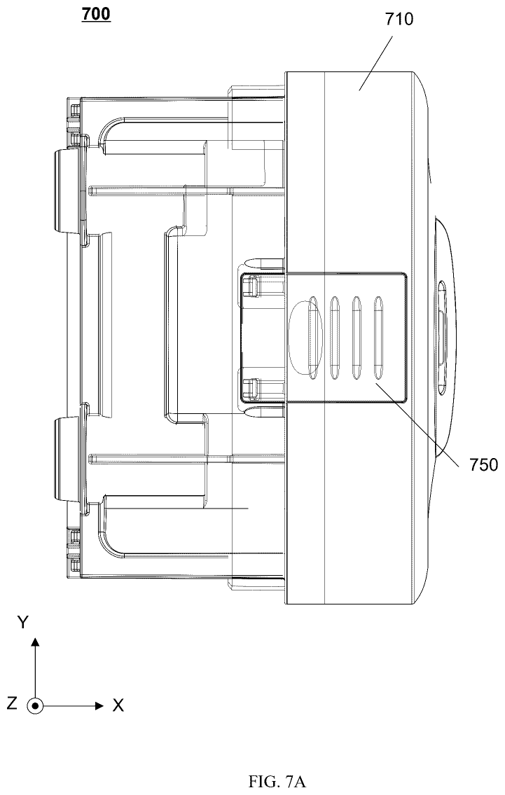

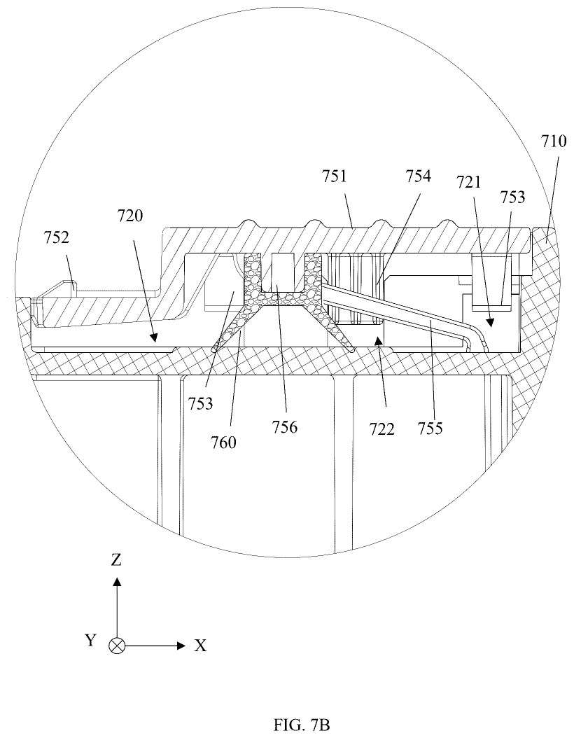

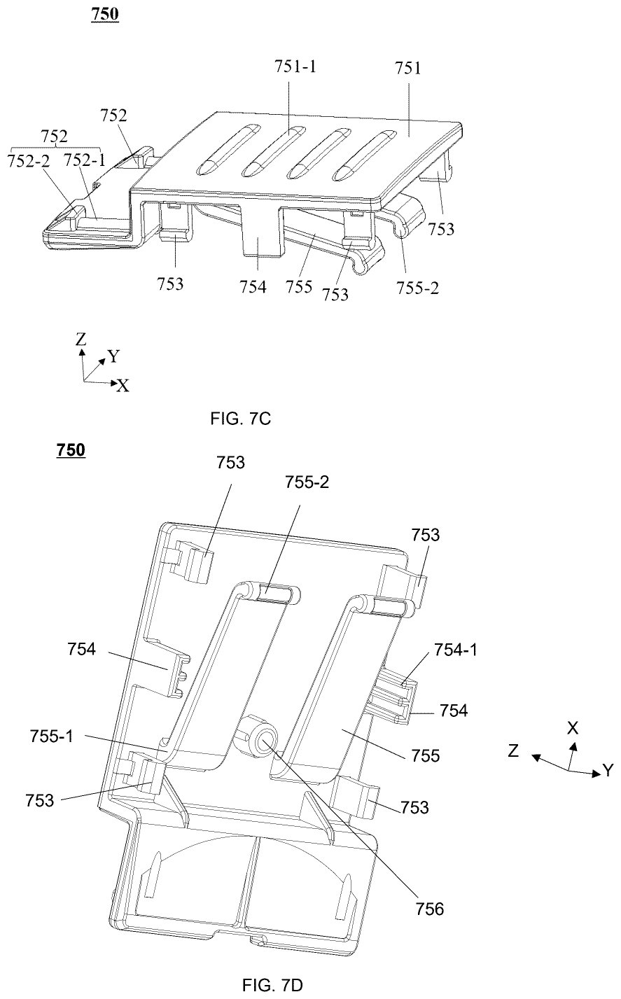

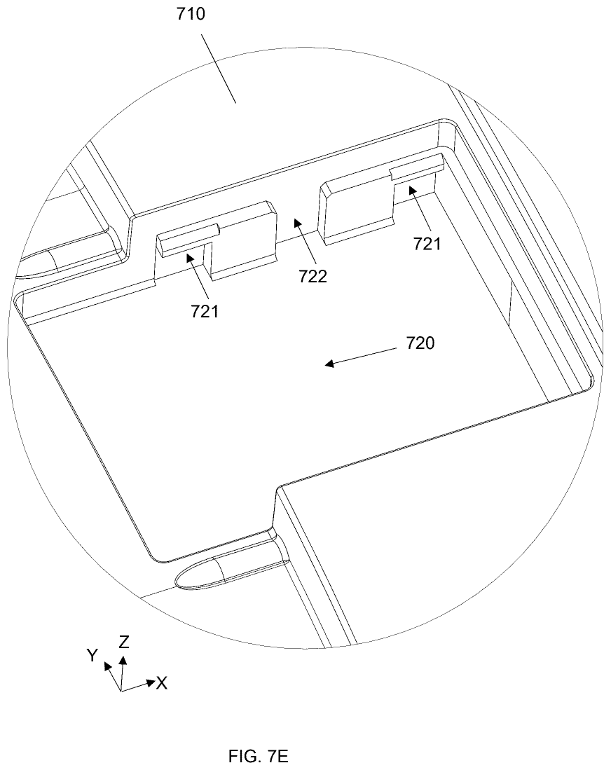

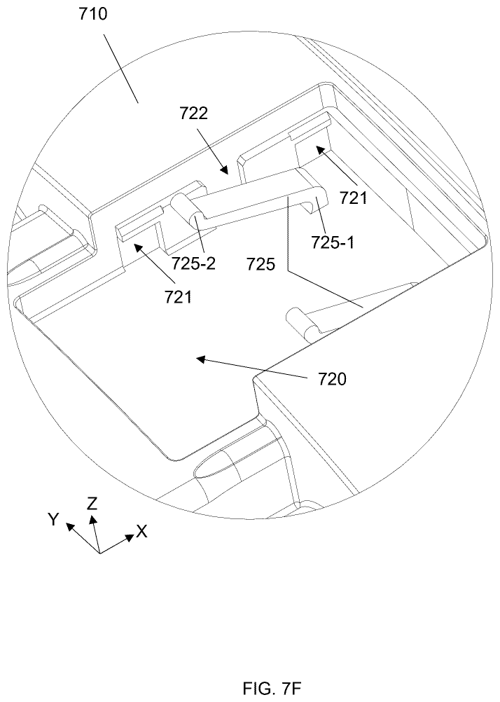

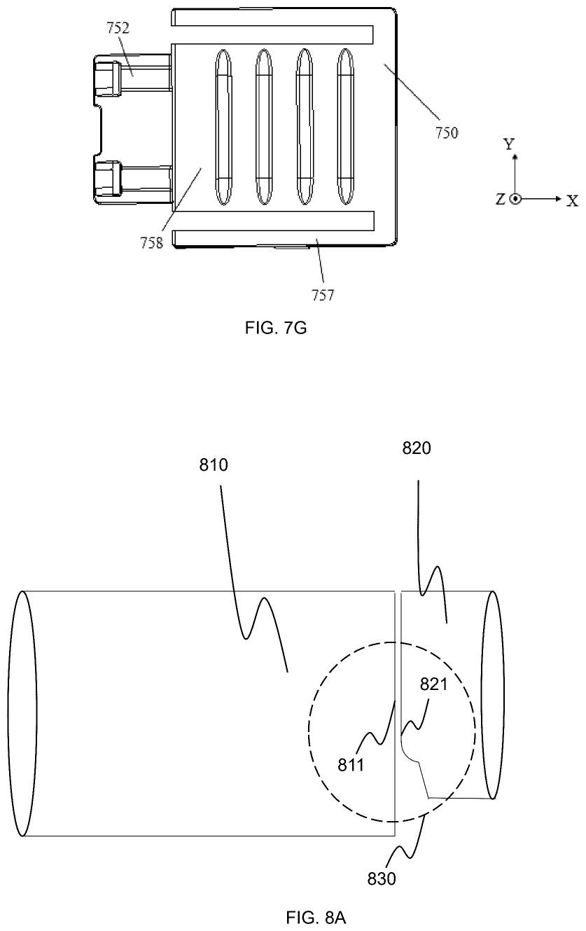

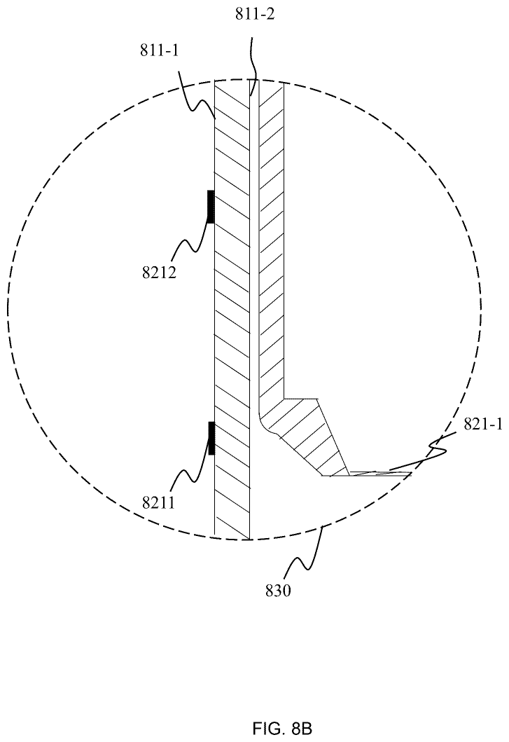

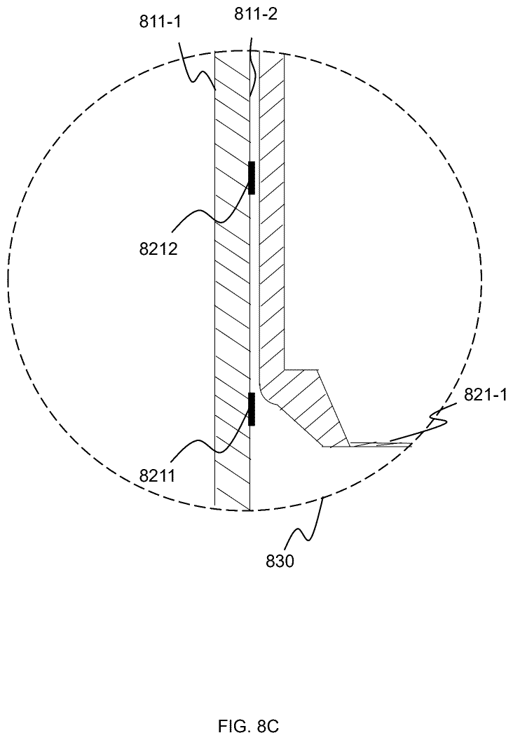

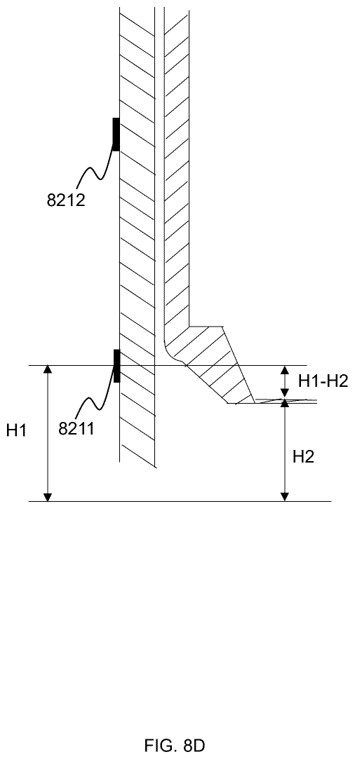

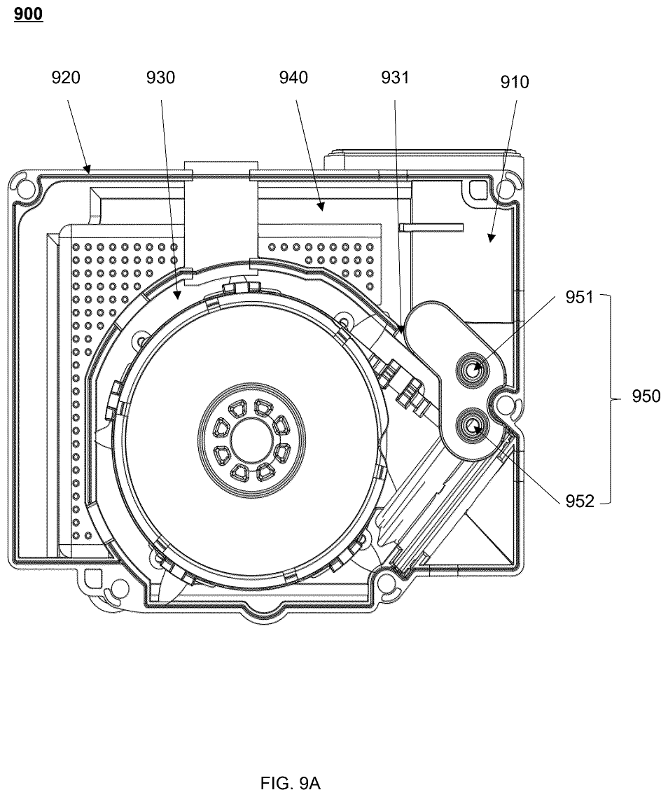

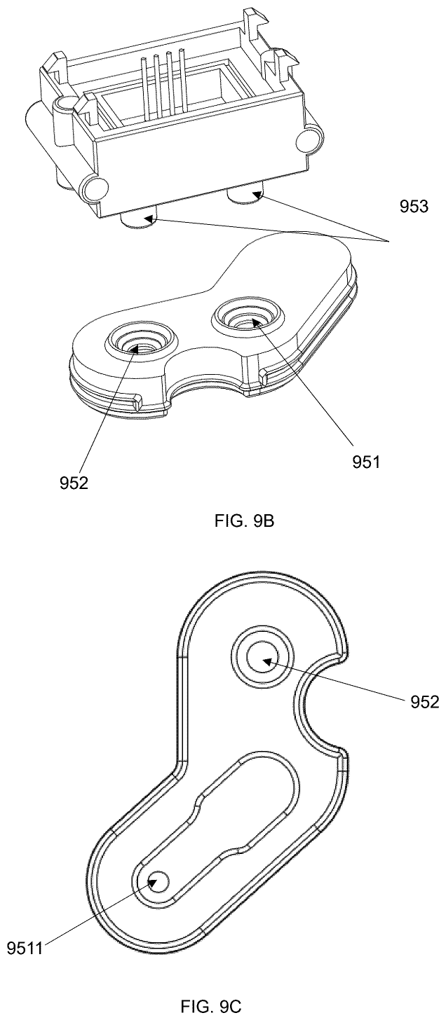

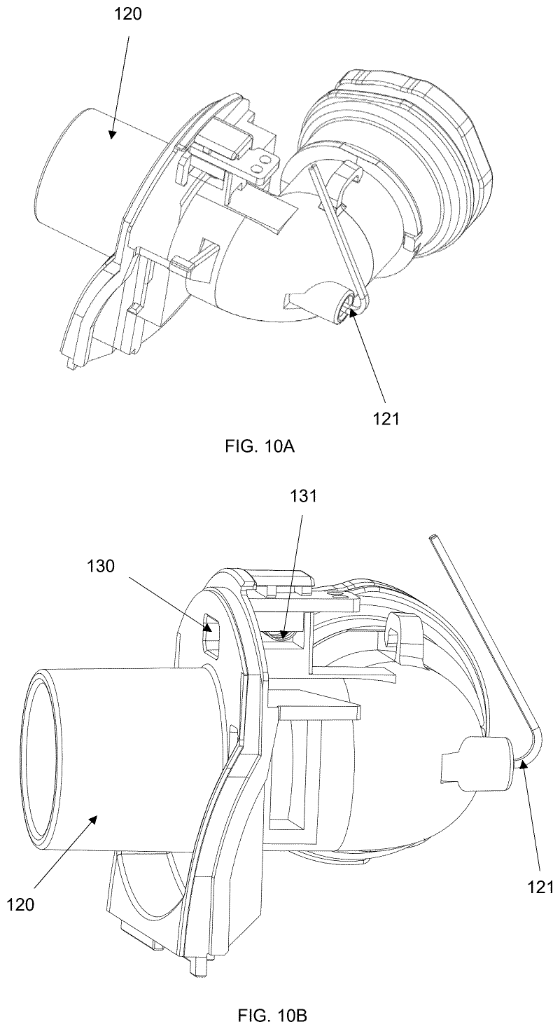

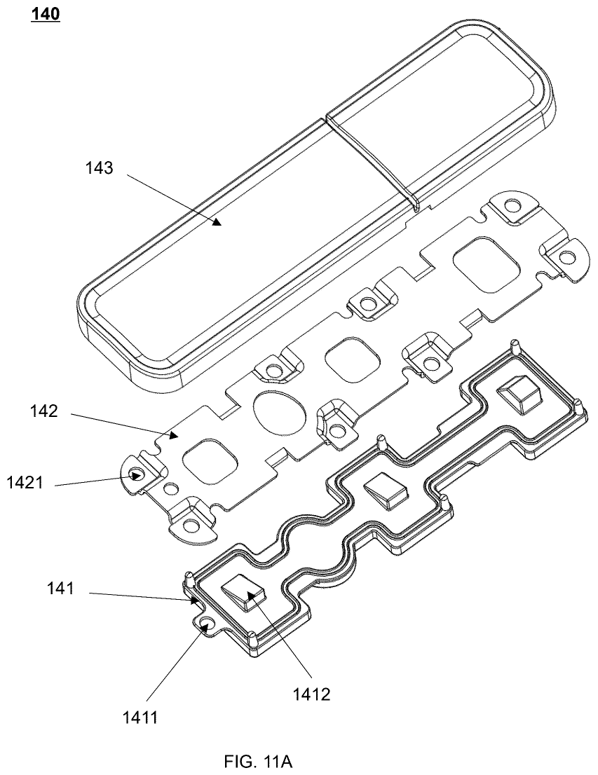

FIG. 1J is a dissembled schematic diagram of specific structures of the reservoir and the heating device of the respiratory ventilation apparatus according to some embodiments of the present disclosure;

FIG. 1K is a schematic diagram illustrating a structure of an elastic supporting structure of the heating device according to some embodiments of the present disclosure;

FIG. 2A is a 3D schematic diagram illustrating a structure of a noise reduction device (without a noise reduction top housing) according to some embodiments of the present disclosure;

FIG. 2B is a schematic top view illustrating a structure of a noise reduction device (without a noise reduction top housing) according to some embodiments of the present disclosure;

FIG. 2C is a schematic top view illustrating a structure of a noise reduction device (without a noise reduction top housing) according to some embodiments of the present disclosure;

FIG. 2D is a schematic top view illustrating a structure of a noise reduction device (without a noise reduction top housing) according to some other embodiments of the present disclosure;

FIG. 3A is a 3D schematic diagram illustrating a structure of a noise reduction device according to some other embodiments of the present disclosure;

FIG. 3B is a 3D schematic diagram illustrating a structure of a noise reduction device (without a noise reduction top housing) according to some other embodiments of the present disclosure;

FIG. 3C is a schematic top view illustrating a structure of a noise reduction device (without a noise reduction top housing) according to some other embodiments of the present disclosure;

FIG. 3D is a schematic front view illustrating a structure of an inner cavity of a gas inlet pipe of a noise reduction device according to some other embodiments of the present disclosure;

FIG. 3E is a schematic diagram illustrating a 3D structure of a porous sound-absorbing plate in a noise reduction device according to some other embodiments of the present disclosure;

FIG. 3F is a schematic diagram illustrating a top-view structure of a porous sound-absorbing plate in a noise reduction device according to some other embodiments of the present disclosure;

FIG. 3G is a 3D schematic diagram illustrating a structure of a noise reduction device (without a noise reduction top housing) according to some other embodiments of the present disclosure;

FIG. 4A is a schematic diagram illustrating a structure of a porous sound-absorbing plate according to some embodiments of the present disclosure;

FIG. 4B is a sectional view illustrating the porous sound-absorbing plate of FIG. 4A along axis AA;

FIG. 4C is a sectional view of a noise reduction structure according to some embodiments of the present disclosure;

FIG. 4D is a schematic diagram illustrating a structure of a first separating component of a porous sound-absorbing plate according to some embodiments of the present disclosure;

FIG. 4E is a schematic diagram illustrating a structure of a second separating component of a porous sound-absorbing plate according to some embodiments of the present disclosure;

FIG. 4F is a schematic diagram illustrating a structure of a third separating component of a porous sound-absorbing plate according to some embodiments of the present disclosure;

FIG. 4G is a schematic diagram illustrating a structure of a fourth separating component and a fifth separating component of a porous sound-absorbing plate according to some embodiments of the present disclosure;

FIG. 4H is a schematic diagram illustrating the noise reduction device (without a noise reduction bottom housing) according to some embodiments of the present disclosure;

FIG. 5A is a schematic diagram illustrating a structure of a main body of a respiratory ventilation apparatus (also referred to as a main body) according to some embodiments of the present disclosure when the main body of the respiratory ventilation apparatus is not connected to a connecting device;

FIG. 5B is a schematic structural diagram illustrating a main body of a respiratory ventilation apparatus when the main body of the respiratory ventilation apparatus is connected to a connecting device by a sealing structure according to some embodiments according to the present disclosure;

FIG. 5C is a schematic diagram illustrating a structure of a first elastic pipe according to some embodiments of the present disclosure;

FIG. 5D is a schematic diagram illustrating the first elastic pipe of FIG. 5C from another angle;

FIG. 5E is a schematic diagram illustrating a section of the first elastic pipe shown in FIG. 5C;

FIG. 5F is a schematic diagram illustrating a structure of a reservoir according to some embodiments of the present disclosure;

FIG. 5G is a schematic diagram illustrating the reservoir of FIG. 5F from another angle;

FIG. 5H is a schematic diagram of a structure of a cover plate without a reservoir according to some embodiments of the present disclosure;

FIG. 51 is a schematic diagram illustrating a structure of a cover plate without a reservoir of FIG. 5H from another angle;

FIG. 51 is a schematic diagram illustrating a structure of a cover plate without a reservoir of FIG. 5H from another angle;

FIG. 5K is a schematic diagram illustrating a structure of disposing an elastic sealing edge at a connection position of a connection between a main body and a connecting device of the respiratory ventilation apparatus through a sealing structure according to some embodiments of the present disclosure;

FIG. 5L is a schematic diagram illustrating a structure of disposing an elastic sealing edge at an elastic pipe when the elastic pipe and a breather pipe of a connecting device are connected according to some embodiments of the present disclosure;

FIG. 5M is a schematic diagram illustrating an enlarged structure of A in FIG. 5L according to some embodiments of the present disclosure;

FIG. 5N is a schematic diagram illustrating a structure of disposing an elastic sealing edge at a breather pipe when an elastic pipe and the breather pipe of a connecting device are connected according to some embodiments of the present disclosure;

FIG. 5O is a schematic diagram illustrating a structure of disposing a plurality of elastic sealing edges on the elastic pipe on the basis of FIG. 5M according to some embodiments of the present disclosure;

FIG. 6A is a schematic diagram illustrating a structure of a reservoir according to some embodiments of the present disclosure;

FIG. 6B is a schematic diagram illustrating a structure of a lower housing of a reservoir according to some embodiments of the present disclosure;

FIG. 6C is a schematic diagram illustrating a structure of an upper housing of a reservoir according to some embodiments of the present disclosure;

FIG. 6D is a schematic diagram illustrating a structure of another upper housing of a reservoir according to some embodiments of the present disclosure;

FIG. 6E is a schematic diagram illustrating a structure of another reservoir upper housing according to some embodiments of the present disclosure;

FIG. 6F is a schematic diagram illustrating a structure of another upper housing of another reservoir according to some embodiments of the present disclosure;

FIG. 6G is a schematic diagram illustrating a structure of another upper housing of a reservoir according to some embodiments of the present disclosure;

FIG. 6H is a schematic diagram illustrating a structure of another upper housing of a reservoir according to some embodiments of the present disclosure;

FIG. 61 is a schematic diagram illustrating a structure of another upper housing of a reservoir according to some embodiments of the present disclosure;

FIG. 7A is a schematic diagram illustrating an exemplary appearance of a reservoir according to some other embodiments of the present disclosure;

FIG. 7B is a schematic diagram illustrating an exemplary mating structure of a press button and a recessed structure according to some embodiments of the present disclosure;

FIG. 7C is a schematic diagram illustrating an exemplary structure of a press button according to some embodiments of the present disclosure;

FIG. 7D is a schematic diagram illustrating an exemplary structure of a press button from another perspective according to some embodiments of the present disclosure;

FIG. 7E is a schematic diagram illustrating an exemplary structure of a recessed structure according to some embodiments of the present disclosure;

FIG. 7F is a schematic diagram illustrating another exemplary recessed structure according to some embodiments of the present disclosure;

FIG. 7G is a schematic diagram illustrating another exemplary structure of a press button according to some embodiments of the present disclosure;

FIG. 8A is a schematic diagram illustrating an exemplary main body and humidifier according to some embodiments of the present disclosure;

FIG. 8B is a schematic diagram illustrating an exemplary liquid level detection device according to some embodiments of the present disclosure;

FIG. 8C is a schematic diagram illustrating an exemplary level detection device according to some other embodiments of the present disclosure;

FIG. 8D is a schematic diagram illustrating an exemplary height difference according to some embodiments of the present disclosure;

FIG. 9A is a schematic diagram illustrating a structure of a noise reduction device disposed with a flow detection device according to some embodiments of the present disclosure;

FIG. 9B is a schematic diagram illustrating an exemplary structure of a flow detection device according to some embodiments of the present disclosure;

FIG. 9C is a schematic diagram illustrating an exemplary structure of a flow detection device according to some embodiments of the present disclosure;

FIG. 10A is a schematic diagram illustrating an exemplary structure of a pipe structure of a respiratory ventilation apparatus according to some embodiments of the present disclosure;

FIG. 10B is a schematic diagram illustrating an exemplary structure of a pipe structure of a respiratory ventilation apparatus from another angle according to some embodiments of the present disclosure;

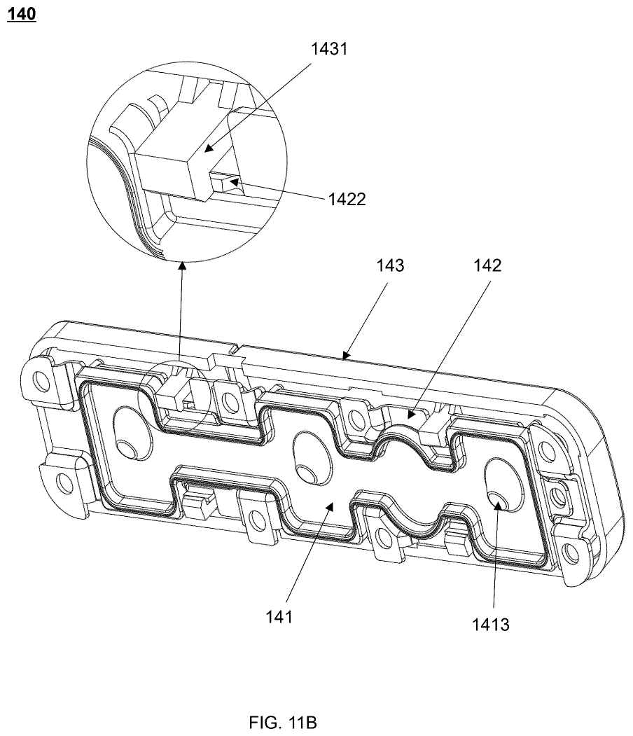

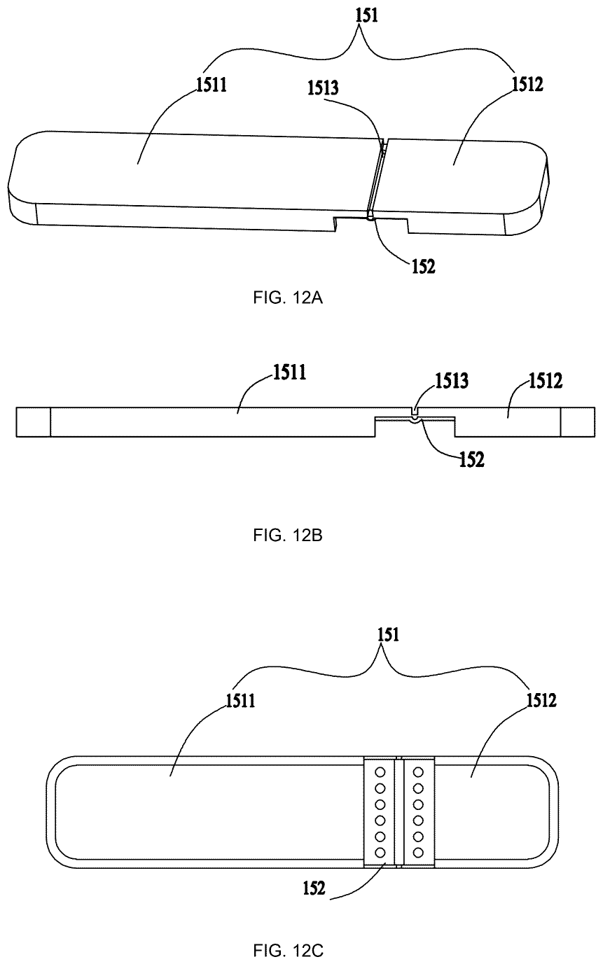

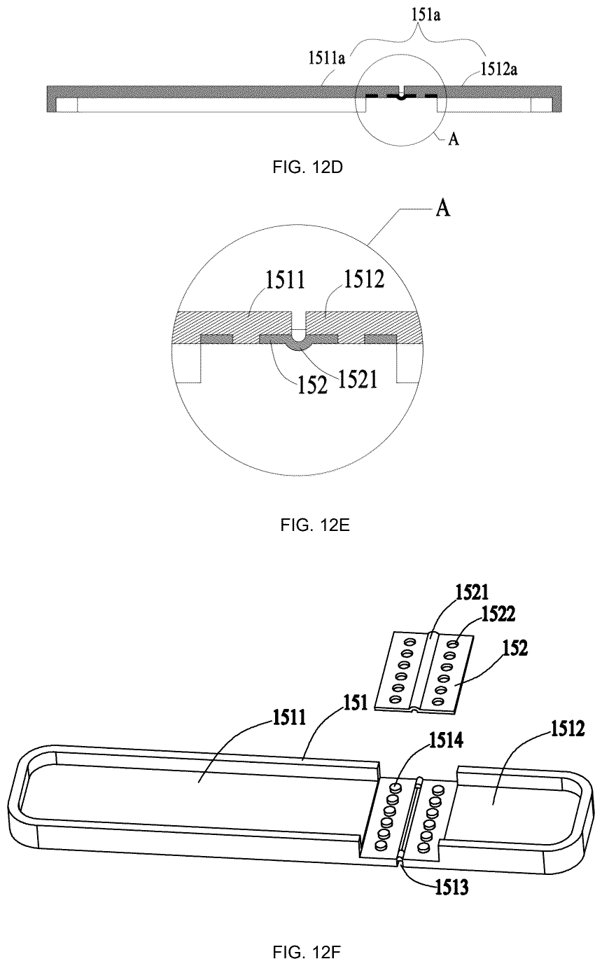

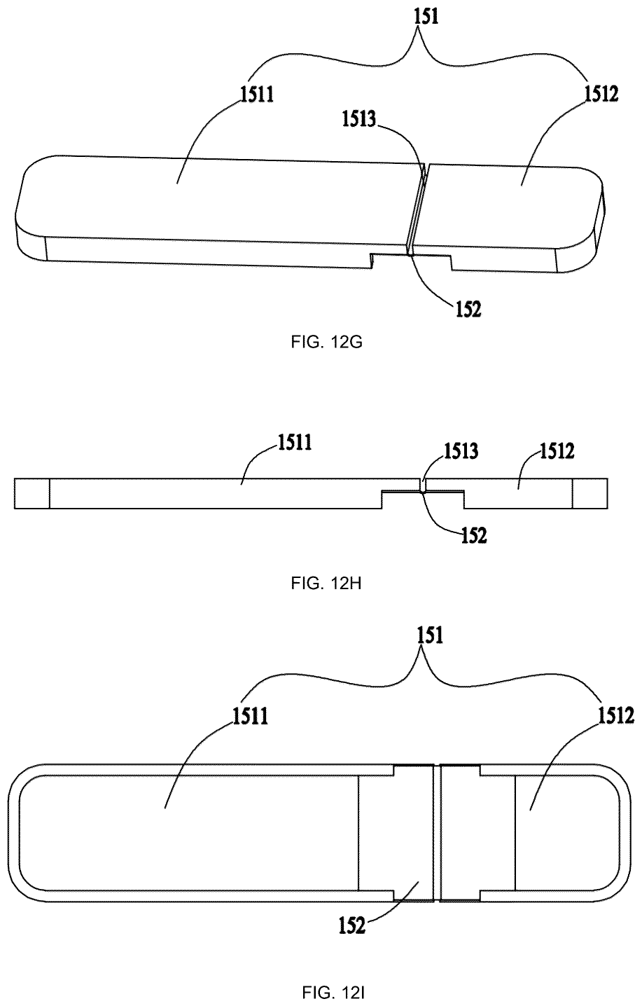

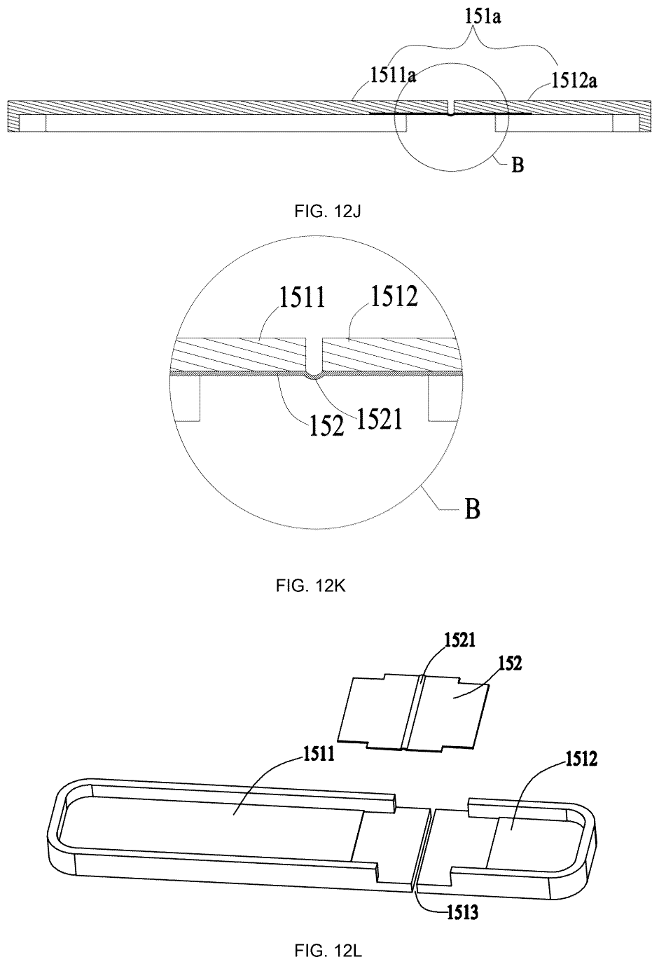

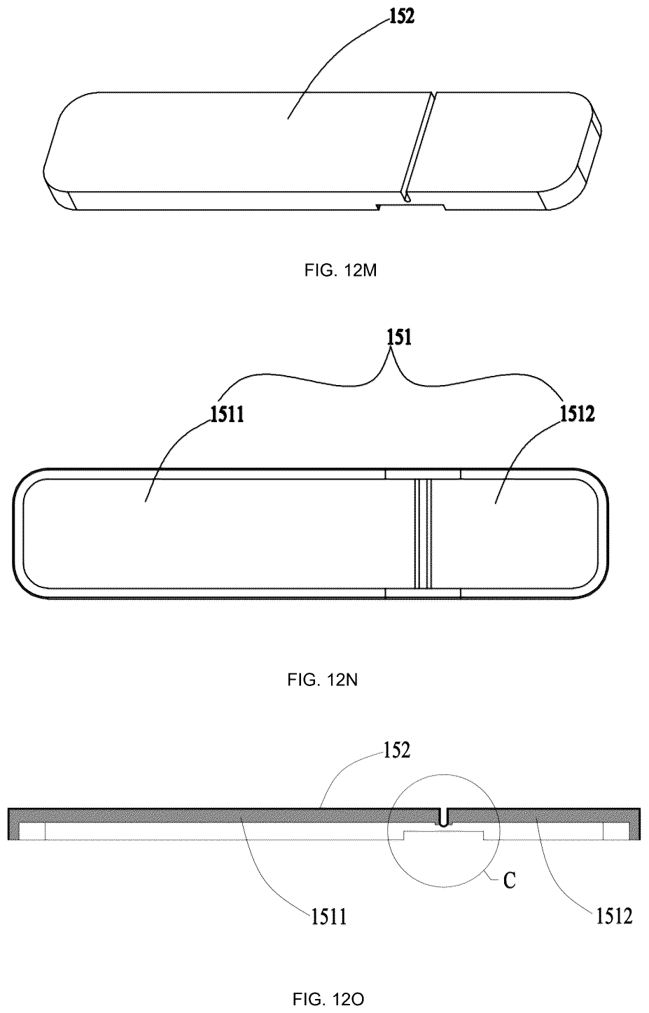

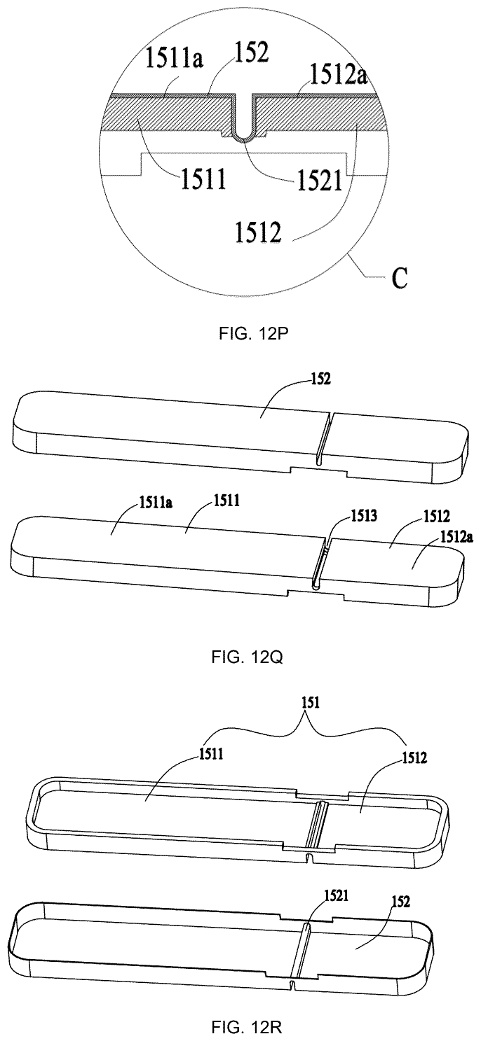

FIG. 11A is a schematic diagram illustrating an exemplary exploded structure of a main body button according to some embodiments of the present disclosure;

FIG. 11B is a schematic diagram illustrating an exemplary structure of a main body case according to some embodiments of the present disclosure;

FIG. 12A is a 3D structural view of a button according to some embodiments of the present disclosure;

FIG. 12B is a main view of the button in FIG. 12A;

FIG. 12C is a bottom view of the button in FIG. 12A;

FIG. 12D is a sectional view of the button in FIG. 12A;

FIG. 12E is an enlarged view of localization A in FIG. 12D;

FIG. 12F is an exploded view of the button in FIG. 12A;

FIG. 12G is a 3D structural view of a button according to some other embodiments of the present disclosure;

FIG. 12H is a main view of the button in FIG. 12G;

FIG. 121 is a bottom view of the button in FIG. 12G;

FIG. 12J is a sectional view of the button in FIG. 12G;

FIG. 12K is an enlarged view of localization B in FIG. 12J;

FIG. 12L is an exploded view of the button in FIG. 12G;

FIG. 12M is a 3D structural view of a button according to some other embodiments of the present disclosure;

FIG. 12N is a main view of the button in FIG. 12M;

FIG. 12O is a bottom view of the button in FIG. 12M;

FIG. 12P is a sectional view of the button in FIG. 12M;

FIG. 12Q is an enlarged view of localization C in FIG. 12P; and

FIG. 12R is an exploded view of the button in FIG. 12P from another perspective.

In the figures: 1000, respiratory ventilation apparatus; 2000, network; 3000, terminal; 4000, processing device; 5000, storage device; 6000, respiration pipe; 7000, user interface; 8000, user.

1100, main body; 110, display screen; 1200, reservoir; 1210, heating device; 1220, elastic seal; 1211, metal heating plate; 1212, spacer; 1212-1, elastic body; 1212-2, hollow support column; 1213, heating bottom plate; 120, pipeline interface; 121, temperature detection device; 130, power supply interface; 131, metal elastic sheet; 140, main body button; 141, silicone layer; 1411, positioning hole; 1412, elastic button; 1413, button patch; 142, metal bracket; 1421, hot melt hole; 1422, protruding end; 143, button panel; 1431, buckle hook.

200, noise reduction device; 210, gas inlet pipe; 211, separating component; 212, gas inlet sub-pipe; 213, gas outlet; 220, noise reduction housing; 230, blower cavity; 231, first gas inlet; 232, second gas inlet; 233, third gas inlet; 234, blower; 235, blower hoisting suspension structure; 235-1, flexible blower cover; 235-2, hoisting column; 235-21, flexible buckle head; 235-3, semi-enclosed structure blower cover; 235-4, support column; 235-5, semi-enclosed structure blower cover; 235-6, side mounting column; 235-61, connection column; 235-62, clamping joint; 236, noise reduction top housing; 236-1, mounting hole; 240, gas channel; 241, first space; 242, second space; 243, third space; 244, fourth space; 245, fifth space; 250, porous acoustic plate.

300, noise reduction device; 310, noise reduction housing; 311, noise reduction top housing; 312, noise reduction bottom housing; 313, main outlet; 320, gas inlet pipe; 321, gas outlet; 322, gas inlet; 330, blower cavity; 331, cavity gas inlet; 332, water collection cavity; 333, cavity gas outlet; 340, gas channel; 350, blower mounting cavity; 360, porous sound-absorbing plate; 361, porous top plate; 362, porous side plate; 363, porous bottom plate; 364, sound-absorbing hole; 365, first region; 366, second region; 367, third region.

400, porous sound-absorbing plate; 411, through hole; 412, second surface; 413, first surface; 414, sidewall of the through hole; 415, second opening; 416, first opening.

4100, noise reduction structure; 4110, noise reduction housing; 4120, porous sound-absorbing plate; 4120-2, second porous sound-absorbing plate; 4120-3, third porous sound-absorbing plate; 4130, sound-absorbing cotton; 4140, noise reduction top housing; 4150, gas inlet; 4200, blower assembly; 4210, blower body; 4211, blower support column; 4220, blower cavity; 4221, blower cavity housing; 4222, cavity gas inlet; 4223, cavity gas outlet.

4120-1, first porous sound-absorbing plate; 41221, first sub-plate; 41222, second sub-plate; 4121, first surface; 41211, upper surface; 41212, lower surface; 4122, second surface; 4123, top housing support column; 4124, first separating component; 4125, second separating component; 4126, third separating component group; 41261, first low third separating component; 41262, first high third separating component; 41263, second low third separating component; 41264, second high third separating component; 4127, fourth separating component group; 41271, first side separating component; 41272, second side separating component; 412722, second surface of the second side separating component; 4128, fifth separating component; 4129, supporting plate.

500, respiratory ventilation apparatus; 510, main body; 511, main body housing; 512, housing top wall; 513, housing bottom wall; 514, first clamping slot; 515, second clamping slot; 520, connecting device; 521, breather pipe; 521-1, gas inlet channel; 5211, first gas inlet end; 5212, first gas outlet end; 521-2, gas outlet channel; 522, stopping portion; 5221, second gas inlet end; 5222, second gas outlet end; 523, guide rib; 524, first stopper; 525, second stopper; 526, top cover; 527, flow-limiting structure; 5271, first flow-limiting pipe; 5272, second flow-limiting pipe; 528, first buckle; 529, second buckle; 530, sealing structure; 531, elastic pipe; 5311, first elastic pipe; 5312, second elastic pipe; 532, annular protrusion; 5321, first sidewall; 5322, second sidewall; 533, limiting groove; 534, limiting protrusion; 535, insertion; 536, elastic sealing edge; 5361, middle section; 5362, tail section; 537, connection gap.

600, reservoir; 610, reservoir housing; 611, reservoir upper housing; 612, reservoir lower housing; 620, heat transfer component; 630, gas inlet channel; 631, reservoir first gas inlet; 632, reservoir first gas outlet; 633, first bending portion; 640, gas outlet channel; 641, reservoir second gas inlet; 642, reservoir second gas outlet; 643, second bending portion; 650, flow guide rib; 651, first flow guide rib; 652, second flow guide rib; 652-1, third bending portion; 660, sealing plate; 661, snap; and 662, protruding portion.

700, reservoir; 710, reservoir housing; 720, recessed structure; 721, limiting groove; 722, guiding groove; 725, second elastic arm; 725-1, second fixed end; 725-2, second free end; 750, pressing button; 751, pressing surface; 751-1, pressing stripe; 752, connection structure; 752-1, connection component; 752-2, buckle protrusion; 753, limiting component; 754, guiding member; 754-1, reinforced protrusion; 755, first elastic arm; 755-1, first fixed end; 755-2, first free end; 756, connection protrusion; 757, fixed structure; 758, elastic piece mechanism; and 760, elastic component.

810, main body of respiratory ventilation apparatus; 820, reservoir of humidifier; 830, portion of structure where the reservoir 820 of the humidifier and the main body 810 of the respiratory ventilation apparatus are connected; 811, sidewall of the main body; 821, sidewall of the humidifier; 811-1, side away from the sidewall of the humidifier; 811-2, side facing the sidewall of the humidifier; 821-1, bottom plate of the humidifier; 8211, first sensor; 8212, second sensor.

900, noise reduction device; 910, gas inlet pipe; 920, noise reduction housing; 930, blower cavity; 931, cavity gas inlet; 940, gas channel; 950, flow detection device; 951, low pressure detection point; 9511, gas hole at a lower end of the low pressure detection point 951 connected to the gas channel 940; 952, high pressure detection point; 953, flow sensor.

150, button; 151, button body; 151a, pressing side; 1511, first button function portion; 1511a, first pressing surface; 1512, second button function portion; 1512a, second pressing surface; 1513, narrow slot; 1514, protrusion; 152, button connection portion; 1521, arc extension; 1522, connection hole.

### DETAILED DESCRIPTION

Exemplary embodiments or implementations will be described herein in detail, examples of which are represented in the accompanying drawings. When the following description relates to the accompanying drawings, unless otherwise indicated, the same numeral in the drawings refers to the same structure or operation The embodiments described in the following exemplary embodiments are not intended to be representative of all embodiments consistent with the present disclosure. Rather, they are only examples of devices and methods that are consistent with some aspects of the present disclosure as detailed in the appended claims.

Terms used in the present disclosure are used solely for the purpose of describing particular embodiments and are not intended to limit the present disclosure. As used in the present disclosure and the appended claims, the singular forms of “a”, “an” and “the” as used in the present disclosure and the appended claims are also intended to include the plural form, unless the context clearly indicates otherwise.

It should be understood that the terms “first,” “second,”, etc., as used in the present disclosure and the claims, do not indicate any order or importance, but are used only to distinguish between different components. Similarly, the words “a” or “one,” etc. do not indicate a quantitative limitation, but rather the presence of at least one component. Unless otherwise noted, “front,” “rear,” “lower,” and/or “upper” and similar terms are used for illustrative purposes only and are not limited to a position or a spatial orientation. The words “include” or “comprise,” etc. mean that the elements or objects present before “include” or “comprise” covers the elements or objects that appear after “include” or “comprise” and the equivalents thereof, while does not exclude other elements or objects.

In modern clinical medicine, a respiratory ventilation apparatus, as an effective means of artificially replacing an autonomous ventilation function, may be a device that may replace, control, or change a normal physiological respiration of human beings, increase a pulmonary ventilation, improve a respiratory function, and reduce a respiratory work consumption. The respiratory ventilation apparatus has been commonly used in a respiratory failure due to various reasons, an anesthesia respiratory management during a major surgery, a respiratory support therapy and an emergency resuscitation, and may occupy a very important position in the field of modern medicine. The respiratory ventilation apparatus may be a medical device that plays a crucial role in preventing and treating the respiratory failure, reduces complications, and save and prolong patients' lives.

A household sleep respiratory ventilation apparatus may be mainly used in individual families, sleep centers and small clinics, and mainly for patients with an apnea syndrome, in the whole process of treatment, the patient may be in a sleeping state, which requires a surrounding environment to be quiet and appropriate. The respiratory ventilation apparatus relies on a blower to pressurize a gas in a pipeline to treat the patient. With an emphasis on the respiratory therapy, performance requirements of the respiratory ventilation apparatus may become increasingly higher. An easily perceived aspect of the performance of the respiratory may be a noise, and the noise may seriously affect the user experience. A main source of noise may be the noise generated by a gasflow. The noise generated by the gas entering from a cavity gas inlet of a blower cavity into the blower cavity may take up a majority of the noise generated. In addition, a vibration, and a rotation of the blower itself may also generate the noise. Therefore, it is necessary to dispose a noise reduction device to reduce the noise generated during the use of the respiratory ventilation apparatus.

It should be understood that the application scenario of the respiratory ventilation apparatus of the present disclosure are only some examples or embodiments of the present disclosure. For those skilled in the art, the present disclosure may be applied to other similar scenarios according to these drawings without creative labor.

The respiratory ventilation apparatus covered by the embodiments of the present disclosure will be described in detail below combining FIG. 1A-FIG. 12R. It should be noted that, the following embodiments are for the purpose of explaining the present disclosure only and do not constitute a limitation of the present disclosure.

FIG. 1A is a schematic diagram illustrating an exemplary system for a respiratory ventilation apparatus according to some embodiments of the present disclosure. A system 10 may be configured to provide a respiratory gas to a user. In some embodiments, the respiratory gas may include natural gas (or atmospheric gas), purified gas, oxygen, atmospheric gas enriched with oxygen, a therapeutic drug, pressurized gas (enriched with oxygen), humidified gas (enriched with oxygen), etc., or a combination thereof As illustrated, the system 10 may include a respiratory ventilation apparatus 1000, a respiration pipe 6000, and a user interface 7000. In some embodiments, the respiratory ventilation apparatus 1000 may be a non-invasive respiratory ventilation apparatus, an oxygen therapy device, or any other breathing assistance device. In some embodiments, the system 10 may further include a network 2000, a terminal 3000, a processing device 4000, and a storage device 5000. It should be noted that one or more ofthe network 2000, the terminal 3000, the processing device 4000, and the storage device 5000 may be omitted. The components in the system 10 may be connected in various ways. Merely by way of example, as illustrated in FIG. 1A, the respiratory ventilation apparatus 1000 may be connected to the processing device 4000 through the network 2000. As another example, the respiratory ventilation apparatus 1000 may be connected to the processing device 4000 directly as indicated by the bi-directional arrow in dotted lines linking the respiratory ventilation apparatus 1000 and the processing device 4000. As a further example, the storage device 5000 may be connected to the processing device 4000 directly or through the network 2000. As still a further example, the terminal 3000 may be connected to the processing device 4000 directly (as indicated by the bi-directional arrow in dotted lines linking the terminal 3000 and the processing device 4000) or through the network 2000.

The respiratory ventilation apparatus 1000 may be configured to treat, prevent, and/or relieve respiratory-related disorders or diseases of a user 8000. In some embodiments, the respiratory ventilation apparatus 1000 may deliver a pressurized respiratory gas to the user 8000 (e.g., a nose and/or a mouth of the user). In some embodiments, the respiratory ventilation apparatus 1000 may include at least one gas inlet and a gas outlet (see the pipeline interface 120 in FIG. 1B). The at least one gas inlet may be configured to introduce outside gas into the respiratory ventilation apparatus 1000. The gas outlet may be configured to expel pressurized gas from the respiratory ventilation apparatus 1000 to a respiration pipe 6000. In some embodiments, the gas outlet may be connected to the respiration pipe 6000. In some embodiments, the respiration pipe 6000 may be connected to a user interface 7000. Therefore, the gas generated by the respiratory ventilation apparatus 1000 may be discharged to the user 8000 through the respiration pipe 6000 and the user interface 7000.

In some embodiments, the respiratory ventilation apparatus 1000 may include a main body 1100. The main body 1100 may include a pneumatic block configured to generate a gas with a high-pressure higher than atmospheric pressure. The pneumatic block may be provided with a blower cavity, and a blower may be mounted in the blower cavity. The blower refers to a device used to increase a gas pressure and deliver the gas to assist the user in breathing. In some embodiments, the respiratory ventilation apparatus 1000 may include a noise reduction device (see FIGS. 2A-2D and 3A-3H). In some embodiments, a porous sound-absorbing plate (see FIGS. 4A-4G) may be configured in the noise reduction device, and the porous sound-absorbing plate may be used to improve a noise reduction effect of the noise reduction device. In some embodiments, the respiratory ventilation apparatus 1000 may include a sealing structure (see FIGS. 5A-5J). The sealing structure may be used for a sealing connection between the main body 1100 and a connecting device. The connecting device may include a breather pipe that receives the high-pressure gas generated by the main body 1100. In some embodiments, the respiratory ventilation apparatus 1000 may include a reservoir (see FIGS. 1H-1J and FIGS. 6A-6I) configured to humidify the pressurized gas. In some embodiments, the reservoir may include a reservoir housing and a pressing button disposed on an outer surface of the reservoir housing (see FIGS. 7A-7G). The pressing button may be configured to be pressed in a direction close to the inside of the reservoir housing under an action of an external force, so that the reservoir may be assembled to or detached from the main body of the respiratory ventilation apparatus. In some embodiments, the respiratory ventilation apparatus 1000 may include a liquid level detection device (see FIGS. 8A-8D), which is used for liquid level detection of a humidifier (e.g., the reservoir) of the respiratory ventilation apparatus. In some embodiments, the noise reduction device in the respiratory ventilation apparatus 1000 may be configured with a flow detection device (see FIGS. 9A-9C). The flow detection device may be mounted in the noise reduction device so that a pressure difference is formed by the gasflow on two sides of the flow detection device along a gasflow direction, and the flow detection is achieved using a pressure difference principle.

In some embodiments, the respiratory ventilation apparatus 1000 may also include one or more controllers. The controller may be connected to one or more components of the respiratory ventilation apparatus 1000 directly or via a network (e.g., a wired network, a wireless network). The controller may control an operation of the one or more components of the respiratory ventilation apparatus 1000. In some embodiments, the controller may be configured to activate the respiratory ventilation apparatus 1000 upon an initiation of the operation. For example, the controller may initiate a random access memory of the respiratory ventilation apparatus 1000, read one or more parameters from one or more storage devices 5000 (e.g., a non-volatile memory) of respiratory ventilation apparatus 1000, and/or initiate a detection module configured to detect one or more parameters related to the system 10. In some embodiments, the parameter may include at least one parameter used to control the pressure of respiratory gas, at least one parameter used to control a humidity of the respiratory gas, at least one parameter used to control a temperature of the respiratory gas, at least one parameter used to control a concentration ratio of a target gas (e.g., oxygen) in the respiratory gas, at least one parameter used to control a flux of the target gas or the respiratory gas, etc. In some embodiments, the controller may be configured to initiate a program that constantly reads information from the detection module, and control the parameter of the respiratory gas using at least the information read from the detection module and one or more of the parameters.

In some embodiments, the respiratory ventilation apparatus 1000 may further include or be equipped with one or more sensors configured to detect parameters related to the respiratory gas, breathed gas of the user 8000, and/or an operation status of the respiratory ventilation apparatus 1000. The parameters related to the respiratory gas may include, for example, the flux of the respiratory gas or the target gas, a concentration ratio of the target gas in the respiratory gas, a flow rate of the respiratory gas, a temperature of the respiratory gas, the humidity of the respiratory gas, etc., or a combination thereof. The parameters related to the operation status of the respiratory ventilation apparatus 1000 may include a running time of the respiratory ventilation apparatus 1000, a time of delay for pressurizing the respiratory gas, a gas leakage of the pressurized respiratory gas, an input voltage of the gas pressurization unit, etc., or a combination thereof.

In some embodiments, the user 8000 may be a patient. In some embodiments, the patient may have one or more respiratory-related disorders or diseases. In some embodiments, the respiratory-related disorders or diseases may be characterized by apneas, hypopneas, or hyperpneas, etc. Exemplary respiratory-related disorders or diseases may include, for example, obstructive sleep apnea (OSA), Cheyne-stokes respiration (CSR), obesity hyperventilation syndrome (OHS), chronic obstructive pulmonary disease (COPD), neuromuscular disease (NMD), chest wall disorders, acute respiratory distress syndrome (ARDS), etc.

In some embodiments, the user interface 7000 may be configured to interface the respiratory ventilation apparatus 1000 to the subject 8000, for example, by providing a flow of respiratory gas (e.g., the gas, a humidified gas enriched with oxygen). In some embodiments, the subject interface 7000 may include a gas channel to guide the respiratory gas. The subject interface 7000 may include a mask, a pipe, etc. For example, the user interface 7000 may be a nasal mask, a full-face mask, a pipe connected to a mouth of the user 8000, a tracheostomy pipe connected to a trachea of the user 8000. In some embodiments, the user interface 7000 may form a sealing connection with a face region of the user 8000 to facilitate the delivery of the respiratory gas at a pressure that has a sufficient variance with an ambient pressure to affect a therapy (e.g., a positive pressure of about 10 cmH₂O).

In some embodiments, the respiration pipe 6000 may be configured to guide the respiratory gas from the respiratory ventilation apparatus 1000 to the user interface 7000. The respiration pipe 6000 may include a gas channel to guide the respiratory gas. In some embodiments, the respiration pipe 6000 may form a sealing connection with the gas outlet of the respiratory ventilation apparatus 1000. In some embodiments, the respiration pipe 6000 may form a sealing connection with the user interface 7000.

In some embodiments, the network 2000 may include any suitable network that facilitates an exchange of information and/or data for the system 10. In some embodiments, one or more components of the system 10 (e.g., the respiratory ventilation apparatus 1000, the terminal 3000, the processing device 4000, or the storage device 5000) may communicate information and/or data with one or more other components of the system 10 via the network 2000. For example, the processing device 4000 may obtain signals from the respiratory ventilation apparatus 1000 via the network 2000.

As another example, the processing device 4000 may obtain a user instruction from the terminal 3000 via the network 2000. In some embodiments, the network 2000 may be any type of a wired or wireless network, or a combination thereof.

In some embodiments, the terminal 3000 may include a mobile device 3000-1, a tablet computer 3000-2, a laptop computer 3000-3, etc. or any combination thereof. In some embodiments, the terminal 3000 may remotely operate the respiratory ventilation apparatus 1000. In some embodiments, the terminal 3000 may operate the respiratory ventilation apparatus 1000 via the wireless connection. In some embodiments, the terminal 3000 may receive information and/or instructions input by the user, and send the received information and/or instruction to the respiratory ventilation apparatus 1000 or to the processing device 4000 via the network 2000. In some embodiments, the terminal 3000 may receive the data and/or information from the processing device 4000. In some embodiments, the terminal 3000 may display information relating to the system 10. In some embodiments, the terminal 3000 may be a part of the processing device 4000. In some embodiments, the terminal 3000 may be omitted. In some embodiments, via the terminal 3000, a user may remotely update software of the respiratory ventilation apparatus 1000, and/or adjust or set one or more parameters of the respiratory ventilation apparatus 1000.

In some embodiments, the processing device 4000 may process the data and/or information obtained from the respiratory ventilation apparatus 1000, the terminal 3000, and/or the storage device 5000. For example, the processing device 4000 may obtain the signals detected by one or more sensors in the respiratory ventilation apparatus 1000, the respiration pipe 6000, and/or the subject interface 7000, and may process and/or analyze the signals to obtain one or more parameters related to the respiratory gas, the breathed gas of the user 8000, and/or the operation status of the respiratory ventilation apparatus 1000.

In some embodiments, the processing device 4000 may include an obtaining unit and a processing unit. The obtaining unit may be configured to obtain information related to the system 10 (e.g., the respiratory ventilation apparatus 1000, the processing device 4000, the storage device 5000, the terminal 3000, etc.). The information may include the signals detected by the detection module, the data read from the storage device 5000, the instruction or data provided by the terminal 3000, etc. In some embodiments, the information may be transmitted to the processing unit for processing. In some embodiments, the obtaining unit may obtain or transmit the information via a tangible transmission media or a carrier-wave transmission media.

In some embodiments, the storage device 5000 may store data and/or instruction. In some embodiments, the storage device 5000 may store data or information obtained from the respiratory ventilation apparatus 1000. For example, the processing device 4000 may determine one or more parameters related to the respiratory gas, the breathed gas of the subject 8000, and/or the operation status of the respiratory ventilation apparatus 1000 based on the signals obtained from one or more sensors of the respiratory ventilation apparatus 1000, the respiration pipe 6000, and/or the subject interface 7000. The determined parameter(s) may be stored in the storage device 5000 for further use or processing. In some embodiments, the storage device 5000 may store data obtained from the terminal 3000 and/or the processing device 4000.

In some embodiments, the storage device 5000 may store the data and/or instruction that the processing device 4000 may execute or use to perform the exemplary method described in the present disclosure.

In some embodiments, the storage device 5000 may be connected to the network 2000 to communicate with one or more components in the system 10 (e.g., the respiratory ventilation apparatus 1000, the processing device 4000, the terminal 3000, etc.). The one or more components in the system 10 may access the data or instruction stored in the storage device 5000 via the network 2000. In some embodiments, the storage device 5000 may be directly connected to or communicate with one or more components in the system 10 (e.g., the respiratory ventilation apparatus 1000, the processing device 4000, the terminal 3000, etc.). In some embodiments, the storage device 5000 may be a part of the processing device 4000. In some embodiments, the storage device 5000 may be a part of the respiratory ventilation apparatus 1000.

FIG. 1B is a schematic diagram illustrating the respiratory ventilation apparatus according to some embodiments of the present disclosure; FIG. 1C is a schematic diagram illustrating a structure of a noise reduction device in the respiratory ventilation apparatus according to some embodiments of the present disclosure; FIG. 1D is a schematic diagram illustrating a top hoisting mounting of a blower in the noise reduction device according to some embodiments of the present disclosure; FIG. 1E is a schematic diagram illustrating a bottom supporting mounting of the blower in the noise reduction device according to some embodiments of the present disclosure; FIG. 1F is a schematic diagram illustrating a sidewall suspending mounting of the blower in the noise reduction device according to some embodiments of the present disclosure; FIG. 1G is a schematic diagram illustrating a three-dimensional (3D) structure of a sectional view along BB direction in FIG. 1F; FIG. 1H is a dissembled schematic diagram of a reservoir and a heating device of the respiratory ventilation apparatus according to some embodiments of the present disclosure; FIG. 11 is a dissembled schematic diagram of the reservoir and the heating device of the respiratory ventilation apparatus according to some other embodiments of the present disclosure; FIG. 1J is a dissembled schematic diagram of specific structures of the reservoir and the heating device of the respiratory ventilation apparatus according to some embodiments of the present disclosure; and FIG. 1K is a schematic diagram illustrating a structure of an elastic supporting structure of the heating device according to some embodiments of the present disclosure.

As shown in FIG. 1B, the embodiments of the present disclosure provide a respiratory ventilation apparatus 1000 including: a main body 1100 may include a pneumatic block, which is configured to generate a gas with a high-pressure higher than atmospheric pressure. The pneumatic block may be provided with a blower cavity, and a blower may be mounted in the blower cavity. The blower refers to a device used to improve a gas pressure and deliver the as. The respiratory ventilation apparatus 1000 may further include a reservoir 1200. The reservoir 1200 may be configured to humidify the pressurized gas, so that the gas breathed in by a user 8000 may be humidified to improve a use experience.

In some embodiments, the main body 1100 may include a display screen 110 used to interact with the user. For example, a main control chip may prompt the user through the display screen 110 about an amount of liquid in the reservoir. For more explanation about the main control chip, please refer to the relevant descriptions of FIG. 8B. The display screen 110 may be configured to display information related to the status of the respiratory ventilation apparatus 1000. The information displayed by the display screen 110 may include, for example, parameters related to breathing gas, the breathed gas of the user 8000 and/or the operation status of the respiratory ventilation apparatus 1000. In some embodiments, the display screen 110 may be configured as a software operation interface for the respiratory ventilation apparatus 1000. In some embodiments, the display screen 110 may be a touch panel.

In some embodiments, the respiratory ventilation apparatus 1000 may further include the pipeline interface 120 connected to an external device (e.g., the user interface 7000). For more descriptions on the pipeline interface 120, please refer to the related descriptions in FIGS. 10A and 10B.

In some embodiments, the respiratory ventilation apparatus 1000 may further include a main body button 140 (referring to related descriptions in FIGS. 11A-11B). The main body button 140 may include a plurality of function buttons to facilitate the user to manipulate the operation of the respiratory ventilation apparatus. In some other embodiments, the respiratory ventilation apparatus 1000 may further include a button 150 (referring to related descriptions in FIGS. 12A-12R). By pressing the button 150, the main body 1100 may be manipulated to realize operations like power on and off control and parameter setting, etc.

In some embodiments, the main body 1100 may include the noise reduction device configured to reduce a noise generated during an operation of the main body; the noise reduction device including a gas inlet structure, a noise reduction housing, a blower cavity, and a gas channel. The gas may enter the gas channel through the gas inlet structure; the gas channel being formed between a sidewall of the noise reduction housing and a sidewall of the blower cavity, and the gas channel being configured to transfer the gas. The blower cavity may be disposed with at least one cavity gas inlet along a gasflow direction, the gas in the gas channel may enter the blower cavity through the cavity gas inlet. In some embodiments, the gas channel may be disposed around an outer wall of the blower cavity. A specific structure of the noise reduction device may be in various forms, such as the noise reduction device 200 shown in FIGS. 2A-2D and the noise reduction device 300 shown in FIGS. 3A-3F. The descriptions of the specific structure is as follows.

In some embodiments, the noise reduction device may include a porous sound-absorbing plate configured to improve a noise reduction effect of the noise reduction device; the porous sound-absorbing plate being disposed with through holes along a thickness direction. The specific structure of the noise reduction device may be in various forms, referring to the related descriptions in FIGS. 4A-4G as follows.

In some embodiments, as shown in FIG. 1C, the main body 1100 may be provided with a blower 234 inside. The blower 234 may be mounted in the noise reduction device 200. The blower 234 may be fixed on a bottom, a top or a sidewall of the noise reduction device 200 through a blower mounting structure. No matter how the blower 234 is mounted in the noise reduction device, a gap between the outer wall of the blower and the inner wall of the noise reduction device may effectively prevent the noise generated when the blower operates from transmitting to the outside.

In some embodiments, the blower mounting structure may include a blower hoisting and suspension structure, one end of the blower hoisting and suspension structure being connected to the blower, and the other end being connected to the top of the noise reduction device. In some embodiments, as shown in FIGS. 1C and 1D, the blower hoisting suspension structure 235 may include a flexible blower cover 235-1 and a plurality of hoisting columns 235-2. In some embodiments, the flexible blower cover 235-1 may be an enclosed structure made of the silicone material. The flexible blower cover 235-1 may wrap at least a part of the structure of the blower 234, and the flexible blower cover 235-1 may have at least one opening in the center of the bottom for mounting the blower. When the blower 234 is mounted so that the gas inlet of the blower 234 is located at the bottom of the mounting position, and when the flexible blower cover 235-1 surrounds the blower 234, an opening with the same size as the gas inlet at the bottom of the blower 234 may be provided on the flexible blower cover 235-1, so as to ensure that the gasflow smoothly enters the blower 234 through the opening at the bottom of the flexible blower cover 235-1, thereby ensuring a normal operation of the blower 234. One ends of the plurality of hoisting columns 235-2 may be fixedly connected to the flexible blower cover 235-1 (e.g., an upper end of the flexible blower cover 235-1), and the other ends of the plurality of hoisting columns 235-2 may be fixedly connected to an upper space of the noise reduction device 200 of the blower (e.g., a noise reduction top housing 236 of the noise reduction device 200). In some embodiments, the upper ends of the plurality of lifting columns 235-2 may be configured with flexible buckle heads 235-21, and the plurality of flexible buckle heads 235-21 may be fixedly connected to the plurality of mounting holes 236-1 on the noise reduction top housing 236 in a detachable (e.g., clamping) way. In some embodiments, as shown in FIG. 1D, the flexible buckle head 235-21 may be a gourd-shaped material made of the flexible material (e.g., the silicone) and may have a certain deformation ability. Outer diameters of the upper and lower ends of the gourd-shaped flexible buckle head 235-21 may be greater than the outer diameter of a middle section, and the outer diameter of the middle section may be slightly greater than an inner diameter of the mounting hole 236-1. When the flexible buckle head 235-21 is mounted with a matching mounting hole 236-1, the gourd-shaped upper end of the flexible buckle head 235-21 may be extruded and deformed to pass through the mounting hole 236-1 under the action of the external force, so that the mounting hole 236-1 is set on the outer side of the middle section of the flexible buckle head 235-21 to form an interference fit. As the outer diameter of the upper and lower ends of the gourd shape is greater than the outer diameter of the middle section, the flexible buckle head 235-21 may be fixedly connected to the mounting hole 236-1 of the noise reduction top housing 236. When the blower 234 needs to be mounted into the noise reduction device 200, the blower 234 may be mounted into the flexible blower cover 235-1 first, and then through the plurality of flexible buckle heads 235-21, the blower 234 may be fixedly connected to the plurality of mounting hole 236-1 of the noise reduction top housing 236, then the noise reduction top housing 236 and a noise reduction bottom housing (e.g., the bottom housing of the noise reduction housing 220) may be connected. When configured in the noise reduction device 200 through the hoisting and suspension, there may be a certain gap between the outer wall of the flexible blower cover 235-1 and the inner wall of the noise reduction shell (including the noise reduction bottom housing and the noise reduction top housing 236). It may be understood that suspending the blower 234 inside the noise reduction device 200 may effectively prevent the vibration noise generated when the blower 234 rotates from being transmitted outward. In some embodiments, there may be at least three hoisting columns 235-2, and there may also be four, five or six hoisting columns 235-2. The plurality of hoisting columns 235-2 may not only ensure a stability of the hoisting of the blower 234, but also further improve the vibration noise reduction effect of the blower 234. In some embodiments, the plurality of hoisting columns 235-2 may be integrally formed on a side or a top surface of the flexible blower cover 235-1. In some other embodiments, the plurality of hoisting columns 235-2 may be connected to the side or the top surface of the flexible blower cover 235-1 by snapping, pasting, or other modes. In some embodiments, the plurality of hoisting columns 235-2 may be directly provided on the side or the top of the blower 234 circumferentially. The blower 234 may be suspended in the noise reduction device 200 through the plurality of hoisting columns 235-2. The hoisting columns 235-2 may be detachably or fixedly connected to the side or the top of blower 234.

In some embodiments, the mounting structure of the blower may be a blower bottom support structure. One end of the blower bottom support structure may be fixedly connected to a bottom wall or the sidewall of the blower 234, and the other end may be fixedly connected to the bottom of the noise reduction device. In some embodiments, as shown in FIG. 1E, the blower bottom support structure 235 may include a semi-enclosed structure blower cover 235-3 and a plurality of support columns 235-4. The center of the bottom of the semi-enclosed structure of the blower cover 235-3 may be at least provided with an opening with the same size as the gas inlet at the bottom of the blower 234 for mounting and supporting the blower 234, so as to ensure that the gasflow is able to smoothly enter the blower 234 through the opening at the bottom of the blower cover 235-3, thereby ensuring the normal operation of the blower 234. The plurality of support columns 235-4 may be fixedly connected to a bottom end surface or the sidewall of the blower cover 235-3. In some embodiments, the plurality of support columns 235-4 may be evenly distributed on the bottom end surface or the sidewall of the blower cover 235-3 around an axis of the blower 234. In some embodiments, there may be at least three support columns 235-4, and there may also be four, five or six support columns 235-4. The plurality of support columns 235-4 may not only ensure the stability of supporting the blower 234, but also improve the vibration noise reduction effect of the blower 234. In some embodiments, the semi-enclosed structure of the blower cover 235-3 and the plurality of support columns 235-4 may be made of flexible silicone material, which is able to eliminate a part of the vibration noise generated by the operation of the blower 234. In some embodiments, the plurality of support columns 235-4 may be integrally formed with the bottom end surface of the blower cover 235-3. In some other embodiments, the plurality of support columns 235-4 may be connected to the bottom end surface of the blower cover 235-3 by snapping, pasting, or other modes. In some embodiments, the plurality of support columns 235-4 may be directly provided at the bottom or the sidewall of the blower 234, and the support columns 235-4 may be connected to the bottom or the sidewall of the blower 234 in a detachable or fixed way.

As shown in FIG. 1D, in the noise reduction device of the present disclosure, a motor part of the blower 234 (made of metal, heavier) may be located at the top in a vertical direction, and a gas inlet blade part of the blower 234 (made of plastic, lighter) may be located at the bottom of the vertical direction. Therefore, the mounting of blower 234 may prone to a danger of being top-heavy. Therefore, using a hanging manner to mount the blower 234 in the noise reduction device 200 enables the mounted blower 234 more stable. As an overall center of gravity of the mounted blower 234 itself is upward (i.e., the side close to the motor), the mode of hanging the blower 234 at the top inside the noise reduction device 200 further stabilizes the center of gravity of the blower 234 compared with the mode of directly fixing the bottom of the blower 234 to the bottom inside the noise reduction device 200 through the plurality of support columns 235-4. Moreover, the hoisting and suspension mode may save an internal space of the noise reduction device 200, and make the noise reduction device smaller in size, so as to achieve a small-volume design of the noise reduction device.

In some embodiments, the blower mounting structure may include a blower sidewall suspension structure. One end of the blower suspension structure may be connected to the blower, and the other end may be connected to the sidewall of the noise reduction device. As shown in FIG. 1F, the blower bottom support structure 235 may include a semi-enclosed structure blower cover 235-5 (similar to the blower cover 235-3 in FIG. 1E) and a plurality of side mounting columns 235-6, and at least an opening with the same size as the gas inlet at the bottom of the blower 234 may be provided at the center of the bottom of the blower cover 235-5, which is used to mount and support the blower 234 while ensuring that the gasflow smoothly enters the blower 234 through the bottom opening of the flexible blower cover 235-5, thereby ensuring the normal operation of the blower 234. In some embodiments, the semi-enclosed structure of the blower cover 235-5 and the plurality of side mounting columns 235-6 may be made of the flexible material (e.g., the silicone). The flexible material of the blower cover 235-5 may eliminate a part of the vibration noise generated by the blower cover 235-5 when the blower 234 is operating. In some embodiments, the blower cover 235-5 of the semi-enclosed structure may be made of a rigid material (e.g., plastic), and at least portion of the plurality of side mounting columns 235-6 connected to the noise reduction device may be made of the flexible material (e.g., the silicone). In some embodiments, the plurality of side mounting columns 235-6 may extend from the upper end of the sidewall or from the sidewall of the semi-enclosed structure of the blower cover 235-5, and the blower 234 may be fixed on an inner sidewall of the noise reduction device 200 through the side mounting column 235-6. In some embodiments, the side mounting column 235-6 may include a connection column 235-61 and a clamping joint 235-62 fixedly connected to the outside of the sidewall of the blower cover 235-5, the clamping joint 235-62 being fixedly connected to the inner sidewall of the noise reduction device in a clamped mode. In some embodiments, the side mounting column 235-6 may be made of the flexible material (e.g., the silicone), that is, the connection column 235-61 and the clamping joint 235-62 may be both made of the flexible material, so that the side mounting column 235-6 as a whole has a certain deformability. In some embodiments, the connection column 235-61 of the side mounting column 235-6 may be made of the rigid material, and the clamping joint 235-62 may be made of the flexible material, so that the clamping joint 235-62 has a certain deformation when combined with the noise reduction device. In some embodiments, the connection column 235-61 of the side mounting column 235-6 and the blower cover 235-5 may be integrally formed, and the clamping joint 235-62 may be detachably connected to the connection column 235-61. In some embodiments, the side mounting column 235-6 may be integrally formed with the blower cover 235-5. During the use, when the blower 234 needs to be mounted into the noise reduction device 200, the blower 234 may be mounted into the blower cover 235-5 of the semi-enclosed structure. The blower cover 235-5 may be used to mount and support the blower 234. Then through the plurality of side mounting columns 235-6, the blower cover 235-5 may be fixedly connected to a plurality of side mounting holes and side mounting slots on the inner sidewall of the noise reduction housing 220. In some embodiments, the clamping joint 235-62 may be flat and button-shaped. In a natural state, the clamping joint 235-62, the connection column 235-61, and the sidewall of the blower cover 235-5 at the connection point may be in an H shape. In some embodiments, the interference fit may be formed between the side mounting columns 235-6 and the side mounting holes or the side mounting slots on the sidewall of the noise reduction housing 220 to ensure that the blower 234 is stably suspended in the noise reduction housing 220. When the blower 234 is configured in the noise reduction device 200 through sidewall suspension, there may be a certain gap between the outer wall of the blower 234 and/or the blower cover 235-5 and the inner wall of the noise reduction housing 220. It may be understood that the blower 234 is suspended in the gas, which effectively prevent the vibration noise generated when the blower 234 rotates from being transmitted outward. In some embodiments, there may be at least three side mounting columns 235-6, and there may also be four, five or six side mounting columns 235-6. The plurality of side mounting columns 235-6 may not only ensure the stability of the hoisting of the blower 234, but also further improve the vibration noise reduction effect of the blower 234.

In some embodiments, the main body 1100 may include a connecting device including a breather pipe receiving the high-pressure gas. When the respiratory ventilation apparatus 1000 works, the external gas may enter the blower cavity through the noise reduction device. The blower pressurized gas may enter the connecting device and finally be delivered to the external device (e.g., the user interface 7000).

In some embodiments, the respiratory ventilation apparatus 1000 may further include a sealing structure used for the sealing connection between the main body 1100 and the connecting device. For specific contents of the sealing structure, please refer to the descriptions in FIGS. 5A-5J as follows.

In some embodiments, the connecting device may include may include the reservoir or a cover plate without a reservoir. In some embodiments, the reservoir may be configured to contain the liquid, and a humidity of the pressurized gas may be increased through the reservoir. For more contents of the reservoir, please refer to the relevant descriptions of FIGS. 6A-6I and FIGS. 7A-7G as follows.

In some embodiments, the reservoir 1200 may further include the pressing button disposed on an outer surface of the reservoir housing. The pressing button may be configured to be pressed in a direction close to the inside of the reservoir housing under the action of an external force, so that the reservoir 1200 may be assembled to or detached from the main body 1100. For more contents of the reservoir, please refer to the relevant descriptions of FIGS. 6A-6I and FIGS. 7A-7G as follows.

In some embodiments, the reservoir 1200 may also include a heating device 1210 used to heat the liquid in the reservoir 1200. In some embodiments, the heating device 1210 may be fixedly connected to the reservoir 1200, that is, the heating device 1210 may be fixed on the reservoir 1200.

The reservoir 1200 shown in the embodiment may be recycled. In some embodiments, the heating device 1210 may be disposed at a bottom or inside the reservoir 1200 and close to the bottom. In some application scenarios, medicine may need to be added to the liquid in the reservoir 1200. The reservoir with medicine may be difficult to clean, and there may be a scenario where one of the heating device 1210 and the reservoir is damaged and the other may be reused. Therefore, in some other embodiments, as shown in FIGS. 1H and 11, the heating device 1210 may be configured to be detachably mounted at a lower end of the reservoir 1200. In some embodiments, the lower end of the reservoir 1200 may be open-shaped. When the heating device 1210 is detachably mounted at the lower end of the reservoir 1200, the heating device 1210 may serve as the bottom wall of the reservoir 1210.

In some embodiments, a sealing connection may be formed between the heating device 1210 and the reservoir 1200 to prevent the liquid in the reservoir 1200 from leaking. In some embodiments, an elastic seal 1220 may be disposed at a connection between the reservoir 1200 and the heating device 1210, and the elastic seal 1220 may seal the connection between the reservoir 1200 and the heating device 1210. In some embodiments, the elastic seal 1220 may be an annular elastic silicone strip, and the annular elastic silicone strip may be fixed on the reservoir 1200 or the heating device 1210 using a glue.

In some embodiments, as shown in FIG. 1H, the elastic seal 1220 may be fixedly connected to the reservoir 1200, and the elastic seal 1220 may also be provided with at least one protruding strip. When the heating device 1210 is mounted on the lower end of the reservoir 1200, a protrusion direction of the protruding strip may be toward the inner sidewall of the heating device 1210. The at least one protruding strip may be used to increase a friction at the connection between the elastic seal 1220 and the heating device 1210, so as to prevent the heating device 1220 from falling off In other embodiments, as shown in FIG. 11, the elastic seal 1220 may be fixedly connected to the heating device 1210, and the elastic seal 1220 may be also provided with at least one protruding strip. When the heating device 1210 is mounted on the lower end of the reservoir 1200, the protrusion direction of the protrusion strip may be toward the outer wall of the connection at the lower end of the reservoir 1200. The at least one protruding strip may be used to increase the friction at the connection between the elastic seal 1220 and the reservoir 1200 to prevent the heating device 1210 from falling off.

By configuring the main body 110 and the reservoir 1200 as separate and detachable connection. The reservoir 1200 may be a reusable reservoir or a disposable reservoir, which solves the problem that the reservoir 1200 is difficult to clean and add water.

In some embodiments, as shown in FIGS. 1J and 1K, the heating device 1210 may be disposed on the main body 1100 at a position corresponding to the reservoir 1200. The heating device 1210 may include a metal heating plate 1211, a spacer 1212 and a heating bottom plate 1213 assembled in sequence from top to bottom. The heating bottom plate 1213 may be used as the bottom housing of the heating device 1210, or may be regarded as a part of the bottom housing of the main body 1100. The heating bottom plate 1213 may be made of a plastic material (e.g., an integrally formed plastic bottom plate). As shown in FIG. 1J, the metal heating plate 1211, the spacer 1212 and the heating bottom plate 1213 may be assembled from top to bottom to form a whole heating device 1210. The heating device 1210 may be fixed through a detachable connection to the bottom of the reservoir 1200, such as a snap connection and a screw connection. An elastic part may be provided on a side of the spacer 1212 corresponding to the metal heating plate 1211, thereby achieving an elastic support for the metal heating plate 1211.

In some embodiments, as shown in FIG. 1K, the elastic part on the spacer 1212 may include a plurality of elastic bodies 1212-1 on one side of the spacer 1212 facing the metal heating plate 1211. At least an upper end region of the elastic body 1212-1 may be made of the flexible material (e.g., the silicone) and may have a certain deformability. The lower ends of the plurality of elastic bodies 1212-1 may be fixedly connected to the surface of the spacer 1212 (the side of the spacer 1212 facing the metal heating plate 1211), and the upper ends of the plurality of elastic bodies 1212-1 may be in contact with the metal heating plate 1211. In some embodiments, there may be four elastic bodies 1212-1 as shown in FIG. 1K, or there may be elastic bodies 1212-1 of other numbers, such as two, three, five, six, etc. The number of the elastic bodies 1212-1 may be designed according to an outer contour shape and a size of the spacer 1212, and the plurality of elastic bodies 1212-1 may be disposed at equal intervals along a contour of the corresponding supported heating plate 1211. In some embodiments, the upper end of the elastic body 1212-1 may be set as a tapered structure, and the lower end of the elastic body 1211-1 may be set as a hollow support column 1212-2. An outline profile section of the support column 1212-2 may be set to circular, rectangular, or oval and other arbitrary shapes, the specific shape depends on the actual space or other needs. The support column 1212-2 may correspond to one side of the heating bottom plate 1213, that is, a side surface of the spacer 1212 facing the heating bottom plate 1213 may be recessed inward, so that the support column 1212-2 forms a hollow structure. The side surface of the heating bottom plate 1213 facing the spacer 1212 corresponding to the support column 1212-2 may be provided with a protrusion 1213-1. When the heating bottom plate 1213 and the spacer 1212 are assembled, the hollow structure of the support column 1212-2 may be assembled and connected to the protrusion 1213-1 accordingly. When the heating device 1210 of this embodiment is mounted on the main body 1100, the deformability of the conical structures of the plurality of elastic bodies 1212-1 may allow the metal heating plate 1211 to have space to move up and down. When the pressing button 750 shown below receives a downward external force to mount the reservoir 1200 to the main body 1100, or to disassemble the reservoir 1200 from the main body 1100, the metal heating plate 1211 may move downward for a certain distance under the pressure of the reservoir 1200 to facilitate a smooth mounting and dissembling of the reservoir 1200. When the heating device 1210 is assembled independently, the supporting column 1212-2 in the spacer 1212 may be first clamped on a cylindrical protrusion 1213-1 on the heating bottom plate 1213 to assemble the heating device 1210 and the main body 1100. During the overall assembly, the main body 1100 may be turned upside down and the metal heating plate 1211 may be placed on the corresponding opening of the main body 1100. The opening of the main body may be smaller than the metal heating plate 1211 in a length direction of the metal heating plate 1211. A limiting groove may be provided at the corresponding edge of the opening of the main body to be placed on the length direction of the metal heating plate 1211, which is falling out of the opening. A width of the opening may be greater than or equal to a width of the metal heating plate 1211, so that the metal heating plate 1211 is able to be completely exposed in the width direction, and under an elasticity of the spacer 1212, a certain degree of torsional variation and displacement may occur. After the metal heating plate 1211 is assembled, the assembled spacer 1212 and the heating bottom plate 1213 may be placed on the metal heating plate 1211. Finally, the heating bottom plate 1213 may be buckled on a corresponding opening of the main body 1100, and may be finally assembled and fixed with screws on the main body 1100.

In some embodiments, the spacer 1212 may adopt the elastic material (e.g., the silicon) as a whole, so as to better protect the metal heating plate 1211 and prevent the metal heating plate 1211 form deformation, which may shorten a serve life of the metal heating plate 1211.

In some embodiments, the respiratory ventilation apparatus may further include the liquid level detection device configured to detect a liquid level of a humidifier (e.g. the reservoir) in the respiratory ventilation apparatus 1000. For specific contents of the liquid level detection device, please refer to the related descriptions in FIGS. 8A-8D as follows.

In some embodiments, the noise reduction device in the respiratory ventilation apparatus may be provided with a flow detection device mounted inside the noise reduction device, so that the gasflow forms a pressure difference on two sides of the flow detection device along the gasflow direction, thereby realizing the flow detection using a pressure difference principle. For specific contents of the flow detection device, please refer to the related descriptions in FIGS. 9A-9C as follows.

FIG. 2A is a 3D schematic diagram illustrating a structure of a noise reduction device (without a noise reduction top housing) according to some embodiments of the present disclosure; and FIG. 2B is a schematic top view illustrating a structure of a noise reduction device (without a noise reduction top housing) according to some embodiments of the present disclosure.

In some embodiments, referring to FIG. 2A and FIG. 2B, the noise reduction device 200 may include a gas inlet structure, a noise reduction housing 220, a blower cavity 230 and a gas channel 240. The gas inlet structure may be configured to connect to an externally delivered gas (e.g., the gas) to direct the gas through the gas inlet structure into the noise reduction device 200. The blower cavity 230 may be configured to accommodate a blower 234. In some embodiments, the noise reduction housing 220 may include a noise reduction top housing and a noise reduction bottom housing, and the noise reduction top housing may be fixedly connected to the noise reduction bottom housing, such as by screw connection. In order to facilitate a demonstration of an internal structure of the noise reduction device 200, neither FIG. 2A nor FIG. 2B is disposed with a noise reduction top housing, and the noise reduction housing 220 in the figures shows only the noise reduction bottom housing. In some embodiments, the noise reduction housing 220 may include only the noise reduction bottom housing, and the noise reduction top housing match the noise reduction bottom housing may be integrated within the main body of the respiratory ventilation apparatus.

Referring to FIG. 2B, the gas channel 240 may be disposed between an inner wall of the noise reduction housing 220 and an outer wall of the blower cavity 230. The gas channel 240 may be configured to transfer the gas input from the gas inlet structure, and the gas advancing along the gas channel 240 within the gas channel 240 may forms a gasflow. The blower cavity 230 may be disposed with at least two cavity gas inlets along a gasflow direction. The gas may enter the gas channel 240 through the gas inlet structure and then enter the blower cavity 230 through the at least two cavity gas inlets. By adopting at least two cavity gas inlets to divert the gas in the gas channel 240 into the blower cavity 230, a turbulence of gas may be reduced. In this way, the noise of the gasflow entering the blower cavity 230 may be effectively attenuated.

In some embodiments, the blower cavity 230 may be disposed with two cavity gas intakes along the gasflow direction. In some embodiments, the two cavity gas inlets may be disposed at both ends of the gas channel 240, allowing the two cavity gas inlets to be farther apart. The gas may enter the gas channel 240 from the cavity intake structure. A portion of the gas may enter the blower cavity 230 through the cavity gas inlet disposed at a beginning end of the gas channel 240, and the other portion of the gas may enter the blower cavity 230 through the other cavity gas inlet disposed at a tail end of the gas channel 240 as it advances along the gas channel 240. As a portion of the gasflow flows through a longer gas channel 240, the noise may be gradually attenuated by the gas channel 240 to provide the noise reduction effect.

In some embodiments, the blower cavity 230 may be disposed with three cavity gas inlets in the gasflow direction. In some embodiments, the three cavity gas inlets may be disposed at the beginning end, a middle end, and the tail end of the gas channel 240. In some embodiments, a distance between any two of the three cavity gas inlets may be designed to be equally spaced or unequally spaced based on conditions such as a gas resistance and a gas pressure within the gas channel 240.

In some embodiments, the gas channel 240 may have at least three spaces in the direction along the gasflow direction, and each space may be distributed in a sequence of a great cavity and a small cavity, with the at least two cavity gas inlets disposed in at least two great cavity spaces adjacent to the small cavities.

In some embodiments, the gas channel 240 may at least include a first space 241, a second space 242, and a third space 243 in the gasflow direction. Two dotted lines are used in FIG. 2B to divide the gas channel 240 into the first space 241, the second space 242, and the third space 243. In some embodiments, as viewed from the top view shown in FIG. 2B, the first space 241 may be disposed at a beginning section of the gas channel 240, and the first space 241 may be formed primarily by a left sidewall of the noise reduction housing 220, the lower sidewall of the noise reduction housing 220, and the outer wall of the blower cavity 230. In some embodiments, the second space 242 may be disposed in an intermediate section of the gas channel 240, and may be formed primarily by the lower sidewall of the noise reduction housing 220 and the outer wall of the blower cavity 230. In some embodiments, the third space 243 may be disposed in a tail end section of the gas channel 240 and may be formed primarily by the lower sidewall of the noise reduction housing 220, the right sidewall of the noise reduction housing 220, and an outer wall of the blower cavity 230. In some embodiments, the second space 242 may be disposed at a position on either side of a midpoint of a total length of the lower sidewall of the noise reduction housing 220, and a length of the second space 242 along the gasflow direction of the gas channel 240 may be ¼ to ⅓ of the total length of the lower sidewall of the noise reduction housing 220. It should be noted that the two dotted lines in the drawing are only approximate divisions of the spaces, which are used only for illustrating the present disclosure, and do not constitute a limitation of the present disclosure. The first space 241 may be greater than the second space 242, and the second space 242 may be smaller than the third space 243. In some embodiments, the first space 241, the second space 242, and the third space 243 may form a “great-small-great” spatial variation in a sectional region when viewed from a top-down perspective.

Relative to the second space 242, the first space 241 and the third space 243 may have a greater top-view sectional region, the gasflow may subject to a smaller resistance in the space with a greater top-view sectional region, and the gasflow may subject to greater resistance in the space with a smaller top-view sectional region. When the gasflow flows in the first space 241, the second space 242, and the third space 243 with the “great-small-great” spatial variation, the noise of different frequencies may be reduced, so that the noise of the gasflow may be gradually attenuated. For the convenience of understanding, the present disclosure refers to the first space 241 and the third space 243, which have greater sectional regions when viewed from above, as great cavities, and the second space 242, which has a smaller sectional region when viewed from above, as a small cavity. In some embodiments, the top-viewed sectional region of the first space 241 may be 1.1 to 5 times the top-viewed sectional region of the second space 242, and the third space 243 may be 1.1 to 5 times the top-viewed sectional region of the second space 242. The blower cavity 230 may have at least a first gas inlet 231 and a second gas inlet 232. The first gas inlet 231 may be disposed in the first space 241, and the second gas inlet 232 may be disposed in the third space 243. It may be appreciated that the first gas inlet 231 and the second gas inlet 232 are disposed in two great cavities. The noise reduction device 200 of the embodiment may use at least two cavity gas inlets (the first gas inlet 231 and the second gas inlet 232) to divert the gasflow into the blower cavity 230, so as to avoid an effect of turbulence of the gasflow, and allow the gasflow to enter the blower cavity 230. In this way, the noise of the gasflow entering the blower cavity 230 may be weakened, and the cavity inlets at different positions may eliminate the noise of different frequency bands, realizing noise reduction. The noise reduction refers to a process of by disposing an abrupt change in the section of the pipeline or a side-connecting resonance cavity in the pipeline, etc., changes in an impedance may be caused by sound waves in the process of propagation, thereby generating an acoustic energy interference, and thus reduces the acoustic energy radiated outward, so as to achieve the purpose of anechoic.

FIG. 2C is a schematic top view illustrating a structure of a noise reduction device (without a noise reduction top housing) according to some embodiments of the present disclosure.

In some embodiments, referring to FIG. 2C, the gas channel 240 may also include a fourth space 244 and a fifth space 245 in a gasflow direction. Three dotted lines are used in FIG. 2C to divide the gas channel 240 into the first space 241, the second space 242, the third space 243, the fourth space 244, and the fifth space 245 in the gasflow direction. It is to be noted that the three dotted lines in the figure are only an approximate division of the spaces, and are only used to illustrate the present disclosure, and do not constitute a limitation of the present disclosure. The fourth space 244 may be smaller than the third space 243, and the fifth space 245 may be greater than the fourth space 244. For more descriptions of the fourth space 244 and the fifth space 245, please refer to the second space 242 and the third space 243 above, which are not repeated here. In this embodiment, the gas channel 240 may have the first space 241, the second space 242, the third space 243, the fourth space 244, and the fifth space 245 in the gasflow direction. The second space 242 and the fourth space 244 may have relatively small top-view sectional regions, and the first space 241, the third space 243, and the fifth space 245 may have relatively great top-view sectional regions. As a result, the first space 241, the second space 242, the third space 243, the fourth space 244, and the fifth space 245 may form a “great-small-great-small-great” spatial variation in a sectional region when viewed from a top-down perspective. The second space 242 and the fourth space 244, which have smaller top-viewed sectional regions, may be small cavities, and the first space 241, the third space 243, and the fifth space 245, which have greater top-viewed sectional regions, may be great cavities.

In some embodiments, referring to FIG. 2C, the blower cavity 230 may further include a third gas inlet 233 disposed in the fifth space 245. That is to say, the blower cavity 230 of the noise reduction device 200 of the embodiment may be provided with three gas inlets, namely the first gas inlet 231 located in the first space 241, the second gas inlet 232 located in the third space 243, and the third gas inlet 233 located in the fifth space 245. The noise reduction device 200 of the embodiment may use the three cavity gas inlets to divert the gas into the blower cavity 230, and the cavity gas inlets at different positions may eliminate the noise in different frequency bands, which further enhances the noise reduction effect.

In some embodiments, the gas channel 240 may also include a sixth space and a seventh space in the gasflow direction (no schematic is given for this embodiment). The sixth space may be smaller than the fifth space and the seventh space may be greater than the sixth space. In this embodiment, the gas channel 240 may form an annular gas channel around a periphery of the blower cavity 230, and the gas channel 240 may be divided into seven spaces in the gasflow direction to form a “great-small-great-small-great-small-great” spatial variation. The blower cavity 230 may also include a fourth gas inlet disposed in the seventh space. That is to say, the blower cavity 230 of the noise reduction device 200 of the embodiment may be disposed with four gas inlets, namely, a first gas inlet 231 disposed in the first space 241, a second gas inlet 232 disposed in the third space 243, a third gas inlet 233 disposed in the fifth space 245, and a fourth gas inlet disposed in the seventh space. The noise reduction device 200 of this embodiment adopts the four cavity gas inlets to divert the gas into the blower cavity 230, and the cavity gas inlets at different positions eliminate the noise in different frequency bands, thereby further enhancing the noise reduction effect.

In some embodiments, referring to FIG. 2A, the gas inlet structure may include a gas inlet pipe 210. The gas inlet pipe 210 may be a hollow pipe structure. In some embodiments, the gas inlet pipe 210 may be a pipe structure with a circular section. In some embodiments, the gas inlet pipe 210 may be a pipe structure with a square section. An interior of the gas inlet pipe 210 may have one or more separating components 211 dividing the gas inlet pipe 210 into two or more gas inlet sub-pipes 212. As the gasflow may be prone to eddy flows when flowing in a circular pipe structure with a great section, which generates more noise. By separating the gas inlet pipe 210 into a plurality of gas inlet sub-pipes 212, the gasflow may be diverted into a plurality of long advective flows, and thus the structural design of the gas inlet pipe 210 may reduce the noise generated when the gas flows.

In some embodiments, the gas inlet pipe 210 may have a “slotted” separating component 211 inside the pipe that divides the gas inlet pipe 210 into two gas inlet sub-pipes 212. In some embodiments, the gas inlet pipe 210 may have a “crossed” separating component 211 inside the pipe that divides the gas inlet pipe 210 into four gas inlet sub-pipes 212.

In some embodiments, referring to FIG. 2A, the separating component 211 inside the pipe of the gas inlet pipe 210 may have a “tic-tac-toe” dividing the gas inlet pipe 210 into nine gas inlet sub-pipes 212.

In some embodiments, the separating component 211 inside the pipe of the gas inlet pipe 210 may also be in the shape of a “concentric circle”, dividing the gas inlet pipe 210 into a plurality of concentric rings of gas inlet sub-pipes 212.

In some embodiments, the separating component 211 inside the pipe of the gas inlet pipe 210 may also be in the shape of a hexagonal honeycomb, dividing the gas inlet pipe 210 into a plurality of hexagonal honeycomb gas inlet sub-pipes 212.

In some embodiments, referring to FIGS. 2B and 2C, the gas inlet pipe 210 may be disposed within the noise reduction housing 220 in a horizontal direction, and the dotted line M in the figure may indicate a centerline of the gas inlet pipe 210 (e.g., if the gas inlet pipe 210 is circular, then the dotted line M may be an axis line of the gas inlet pipe 210), the dotted line N may be parallel to an outer wall of the blower cavity 230 disposed in the first space 241 or parallel to a tangential direction of the outer wall of the blower cavity 230, and an angle between the dotted line M and the dotted line N may be 0. The angle θ may be less than 90°, and the first space 241 may be approximate triangular in the top view perspective. The flow gasflow direction in the gas inlet pipe 210 may be along the dotted line M from the external world to the first space 241. The gasflow may hit an outer wall of the blower cavity 230 disposed in the first space 241, and the gasflow may flow along the direction of the dotted line N to the second space 242, so that the gasflow may be turned in the first space 241, and a turning angle of the gasflow may be greater than 90°. In some embodiments, the gasflow undergoes the turning within the first space 241 at an angle of 180°-θ. During a transmission of the gasflow, when the gasflow changes the direction, the sound waves of the gasflow may produce some mutual cancellation, which eliminate some of the noise in the frequency band, and thus reduce the noise during the transmission of the gasflow.

It should be noted that the actual flow of the gasflow in the gas inlet pipe 210 and gas channel 240 may be complex, and the two dotted lines in FIG. 2B and FIG. 2C are merely exemplary flow references drawn to facilitate a better understanding of the present disclosure, which are only to be used to explain the present disclosure and do not constitute a limitation of the present disclosure.

In some embodiments, referring to FIG. 2A-FIG. 2C, an end face of the gas outlet 213 of the gas inlet pipe 210 may be vertical to a center axis of the gas inlet pipe 210. In some embodiments, a bottom surface of the noise reduction housing 220 may be horizontal and the gas inlet pipe 210 may be disposed within the noise reduction housing 220 parallel to the bottom surface. The end surface of the gas outlet 213 of the gas inlet pipe 210 may be vertical to the bottom surface of the noise reduction housing 220. At the same time, the sidewalls of the noise reduction housing 220 (including the inner wall and the outer wall) and the outer wall of the blower cavity 230 may be also vertical to the bottom surface of the noise reduction housing 220. An approximate triangular space may be formed between the end face of the gas outlet 213 of the gas inlet pipe 210, the sidewall of the noise reduction housing 220, and the outer wall of the blower cavity 230. The gasflow may come out from the gas outlet 213 of the gas inlet pipe 210, and may be blocked by the sidewalls of the noise reduction housing 220 and the outer wall of the blower cavity 230. As a result, a portion of the gasflow may be diverted to flow toward the first gas inlet 231 and, another portion may be diverted to flow along the gas channel 240 to flow toward the second gas inlet 232 and/or the third gas inlet 233. In this embodiment, the gasflow undergoes at least two flow turns within the first space 241, and the sound waves of the gasflow during the change of direction may be partially cancelled out, which eliminate some of the noise in the frequency band, and thus reduce the noise when the gasflow is transmitted.

In some embodiments, the space between the end face of the gas outlet 213 of the gas inlet pipe 210 and the outer wall of the blower cavity 230 may be smaller. When the gasflow flows out of the gas outlet 213 of the gas inlet pipe 210, two turning diverts may occur rapidly in the space of the smaller triangular shape, and the sound waves rapidly turning in the small triangular shape of the space may cancel each other out, which effectively eliminating partial of the frequency band noise.

In some embodiments, a height of the highest position of the gas outlet 213 of the gas inlet pipe 210 in the vertical direction may be less than a height of the lowest positions of the first gas inlet 231 and the second gas inlet 232 in the vertical direction. In some embodiments, referring to FIG. 2A, the gas inlet pipe 210 may be disposed near the bottom surface of the noise reduction housing 220.

In some embodiments, referring to FIG. 2A, the height of the highest position of the gas outlet 213 of the gas inlet pipe 210 in the vertical direction may be less than the height of the lowest positions of the first gas inlet 231, the second gas inlet 232, and the third gas inlet 233 in the vertical direction.

Referring to FIG. 2A, when the height of the highest position of the gas outlet 213 of the gas inlet pipe 210 in the vertical direction is less than the height of the lowest positions of the first gas inlet 231, the second gas inlet 232, and/or the third gas inlet 233 in the vertical direction, a portion of the gasflow from the gas inlet pipe 210 may turn upward to flow toward the first gas inlet 231, and a portion of the gasflow may turn rightward to flow along the gas channel 240 to the second gas inlet 232 and/or the third gas inlet 233 because of an obstruction of the sidewall of the noise reduction housing 220 and the outer wall of the blower cavity 230. Such a design of the structure may allow the gasflow to change the direction of the gas sound waves in the space formed between the end face of the gas outlet 213 of the inlet pipe 210, the sidewall of the noise reduction housing 220, and the outer wall of the blower cavity 230, and may reduce the noise by the reflection or the interference of the sound waves.

In some embodiments, a blower 234 may be disposed within the blower cavity 230. A base portion of the blower 234 may be disposed in a lower portion of the blower cavity 230, and a motor portion of the blower 234 may be disposed in an upper portion of the blower cavity 230. The base portion of the blower 234 may be configured to support and stabilize the motor portion of the blower 234, and may reduce a vibration of the motor portion to achieve a noise reduction.

In some embodiments, the first gas inlet 231 and the second gas inlet 232 may be disposed vertically at a height close to the motor portion of the blower 234 in the blower cavity 230 and away from the base portion of the blower 234, that is, the gas inlet at the bottom away from the blower 234. When the gasflow passes through the first gas inlet 231 and the second gas inlet 232 and enters the blower cavity 230, the gasflow flows from top to bottom to first cool the motor portion of the blower 234 whose height in a vertical direction is similar to the gas inlets, so as to make the blower 234 maintain an appropriate temperature range, thereby maintaining a better working performance. The gasflow may ultimately enter the blower 234 through a gas inlet of the blower 234 disposed below the motor portion.

In some embodiments, the first gas inlet 231, the second gas inlet 232, and the third gas inlet 233 may be disposed vertically at a height close to the motor portion of the blower 234 in the blower cavity 230, and away from the base portion of the blower 234. When the gasflow enters the blower cavity 230 through the first gas inlet 231, the second gas inlet 232, and the third gas inlet 233, the gasflow may flow from the top to the bottom to cool the blower 234, so that the blower 234 may be maintained at a suitable temperature range, thereby maintaining a better working performance.

FIG. 2D is a schematic top view illustrating a structure of a noise reduction device (without a noise reduction top housing) according to some other embodiments of the present disclosure.

In some embodiments, a porous acoustic plate 250 and/or a sound-absorbing cotton may be disposed in the gas channel 240, referring to FIGS. 3E to 3F, and FIGS. 4A to 4F, and the related illustrations for the specific structure of the porous acoustic plates 250, which will not be repeated here.

In some embodiments, the noise reduction device 200 may further include a main outlet (not shown in the figure), which is connected to a breather pipe (as the gas inlet channel 521-1 shown below) of a reservoir or a cover plate without a reservoir inside the respiratory ventilation apparatus disposed at a downflow of a gas path of the noise reduction device 200 for transferring pressurized high pressure gas. Ultimately, the main outlet may be connected to an external user interface 7000. The gas pressurized by the blower may be used to replace, control, or modify a spontaneous respiratory movement of a human body. In some embodiments, the main outlet may communicate through a sealing structure with the breathing pipe of a connecting device (including a reservoir or a cover plate without a reservoir) at the downflow of the gas path, which effectively avoid a gas leakage at the connection. Referring to FIGS. 5A to 51 and the related descriptions for the specific structure of the sealing structure, which are not repeated here.

In the embodiment above, the gas channel of the noise reduction device in the respiratory ventilation apparatus adopts the design where the great and small cavities are spaced apart. In this way, when the gas flows through various spaces with different sizes of the top-down sectional region in the gas channel, the noise of different frequencies may be absorbed, so that the noise of the gasflow flow may be gradually attenuated. The noise reduction device may adopt at least two cavity gas inlets to divert the gas into the blower cavity, which reduce an impact of the turbulence in the gasflow, attenuates the noise of the gasflow into the blower cavity, and the cavity gas inlets at different positions may eliminate the noise of different frequency bands. The gas inlet pipe is separated may be divided into the plurality of gas inlet sub-pipes, which diverts the gasflow into a plurality of long advective flows. The structural design may reduce the noise generated by the gasflow in the gas inlet pipe. When the gas enters the first space from the gas inlet pipe, the gasflow turns at an angle of more than 900 within the first space, and when the gasflow changes direction, some of the sound waves of the gasflow may be mutually cancelled, which eliminate some of the noise of the frequency band, thereby in turn reducing the noise when the gasflow is transmitted. The at least two cavity gas inlets of the noise reduction device may be disposed vertically at a height close to the motor portion of the blower in the blower cavity, and the gasflow may flow from the top to the bottom to cool the blower, so that the blower may be maintained at an appropriate temperature range, thereby maintaining a better working performance.

FIG. 3A is a 3D schematic diagram illustrating a structure of a noise reduction device according to some other embodiments of the present disclosure; and FIG. 3B is a 3D schematic diagram illustrating a structure of a noise reduction device (without a noise reduction top housing) according to some other embodiments of the present disclosure.

In some embodiments, referring to FIGS. 3A and 3B, a noise reduction device 300 may include a noise reduction housing 310, a gas inlet structure, and a blower cavity 330. The gas inlet structure may be in a form of a gas inlet pipe 320. The blower cavity 330 may be disposed within the noise reduction housing 310. A gas channel 340 may be formed between an inner wall of the noise reduction housing 310 and an outer wall of the blower cavity 330. The gas inlet pipe 320 may be configured to connect to an externally delivered gas (e.g., gas) to direct the gas through the gas inlet pipe 320 into the noise reduction device 300. The gas channel 340 may be configured to transfer the gas transmitted from the gas inlet pipe 320. The blower cavity 330 may be configured to accommodate a blower. In some embodiments, referring to FIG. 3A, the noise reduction housing 310 may include a noise reduction top housing 311 and a noise reduction bottom housing 312. The noise reduction top housing 311 may be fixedly connected to the noise reduction bottom housing 312 by a screw connection, etc., and the blower cavity 330 and the gas channel 340 may be integrated within the noise reduction housing 310.

FIG. 3C is a schematic top view illustrating a structure of a noise reduction device (without a noise reduction top housing) according to some other embodiments of the present disclosure; and FIG. 3D is a schematic front view illustrating a structure of an inner cavity of a gas inlet pipe of a noise reduction device according to some other embodiments of the present disclosure.

In order to facilitate a demonstration of an internal structure of the noise reduction device 300, FIG. 3B is not mounted with the noise reduction top housing 311. Referring to FIGS. 3B and 3C, the gas channel 340 may be disposed around an outer wall of the blower cavity 330. In some embodiments, a blower mounting cavity 350 may be disposed inside the blower cavity 330. The blower mounting cavity 350 may be configured to mount a blower.

In some embodiments, a cavity formed by the outer wall of the blower cavity 330, the inner wall of the noise reduction housing 310, the upper surface of the noise reduction bottom housing 312, and the lower surface of the noise reduction top housing 311 may be the gas channel 340. In some embodiments, the gas channel 340 may also be a gas channel structure independently disposed between the inner wall of the noise reduction housing 310 and the outer wall of the blower cavity 330. Referring to FIG. 3C, an external gas (e.g., gas) may enter the gas channel 340 from the gas inlet pipe 320, and form a gasflow with a certain flow direction (as indicated by the dotted arrow in FIG. 3C) within the gas channel 340. It should be noted that an actual flow direction in the gas channel 340 may be complex, and the dotted arrows in FIG. 3C are merely exemplary flow direction references drawn for the purpose of facilitating a better understanding of the present disclosure, and do not constitute a limitation of the present disclosure.

In some embodiments, referring to FIG. 3C, from a top-down perspective, an outer wall section of the blower cavity 330 may be a portion of a circle, an inner wall section of the noise reduction housing 310 may be a portion of a subcircular shape, and the gas channel 340 may be a portion of a concentric ring.

A shape of the gas channel 340 may be determined primarily by a shape of the outer wall of the blower cavity 330 and a shape of the inner wall of the noise reduction housing 310. In some embodiments, a shape of the gas channel 340 may also be a section of a cavity formed by an inner circle and outer square, as viewed from the top-down perspective. In some embodiments, the shape of the gas channel 340 may also be the section of the cavity formed by an inner square and an outer circle. In some embodiments, the shape of the gas channel 340 may also be the section of the cavity formed by two other forms of polygon inside and outside.

The gas channel 340 may not only be configured to transmit the gas entering from the gas inlet pipe 320 into the blower cavity, but may also be configured to reduce the noise generated by the gasflow. The noise may be gradually reduced by the gas channel when the gas is flowing through the gas channel 340, so as to perform the noise reduction. The annular gas channel may be designed to lengthen a length of the gas channel 340, and the longer the length of the gas channel 340, the better the noise reduction effect. By disposing the gas channel 340 around the outside of the blower cavity 330, when the annular gas channel 340 attenuates the noise generated by the gasflow inside the gas channel 340, it may attenuate the noise generated by the operation of the blower within the blower cavity 330.

In some embodiments, the gas inlet pipe 320 may have a gas outlet 321. The gas outlet 321 may be disposed at a beginning end of the gas channel 340, and the blower cavity 330 may be disposed with the cavity gas inlet 331. The cavity gas inlet 331 may be disposed on a sidewall of the blower cavity 330 at the tail end of the gas channel 340. The cavity gas inlet 331 may allow the gas channel 340 to communicate with an inside of the blower cavity 330. The external gas (e.g., gas) may enter through the gas inlet pipe 320, the gas may flow into the gas channel 340 through the gas outlet 321, and the gasflow may flow along an arcuate (which may be a portion of the circle) gas channel 340 and then through the cavity gas inlet 331 into the blower cavity 330.

A magnitude of an angle between a first connecting line connecting a center of the gas channel 340 and the gas outlet 321 and a second connecting line between the cavity gas inlet 331 and the center of the gas channel 340 along a direction of extension of the gas channel 340 may determine the length of the gas channel 340. The longer the length of the gas channel 340, the better the noise reduction effect of the noise reduction device. Thus, the magnitude of an angle between the first connecting line connecting the center of the gas channel 340 and the gas outlet 321 and the second connecting line between the cavity gas inlet 331 and the center of the gas channel 340 along the direction of extension of the gas channel 340 may be designed based on a length required by the gas channel 340. The length of the gas channel 340 may be designed based on a level of noise reduction required, and may also be specifically adjusted based on a gas resistance, a gas pressure, and other requirements of the gasflow in the gas channel 340.

In some embodiments, referring to FIG. 3C, the center of the circle of the blower cavity 330 and the center of the circle of the gas channel 340 may coincide in atop-down view. An angle between the first connecting line (as dotted line M in FIG. 3C) connecting the gas outlet 321 of the gas inlet pipe 320 and the center (i.e., the center of the circle) of the gas channel 340 and the second connecting line (as dotted line N in FIG. 3C) connecting the cavity gas inlet 331 and the center (i.e., the center of the circle) of the gas channel 340 along the direction of extension of the gas channel 340 may be greater than 180°. It may also be understood that the arc formed by the gas channel 340 along the outer wall of the blower cavity 330 may be greater than half of the circle. As shown in the embodiment, the gas channel 340 of the noise reduction device 300 may be in a form of an arc that is slightly greater than half of the circle, and the extended arcuate gas channel 340 may have a sufficient noise reduction effect, and may also allow the noise reduction device 300 to have a smaller size.

In some embodiments, the angle between the first connecting line (as dotted line M in FIG. 3C) connecting the gas outlet 321 of the gas inlet pipe 320 and the center (i.e., the center of the circle) of the gas channel 340 and the second connecting line (as dotted line N in FIG. 3C) connecting the cavity gas inlet 331 and the center (i.e., the center of the circle) of the gas channel 340 along the direction of extension of the gas channel 340 may be greater than 240°. It may also be appreciated that the arc formed by the gas channel 340 along the outer wall portion of the blower cavity 330 may be greater than two-thirds of the circle.

In some embodiments, the angle between the first connecting line (as dotted line M in FIG. 3C) connecting the gas outlet 321 of the gas inlet pipe 320 and the center (i.e., the center of the circle) of the gas channel 340 and the second connecting line (as dotted line N in FIG. 3C) connecting the cavity gas inlet 331 and the center (i.e., the center of the circle) of the gas channel 340 along the direction of extension of the gas channel 340 may be greater than 270°. It can also be appreciated that the arc formed by the gas channel 340 along the outer wall portion of the blower cavity 330 may be greater than three-fourths of the circle. As shown in FIG. 3C, the cavity gas inlet 331 may be close to the cavity gas outlet 333, and an outer portion of the outer wall of the blower cavity 330 may be the gas channel 340 except for a small portion connecting to the cavity gas outlet 333. The noise reduction device 300 shown in the embodiment has the longest gas channel 340 and correspondingly the best noise reduction effect.

In some embodiments, referring to FIG. 3B, the gas inlet pipe 320 may have a bending structure, such that when the external gas enters from the gas inlet pipe 320, the gasflow within the gas inlet pipe 320 needs to be turned to flow into the gas channel 340, which effectively attenuates a whistling generated by a friction between the gas inlet pipe 320 and the gas. When the gasflow changes direction, the sound waves of the gasflow may generate some mutual cancellation, which eliminate some of the noise in the frequency band, and thus reduce the noise when the gasflow is transmitted.

In some embodiments, referring to FIG. 3B, the bending structure may include bending upwardly such that the gas inlet 322 of the gas inlet pipe 320 may be disposed at a different height from the gas outlet 321, the gas inlet 322 of the gas inlet pipe 320 being lower than the height of the gas outlet 321. In some embodiments, the bending structure may include bending downward such that the gas inlet 322 of the gas inlet pipe 320 is disposed at the different height from the gas outlet 321, the gas inlet 322 of the gas inlet pipe 320 being higher than the gas outlet 321.

In some embodiments, the gas inlet pipe 320 may have the bending structure that allows external gas to enter the gas inlet pipe 320, and a section of the gas inlet 322 may have a first gasflow direction. When the gas passes through the bending structure, the gasflow may be turned, and a section at the gas outlet 321 may have a second gasflow direction. The first gasflow direction of the gas inlet 322 may be different from the second gasflow direction of the gas outlet 321. That is, the gasflow may undergo a turning of the gasflow direction in the gas inlet pipe 320.

In some embodiments, referring to FIG. 3B, the bending structure may include bending upward such that the gas outlet 321 may be higher than the gas inlet 322. In some embodiments, the bending structure of the gas inlet pipe 320 may enable the gas inlet pipe 320 to include a horizontal direction section and a vertical direction section. The gas inlet 322 may be disposed in the horizontal direction section and the gas outlet 321 may be disposed in the vertical direction section. The first gasflow direction may be a horizontal direction; the second gasflow direction may be a vertical direction. When the gas enters through the gas inlet 322 of the gas inlet pipe 320, it may first pass through the horizontal direction section to form the first gasflow direction, and then through the bending structure where the gasflow turns upwardly and flows to the vertical direction section to form the second gasflow direction, and finally, the gas may flow to the gas channel 340 through the gas outlet 321.

In some embodiments, the bending structure may include one or more bending, so that after the outside gas enters the gas inlet pipe 320, the gasflow may turn several times inside the gas inlet pipe 320. Under a premise that conditions like a gas resistance, a gas pressure, etc. are satisfied, by disposing a multiple turning structure, a better noise reduction effect may be implemented. For example, the bending structure may include two bends, and when the outside gas enters the gas inlet 322 of the gas inlet pipe 320, the gasflow direction may be horizontal, and after the first bending, the gasflow direction turns vertical, and after the second bending, the gasflow direction turns to horizontal. For another example, the bending structure may include three bendings. When the outside gas enters the gas inlet 322 of the gas inlet pipe 320 in the horizontal direction, the gas turns to a vertical direction after the first bending, and turns to a horizontal direction after the second bending, and after the third bending, the gas turns to the vertical direction again.

FIG. 3D is a schematic front view illustrating a structure of an inner cavity of a gas inlet pipe of a noise reduction device according to some other embodiments of the present disclosure. In some embodiments, referring to FIG. 3D, the gas inlet pipe 320 may be a circular pipe including a horizontally oriented section and a vertically oriented section. The gas inlet 322 may be disposed at an initial end of the horizontally oriented section, and may have an inner wall that is circular in a front view direction. In some embodiments, the end of the gas inlet 322 may be disposed perpendicular to the horizontally oriented section of the gas inlet pipe 320, that is, disposed in a vertical plane. The gas outlet 321 may be disposed at the end of the vertically oriented section, and may have an inner wall that is circular in a top-down direction. In some embodiments, the end of the gas outlet 321 may be disposed vertical to the vertically oriented section of the gas inlet pipe 320, that is, on a horizontal plane.

In some embodiments, an overall height of the vertically oriented section of the gas inlet pipe 320 (referring to H in FIG. 3D) may be 2 to 2.8 times a diameter of the horizontally oriented section of the gas inlet pipe 320 (referring to the inner diameter D of the gas inlet 322 in FIG. 3D). In some embodiments, the overall height of the vertically oriented section of the gas inlet pipe 320 (referring to H in FIG. 3D) may be 2.2 to 2.6 times the diameter of the horizontally oriented section of the gas inlet pipe 320 (referring to the inner diameter D of the gas inlet 322 in FIG. 3D). In some embodiments, the overall height of the vertically oriented section of the gas inlet pipe 320 (referring to H in FIG. 3D) may be 2.4 times the diameter of the horizontally oriented section of the gas inlet pipe 320 (referring to the inner diameter D of the gas inlet 322 in FIG. 3D).

In some embodiments, the gas inlet pipe 320 may include a plurality of bending structures, and an overall height of the vertically oriented section of the gas inlet pipe 320 may be a total height of the plurality of bend sections in the vertical direction.

In some embodiments, a ratio of the overall height of the vertically oriented section of the gas inlet pipe 320 to the diameter of the horizontally oriented section of the gas inlet pipe 320 may be specifically adjusted based on requirements such as a gas resistance, a gas pressure, etc.

FIG. 3E is a schematic diagram illustrating a 3D structure of a porous sound-absorbing plate in a noise reduction device according to some other embodiments of the present disclosure. In some embodiments, a porous sound-absorbing plate 360 and/or a sound-absorbing cotton may be disposed in the gas channel 340, and a number of sound-absorbing holes 364 may be disposed in a thickness direction of the porous sound-absorbing plate 360 for further noise reduction.

In some embodiments, the sound-absorbing cotton may be disposed in the gas channel 340 (no accompanying figures are given for this embodiment). The sound-absorbing cotton may be fitted to an inner wall (including sidewalls, a top surface, and a bottom surface) of the noise reduction housing 310 and/or the outer walls of the blower cavity 330. When a gasflow flows in the gas channel 340, the sound-absorbing cotton disposed on at least one of the surfaces around the gas channel 340 may absorb a noise generated during the flow of the gasflow as the gasflow flows through the gas channel 340.

In some embodiments, the sound-absorbing cotton may be fitted to the bottom surface of the noise reduction housing 310. The gasflow in the gas inlet pipe 320 may pass through the bending structure to elevate a gasflow flow direction, so that the gasflow may be vertically oriented when it comes out of the gas outlet 321. Then the gasflow may reflect after encountering atop wall of the gas channel 340 (i.e., the lower surface of the noise reduction top housing 311), so as to enable the gasflow to flow downward to the sound-absorbing cotton below, which facilitates an absorption of more sound waves by the sound-absorbing cotton for noise reduction of the sound generated by the gasflow.

In some embodiments, the sound-absorbing cotton may be a non-porous sound-absorbing cotton. The non-porous sound-absorbing cotton may be disposed around the gas channel 340. When the gasflow flows in the gas channel 340, the non-porous sound-absorbing cotton may not only absorb a portion of the noise, but also reflect the sound waves. The reflected sound waves may be absorbed and weakened again by the non-porous sound-absorbing cotton of the other orientations, so as to achieve a better noise reduction effect.

In some embodiments, the porous sound-absorbing plate 360 may also be placed in the gas channel 340, with a plurality of sound-absorbing holes 364 being disposed in the thickness direction of the porous sound-absorbing plate 360. When the gasflow in the gas channel 340 flows through the porous sound-absorbing plate 360, at least a portion of the sound waves of the gasflow may enter the sound-absorbing holes 364 to reduce the noise of the gasflow.

The sound-absorbing holes 364 of different apertures on the porous sound-absorbing plate 360 may have different effects on eliminating the gasflow noise in different frequency ranges. The sound-absorbing holes 364 of smaller apertures may be suitable for eliminating high-frequency noises, and the sound-absorbing holes 364 of greater apertures may be suitable for eliminating low-frequency noises. Based on this, in some embodiments, the porous sound-absorbing plate 360 may include a plurality of regions, and the apertures of the sound-absorbing holes 364 on the various regions may be disposed in accordance with a preset rule. For example, the sound-absorbing holes 364 of a plurality of apertures may eliminate gasflow noises in different frequency band ranges, and thus the noise reduction effect of the noise reduction structure may be effectively guaranteed.

In some embodiments, referring to FIG. 3E, the plurality of regions of the porous sound-absorbing plate 360 may include a porous top plate 361, a porous side plate 362, and a porous bottom plate 363. The plurality of porous top plates 361, the plurality of porous side plates 362, and the plurality of porous bottom plates 363 may be connected to form a three-way enclosing structure. The gas that enters from the gas inlet pipe 320 may form a gasflow within the gas channel 340. A number of sound-absorbing holes 364 may play a role in absorbing the sound when the gasflow passes through the porous sound-absorbing plate 360 to achieve the purpose of noise reduction. In some embodiments, the apertures of the sound-absorbing holes 364 of the porous top plate 361, the porous side plate 362, and the porous bottom plate 363 may be the same.

In some embodiments, the apertures of the sound-absorbing holes 364 of the porous top plate 361, the porous side plate 362, and the porous bottom plate 363 may also be different. In some embodiments, the apertures of the sound-absorbing holes 364 in the porous side plates 362 may be greater than the apertures of the sound-absorbing holes 364 in the porous top plate 361 and/or the porous side plates 362. In some embodiments, the aperture of the sound-absorbing hole 364 on the porous top plate 361 may be smaller than the aperture of the sound-absorbing hole 364 on the porous bottom plate 363.

In some embodiments, the porous top plate 361, the porous side plate 362, or the porous bottom plate 363 may each be divided into a plurality of regions. In each region, the aperture of the sound-absorbing holes 364 may be a set value according to an actual noise reduction frequency band.

In some embodiments, the plurality of regions of the porous acoustic plate 360 may be the plurality of regions that are spatially divided in a vertical direction along the gasflow direction within the porous acoustic plate 360. Each of the regions may include the porous top plate 361, the porous side plate 362, and the porous bottom plate 363.

FIG. 3F is a schematic diagram illustrating a top-view structure of a porous sound-absorbing plate in a noise reduction device according to some other embodiments of the present disclosure. In some embodiments, referring to FIG. 3F, the porous sound-absorbing plate 360 may include three regions, a first region 365, a second region 366, and a third region 367. Apertures of the sound-absorbing holes 364 in the first region 365, the second region 366, and the third region 367 may be set in accordance with a preset rule. In some embodiments, the apertures of the sound-absorbing holes 364 in the first region 365, the second region 366, and the third region 367 may be the same.

In some embodiments, the apertures of the sound-absorbing holes 364 in the first region 365, the second region 366, and the third region 367 may also be different. In some embodiments, the apertures of the sound-absorbing holes 364 in the second region 366 may be greater than the apertures of the sound-absorbing holes 364 in the first region 365 and/or the third region 367. In some embodiments, the apertures of the sound-absorbing holes 364 in the first region 365 may be greater than the apertures of the sound-absorbing holes 364 in the third region 367. In some embodiments, the porous sound-absorbing plate 360 may also include more than three (e.g., four, five, six, etc.) regions, with the apertures of the sound-absorbing holes 364 in each region having different apertures.

In some embodiments, the size of the apertures of the sound-absorbing holes 364 in the various regions may be set according to the frequency band of the noise of the gasflow passing through the porous acoustic plate 360.

In some embodiments, the porous acoustic plate 360 may also have sound-absorbing holes 364 of different apertures distributed irregularly regardless of region.

In some embodiments, a plate thickness of the porous acoustic plate 360 may be 1.5 mm, and the apertures of the sound-absorbing holes 364 of the porous acoustic plate 360 may be less than or equal to the plate thickness of the porous acoustic plate 360.

In some embodiments, the porous acoustic plate 360 may be disposed with sound-absorbing holes 364 with different apertures. The sound-absorbing holes 364 with different aperture diameters may absorb the noise in different frequency bands during the gasflow. In some embodiments, the apertures of the sound-absorbing holes 364 of the porous sound-absorbing plate may be of a variety of apertures such as 0.6 mm, 0.9 mm, 1.0 mm, 1.2 mm, 1.5 mm, etc.

In some embodiments, the porous acoustic plate 360 and the sound-absorbing cotton may be disposed in the gas channel 340. The sound-absorbing cotton and the porous sound-absorbing plate 360 may be sequentially fitted to the inner wall of the noise reduction housing 310. That is, the inner and outer walls of the sound-absorbing cotton are fitted to the outer wall of the porous sound-absorbing plate 360 and the inner wall of the noise reduction housing 310, respectively. When the gasflow flows in the gas channel 340, the porous sound-absorbing plate 360 may absorb a portion of the noise, and a portion of the noise may pass through a plurality of sound-absorbing holes 364 of the porous sound-absorbing plate to be absorbed by the sound-absorbing cotton, resulting in a better noise reduction effect.

In some embodiments, referring to FIG. 3A, the cavity gas inlet 331 may be disposed on a sidewall of the blower cavity, at the tail end of the gas channel 340 and near the top of the gas channel 340. The gas channel 340 and the interior of the blower cavity 330 may communicate through the cavity gas inlet 331. The cavity gas inlet 331 may be disposed vertically at a height close to a motor portion of a blower in the blower cavity 330 such that the gasflow passes through the cavity gas inlet 331 into the blower cavity 330. When the gasflow enters the blower cavity 330 through the cavity gas inlet 331, the gasflow may flow from top to bottom to dissipate a heat of the motor portion of the blower, so that the blower may maintain a suitable temperature range and maintain a better working performance. The gasflow may circulate from top to bottom, and finally enters the blower from the gas inlet below the blower motor. The blower may pressurize the incoming gas.

In some embodiments, a plurality of cavity gas inlets 331 may be disposed on the sidewall of the blower cavity. For example, referring to FIG. 3G, two cavity gas inlets 331 may be disposed. One cavity gas inlet 331 may be disposed at the beginning end of the gas channel 340, and close to the top of the gas channel 340 in a height direction; the other cavity gas inlet 331 may be disposed at the tail end of the gas channel 340, and close to the middle of the gas channel 340 in the height direction. In some embodiments, the arrangement of disposing a flow collection module on two sides of the cavity gas inlet 331 that is located at the beginning end of the gas channel 340 helps to achieve a stable flow, so as to improve a flow measurement accuracy.

In some embodiments, referring to FIGS. 3B and 3C, the noise reduction device 300 may further include a water collection cavity 332, the blower cavity 330 may further include a cavity gas outlet 333, and the noise reduction housing 310 may be disposed on a main outlet 313. The cavity gas outlet 333 may communicate with the main outlet 313 through the water collection cavity 332. The main outlet 313 may be disposed vertically at a height higher than the water collection cavity 332, so that the gas may enter the water collection cavity 332 from the cavity gas outlet 333 and then lifted up to the main outlet 313 to flow out. The main outlet 313 may communicate with a breather pipe of a reservoir or a cover plate without a reservoir (the gas inlet channel 521-1 shown below) located at a downstream of a gas path of the noise reduction device 300 inside the respiratory ventilation apparatus. The main outlet 313 may be configured to transfer pressurized gas, and ultimately to communicate with an external user interface 7000. The gas pressurized by the blower may be used to replace, control, or change a spontaneous breathing movement of a human body.

In some embodiments, when the downflow of the gas path of the noise reduction device 300 is a reservoir, the main outlet 313 may be in sealing connection with the gas inlet channel 630 of the reservoir in FIG. 6A by a sealing structure. For specific structures regarding the reservoir, please refer to relative contents in FIGS. 6A to 61 and FIGS. 7A-7G below. A reservoir upper housing 611 of the reservoir and a reservoir lower housing 612 of the reservoir may be internally hollow, and the two may cooperate to form a cavity of the reservoir for holding liquid. The gas inlet channel 630 and a gas outlet channel 640 may be both disposed in the reservoir upper housing 611. When the respiratory ventilation apparatus is placed horizontally upward (i.e., in a correct state of use), the gas inlet channel 630 may be disposed above the reservoir cavity. After flowing out from the cavity gas outlet 333, the gas pressurized by the blower may be lifted upwardly into the gas inlet channel 630 of the reservoir through the main outlet 313. The gas inlet channel 630 of the reservoir may be disposed on top of the cavity of the reservoir. In this way, the liquid in the reservoir may not flow back into the main body.

The respiratory ventilation apparatus may be disposed in a fixed direction (i.e., the reservoir cavity of the reservoir may be disposed at the bottom, and the gas inlet channel may be disposed at the top of the reservoir cavity), and if there is an incorrect position of placement (e.g., 900 sideways) in the process of use, the performance of the respiratory ventilation apparatus may be affected. Negative effects may include: not being able to run at full power or generating strange noises, or even causing damage to the blower due to the return of liquid in the reservoir. Based on this, in some embodiments, the main body may be mounted with a position and attitude detection device configured to detect whether the respiratory ventilation apparatus is in a correct positional attitude, and if an incorrect position and attitude is detected, an alarm message may be issued (e.g., an alarm light may flash, a voice alarm may be issued, etc.).

In some embodiments, the cavity gas outlet 333 may be disposed in the vertical direction at a height higher than the bottom surface of the water collection cavity 332, and specifically, a lowermost end of the cavity gas outlet 333 in the vertical direction may have a certain height from the bottom surface of the water collection cavity 332, so that a recess-type cavity may be formed from the bottom surface of the water collection cavity 332 to the lower end of the cavity gas outlet 333. When there is a return flow of water from the reservoir, the cavity of the water collection cavity 332 may be used for temporarily storing a portion of the water, preventing the water from flowing into the blower cavity 330, and serving to protect the blower in the blower cavity 330. In some embodiments, the main outlet 313 may be disposed on the noise reduction top housing 311, the height of the main outlet 313 may be higher than the cavity gas outlet 333. The gas coming out of the cavity gas outlet 333 may be lifted within the water collection cavity 332 before entering the other structures of the respiratory ventilation apparatus through the main outlet 313, and the gas may be noise-reduced again as it is lifted (i.e., turned) within the water collection cavity 332. Therefore, the disposing of the water collection cavity 332 may not only prevent the water from flowing back into the blower cavity 330, but also help to reduce the noise. A shape of a cross-section of a pipeline of the main outlet 313 may be designed according to the actual situation, e.g., circular, oval, or oblate, which is not limited here.

In the noise reduction device in the respiratory ventilation apparatus of the above embodiment, the gas channel may be disposed around the outer side of the blower cavity, and while the annular gas channel attenuates the noise generated by the gasflow flowing inside thereof, the annular gas channel may also attenuate the noise generated by the blower operation inside the blower cavity. The bending structure of the gas inlet pipe may be designed so that the gasflow in the gas inlet pipe needs to be turned to flow into the gas channel, which effectively attenuates a whistling sound generated by a friction between the gas inlet pipe and the gas, and the sound waves of the gasflow may generate some mutual cancelling when the gasflow is changing direction, so as to eliminate some of the noise in the frequency band, and reduce the noise of the gasflow transmission. The gas outlet of the gas inlet pipe may be vertically upward, so that the gasflow from the outlet may be vertically upward, and the gasflow may meet the top wall of the gas channel (i.e., the lower surface of the top housing) and then bounce, so that the gasflow may flow downward to the sound-absorbing cotton below, and the sound-absorbing cotton may absorb more noise. In this way, the noise reduction of the sound generated by the flow of gas may be implemented. The porous sound-absorbing plate and/or the sound-absorbing cotton may be placed in the gas channel of the noise reduction device to further reduce the noise. The position of the cavity gas inlet may be at a height close to the motor portion of the blower in the blower cavity, and the gasflow may flow from top to bottom to cool the blower, so that the blower may maintain a proper temperature range and maintain a better working performance.

In some embodiments, the noise reduction device may be mounted on the main body by snap-fit. For example, at least one buckle may be disposed on the housing of the noise reduction device, and at least one slot may be disposed correspondingly on the main body. When the noise reduction device is mounted inside the main body, the buckle may be connected to the corresponding slot, so as to quickly position the noise reduction device and fixedly mount the noise reduction device inside the main body.

FIG. 4A is a schematic diagram illustrating a structure of a porous sound-absorbing plate according to some embodiments of the present disclosure.

In some embodiments, a porous acoustic plate may be a porous sound-absorbing plate 400. In some embodiments, the porous sound-absorbing plate 400 refers to a thing plate disposed with a plurality of micro through holes 411. In some embodiments, the through holes 411 may be disposed in an array along a thickness direction of the porous sound-absorbing plate 400 through the porous sound-absorbing plate 400. For example, the through holes 411 on the porous sound-absorbing plate 400 of FIG. 4A may be disposed in an array of 7 per column, totaling 16 columns.

It should be noted that the present disclosure does not limit the dispose of the through holes (e.g., a row spacing, a column spacing, and an array shape of the through holes 411), and the dispose may be adjusted according to the actual needs.

In some embodiments, the porous sound-absorbing plate 400 may serve as a noise reduction structure for the gasflow. One end of the porous sound-absorbing plate 400 may be in contact with the gasflow, so that the gasflow may pass through the through holes into a sound-absorbent material (e.g., a sound-absorbent cotton) or other components (e.g., a noise reduction housing, as described above), which converts a mechanical energy of the gasflow into a thermal energy, thereby reducing the noise generated by a mechanical vibration of the gas.

In some embodiments, the through hole 411 may include through hole of a first type as well as through hole of a second type. Wherein an aperture of through hole of the first type may be smaller than an aperture of through hole of the second type. As shown in FIG. 4A, the through holes in even-numbered columns of the porous sound-absorbing plate 400 may be different in size from the through holes in odd-numbered columns. The through holes 411 in the even-numbered columns may be through hole of the first type and the through holes 411 in the odd-numbered columns may be through hole of the second type.

In some embodiments, the sizes of the through holes 411 may be determined based on sound-absorbing requirements of the porous sound-absorbing plate. For example, through hole of the first type may be 0.8 mm and through hole of the second type may be 1.2 mm. Through hole of the first type as well as through hole of the second type differentiated based on this mode may absorb noise of different frequencies, thereby enriching a sound-absorbing range of the porous sound-absorbing plate.

FIG. 4B is a sectional view illustrating the porous sound-absorbing plate of FIG. 4A along axis AA.

As shown in FIG. 4B, an end of the porous sound-absorbing plate 400 in contact with a gasflow may be denoted as the first surface 413, an end in contact with a sound-absorbing material may be denoted as a second surface 412, an opening of the through hole 411 on the first surface 413 may be denoted as the first opening 416, an opening of the through hole 411 on the second surface 412 may be denoted as a second opening 415, and a surface of the through hole 411 that connects the first opening 416 and the second opening 415 within the porous sound-absorbing plate 400 may be denoted as the sidewall of the through hole 414.

In some embodiments, an aperture of the first opening 416 may be smaller than an aperture of the second opening 415, so that the sidewall of the through hole 414 forms a reflective slope toward the sound-absorbing material. When a gasflow enters the sound-absorbing material along the through hole 411, a portion of the gasflow may be reflected by the sound-absorbing material, and that portion of the reflected gasflow may flow directly out of the through hole 411 when the first opening 416 is the same size as the second opening 415. And based on the reflective slope formed by the sidewall of the through hole 414 toward the sound-absorbing material in the present disclosure, that portion of the reflected gasflow may be rebounded again by the sidewall of the through hole 414 and thus enter the sound-absorbing material again, thereby improving an efficiency of noise reduction.

In some embodiments, the sidewall of the through hole 414 may be in a preset shape in a section of the porous sound-absorbing plate 400 in the thickness direction. The preset shape may include a straight line, a curve, or a combination thereof. The preset shape may be determined based on the apertures of the first opening 416 and the second opening 415. For example, if a difference between the aperture of the first opening 416 and the aperture of the second opening 415 is relatively small, in order to minimize a processing difficulty, the pre-determined shape may be the straight line, i.e., the sidewall of the through hole 414 may communicate with the first opening 416 and the second opening 415 in a straight line shaped lateral side. For another example, if the difference between the aperture of the first opening 416 and the aperture of the second opening 415 is great, the preset shape may be the curve to increase a reflectivity of the sidewall of the through hole 414.

In some embodiments, the gasflow may enter the sound-absorbing material through the first opening 416, and a sound-absorbing performance of the porous sound-absorbing plate 400 may be related to the diameter of the first opening 416. A sound-absorbing constant K of the porous sound-absorbing plate 400 may be a function of a diameter d of the first opening 416 as well as a resonant frequency f₀ of the porous sound-absorbing plate. The acoustic absorption constant of the porous sound-absorbing plate 400 may be

$$K = d \times \sqrt{{f}_{0} / 10} .$$

When a relative acoustic resistance r of the porous sound-absorbing plate 400 is 1, K may be smaller than 10. A porous sound-absorbing plate sound absorber may have a wide band nature, which is near a limit of the porous sound-absorbing plate sound absorber. A required cavity depth may be about one-quarter wavelength. When K is greater than 2, the nature of the porous sound-absorbing plate sound absorber may be basically the same as narrower band but the cavity is shallow; because of the increase in the value of K, the depth of the cavity decreases faster; in the value of K is greater than 1, it is possible that the frequency band is still quite wide, but the depth of the cavity has been greatly reduced.

When the value of r is relatively great (e.g., r>1), the maximum sound-absorbing coefficient needs to be reduced, and a bandwidth may be determined by the values of k×\sqrt{r} and (1+r). In other words, a greater value of r may reduce the sound-absorbing coefficient, while widening the sound-absorbing band, a multi-sound-absorbing band nature of the porous sound-absorbing plate sound absorber may greatly enhance a performance of wideband design.

Based on the above constraints, the resonance frequency f₀ of the porous sound-absorbing plate and the relative acoustic resistance r of the porous sound-absorbing plate may be determined experimentally, and the sound-absorbing constant k of the porous sound-absorbing plate 400 may be determined based on sound-absorbing requirements of the porous sound-absorbing plate 400, thereby determining the aperture of the first opening 416.

In some embodiments, the aperture of the first opening 416 may be generally smaller than the thickness of the porous sound-absorbing plate 400. The thickness of the porous sound-absorbing plate 400 for the noise reduction may be generally 1.5 mm, the aperture of the first opening 416 may take a value in the range of 0.8 mm to 1.4 mm.

In some embodiments, the porous sound-absorbing plate 400 may be disposed with a first porous sound-absorbing plate as well as a second porous sound-absorbing plate based on a gasflow direction. The first porous sound-absorbing plate may be a portion of the porous sound-absorbing plate disposed along a first plane, and the second porous sound-absorbing plate may be a portion of the porous sound-absorbing plate disposed vertical to (or nearly vertical to) the first plane. The first plane may be a parallel plane of the plane formed by the gasflow. For example, in the space of FIG. 4D, where the gasflow is moving in a horizontal plane, the first plane may be the xy plane and the parallel plane thereof.

Considering that different through hole apertures of the porous sound-absorbing plate may have different effects in eliminating the gasflow noise in different frequency band ranges, smaller through hole apertures may be suitable for eliminating a high-frequency noise, and greater through hole apertures may be suitable for eliminating a low-frequency noise. In some embodiments, the through hole aperture on the first porous sound-absorbing plate may be smaller than the through hole aperture on the second porous sound-absorbing plate. The through hole on the first porous sound-absorbing plate may be a first type through hole and the through hole on the second porous sound-absorbing plate may be a second type through hole. Based on this, by designing the apertures of the through holes on the first porous sound-absorbing plate and the apertures of the through holes on the second porous sound-absorbing plate as apertures of different sizes, the noise in different frequency band ranges may be eliminated, thereby effectively ensuring a noise reduction effect of the noise reduction structure.

In some other embodiments, the second surface 412 of the porous sound-absorbing plate 400 may be a sealed back plate, and a semi-sealed cavity may be disposed in the porous sound-absorbing plate 400 close to the second surface 412. The plurality of through holes 411 on the porous sound-absorbing plate 400 may communicate with the cavity. When the gasflow in the gas channel passes through the porous sound-absorbing plate 400, the noise sound waves may enter the cavity inside the porous sound-absorbing plate 400 from the through holes 411 and may be eliminated, which effectively improves the noise reduction effect of the porous sound-absorbing plate 400.

In some embodiments, when the gasflow to be noise-reduced has a complex spatial structure in the space, the gasflow to be noise-reduced may be split in sections, so that each section of gasflow flow in the first plane corresponding to the section of gas path.

In some embodiments, the porous sound-absorbing plate may further include a separating component disposed on the first surface of the porous sound-absorbing plate. The separating component may include two separating component surfaces disposed opposite to each other, and the separating component surfaces may be in contact with the gasflow, so as to realize effects of guiding, diverting, etc., and thereby improving the noise reduction efficiency of the porous sound-absorbing plate.

In some embodiments, the porous sound-absorbing plate may include a splitting component.

When the gasflow flows through the splitting component, the gasflow flows through both separating component surfaces of the splitting component. In some embodiments, the splitting component may be disposed generally parallel (or approximately parallel) to the gasflow direction, so that the gasflow direction substantially coincides with a disposing direction of the splitting component to allow the gasflow to flow simultaneously through both surfaces of the splitting component. The splitting component may realize the effect of diverting the gasflow according to disposing requirements.

In some embodiments, the splitting component may include at least one first separating component disposed on the first surface. An angle between the disposing direction of the at least one first separating component and the gasflow direction may satisfy a first threshold condition. The first threshold condition may reflect a parallel or near-parallel angular threshold condition. For example, the first threshold condition may be +10°, i.e., the angle between the disposing direction of the first separating component and the gasflow direction may be in the +10° range, then it may be determined that the disposing direction of the first separating component and the gasflow direction satisfy the first threshold condition, and the first separating component and the gasflow are nearly parallel with each other.

In some embodiments, to comb the gasflow at the gas inlet, the at least one first separating component may be disposed at an inflow end of the gasflow.

Based on the above first separating component, when the gasflow flows through the first separating component, the gasflow flows through both separating component surfaces of the first separating component simultaneously, forming at least two gas paths to minimize the sectional region of the gasflow in the gas path. For practical applications of the first separating component, please refer to FIG. 4D and the related descriptions, which are not repeated here.

In some embodiments, the porous sound-absorbing plate may include a guiding component.

When the gasflow flows through the guiding component, the gasflow flows sequentially through both separating component surfaces of the guiding component. The separating component surface through which the gasflow flows preferentially may be denoted as the first surface of the separating component, and the opposing surface of the first surface may be denoted as the second surface. In some embodiments, the guiding component may change a trajectory of the gasflow, thereby extending the path of the gasflow to make a full contact with the porous sound-absorbing plate, and improving the noise reduction efficiency of the porous sound-absorbing plate. For example, the disposing direction of the guiding component may have a significant angle (e.g., vertical, or relatively vertical) with the gasflow direction, so that when flows through the guiding component, the gasflow may be turned by the guiding component due to the angle of the guiding component disposed. For another example, the guiding component may divide the gas channel in which the gasflow is located (e.g., the guiding component may be disposed across the gas channel), so that the gas channel may be split into a plurality of sub-gas channels, and the gasflow may flow sequentially through the sub-gas channels.

In some embodiments, the guiding component may include a second separating component disposed on the first surface. An angle between the disposing direction and the gasflow direction may satisfy a first threshold condition. One end of the second separating component faces the inflow end of the gasflow.

Based on the above second separating component, when the gasflow flows through the second separating component, the gasflow may flow along a first separating component surface of the second separating component and may turn and flow along a second separating component surface of the second separating component upon reaching the end of the second separating component. For the practical applications of the second separating component, please refer to FIG. 4E and the related descriptions, which are not repeated here.

In some embodiments, the guiding component may further include a third separating component group disposed on the first surface. An angle between the disposing direction of the third separating component group and the gasflow direction may satisfy a second threshold condition. The second threshold condition refers to an angle threshold condition indicating either vertical or a significant angle. For example, the second threshold condition may be [−150°, −30° ]U [30°, 150°], i.e., the angle between the disposing direction of each separating component in the second separating component group and the gasflow direction is within the above-described range. Then it may be determined that the disposing direction of each separating component in the second separating component group and the gasflow direction satisfies the second threshold condition, and the disposing direction of each separating component in the second separating component group and the gasflow direction are approximately vertical or at a significant angle.

In some embodiments, the third separating component group may include at least one high third separating component and at least one low third separating component. The high third separating component may be adjacent to the low third separating component, and they may be staggered in height.

The height may be understood as an extension direction in the porous sound-absorbing plate. For example, the height direction may be the Z-axis direction in FIGS. 4D to 4G. The staggered disposing may be understood as the high porous sound-absorbing plate being disposed adjacent to the low porous sound-absorbing plate in the preset direction (e.g., the X-axis, Y-axis direction of FIGS. 4D-4G) in successive alternation.

Based on the above third separating component group, when the gasflow flows through the adjacent high third separating component and the low third separating component in sequence, the gasflow may flow along the first surface of the high third separating component and, upon reaching the end of the high third separating component, the gasflow may enter a gasflow space formed by the second surface of the high third separating component and the first surface of the low third separating component. The gasflow may flow out at the end of the low third separating component along the second surface of the third separating component. For the practical applications of the third separating component, please refer to FIG. 4F and the related descriptions, which are not repeated here.

In some embodiments, the porous sound-absorbing plate may often bend with the gasflow in the practical applications. Considering that the first porous sound-absorbing plate is generally used as the top and bottom surfaces of the gasflow, the second porous sound-absorbing plate in the porous sound-absorbing plate may be disposed with a porous sound-absorbing plate bending along the gasflow.

In some embodiments, in order to eliminate the noise at the porous sound-absorbing plate bending, the second porous sound-absorbing plate may include a fourth separating component group disposed at the bending position of the porous sound-absorbing plate. An angle between the disposing direction of the fourth separating component group and the gasflow direction may satisfy the second threshold condition. The fourth separating component group may be regarded as a kind of guiding component.

In some embodiments, the fourth separating component group may include at least one first side separating component and at least one second side separating component, the first side separating component being disposed on one side of the bending position of the porous sound-absorbing plate, and the second side separating component being disposed on the other side of the bending position of the porous sound-absorbing plate. The first side separating component may be adjacent and staggered in the disposing direction of the second porous sound-absorbing plate. There may be a staggering portion between the adjacent first side separating component and the second side separating component in the disposing direction.

Based on the above fourth separating component group, when the gasflow flows through the adjacent first side separating component and the second side separating component, the gasflow may flow along the first surface of the first side separating component and turns and enters the gasflow space formed by the staggering portion when it reaches the end of the first side separating component, and finally reach the first surface of the second side separating component and flows out. For practical applications of the fourth separating component group, please refer to FIG. 4G and the related descriptions, which are not repeated here.

In some embodiments, the second porous sound-absorbing plate may further include a fifth separating component disposed in the staggering portion. An angle between the disposing direction of the fifth separating component and the gasflow direction may satisfy the first threshold condition.

Based on the above fifth separating component, when the gasflow flows in the staggering portion between the adjacent first side separating component and the second side separating component, the gasflow may flow through both separating component surfaces of the first separating component at the same time, forming at least two gasflow flow paths to guide the gasflow. For practical applications of the fifth separating component, please refer to FIG. 4G and the related descriptions, which are not repeated here.

To further illustrate the application of the porous sound-absorbing plate in a real scenario, the present disclosure also provides a noise reduction structure involving the porous sound-absorbing plate. The noise reduction structure may be applied to a blower within a main body of a respiratory ventilation apparatus. The noise reduction structure may include a noise reduction housing, a porous sound-absorbing plate, and a sound-absorbing material. The noise reduction housing may be configured to accommodate the noise reduction structure as well as the blower. The porous sound-absorbing plate may be disposed in a gas channel between the blower cavity and a housing of a noise reduction device, and the blower cavity may be disposed in the noise reduction device. The gas channel may be configured to direct the external gasflow to the blower cavity. The sound-absorbing material may be disposed between the porous sound-absorbing plate and the housing.

FIG. 4C is a sectional view of a noise reduction structure according to some embodiments of the present disclosure.

As shown in FIG. 4C, the embodiments of the present disclosure provides the noise reduction structure. The noise reduction structure 4100 may be applied to a blower assembly 4200. The noise reduction structure 4100 may include a noise reduction housing 4110, a porous sound-absorbing plate 4120, and a sound-absorbing cotton 4130.

In embodiments of the present disclosure, the blower assembly 4200 may include a blower integrated in a respiratory ventilation apparatus. The blower in the respiratory ventilation apparatus may be a device configured to increase a gas pressure and deliver the gas to assist the user in breathing. In some embodiments, the blower assembly 4200 may include a blower body 4210, and a blower cavity 4220. The blower body 4210 may be disposed in the noise reduction structure 4100 through the blower cavity 4220, and the blower cavity 4220 may be configured to accommodate and fix the blower body 4210.

The blower cavity 4220 may include a blower cavity housing 4221, a cavity gas inlet 4222 and a cavity gas outlet 4223. The blower cavity housing 4221 may be a main structure of the blower cavity 4220 configured to accommodate the blower body 4210. The cavity gas inlet 4222 and the cavity gas outlet 4223 may be disposed on the blower cavity housing 4221. The cavity gas inlet 4222 may communicate with the noise reduction structure 4100 to introduce the external gas. The gas outlet 4223 may communicate with the internal gas channel of the main body and ultimately with the user interface 7000 to export the pressurized gasflow. During operation, the external gas may enter the cavity gas inlet 4222 through the noise reduction structure 4100, and after being pressurized by the blower body 4210, the external gas may flow out of the cavity gas outlet 4223.

It should be noted that there is no limitation on the type of the blower body 4210 in the present disclosure, and in the accompanying drawings of the present disclosure, the blower body 4210 may be depicted as a centrifugal blower as an example. When changing a type of the blower body 4210, it is only necessary to adaptively adjust a morphology of the blower cavity 4220, which does not affect the noise reduction structure 4100. For example, the blower body 4210 in FIG. 4C may also be replaced with an axial flow blower. At this time, only an orientation of the cavity gas outlet 4223 (e.g., upwardly oriented) needs to be changed to set the cavity gas outlet 4223 along the direction of the blower outlet.

The blower cavity housing 4221 may be a structure for restricting an entry and output of the external gas. In some embodiments, the blower cavity housing 4221 may also be a spatial structure composed of a plurality of portions (e.g., an upper housing, a lower housing, etc.). A shape of the structure is not limited, which may be square, circular, or other irregularly shapes, etc.

In some embodiments, the cavity gas inlet 4222 may be disposed on a side of the blower cavity housing 4221 that faces the gas channel to allow the external gas to enter the blower cavity. There may be more than one cavity gas inlets 4222, and a number and positions of the cavity gas inlet 4222 are not limited in the present disclosure.

In some embodiments, as shown in FIG. 4C, the cavity gas inlet 4222 may be uniformly disposed on the blower cavity housing 4221 along the gas channel, so that when the gasflow flows in the gas channel. The gasflow may enter the blower cavity dispersedly into the blower cavity to reduce a volume of the gasflow entering the gas inlet, thereby reducing the noise generated. The noise reduction housing 4110 may be a structure for accommodating the noise reduction structure as well as the blower. For example, the noise reduction structure 4100 as well as the blower assembly 4200 may be taken as a component of the blower as a whole through the noise reduction housing 4110, and may be integrally disposed in the respiratory ventilation apparatus. In some embodiments, the noise reduction housing 4110 may be a spatial structure composed of a plurality of portions (e.g., an upper housing, a lower housing, etc.). A shape of the noise reduction housing 4110 is not limited, which may be square, circular, or other irregular shape, etc. In some embodiments, the structural shape and size of the noise reduction housing 4110 may be determined based on the shapes and sizes of the noise reduction structure 4100 and the blower assembly 4200.

In some embodiments, the noise reduction housing 4110 may be configured to accommodate and fix the blower assembly 4200 and other components of the noise reduction structure 4100. For example, the porous sound-absorbing plate 4120 may be disposed on the noise reduction housing 4110 in through a fixed connection. Furthermore, for example, the sound-absorbing cotton 4130 may be buckle-fitted between the porous sound-absorbing plate 4120 and the housing. The fixed connection refers to a connection that fixes the portions or components without any relative movement. The fixed connection may include a non-detachable connection (e.g., a weld, an integration, etc.), a detachable connection (e.g., a threaded connection, a buckle connection, and other modes for assembling, etc.), and a direct connection (e.g., a disposing of the components on the corresponding sizes based on preset sizes, and a fixed assembling through clamping or abutting, etc.).

In some embodiments, the noise reduction housing 4110 may form the gas channel with the blower cavity. The gas channel refers to a channel where the gas inside the noise reduction housing 4110 flows toward the blower assembly 4200 after the gas outside the noise reduction housing 4110 enters the noise reduction 4110.

In some embodiments, a space between the noise reduction housing 4110 and the blower cavity housing 4221 may be noted as the gas channel. A beginning of the gas channel may be a gas inlet 4150 on the noise reduction housing 4110, and an end of the gas channel may be the cavity gas inlet 4222 disposed on the blower cavity housing 4221. The gas channel may extend a circuit of the gasflow as it enters the blower assembly 4200, thereby reducing the noise.

In some embodiments, the porous sound-absorbing plate 4120 may be disposed in the gas channel between the blower cavity in which the blower body 4210 is disposed and the noise reduction housing 4110. When the gasflow in the gas channel passes through the porous sound-absorbing plate 4120, based on a sound-absorbing feature of the micropores, the noise of the gasflow passing the gas channel may be significantly reduced.

In some embodiments, referring to FIG. 4C, the porous sound-absorbing plate 4120 may include a first surface 4121, a second surface 4122, and through holes. The first surface 4121 refers to a side of the porous sound-absorbing plate 4120 facing the gas channel; the second surface 4122 refers to a side of the porous sound-absorbing plate 4120 facing the sound-absorbing cotton or a side facing the respiratory ventilation apparatus housing side, i.e., the other side of the porous sound-absorbing plate. For more contents about the first surface, the second surface, and the through hole, please refer to the relevant descriptions of FIG. 4A, which are not repeated here.

In some embodiments of the present disclosure, when the gasflow passes through the through holes, a viscous dissipation may happen to eliminate a portion of mid-low frequency noise in the gasflow, thereby reducing the noise. In addition, based on the fact that different apertures may have different effects on eliminating the noise of the gasflow in different frequency band ranges, it may be possible to reduce the noise in different frequency bands by designing different apertures of the through holes on the first surface and on the second surface. As a result, the noise at different frequency bands may be reduced, so as to better eliminate the noise and secure the user experience.

In some embodiments, considering the gasflow direction in the gas channel, the porous sound-absorbing plate 4120 may be further divided into a first porous sound-absorbing plate 4120-1 (not shown in FIG. 4C, referring to FIGS. 4D-4G), a second porous sound-absorbing plate 4120-2, and a third porous sound-absorbing plate 4120-3. The first porous sound-absorbing plate 4120-1 as well as the third porous sound-absorbing plate 4120-3 may be porous sound-absorbing plates that are approximately parallel to the first plane. The second porous sound-absorbing plate 4120-2 may be a porous sound-absorbing plate that is approximately vertical to the first plane. The first plane may be an xy plane. In some embodiments, the first porous sound-absorbing plate 4120-1 may be denoted as the top surface of the porous sound-absorbing plate to be in contact with the top surface of the gas channel, and the third porous sound-absorbing plate 4120-3 may be denoted as the bottom surface of the porous sound-absorbing plate 4120 to be in contact with the bottom surface of the gas channel. The second porous sound-absorbing plate 4120-2 may be denoted as a side of the porous sound-absorbing plate 4120.

In some embodiments, the first porous sound-absorbing plate 4120-1 or the second porous sound-absorbing plate 4120-2 may be flat or curved into a curved surface. For example, the second porous sound-absorbing plate 4120-2 in FIG. 4D may be in an L-shape. As an alternative embodiment, the second porous sound-absorbing plate may also be curved.

In some embodiments, see FIG. 4C, in the gas channel formed between an inner wall of the noise reduction housing and an outer wall of the blower cavity, generally only the gas channel formed between the porous sound-absorbing plate 4120 and the outer wall of the blower cavity may be an effective gas channel, and the other portions may be generally filled with an impedance sound-insulating material (e.g., the sound-absorbing cotton) to reduce the noise. Then a morphology of the porous sound-absorbing plate 4120 may be determined according to the design of the gas channel, i.e., the shape of the first porous sound-absorbing plate 4120-1 or the second porous sound-absorbing plate 4120-2 may be matched with the shape of the gas channel and may be disposed along the extension direction of the gas channel. For example, when the gas channel is designed as a linear gas channel, the porous sound-absorbing plate 4120 may be presented as a square channel. The second porous sound-absorbing plate 4120-2 may be the sidewall of the channel, and the first porous sound-absorbing plate 4120-1 may be the top or the bottom surface of the channel. For another example, the first porous sound-absorbing plate 4120-1 and the second porous sound-absorbing plate 4120-2 may also be disposed in only a portion of the gas channel and without being bent to reduce the processing difficulty.

It should be noted that the present disclosure does not limit the shape and processing mode of the first porous sound-absorbing plate or the second porous sound-absorbing plate. The first porous sound-absorbing plate or the second porous sound-absorbing plate may be flat or curved into a curved surface. The first porous sound-absorbing plate and the second porous sound-absorbing plate may be integrally molded or independently molded and then combined.

The sound-absorbing cotton may be a structure for absorbing the noise of the gasflow. In some embodiments, the sound-absorbing cotton may be disposed between the porous sound-absorbing plate 4120 and the inner wall of the noise reduction housing to cooperate with the porous sound-absorbing plate 4120 in absorbing the noise, so that a double noise reduction may be realized. It should be noted that the sound-absorbing cotton may be only used as an example of a commonly used sound-absorbing material, and the sound-absorbing cotton may be replaced with other sound-absorbing materials in practical applications.

The present disclosure does not limit the structural shape of the sound-absorbing cotton. The sound-absorbing cotton may be of a regular shape (e.g., a rectangular, etc.), or an irregular shape (e.g., a curved shape, etc.), etc. In some embodiments, the structural shape of the sound-absorbing cotton may be determined based on a gap between the porous sound-absorbing plate 400 and the inner wall of the noise reduction housing.

In some embodiments, the sound-absorbing cotton may be an integrated structure or a structure that combines a plurality of sub-structures, which may be determined based on actual circumstances.

In some embodiments, as shown in FIGS. 4A-4B, when the gas flows within the gas channel, a portion of the gasflow may reach the sound-absorbing cotton through the through holes 411 in the micro-perforated plate 400, and this portion of the gasflow, as it passes through the through holes 411, may undergo a viscous dissipation, which eliminates some of the high-frequency noise in the gasflow. As a result, the noise generated by the gasflow that reaches the sound-absorbing cotton may be partially absorbed by the sound-absorbing cotton, and partially reflected again through the through hole 411 of the porous sound-absorbing plate 400 (which also eliminates a portion of the noise through the viscous dissipation) and again enters the gas channel. The residual high-frequency noise in the gasflow that returns to the gas channel may be counteracted and overlapped with a high frequency by the other gasflows in the gas channel. The process may go round and round as described above. In addition, given the fact that the aperture of the through hole 411 at the first surface 413 is smaller than the aperture of the through hole on the second surface 412, there may be a certain slope in the sidewall of the through hole 411. As a result, the noise returning from the sound-absorbing cotton may be reflected by the sidewall of the through hole 414, thereby reducing the noise reflected back into the gas channel.

FIG. 4D is a schematic diagram illustrating a structure of a first separating component of a porous sound-absorbing plate according to some embodiments of the present disclosure.

As shown in FIG. 4D, in some embodiments, the porous sound-absorbing plate 4120 may further include at least one first separating component 4124 disposed at a gas inlet of a housing. The first separating component 4124 may be disposed in a gasflow direction (shown by the dotted arrows) within a gas channel.

The first separating component refers to a structure disposed at the gas inlet of the gas channel for separating the gasflow within the gas channel. A structural shape of the first separating component plate may have an unlimited shape, and may be a rectangular plate, a curved plate, etc. In some embodiments, the structural shape of the first separating component 4124 may be adaptively designed based on the shape of a gap between the porous sound-absorbing plate 4120 and an outer wall of a blower cavity. For example, the first separating component 4124 may rest against the outer wall of the blower cavity.

In some embodiments, a number of the first separating component 4124 disposed may not be limited. There may be only one first separating component 4124, or there may be a plurality of first separating components 4124. The specific number may be determined based on an experimentation, a simulation modeling, etc. In some embodiments, the first separating component 4124 may be fixedly disposed on the porous sound-absorbing plate 4120 (e.g., a second porous sound-absorbing plate at a side of the gas channel). For example, the first separating component 4124 may be disposed on the porous sound-absorbing plate 4120 through a non-detachable connection (e.g., a heat fused connection, etc.); and for another example, the first separating component 4124 may be disposed on the porous sound-absorbing plate 4120 through a detachable connection (e.g., a buckle connection, etc.) on the porous sound-absorbing plate 4120 to adjust the number of the first separating components based on actual needs.

In some embodiments of the present disclosure, by providing the plurality of first separating components at a gas inlet of the porous sound-absorbing plate 4120, with the plurality of first separating components spaced apart in a vertical direction along the gasflow direction in the gas channel, the gasflow flowing through the first separating components may be separated into at least two sub-gasflows, thereby reducing a sectional region of each sub-gasflow, and realizing a combing of the gasflow. By forcibly separating a turbulent flow into a laminar flow, a formation of a turbulence phenomenon may be avoided, and at the same time, the noise generated by the turbulence phenomenon may be avoided.

FIG. 4E is a schematic diagram illustrating a structure of a second separating component of a porous sound-absorbing plate according to some embodiments of the present disclosure.

As shown in FIG. 4E, in some embodiments, the porous sound-absorbing plate 4120 may include a second separating component 4125. The second separating component 4125 may be disposed within the gas channel along a gasflow direction. A surface of the separating component labeled 4125 in FIG. 4E may be a second surface of the second separating component 4125. An opposite side of the second surface may be a first surface of the second separating component 4125.

The gasflow direction within the gas channel refers to the gasflow direction within the gas channel based on the pre-existing porous sound-absorbing plate (and the separating component plate) structure at a position of a current separating component. It should be noted that the gasflow directions within the gas channels during subsequent separating components dispositions in the present disclosure are consistent with the definition herein, which are all related to the dispose position of the respective separating components.

In some embodiments, the second separating component 4125 may be held against the outer wall of the blower cavity and disposed along a course of the second porous sound-absorbing plate 4120-2, and an end of the second separating component 4125 may be spaced apart from the end of the gas channel. Based on the second separating component 4125, the gas channel may be separated into a first sub-gas channel and a second sub-gas channel at the position of the second separating component 4125.

The first sub-gas channel and the second sub-gas channel may be two sub-gas channels obtained by splitting an effective portion of the gas channel by the second separating component 4125. The first sub-gas channel and the second sub-gas channel may be connected at the end of the second separating component 4125.

In some embodiments, as shown in FIG. 4E, the first sub-gas channel may be disposed below the second separating component 4125, the second sub-gas channel may be disposed above the second separating component 4125, and the gas inlet 4150 of the gas channel may be connected to the first gas channel. Based on the first sub-gas channel as well as the second gas channel, when the external gasflow enters the gas channel, for the gasflow direction within the gas channel, please refer to the wind flow of the arrows in FIG. 4E. The external gas may enter the first sub-gas channel along the gas inlet of the gas channel and then enter the second sub-gas channel at the end of the second separating component 4125, and then enter the blower cavity through the cavity gas inlet 4222 of the blower cavity.

In some embodiments, the cavity gas inlets of the blower cavity (e.g., the first gas inlet 231, the second gas inlet 232, and the third gas inlet 233 in FIG. 2C) may be adjusted correspondingly based on the second gas channel. For example, the cavity inlet of the blower cavity may be disposed at the second gas channel.

The first sub-gas channel and the second sub-gas channel formed by the second separating component 4125 may further extend a length of a path in which the external gasflow enters the blower, improve the noise reduction efficiency of the individual through hole, and thus reduce the noise of the blower. Additionally, a section of the gas channel divided by the second separating component 4125 may be smaller than a section of the original gas channel, which avoids the turbulence and the turbulence noise in the gas channel with a great section.

It is noted that, based on a technical effect of the second separating component 4125, the second separating component 4125 may include a plurality of parallel (or near-parallel) disposed separating components, each of which is staggered so as to further extend the length of path in which the external gasflow enters the blower.

FIG. 4F is a schematic diagram illustrating a structure of a third separating component of a porous sound-absorbing plate according to some embodiments of the present disclosure.

In some embodiments, the microporous plate 4120 may also include at least two third separating components, and each of the at least two third separating components may be disposed vertical to a gasflow direction in a gas channel. The separating components may be in an alternative contact with an upper surface 41211 or a lower surface 41212 of the gas channel, so that at least one of the at least two third separating components may be in contact with the upper surface 41211 of the gas channel, and at least one of the third separating components may be in contact with the lower surface 41212 of the gas channel.

As shown in FIG. 4F, the at least two third separating components may form a third separating component group 4126, and the third separating component group 4126 may further include a first low third separating component 41261, a first high third separating component 41262, a second low third separating component 41263 and a second high third separating component 41264. The first low third separating component 41261 and the second low third separating component 41263 may be in contact with a lower surface 41212 of the gas channel. The first high third separating component 41262 and the second high third separating component 41264 may be in contact with the upper surface 41211 of the gas channel. In some embodiments, a first surface of the third separating component may be a surface oriented in a positive X-axis direction/negative Y-axis direction of each third separating component, and a second surface of the third separating component may be a surface oriented in a negative X-axis direction/positive Y-axis direction of each third separating component.

The third separating component refers to a structure that alternately contacts the upper surface or the lower surface of the gas channel for separating the gasflow within the gas channel. As shown in FIG. 4F, the upper surface 41211 of the gas channel refers to a first surface disposed above the gas channel, i.e., an upper inner surface of the first surface (e.g., the inner wall of the first porous sound-absorbing plate 4120-1 on a top surface); the lower surface 41212 of the gas channel refers to the first surface disposed below the gas channel, i.e., a lower inner surface of the first surface (e.g., the inner wall of the third porous sound-absorbing plate 4120-3 on a bottom surface). In some embodiments, the third separating component group 4126 may have an unlimited structural shape, which may be a rectangular plate, a curved plate, etc.

In some embodiments, “the third separating component being alternately in contact with the upper surface or the lower surface of the gas channel” may be understood as a third separating component group 4126 may be alternately connected to the upper surface 41211 and the lower surface 41212 of the gas channel in sequence. Exemplarily, if three third separating component plates are sequentially disposed in a direction vertical to the gasflow direction in the gas channel (denoted as A31, A32, and A33, respectively), a first third separating component plate (A31) may be connected to the upper surface 1211 of the gas channel, a second third separating component plate (A32) may be connected to the lower surface 41212 of the gas channel, and a third separating component plate (A33) may be connected to the upper surface 41211 of the gas channel. Alternatively, the first third separating component plate (A31) may be connected to the lower surface 41212 of the gas channel, the second third separating component plate (A32) may be connected to the upper surface 41211 of the gas channel, and the third separating component plate (A33) may be connected to the lower surface 41212 of the gas channel.

In some embodiments, the third separating component group 4126 may be integrally molded with the porous sound-absorbing plate 4120, or they may be fixedly connected, etc. For another example, the third separating component group 4126 may further be connected to the porous sound-absorbing plate 4120 through a fastener connection, a buckle connection, etc.

In some embodiments, a size of each third separating components of the third separating component group 4126 and/or a distance between the third separating components of the third separating component group 4126 may be matched to the size of the gas channel, so that sections of a gasflow pathway in the gas channel at each third separating component may be basically the same.

In some embodiments, “a size of each third separating components of the third separating component group and/or a distance between the third separating components of the third separating component group may be matched to the size of the gas channel” may be understood as a height H, a width L, and a spacing D of each third separating component within the third separating component group 4126 match a performance requirement of the gas channel. For example, the performance requirement of the gas channel may be that the gasflow inside the gas channel is continuous and stable, and in order to be compatible with this design requirement, the heights H of the third separating components may be the same, the widths L of the third separating components may be identical to a width W of the gas channel, and the spacings D between the plurality of third separating components may be the same. As a result, when the gasflow in the gas channel (shown by dotted arrows) passes through each third separating component, the section of the gasflow pathway at each third separating component may be basically the same, i.e., a volume of the gasflow into a region formed by each of the two third separating components may be basically of the same magnitude as the volume of the gasflow out of the region.

In some embodiments, the spacing D between the third separating component plates may also be different, and the specific distance D may be determined based on a current gasflow volume. For example, when a distance between the second porous sound-absorbing plate 4120-2 and an outer wall of the blower cavity is farther away (i.e., the width W of the gas channel therein is greater), the spacing D of the third separating component plate may be smaller, and when the distance between the second porous sound-absorbing plate 4120-2 and the outer wall of the blower cavity is closer (i.e., the width W of the gas channel therein is smaller), the spacing D of the third separating component plate may be greater. As a result, the volume of the gasflow at each position may be substantially the same.

The height of the third separating component may not be unlimited, which may be determined based on experiments, simulations, etc. In some embodiments, the height of the third separating component may be determined based on a section of the cavity inlet of the blower cavity. Exemplarily, the height of the third separating component may be a ratio of a sectional region of the cavity gas inlet of the blower cavity to the width of the third separating component (i.e., the width of the gas channel).

In some embodiments of the present disclosure, by staggering the third separating component in the gasflow direction vertical to the gas channel, the gasflow in the gas channel may be made to sequentially pass above and below (or, below and above) the third separating component to extend the path of the gasflow. Moreover, based on the match of the size of the third separating component and/or the distance between the third separating components and the size of the gas channel, the path of the gasflow in the gas channel may have essentially the same section at the third separating component, so as to maximize a use of the through holes of the porous sound-absorbing plate, and achieve a better noise reduction effect.

FIG. 4G is a schematic diagram illustrating a structure of a fourth separating component and a fifth separating component of a porous sound-absorbing plate according to some embodiments of the present disclosure.

As shown in FIG. 4G, in some embodiments, the porous sound-absorbing plate 4120 may further include at least two fourth separating components disposed at corners of the gas channel. The at least two fourth separating components may form a fourth separating component group 4127, and the fourth separating component group 4127 may specifically include a first side separating component 41271 and a second side separating component 41272. The surface to which reference number 41271 in FIG. 4G points may be a first surface of the first side separating component 41271, and the opposite side of the surface to which reference number 41271 points may be a second surface of the first side separating component 41271. The surface to which reference number 41272 in FIG. 4G points may be a first surface of the second side separating component 41272, and the opposite side of the surface to which reference number 41272 points may be a second surface of the second side separating component 41272.

The fourth separating component refers to a structure disposed at the corner of a gas channel to separate a gasflow. In some embodiments, the fourth separating component may be staggered along a direction vertical to the gasflow direction within the gas channel on both sides of the corner, so that the gasflow in the gas channel (shown by the dotted arrows) flows from above one of the fourth separating components of the fourth separating component group 4127 (e.g., the first side separating component 41271) to below another one of the fourth separating component group 4127 (e.g., the second side separating component 41272). The two adjacent fourth separating components may form an angle and may have an overlapping portion. That is, the gasflow may first flow through the first surface of the first side separating component 41271 and turn into the staggering portion above the first side separating component 41271 to flow through the second surface of the second side separating component 41272, thereby flowing out from below the second side separating component 41272.

In some embodiments, the “staggered on both sides of the corner” may be understood as the fourth separating component group 4127 being sequentially and alternately connected to the porous sound-absorbing plates on both sides of the corner. For example, for a first sub-plate 41221 and a second sub-plate 41222 of the second porous sound-absorbing plate 4120-2, a corner may be formed between the first sub-plate 41221 and the second sub-plate 41222. The first side separating component 41271 in the fourth separating component plate group 4127 may be connected to the first sub-plate 41221, and the second side separating component plate 41272 may be connected to the second sub-plate 41222. “Staggered up and down” may be understood as that there is a height difference between each fourth separating component in the fourth separating component group 4127. For example, the second side separating component 41272 may be disposed above the first side separating component 41271.

In some embodiments, the porous sound-absorbing plate 4120 may also include a supporting plate 4129 that is simultaneously coupled to each fourth separating component for supporting the fourth separating components. As shown in FIG. 4G, opposing edges of the edges of the fourth separating component that are connected to the porous sound-absorbing plate may be connected to the supporting plate 4129.

An upside of the fourth separating component refers to the upside of the fourth separating component in the at least two fourth separating components that is disposed at a lower position. As shown in FIG. 4G, the upside of the fourth separating component may be the upside of the first side separating component 41271, i.e., the position between the first side separating component 41271 and the first porous sound-absorbing plate 4120-1.

A lower side of the fourth separating component refers to the lower side of the fourth separating component in the at least two fourth separating components that is disposed at an upper position. As shown in FIG. 4G, the lower side of the fourth separating component may be the lower side of the second side separating component 41272, i.e., the position between the second side separating component 41272 and the third porous sound-absorbing plate 4120-3.

In some embodiments, “two adjacent fourth separating components forming an angle” may be understood as the angle formed by two of the adjacent fourth separating components of the fourth separating component group 4127 (i.e., the first side separating component 41271 and the second side separating component 41272 of FIG. 4G) when staggered up and down at a corner. In some embodiments, the angle may be determined based on historical data, simulations, etc. For example, the angle may be 90°, etc.

In some embodiments, the “overlapping portion” may be understood as the overlapping portion of the projections in a direction of the staggered disposition when two adjacent fourth separating components of the fourth separating component group 4127 (i.e., the first side separating component 41271 and the second side separating component 41272) are staggered up and down. That is, a height difference between the fourth separating components may be less than the length of the fourth separating component itself. This overlapping portion may guide the gasflow to enter the lower side of another fourth separating component (e.g., the second side separating component 41272) as it passes over the upside of one of the fourth separating components (e.g., the first side separating component 41271) of the fourth separating component group 4127, so as to change the gasflow direction.

In some embodiments of the present disclosure, by disposing at least two fourth separating components at the corners and disposing them staggered along the direction vertical to the gasflow direction in the gas channel on each side of the corners, the gasflow in the gas channel may pass through the upside of one of the at least two fourth separating components and pass through the lower side of the other of the at least two fourth separating components to change the gasflow direction. In this way, it may be equivalent to extend the length of the gas channel to a certain extent, so that the gasflow may be made to pass through the through holes on the porous sound-absorbing plate as much as possible to improve the noise reduction effect.

As shown in FIG. 4G, in some embodiments, the porous sound-absorbing plate 4120 may also include a fifth separating component 4128.

The fifth separating component refers to a structure disposed between at least two of the fourth separating components along the gasflow direction within the gas channel. In some embodiments, the fifth separating component 4128 may be disposed between each fourth separating component of the fourth separating component group 4127 and along the gasflow direction within the gas channel. A length of the fifth separating component 4128 along the gasflow direction may be the same as the length of the overlapping portion between the two adjacent fourth separating components. That is, in the overlapping portion between the two fourth separating components, one end of the fifth separating component 4128 may be connected to one fourth separating component (e.g., the first side separating component 41271), and the other end may be connected to the first fourth separating component (e.g., the second side separating component 41272) of the overlapping portion, and the fifth separating component 4128 may be disposed along the gasflow direction within the gas channel.

It should be noted that herein, the “gasflow direction within the gas channel” may be understood as the gasflow direction after changing the direction of the original gasflow due to a guiding effect of the two adjacent fourth separating components (i.e., the first side separating component 41271 and the second side separating component 41272 in FIG. 4G) of the fourth separating component group 4127. As shown in FIG. 4G, the gasflow direction in the gas channel (shown by the dotted arrows) may be changed from an original horizontal to the left to a vertical downward direction when the gasflow passes through the two adjacent fourth separating components. Here, the “gasflow direction in the gas channel” may be the vertical downward gasflow direction.

In some embodiments of the present disclosure, by disposing the fifth separating component between the at least two fourth separating components and disposing the fifth separating component in the gasflow direction within the gas channel to separate the gasflow when the gasflow flows in from the upside of one of the at least two fourth separating components, a turbulence of the gasflow at the corners may be avoided, and thus the gasflow in the gas channel may be guaranteed, so that a foundation may be laid for the gasflow to enter the through holes of the porous sound-absorbing plate to a maximum extent. Additionally, the adjacent fourth separating components and the fifth separating component in between may form a great section-small section-great section noise reduction structure, which reduces the noise based on a resistance principle.

In some embodiments, as shown in FIG. 4H, the noise reduction structure 4100 may also include a noise reduction top housing 4140. In order to more clearly observe the arrangement of the porous sound-absorbing plate 4120 and the sound-absorbing cotton 4130 inside the noise reduction housing, the noise reduction bottom housing is not shown in FIG. 4H (in this embodiment, the noise reduction structure 4100 is composed of a noise reduction bottom housing and a noise reduction top housing 4140 that are cooperatively connected). As shown in FIG. 4H, the sound-absorbing cotton 4130 may be disposed on the side of the noise reduction top housing 4140 facing the blower body 4210, and the sound-absorbing cotton 4130 may be disposed on the side of the noise reduction bottom housing facing the blower body 4210. It may also be understood as the sound-absorbing cotton 4130 being disposed on the upper and lower sides of the blower body 4210. Moreover, the sound-absorbing cotton 4130 may also be provided on the side of the corresponding blower body 4210 to semi-enclose the side of the blower. For example, the sound-absorbing cotton 4130 may be provided circumferentially along the direction of the gas inlet of the blower body 4210. Specifically, the sound-absorbing cotton 4130 may be provided, for example, in a full circle, a half circle or a part of a full circle circumferentially. The sound-absorbing cotton 4130 corresponding to the bottom, the top and the circumference of the blower may absorb the noise in a noise reduction device within a great space range. For example, it may absorb the noise generated by the gasflow in the gas channel and the vibration noise generated by the operation of the blower body 4210.

In some embodiments, the lower end of the blower body 4210 may be configured with a plurality of blower support columns 4211, and the sound-absorbing cotton 4130 configured above the noise reduction bottom housing may be provided with the through holes with a number and sizes corresponding to the plurality of blower support columns 4211. When the blower body 4210 is mounted into the noise reduction housing, the lower ends of the plurality of blower support columns 4211 may pass through the through holes on the sound-absorbing cotton 4130 and may abutted or fixedly connected to the upper end face of the noise reduction bottom housing. This structural design may make the sound-absorbing cotton 4130 better absorb the vibration noise generated by the operation of the blower body 4210.

In some embodiments, as shown in FIG. 4H, the porous sound-absorbing plates 4120 arranged along the gas channel extension direction may not be higher than half the height of the gas channel. The upper end of the porous sound-absorbing plate 4120 may be configured with a plurality of top housing support columns 4123. The lower end of the top housing support column 4123 may be fixedly connected to the upper end of the porous sound-absorbing plate 4120, and the upper end of the top housing support column 4123 may be fixedly connected to the noise reduction top housing 4140. In order to facilitate the mounting and later disassembly and maintenance of the blower body 4210, at least one end of the top housing support column 4123 may be detachably fixedly connected to the porous sound-absorbing plate 4120 or the noise reduction top housing 4140 (e.g., modes like snaping, pin, screwing, etc. may be used). In some embodiments, the lower ends of the plurality of top housing support columns 4123 may be integrally formed on the porous sound-absorbing plate 4120, or may be fixedly connected to the porous sound-absorbing plate 4120 by non-detachable modes such as pasting and riveting. The upper end of the plurality of top housing support column 4123 may be fixedly connected to the noise reduction top housing 4140 using detachable connection modes such as snaping, pin, and screwing. In some embodiments, the upper ends of the plurality of top housing support columns 4123 may be fixedly connected to the noise reduction top housing 4140 in a non-detachable connection mode, and the lower ends may be fixedly connected to the porous sound-absorbing plate 4120 in a detachable connection mode. In some embodiments, the upper and lower ends of the plurality of top housing support columns 4123 may be fixedly connected to the porous sound-absorbing plate 4120 in the detachable connection mode.

A respiratory ventilation apparatus may include a main body and a reservoir or a cover plate without a reservoir connected to the main body. In actual use, in order to ensure that the user uses the respiratory ventilation apparatus for a long time (e.g., 10h), as well as to reduce a stimulation of the respiratory mucosa by cold and dry gas, most of the respiratory ventilation apparatus are equipped with the reservoir. By heating the liquid in the reservoir, water vapor may be produced, making the gas inhaled into the user's body warm and moist. The cover plate without a reservoir may be used when the user uses the respiratory ventilation apparatus for a short period of time (e.g., 2 hours), or when there is no need to heat or humidify the gas. The main body and the reservoir of a split respiratory ventilation apparatus may be disassembled and assembled, which makes it easier for carry and more convenient to clean the reservoir and add liquid. In order to make the reservoir and the main body more firmly connected and to have a better sealing performance, a sealing structure is proposed in the following embodiment for the sealing connection between the main body and a connecting device (including the reservoir and the cover plate without a reservoir).

FIG. 5A is a schematic diagram illustrating a structure of a main body of a respiratory ventilation apparatus (also referred to as a respiratory ventilation apparatus main body) according to some embodiments of the present disclosure when the main body of the respiratory ventilation apparatus is not connected to a connecting device; FIG. 5B is a schematic structural diagram illustrating a main body of a respiratory ventilation apparatus when the main body of the respiratory ventilation apparatus is connected to a connecting device by a sealing structure according to some embodiments according to the present disclosure; FIG. 5C is a schematic diagram illustrating a structure of a first elastic pipe according to some embodiments of the present disclosure; FIG. 5D is a schematic diagram illustrating the first elastic pipe of FIG. 5C from another angle; and FIG. 5E is a schematic diagram illustrating a section of the first elastic pipe shown in FIG. 5C.

In some embodiments, combining FIGS. 5A to 5C, a respiratory ventilation apparatus 500 may include a main body 510, a connecting device 520, and a sealing structure 530. The main body 510 may be configured to generate a high pressure gas above an atmospheric pressure. The connecting device 520 may include a breather pipe 521 that receives the high pressure gas. The sealing structure 530 may be configured to sealingly connect the main body 510 and the connecting device 520. In some embodiments, two ends of the sealing structure 530 may be respectively detachably connected to the main body 510 and the connecting device 520. In some embodiments, one end of the sealing structure 530 may be fixedly connected to the corresponding main body 510 and the connecting device 520, and the other end may be detachably connected to the corresponding main body 510 and the connecting device 520. The sealing structure 530 may include an elastic pipe 531 (e.g., a first elastic pipe 5311 and/or a second elastic pipe 5312 in FIG. 5B). The main body 510 and the connecting device 520 connected to the elastic pipe 531 may be disposed with an annular protrusion 532. The annular protrusion 532 may be configured for a sealing connection between the elastic pipe 531 and the main body 510 or the connecting device 520. When the elastic pipe 531 is connected to the breather pipe 521, the high pressure gas generated on one side of the main body 510 may enter the breather pipe 521 through the elastic pipe 531. In some embodiments, when the breather pipe 521 is connected to the elastic pipe 531, the annular protrusion 532 may be squeezed to generate a great frictional resistance between the breather pipe 521 and the elastic pipe 531. The frictional resistance may limit a separation of the breather pipe 521 from the elastic pipe 531, and act as a seal for the connection between the elastic pipe 531 and the breather pipe 521.

In some embodiments, when the elastic pipe 531 is connected to the corresponding end of the main body 510, the end of the elastic pipe 531 may be inserted into the corresponding end of the main body 510, or the corresponding end of the main body 510 may be inserted into the corresponding end of the elastic pipe 531. In the same way, when the elastic pipe 531 is connected to the connecting device 520, a mating mode of the breather pipe 521 and the elastic pipe 531 may include extending the breather pipe 521 into the elastic pipe 531, or extending the elastic pipe 531 into the breather pipe 521. Merely as an example, taking the breather pipe 521 extending into the elastic pipe 531 as an example, the annular protrusion 532 may be disposed on an inner wall of the elastic pipe 531, or may be disposed on an outer wall of the breather pipe 521. During a process of extending the breather pipe 521 into the elastic pipe 531, the annular protrusion 532 may be squeezed and deformed by the inner wall of the elastic pipe 531 and the outer wall of the breather pipe 521, thereby generating a great frictional resistance. The frictional resistance may restrict the breather pipe 521 from detaching from the elastic pipe 531, and ensure that the breather pipe 521 is firmly fixed in the elastic pipe 531. In addition, as the annular protrusion 532 may fill a gap between an outer wall of the breather pipe 521 and the inner wall of the elastic pipe 531 after being squeezed, the annular protrusion 532 may seal the connection between the elastic pipe 531 and the breather pipe 521, so as to ensure a sealing performance of the connection between the main body 510 and the connecting device 520.

In some embodiments, as shown in FIG. 5K, when the elastic pipe 531 is connected to the main body 510 or the connecting device 520, an elastic sealing edge 536 may be disposed on a pipe wall of the elastic pipe 531, and/or on a corresponding connecting pipe wall of the main body 510 or the connecting device 520 connected to the elastic pipe 531. The elastic sealing edge 536 may have an edge with a preset width. When the elastic pipe 531 is connected to the main body 510 or the connecting device 520, the elastic sealing edge 536 located between the pipe wall of the elastic pipe 531 and the connecting pipe wall of the main body 510 or the connecting device 520 may be squeezed and deformed. As the elastic sealing edge 536 has an edge with a preset width, when the elastic sealing edge 536 is squeezed and deformed, a contact surface seal may be formed between the edge with the preset width and the pipe wall surface in contact with the edge, so that a face seal may be formed on a circumferential pipe wall at the connection between the elastic pipe 531 and the main body 510 or between the elastic pipe 531 and the connecting device 520, thereby improving a sealing performance when the elastic pipe 531 is connected to the main body 510 or the connecting device 520. The preset width may be a distance H between a tail section 5362 of a free end of the elastic sealing edge 536 and the pipe wall of the pipe disposed with the elastic sealing edge 536 as shown in FIG. 5K. Specifically, the elastic sealing edge 536 may extend circumferentially on the pipe wall where the elastic sealing edge 536 is disposed, and the elastic sealing edge 536 may be disposed on an inner wall or an outer wall of the connecting pipe. The elastic sealing edge 536 may be located within a connection gap 537 so as to seal the connection gap 537. Specifically, the elastic sealing edge 536 may be disposed on an axial end surface of the connecting pipe (as shown in FIG. 5K and FIG. 5N), or may be disposed on an axial pipe wall of the connecting pipe apart from the end surface (as shown in FIG. 5O). There may be one or more elastic sealing edges 536 (as shown in FIG. 5O), and the elastic sealing edges 536 may be spaced apart along an axis direction of the pipe wall where the elastic sealing edges 536 are disposed.

In the above embodiment, as shown in FIG. 5M, to limit the axial connecting position between the elastic pipe 531 and the main body 510 or between the elastic pipe 531 and the connecting device 520, and to better implement the seal when the elastic pipe 531 is connected to the main body 510 or the connecting device 520, a stopping portion 522 may be disposed around the outer wall of any connecting pipe. It may be understood that in FIG. 5M, when the breather pipe 521 of the connecting device 520 is inserted into the elastic pipe 531, the outer wall of the breather pipe 521 may be disposed with the stopping portion 522, the stopping portion 522 may be abut against the end surface of the elastic pipe 531, and an edge of the stopping portion 522 may exceed an edge of a connection hole of the elastic pipe 531. By disposing the stopping portion 522, not only the position of the axial connection between the elastic pipe 531 and the breather pipe 521 is limited, a sealing performance of the connection between the elastic pipe 531 and the breather pipe 521 may be further optimized, so as to avoid the situation where when the elastic pipe 531 and the breather pipe 521 are connected, the gas flow may overflow from the connection gap 537 between the outer wall of the breather pipe 521 and the inner wall of the elastic pipe 531.

In the above embodiment, the elastic sealing edge 536 may include a fixed end and a free end. The fixed end may be connected to the pipe wall of the connecting pipe, the free end may be far away from the pipe wall of the connecting pipe, and the free end may extend in a direction away from the fixed end along an arc curve. Specifically, the free end may extend obliquely toward the center direction of the axial of the connecting pipe with a greater inner diameter. As shown in FIG. 5M, the hole diameter of the elastic pipe 531 may be greater than the outer diameter of the breather pipe 521, and the free end of the elastic sealing edge 536 may incline toward the center of the elastic pipe 531.

As a ventilation treatment device is used to generate and provide a ventilation gasflow above atmospheric pressure to a patient, a high pressure gasflow (the ventilation gasflow above atmospheric pressure) may flow in the connecting pipe. When the elastic pipe 531 connects the breather pipe 521, the elastic sealing edge 536 may separate the high pressure gasflow from the external atmospheric pressure outside the connection gap 537 in the connection gap 537 at the connection between the elastic pipe 531 and the breather pipe 521. Therefore, there may be a pressure difference between two sides of the elastic sealing edge 536. To avoid a problem of unsealing caused by blowing the free end of the elastic sealing edge 536 outward when a gas path is at high pressure, the free end of the elastic sealing edge 536 may be set to extend obliquely in the direction away from the external atmospheric pressure (e.g., extend obliquely along a certain arc curve), that is, extend toward the inner center direction of the elastic pipe 531. As a result, the high-pressure gasflow flowing in the connection gap 537 may exert a force in a downward direction (that is, toward the inside of the pipeline) on the free end of the elastic sealing edge 536 that is in contact with the pipe wall, thus ensuring the sealing performance of the elastic sealing edge 536. More specifically, the free end of the elastic sealing edge 536 may include a middle section 5361 and a tail section 5362. The middle section 5361 may be close to one side of the fixed end. When the connecting pipe is connected, the elastic sealing edge 536 may move and deform, so that at least the middle section 5361 is in contact with the corresponding pipe wall surface, thereby forming a contact surface seal in the circumferential direction of the pipe wall. Preferably, a thickness of the elastic sealing edge 536 from the fixed end to the middle section 5361 and the tail section 5362 of the free end may gradually decrease. Therefore, the free end of the elastic sealing edge 536, especially the tail section 5362, may have a smaller thickness and may be prone to deformation. In this way, a sealing effect may be achieved in a smaller space, so under the same volume, more space may be spared for other components. Correspondingly, if the connecting pipe stops ventilating, the elastic sealing edge 536 may not be affected by the high pressure of the circulating gasflow. A tightening effect between the elastic sealing edge 536 and the contacting pipe wall may be eliminated through an elastic recovery ability of the elastic sealing edge 536. In this way, the connecting pipe can be easily separated without applying too much tension.

In some embodiments, the breather pipe 521 may include a gas inlet channel 521-1 and a gas outlet channel 521-2. The gas may enter the gas inlet channel 521-1 through a corresponding elastic pipe 531 (e.g., a first elastic pipe 5311 communicating the gas inlet channel 521-1), and then discharged to an external interface of the respiratory ventilation apparatus 500 through the gas outlet channel 521-2 via the corresponding elastic pipe 531 (e.g., the second elastic pipe 5312 communicating the gas outlet channel 521-2) for an inhalation by the user.

In some embodiments, the main body 510 of the respiratory ventilation apparatus 500 may include a blower (not shown in the figure), and the blower may be configured to pressurize the inhaled gas to generate the high-pressure gas. The generated high-pressure gas may enter the connecting device 520 through the sealing structure 530. The gas may be circulated through the connecting device 520 and provided to the user for inhalation. In some embodiments, a gas pressure of the high pressure gas may be equivalent to a pressure of a water column with a height of 4 cm to 40 cm. In some embodiments, a noise reduction device may also be disposed within the main body 510 of the respiratory ventilation apparatus 500, and the blower may be disposed within the noise reduction device to reduce the noise of the blower. A gas outlet of the noise reduction device may be connected to the elastic pipe 531. When the sealing structure 530 is connected to the connecting device 520, the high pressure gas generated by the blower may enter the gas inlet channel 521-1 through the sealing structure 530 and may be discharged out of the gas outlet channel 521-2 to an external interface of the respiratory ventilation apparatus 500 for inhalation by the user.

In some embodiments, a number of the elastic pipe 531 may be one, and the elastic pipe 531 may be selectively mated with either the gas inlet channel 521-1 or the gas outlet channel 521-2. In some embodiments, the elastic pipe 531 may be an integrated structure or a multi-sectioned detachable connection structure.

In some embodiments, the elastic pipe 531 may include a first elastic pipe 5311 and a second elastic pipe 5312. The first elastic pipe 5311 may be configured to communicate with the gas inlet channel 521-1, and the second elastic pipe 5312 may be configured to communicate with the gas outlet channel 521-2. For ease of description, FIGS. 5D to 5E exemplarily illustrate the structure of the first elastic pipe 5311, and the present disclosure will use the first elastic pipe 5311 as an example when describing the elastic pipe 531, and the second elastic pipe 5312 may have the same or similar structure as the first elastic pipe 5311, which is not repeated herein.

The annular protrusion 532 refers to a protruding structure disposed circumferentially around the elastic pipe 531 or the breather pipe 521. In some embodiments, the annular protrusion 532 may be disposed on the breather pipe 521 or the elastic pipe 531. For example, when the breather pipe 521 is mated to the elastic pipe 531 in such a way that the breather pipe 521 extends into the elastic pipe 531 (e.g., the gas inlet channel 521-1 extends into the first elastic pipe 5311), the annular protrusion 532 may be disposed on an outer wall of the breather pipe 521 (e.g., the outer wall of the gas inlet channel 521-1). In a process of the breather pipe 521 extending into the elastic pipe 531, the annular protrusion 532 may generate the frictional resistance between the annular protrusion 532 and an inner wall of the elastic pipe 531, or the annular protrusion 532 may be disposed on the inner wall of the elastic pipe 531, and in a process of the breather pipe 521 extending into the elastic pipe 531, the annular protrusion 532 may generate the frictional resistance between the annular protrusion 532 and the outer wall of the breather pipe 521. For another example, when the breather pipe 521 is mated to the elastic pipe 531 in such a way that the elastic pipe 531 extends into the breather pipe 521 (e.g., the first elastic pipe 5311 extends into the gas inlet channel 521-1), the annular protrusion 532 may be disposed on the inner wall of the breather pipe 521. In the process of the elastic pipe 531 extending into the breather pipe 521, the annular protrusion 532 may generate the frictional resistance with the outer wall of the elastic pipe 531, or the annular protrusion 532 may be disposed on the outer wall of the elastic pipe 531, and in the process of the elastic pipe 531 extending into the breather pipe 521, the annular protrusion 532 may generate the frictional resistance.

In some embodiments, the outer wall of the breather pipe 521 (e.g., the gas outlet channel 521-2 and/or the gas inlet channel 521-1) or the outer wall of the elastic pipe 531 may be disposed with annular recesses (not shown in the figures), and the annular recesses may be coupled with the annular protrusions 532. For example, the annular recess may be disposed in the inner wall of the elastic pipe 531 when the annular protrusion 532 is disposed in the outer wall of the breather pipe 521. For another example, when the annular protrusion 532 is disposed on the inner wall of the breather pipe 521, the annular recess may be disposed on the outer wall of the elastic pipe 531. For another example, when the annular protrusion 532 is disposed on the outer wall of the elastic pipe 531, the annular recess may be disposed on the inner wall of the breather pipe 521. For another example, when the annular protrusion 532 is disposed on the inner wall of the elastic pipe 531, the annular recess may be disposed on the outer wall of the breather pipe 521. In some embodiments, the breather pipe 521 may be inserted into the elastic pipe 531 (e.g., the gas outlet channel 521-2 may be inserted into the second elastic pipe 5312 and/or the gas inlet channel 521-1 may be inserted into the first elastic pipe 5311 to make the annular protrusion 532 disposed within the annular recess), or when the elastic pipe 531 is inserted into the breather pipe 521, the annular recess may limit a movement of the annular protrusion 532, thereby limiting a relative movement of the elastic pipe 531 and the breather pipe 521, and further improving a firmness and a sealing performance of the connection between the main body 510 and the connecting device 520.

In some embodiments, the annular recess may be an annular opening disposed on the breather pipe 521 (e.g., the gas outlet channel 521-2 and/or the gas inlet channel 521-1) or the elastic pipe 531.

In some embodiments, the annular recess may be a recessed structure disposed on a surface of the breather pipe 521 (e.g., the gas outlet channel 521-2 and/or the gas inlet channel 521-1) or the elastic pipe 531. Merely by way of example, the recessed structure may include a recessed sidewall. The recessed sidewall may be an annular rib disposed on the outer wall of the breather pipe 521, and two adjacent annular ribs may define the annular recess or a portion of the annular recess. Taking the gas inlet channel 521-1 as an example, a number of the annular protrusion 532 may be at least two, a number of the annular ribs may be at least two. The at least two annular ribs and the at least two annular protrusions 532 may be disposed at the same spacing on the outer wall of the gas inlet channel 521-1 and the inner wall of the first elastic pipe 5311, respectively. During the insertion of the gas inlet channel 521-1 into the first elastic pipe 5311, the annular rib may be inserted into a gap formed by the two adjacent annular protrusions 532, and the annular protrusions 532 may be inserted into the annular recess formed by the two adjacent annular ribs. In this way, the firmness, and the sealing performance of the connection between the main body 510 and the connecting device 520 may be further improved. In some embodiments, the annular rib may have a certain elasticity so that when the annular protrusions 532 are squeezed with the annular ribs, the annular ribs may be able to deform, so that the annular protrusions 532 may enter the annular recesses. In some embodiments, the annular protrusion 532 and the annular rib may be the same or similar.

In some embodiments, the annular protrusion 532 may be in a form of a strip, and as shown in conjunction with FIG. 5A-FIG. 5E, a sectional shape of the annular protrusion 532 may include a triangle, a rectangle, a hemisphere, etc. In some specific embodiments, the sectional shape of the annular protrusion 532 may be triangular. In some embodiments, the annular protrusion 532 may be in the shape of a bar, a cone, a sphere, a hemisphere, or a column, etc.

In some embodiments, the annular protrusion 532 may be continuous. For example, the annular protrusion 532 may be in a strip shape, and the strip-shaped annular protrusion 532 may be disposed in a spiral around the inner wall of the elastic pipe 531. At this time, the annular protrusion 532 may be regarded as continuous. In some embodiments, the annular protrusion 532 may be a sectioned protruding structure. For example, the annular protrusion 532 may include a plurality of sub-protruding structures, and the plurality of sub-protruding structures may be spaced apart around an inner wall of the elastic pipe 531. In some embodiments, a shape of the sub protruding structure may include a bar, a cone, a sphere, a hemisphere, or a column, etc.

In some embodiments, a number of the annular protrusion 532 may be one. For example, when the number of annular protrusion 532 is one, the annular protrusion 532 may be in the form of a strip, and the strip-shaped annular protrusions 532 may be disposed in the spiral around the inner wall of the elastic pipe 531.

In some embodiments, the number of the annular protrusions 532 may be more than one, and the plurality of annular protrusions 532 may be disposed at equal intervals along a length direction of the elastic pipe 531 or the breather pipe 521 to enhance the sealing performance and firmness of the connection between the main body 510 and the connecting device 520. The length direction of the elastic pipe 531 or the breather pipe 521 may be indicated by the arrow X in FIG. 5A. In some embodiments, the plurality of annular protrusions 532 may all be in the form of strips, and a number of strip-shaped annular protrusions 532 may be disposed in a helical pattern, i.e., the number of annular protrusions 532 may be spaced apart in a helical trajectory. Merely by way of example, in the embodiment shown in FIG. 5C, the number of annular protrusions 532 of the first elastic pipe 5311 may be two. In some embodiments, the plurality of annular protrusions 532 may be the same or different. Merely by way of example, in the embodiment shown in FIG. 5C, the two annular protrusions 532 may be of the same shape and size. In another embodiment, the annular protrusion 532 may be disposed on the inner wall of the elastic pipe 531, and the annular protrusion 532 may include a first annular protrusion and a second annular protrusion. The first annular protrusion may be near an insertion (e.g., an insertion 535 of the first elastic pipe 5311 in FIG. 5D), and the second annular protrusion may be away from the insertion. An inner diameter of the elastic pipe (e.g., the first elastic pipe 5311 of FIG. 5D) may be gradually narrowed from the side near the insertion to the side away from the insertion, and the first annular protrusion may have a height and width that are greater than the height and width of the second annular protrusion to enable both the first annular protrusion and the second annular protrusion to touch the outer wall of a gas inlet pipe (e.g., the gas inlet channel 521-1 in FIG. 5A) and/or a gas outlet pipe (e.g., the gas outlet channel 521-2 in FIG. 5A). The height of the annular protrusion 532 may be a size of the annular protrusion 532 in a radial direction of the elastic pipe 531. The radial direction of the elastic pipe 531 refers to the direction of any line passing a central axis of the elastic pipe 531 on a plane formed by an arrow Z and an arrow Y in FIG. 5C. The width of the annular protrusion 532 refers to a size of the annular protrusion 532 in the length direction of the elastic pipe 531, as shown by an arrow X in FIG. 5C. In some embodiments, the length direction of the main body 510 may be parallel to the length direction of the elastic pipe 531, the thickness direction of the main body 510 may be indicated by the arrow Z, and the width direction of the main body 510 may be indicated by the arrow Y.

In some embodiments, the annular protrusion 532 may protrude in a direction away from an insertion direction of the elastic pipe 531 and the breather pipe 521. For example, when the mating mode of the breather pipe 521 with the elastic pipe 531 is such that the breather pipe 521 extends into the elastic pipe 531 (e.g., the gas inlet channel 521-1 extends into the first elastic pipe 5311), the annular protrusion 532 may protrude in the direction away from the insertion 535 of the elastic pipe 531, that is, extends at an angle in a direction away from the insertion 535 of the elastic pipe 531, so as to enable the breather pipe 521 to be smoothly extended into the elastic pipe 531 through the insertion 535, and to effectively prevent the breather pipe 521 from detaching from the elastic pipe 531. In this way, the firmness, and the sealing performance of the main body 510 connected to the connecting device 520 may be further improved. Merely by way of example, as shown in FIG. 5E, taking the first elastic pipe 5311 as an example, the annular protrusion 532 of the first elastic pipe 5311 may include a first sidewall 5321 and a second sidewall 5322. The first sidewall 5321 may be close to the insertion 535 (i.e., an opening of the first elastic pipe 5311 for the insertion of the gas inlet channel 521-1), the second sidewall 5322 may be far away from the insertion 535 of the first elastic pipe 5311, and a radial length (i.e., the diameter) of the first sidewall 5321 may be greater than the radial length (i.e., the diameter) of the second sidewall 5322, so that the two sidewalls of the annular protrusion 532 may have a non-symmetrical structure and may be inclined toward the internal direction of the main body 510. During the insertion of the gas inlet channel 521-1 into the first elastic pipe 5311, as the annular protrusion 532 protrudes in an inclined way, the frictional resistance generated by the end of the annular protrusion 532 and the outer wall of the gas inlet channel 521-1 may have a smaller component force in an insertion movement direction in the gas inlet channel 521-1, and the gas inlet channel 521-1 may be more smoothly inserted into the first elastic pipe 5311. During a withdrawal of the gas inlet channel 521-1 from the first elastic pipe 5311, the frictional resistance generated by the end of the annular protrusion 532 and the outer wall of the gas inlet channel 521-1 may have a greater component force in a pull-out disengagement movement direction of the gas inlet channel 521-1, thereby effectively preventing the gas inlet channel 521-1 from disengaging from the first elastic pipe 5311.

In some embodiments, the annular protrusion 532 and the elastic pipe 531 or the breather pipe 521 may be independently fabricated before assembly. In some embodiments, the annular protrusion 532 and the elastic pipe 531 or the breather pipe 521 may be made by one-piece molding, such as injection molding. In some embodiments, the elastic pipe 531 may be a rubber pipe, a silicone pipe, a plastic pipe, etc.

In some embodiments, the limiting groove 533 is provided in an outer wall of the elastic pipe 531 or an outer wall of the breather pipe 521 (e.g., the gas inlet channel 521-1 and/or the gas outlet channel 521-2), and the main body 510 may include a limiting structure (not shown in the figures). The limiting structure may be adapted to fit with the limiting groove 533, and when the limiting structure is mated with the limiting groove 533, it may limit a movement range of the elastic pipe 531 or the breather pipe 521 in a radial direction. For example, the limiting groove 533 may be provided on the outer wall of the elastic pipe 531, and when the breather pipe 521 extends into the elastic pipe 531, the limiting groove 533 may be used to restrict the movement range of the elastic pipe 531 in the radial direction. For example, the limiting groove 533 may be provided on an outer wall of the breather pipe 521, and when the elastic pipe 531 extends into the breather pipe 521, the limiting groove 533 may be used to limit the movement range of the breather pipe 521 in the radial direction Merely by way of example, shown in FIGS. 5B-5E, taking the first elastic pipe 5311 as an example, the limiting groove 533 is provided on an outer wall of the first elastic pipe 5311, which is arranged along the first elastic pipe 5311 in a circumferential direction. The limiting structure may include a notch opened on a card plate (not shown in the figures) connected to the main body housing 511, and the limiting groove 533 of the first elastic pipe 5311 is able to snap into a notch of the card plate. In the process of extending the gas inlet channel 521-1 into the first elastic pipe 5311, a force generated by contact between the gas inlet channel 521-1 and the annular protrusion 532 may cause the first elastic pipe 5311 to move in the radial direction. At this time, the limiting structure may constrain the movement range of the first elastic pipe 5311 in the radial direction.

In some embodiments, the outer diameter of the limiting groove 533 is smaller than the inner diameter of the limiting structure, thereby reserving a certain movement space for the elastic pipe 531 or the breather pipe 521, and facilitating the breather pipe 521 to be inserted smoothly into the elastic pipe 531, or the elastic pipe 531 smoothly inserted into the breather pipe 521. Merely by way of example, taking extending the gas inlet channel 521-1 into the first elastic pipe 5311 as an example, when a center axis of the gas inlet channel 521-1 is slightly offset from a center axis of the first elastic pipe 5311. For example, when the center axis of the gas inlet channel 521-1 is slightly offset upward in the radial direction of first elastic pipe 5311 (e.g., a Z-axis), the annular protrusion 532 may contact with an upper portion of the outer wall of the gas inlet channel 521-1, and then a force is generated. Since an outer diameter of the limiting groove 533 is smaller than an inner diameter of the limit structure, there is a gap between the limiting structure and the limiting groove 533, which reserves a certain movement space for the first elastic pipe 5311, and thus the generated force may cause the first elastic pipe 5311 to move along the radial direction (e.g., the Z-axis). Ultimately, the center axis of the first elastic pipe 5311 is aligned with the center axis of the gas inlet channel 521-1, so that the gas inlet channel 521-1 is smoothly inserted into the first elastic pipe 5311. In some embodiments, a ratio of the outer diameter of the limiting groove 533 to the inner diameter of the limiting structure may be within a range of 0.5 to 1. In some embodiments, a ratio of the outer diameter of the limiting groove 533 to the inner diameter of the limiting structure may be within a range of 0.6 to 1. In some embodiments, a ratio of the outer diameter of the limiting groove 533 to the inner diameter of the limiting structure may be within a range of 0.7 to 1.

In some embodiments, the limiting groove 533 includes one or more limiting protrusions 534, the one or more limiting protrusions 534 may be provided along an outer wall of the limiting groove 533 in a circumferential direction. A distance between an outer wall of a limiting protrusion 534 and a center axis of the limiting groove 533 may be greater than or equal to the inner diameter of the limiting structure. Merely by way of example, during a process of extending the gas inlet channel 521-1 into the first elastic pipe 5311, when the center axis of the gas inlet channel 521-1 is slightly offset (e.g., upwardly offset along the radial direction (e.g., the Z-axis)) from the center axis of the first elastic pipe 5311, the first elastic pipe 5311 may contact with the upper portion of the outer wall of the gas inlet channel 521-1 to generate a force to cause the limiting protrusion 534 to deform, ensuring that the center axis of the gas inlet channel 521-1 is coaxial with the center axis of the first elastic pipe 5311, so as to make it easier for the gas inlet channel 521-1 to extend into the first elastic pipe 5311. In some embodiments, the limiting protrusion 534 may be made of rubber, plastic, silicone, or the like.

In some embodiments, a portion of the breather pipe 521 (e.g., the gas inlet channel 521-1 and/or the gas outlet channel 521-2) that is connected to the sealing structure 530 (e.g., the elastic pipe 531) has a graduated diameter. Merely by way of example, shown in conjunction with FIGS. 5A, 5B, 5F, and 5H, the gas inlet channel 521-1 includes the first gas inlet end 5211 and the first gas outlet end 5212. The gas outlet channel 521-2 includes the second gas inlet end 5221 and the second gas outlet end 5222. The first gas inlet end 5211 is used to be inserted into the first elastic pipe 5311, and high-pressure gas may be discharged out of the gas inlet channel 521-1 through the first gas outlet end 5212. The second gas outlet end 5222 is used to be inserted into the second elastic pipe 5312 and connected to an external interface of the respiratory ventilation apparatus 500. The gas discharged from the first gas outlet end 5212 may enter the gas outlet channel 521-2 through the second gas inlet end 5221 and be discharged to the external interface of the respiratory ventilation apparatus 500 through the second gas outlet end 5222. An outer diameter of the first gas inlet end 5211 gradually decreases from a direction close to the first gas outlet end 5212 to a direction far away from the first gas outlet end 5212, allowing the first gas inlet end 5211 to be smoothly inserted into the first elastic pipe 5311. An outer diameter of the second gas outlet end 5222 is gradually reduced from a direction close to the second gas inlet end 5221 to a direction away from the second gas inlet end 5221, allowing the second gas outlet end 5222 to be smoothly inserted into the second elastic pipe 5312, reducing a frictional resistance from the annular protrusion 532 when inserting the gas inlet channel 521-1 into the first elastic pipe 5311 and a frictional resistance from the annular protrusion 532 when inserting the gas outlet channel 521-2 into the second elastic pipe 5312. As another example, as shown in FIG. 5E, an inner diameter of the first elastic pipe 5311 gradually decreases in a direction from close to the insertion 535 of the first elastic pipe 5311 to away from the insertion 535 of the first elastic pipe 5311, allowing a gas inlet pipe (e.g., the gas inlet channel 521-1 in FIG. 5A) to be smoothly inserted into the first elastic pipe 5311.

In some embodiments, length directions of the first gas inlet end 5211 and the second gas outlet end 5222 may be parallel to a length direction of the elastic pipe 531. Length directions of the first gas outlet end 5212 and the gas second inlet end 5221 may be parallel to the length direction of the elastic pipe 531. Radial directions of the first gas outlet end 5212 and the second gas inlet end 5221 refer to a direction of any straight line in a plane formed by an arrow Z and an arrow Y that passes over the center axis of the gas inlet channel 521-1 and the gas outlet channel 521-2. The length directions of the first gas outlet end 5212 and the second gas inlet end 5221 may be parallel to the length direction of the elastic pipe 531. The length directions of the first gas outlet end 5212 and the second gas inlet end 5221 may be parallel to a width direction of the top cover 526, which is indicated by an arrow Y. The radial directions of the first gas outlet end 5212 and the second gas inlet end 5221 refer to the direction of any straight line in the plane formed by the arrow Z and an arrow X that passes over the center axis of the gas inlet channel 521-1 and the gas outlet channel 521-2.

In some embodiments, a ratio of a maximum value of the outer diameter of the first gas inlet end 5211 to a minimum value of the outer diameter of the first gas inlet end 5211 may be within a range of 2 to 1.05. In some embodiments, the ratio of a maximum value of the outer diameter of the first gas inlet end 5211 to a minimum value of the outer diameter of the first gas inlet end 5211 may be within a range of 2 to 1.1. In some embodiments, the ratio of a maximum value of the outer diameter of the first gas inlet end 5211 to a minimum value of the outer diameter of the first gas inlet end 5211 may be within a range of 2 to 1.5. In some embodiments, a ratio of a maximum value of an outer diameter of the second gas outlet end 5222 to a minimum value of the outer diameter of the second gas outlet end 5222 may be the same as or similar to the first gas inlet end 5211.

In some embodiments, a portion of the breather pipe 521 (e.g., the gas inlet channel 521-1 and/or the gas outlet channel 521-2) that is connected to the sealing structure 530 (e.g., the elastic pipe 531) is provided with a chamfer for further minimizing a frictional resistance produced by extending the breather pipe 521 into the elastic pipe 531, or a frictional resistance produced by extruding the annular protrusion 532 when extending the elastic pipe 531 into the breather pipe 521. Merely by way of example, as shown in FIG. 5I, an end surface of the first gas inlet end 5211 of the gas inlet channel 521-1 is provided with a rounded chamfer. As another example, an end face of the first gas inlet end 5211 of the gas inlet channel 521-1 is provided with a ramp chamfer. Merely by way of example, as shown in FIG. 5E, the insertion 535 of the first elastic pipe 5311 is provided with a rounded chamfer, which not only enlarges a size of the insertion 535 of the first elastic pipe 5311, but also reduces a frictional resistance between the insertion 535 of the first elastic pipe 5311 and the gas inlet pipe (e.g., the gas inlet channel 521-1 in FIG. 5A), and also makes it easier for the gas inlet pipe to find a correct position for easy introduction when extending into the first elastic pipe 5311.

In some embodiments, shown in conjunction with FIGS. 5B and 5F, a first stopper 524 is provided on an outer wall of the breather pipe 521 (e.g., the gas inlet channel 521-1 and/or the gas outlet channel 521-2), and the first stopper 524 may limit a length for the gas inlet channel 521-1 and/or the gas outlet channel 521-2 to be inserted into a corresponding elastic pipe 531. Merely by way of example, the first stopper 524 may be a stop ring provided on an outer wall of the gas inlet channel 521-1 and the gas outlet channel 521-2. With the outer diameter of the stop ring being greater than an inner diameter of the first elastic pipe 5311 (e.g., the inner diameter of the insertion 535 in FIG. 5D) and the inner diameter of the second elastic pipe 5312, when the gas inlet channel 521-1 and the gas outlet channel 521-2 are inserted a certain distance inside the first elastic pipe 5311 and the second elastic pipe 5312 respectively. The stop ring resists against an end face of the insertion of the first elastic pipe 5311 and the second elastic pipe 5312, thereby restricting the gas inlet channel 521-1 and the gas outlet channel 521-2 from extending further. In some embodiments, a manner for mating the breather pipe 521 to the elastic pipe 531 is when the elastic pipe 531 extends into the breather pipe 521, the first stopper 524 may be provided on the outer wall of the elastic pipe 531.

In some embodiments, shown in conjunction with FIGS. 5A-5G, the top cover 526 of the connecting device 520 is provided with a second stopper 525, and the second stopper 525 may limit a length for the breather pipe 521 (e.g., the gas inlet channel 521-1 and/or the gas outlet channel 521-2) to be inserted into a corresponding elastic pipe 531. Merely by way of example, the main body 510 may include the main body housing 511, the first elastic pipe 5311 and the second elastic pipe 5312 are provided in the main body housing 511. The second stopper 525 may include tabs provided an upper surface and a lower surface of the top cover 526, and outer diameters of the tabs are larger than an inner diameter of the main body housing 511. Therefore, when the gas inlet channel 521-1 and the gas outlet channel 521-2 are extended for a certain distance into the first elastic pipe 5311 and the second elastic pipe 5312 respectively. The second stopper 525 may be resisted against the main body housing 511, thereby restricting the gas inlet channel 521-1 and the gas outlet channel 521-2 from extending further.

In some embodiments, the connecting device 520 includes a reservoir or a cover plate without a reservoir.

In some embodiments, shown in conjunction with FIGS. 5F-5G, when the connecting device 520 is the reservoir, a liquid is stored in the reservoir, the first gas outlet end (not shown in the figures) of the gas inlet channel 521-1 is connected to an interior of the reservoir, and the second gas inlet end (not shown in the figures) of the gas outlet channel 521-2 is connected to the interior of the reservoir. Gas pressurized by a blower may enter the interior of the reservoir through the gas inlet channel 521-1 and move to a top of a liquid surface in the reservoir, and may be discharged through the gas outlet channel 521-2 for inhalation by a user. In some embodiments, the reservoir may also be provided with a heating device or a heat conducting device, and the heating device or the heat conducting device may cause the liquid in the reservoir to heat up and produce water vapor to make gas inhaled into the user warm and moist. Further descriptions regarding the reservoir may be found in FIGS. 6A-6I and FIGS. 7A-7G and related descriptions thereof, and may not be repeated here.

In some embodiments, shown in conjunction with FIGS. 5H-5J, when the connecting device 520 is a communication structure connected to the breather pipe 521, such as the cover plate without a reservoir, the first gas outlet end 5212 of the gas inlet channel 521-1 is connected to the gas outlet channel 521-2 of the second gas inlet end 5221. The gas pressurized by the blower passes through the gas inlet channel 521-1 into the gas outlet channel 521-2 and is discharged to the external interface of the respiratory ventilation apparatus (e.g., the respiratory ventilation apparatus 500 in FIG. 5A) for inhalation by the user.

In some embodiments, the guide rib 523 is provided on an outer wall of the breather pipe 521 (e.g., the gas inlet channel 521-1 and/or the gas outlet channel 521-2) of the connecting device 520 or the outer wall of the elastic pipe 531, facilitating positioning the breather pipe 521 during an insertion of the breather pipe 521 into the elastic pipe 531, or positioning the elastic pipe 531 during an insertion of the elastic pipe 531 into the breather pipe 521. Merely by way of example, as shown in conjunction with FIG. 5A, and FIGS. 5H-5J, the main body housing 511 may include the housing top wall 512 and the housing bottom wall 513. The cover plate without a reservoir may include the top cover 526, the gas inlet channel 521-1 and the gas outlet channel 521-2 are provided on the top cover 526, and the guide rib 523 is provided at the top of the outer wall of the gas inlet channel 521-1, and the guide rib 523 extends along a direction of a center axis of the gas inlet channel 521-1. In a process of installing the cover plate without a reservoir, the gas inlet channel 521-1 needs to be inserted into the main body housing 511 to move a certain distance before it can be extended into the first elastic pipe (e.g., the first elastic pipe 5311 in FIG. 5B). The gas inlet channel 521-1 may offset during movement, causing the gas inlet channel 521-1 to not be aligned with the first elastic pipe and thus not be able to extend into the first elastic pipe. After the guide rib 523 is provided on the gas inlet channel 521-1, the guide rib 523 may be made to resist against the housing top wall 512 to help the user orient the gas inlet channel 521-1. When the guide rib 523 on the gas inlet channel 521-1 resists against the housing top wall 512, it indicates that the gas inlet channel 521-1 is aligned with the first elastic pipe. At this time, it is only necessary to control the gas inlet channel 521-1 to continue to move into the first elastic pipe to mate with the first elastic pipe, effectively improving installation efficiency of the cover plate without a reservoir. As another example, the top of the outer wall of the gas inlet channel 521-1 and the top of the outer wall of the outlet channel 521-2 are provided with guiding ribs 523. When installing the cover plate without a reservoir, it is possible to make the guide rib 523 of the gas inlet channel 521-1 and the guide rib 523 of the gas outlet channel 521-2 respectively resist against the housing top wall 512 to realize guided positioning.

In some embodiments, the guide rib 523 is removably connected to the breather pipe 521 (e.g., the gas inlet channel 521-1 and/or the gas outlet channel 521-2) or the elastic pipe 531. A removable connection manner includes bonding, screw connection, magnetic connection, etc. In some embodiments, the guide rib 523 is fixedly connected to the breather pipe 521 (e.g., the gas inlet channel 521-1 and/or the gas outlet channel 521-2) or the elastic pipe 531. Merely by way of example, the guide rib 523, the gas inlet channel 521-1, the gas outlet channel 521-2, or the elastic pipe 531 are fabricated by way of one-piece molding.

In some embodiments, the inner wall of the main body housing 511 is provided with a guiding groove (not shown in the figures) adapted to the guide rib 523, and the guiding groove may improve guiding and positioning accuracy of the cover plate without a reservoir when the guiding groove is mated to the guiding rib 523.

In some embodiments, as shown in FIG. 5H, a flow-limiting structure 527 is provided between the gas inlet channel 521-1 and the gas outlet channel 521-2 of the cover plate without a reservoir for adjusting a gas resistance between the gas inlet channel 521-1 and the gas outlet channel 521-2 to be close to a gas resistance of a gas inlet pipe and a gas outlet pipe of the reservoir of the embodiment. In some embodiments, a flow-limiting structure 527 may include a first flow-limiting pipe 5271 and a second flow-limiting pipe 5272, the first flow-limiting pipe 5271 is connected to the first gas outlet end 5212 of the gas inlet channel 521-1, and the second flow-limiting pipe 5272 is connected to the second gas inlet end 5221 of the gas outlet channel 521-2. The first flow-limiting pipe 5271 is connected to the second flow-limiting pipe 5272. An inner diameter of the first flow-limiting pipe 5271 is gradually decreased from a side away from the second flow-limiting pipe 5272 to a side close to the second flow-limiting pipe 5272. An inner diameter of the second flow-limiting pipe 5272 is gradually decreased from a side away from the first flow-limiting pipe 5271 to a side close to the first flow-limiting pipe 5271. In some embodiments, a maximum value of the inner diameter of the first flow-limiting pipe 5271 is the same as the inner diameter of the gas inlet channel 521-1. A ratio of the maximum value of the inner diameter of the first flow-limiting pipe 5271 to a minimum value of the inner diameter of the gas inlet channel 521-1 may be within a range of 0.9 to 0.05. The ratio of the maximum value of the inner diameter of the first flow-limiting pipe 5271 to the minimum value of the inner diameter of the gas inlet channel 521-1 may be within a range of 0.9 to 0.075. The ratio of the maximum value of the inner diameter of the first flow-limiting pipe 5271 to the minimum value of the inner diameter of the gas inlet channel 521-1 may be within a range of 0.9 to 0.1. In some embodiments, the second flow-limiting pipe 5272 is the same or similar to the first flow-limiting pipe 5271.

In some embodiments, the flow-limiting structure 527 may include a third flow-limiting pipe (not shown in the figures), the third flow-limiting pipe may be connected to the first gas outlet end 5212 of the gas inlet channel 521-1 and the second gas inlet end 5221 of the gas outlet channel 521-2. An inner diameter of the third flow-limiting pipe is smaller than the inner diameter of the gas inlet channel 521-1 and the inner diameter of the gas outlet channel 521-2. In some embodiments, a minimum value of the inner diameter of the third flow-limiting pipe is the same or similar to the minimum value of the inner diameter of the first flow-limiting pipe 5271.

In some embodiments, the connecting device 520 may include a locking device, and the locking device may be used to lock a mated connecting device 520 with the main body 510 to prevent the connecting device 520 from detaching from the main body 510. In some embodiments, the locking device may include one or more snap-in structures, the snap-in structures are used for engaging with the main body 510. In some embodiments, shown in conjunction with FIG. 5A as well as FIGS. 5F-5J, the snap-in structure may include a first buckle 528, an upper surface of the top cover 526 of the connecting device 520 is provided with a mounting slot. The first buckle 528 is provided in the mounting slot, and the housing top wall 512 of the main body 510 is provided with a first clamping slot 514 adapted to the first buckle 528. When it is necessary to provide the main body 510 with the connecting device 520, the first buckle 528 may be snapped into the first clamping slot 514 from an inner side of the main body housing 511, thereby securely snapping the main body 510 to the connecting device 520. When it is necessary to dismantle the main body 510 and the connecting device 520, the first buckle 528 may be triggered to exit the first clamping slot 514, thereby unlocking the main body 510 and the connecting device 520. In some embodiments, the snap-in structure may include a pressing button, the first buckle 528, and an elastic portion provided on the upper surface of the top cover 526. The pressing button is connected to the first buckle 528, and the elastic portion is provided between the upper surface of the top cover 526 and the pressing button. The pressing button may be pressed downward under an external force, causing the first buckle 528 to move downward to facilitate a process of assembling the connecting device 520 to the main body 510, or causing the first buckle 528 to be disassembled from the first clamping slot 514 to facilitate a process of detachment between the connecting device 520 and the main body 510. When the external force is released, the elastic portion may cause the pressing button to rebound upwardly, causing the connecting device 520 to snap onto the main body 510, or to reset after disassembling. In some embodiments, a count of the first buckle 528 may be one or more, and a count of the first clamping slot 514 may be the same as the count of the first buckle 528. For example, in the embodiment shown in FIG. 5F, there are two first buckles 528, and correspondingly, the count of the first clamping slot 514 may be two. Further descriptions regarding the pressing button may be found in FIGS. 7A-7G and related descriptions, which may not be repeated here.

In some embodiments, the snap-in structure may include a second buckle 529, and the second buckle 529 may be provided on a lower surface of the top cover 526 of the connecting device 520, and the housing bottom wall 513 of the main body 510 is provided with a second clamping slot 515 adapted to the second buckle 529. When it is necessary to provide the main body 510 with the connecting device 520, the second buckle 529 may be snapped from an inner side of the main body housing 511 into the second clamping slot 515, so as to securely snap the main body 510 to the connecting device 520, thus, the main body 510 is securely snapped to the connecting device 520. When it is necessary to disassemble the main body 510 and the connecting device 520, the second buckle 529 may be triggered to exit the second clamping slot 515, thereby unlocking the main body 510 and the connecting device 520. In some embodiments, a count of the second buckle 529 may be one or more, and a count of the second clamping slot 515 may be the same as the count of the second buckle 529. For example, in the embodiment shown in FIG. 5G, there are two second buckles 529, and correspondingly, the count of the second clamping slot 515 may be two.

In some embodiments, the snap-in structure may include both the first buckle 528 and the second buckle 529, thereby improving a locking effect of the main body 510 with the connecting device 520.

In some embodiments, the locking device may also include a magnetic component. Merely by way of example, the upper surface of the top cover 526 of the connecting device 520 may be provided with a magnetic portion, and the housing top wall 512 of the main body housing 511 is provided with a magnet when the gas inlet channel 521-1 and the gas outlet channel 521-2 are extended into corresponding elastic pipes 531, the magnet may be attracted to the magnetic portion, thereby realizing locking the main body 510 and the connecting device 520.

The respiratory ventilation apparatus provided in the above embodiment is provided with the annular protrusion on the breather pipe or the elastic pipe of the sealing structure, so that during a process of mating the breather pipe of the connecting device and the elastic pipe of the sealing structure, the annular protrusion is allowed to be deformed by squeezing. After deformation, the annular protrusion produces a large friction force between the breather pipe and the elastic pipe to limit separation of the breather pipe and the elastic pipe, which not only ensures sealing performance and firmness of connecting the breather pipe and the elastic pipe, but also simplifies assembly operations, thereby effectively improving assembly efficiency.

Static reflux, as well as dynamic reflux, may be present when the respiratory ventilation apparatus is currently in use. The static reflux may include a possibility of fluid flooding a reservoir first gas inlet or a reservoir second gas inlet when the respiratory ventilation apparatus is placed at an inclined position. The dynamic reflux may include when the respiratory ventilation apparatus is transported or moved, which may cause an interior of the reservoir to swing back and forth, colliding with a sidewall of the reservoir, generating liquid splashes, or the like. The static reflux, as well as the dynamic reflux, may cause water from the reservoir inside the respiratory ventilation apparatus to reflux into various gas channels, thereby affecting normal use of the respiratory ventilation apparatus or causing a patient to chock, which, in severe cases, may endanger a patient's life. Based on this, some embodiments of the present disclosure provide a reservoir, which may reduce a possibility of the water in the reservoir refluxing into the gas channels by means of a reflux prevention component, ensuring the normal use of the respiratory ventilation apparatus, and avoiding a risk for the patient to chock.

FIG. 6A is a schematic diagram illustrating a structure of a reservoir according to some embodiments of the present disclosure; FIG. 6B is a schematic diagram illustrating a structure of a lower housing of a reservoir according to some embodiments of the present disclosure; and FIG. 6C is a schematic diagram illustrating a structure of an upper housing of a reservoir according to some embodiments of the present disclosure.

As shown in FIGS. 6A-6C, a reservoir 600 may include a reservoir housing 610, a heat transfer component 620, a gas inlet channel 630, a gas outlet channel 640, and a reflux prevention component. The reservoir housing 610 may include a reservoir upper housing 611 and a reservoir lower housing 612. The reservoir upper housing 611 and the reservoir lower housing 612 are internally hollow, and the two may cooperate to form a reservoir cavity for accommodating liquid. The gas inlet channel 630 and the gas outlet channel 640 are both provided on the reservoir upper housing 611, and the heat transfer component 620 is provided on the reservoir lower housing 612.

The reservoir 600 may generate steam from the liquid in the reservoir cavity through heat transfer, and the steam may be water steam from pure water, and may also be steam from a mixture of pure water and a medicinal liquid. The reservoir 600 may mix the steam with gas input from a main body of the respiratory ventilation apparatus through the gas inlet channel 630 to generate mixed gas, and output the mixed gas through the gas outlet channel 640, allowing a patient to breathe gas that is humidified or with medicinal steam.

The reservoir housing 610 may be of a plurality of shapes. For example, the reservoir housing 610 may be a rectangular, a square, a cylindrical, a conical, or other shapes. The reservoir cavity in the reservoir housing 610 may be configured to accommodate the liquid, and the reservoir cavity may be of a plurality of shapes including a plurality of wall-like structures. A material of the reservoir housing 610 may be polycarbonate, polystyrene, polypropylene, polymethylmethacrylate, or any other feasible material.

The heat transfer component 620 may be configured to conduct heat to the liquid in the reservoir cavity, thereby converting the liquid from a liquid state to a steam state. When the liquid is converted to the steam, the steam may rise to a space above the reservoir cavity. The heat transfer component 620 may be a plurality of contact or contactless heat transfer components capable of conducting heat to the liquid. The heat transfer component 620 may be provided on various locations in the reservoir lower housing 612. As shown in FIG. 6B, the heat transfer component 620 may be provided on a bottom of the reservoir lower housing 612. In some embodiments, the heat transfer component 620 may also be provided on other locations of the reservoir lower housing 612. For example, the heat transfer component 620 may also be provided on one or more sidewalls of the reservoir lower housing 612.

In some embodiments, as shown in FIG. 6B, a guide rib 612-1 may be disposed on a side of the reservoir lower housing 612. The guide rib 612-1 may extend along a mounting direction of the main body and the reservoir 600 of the respiratory ventilation apparatus. A guide groove matching the guide rib 612-1 may be disposed on the main body of the respiratory ventilation apparatus. When the reservoir 600 is mounted on the main body of the respiratory ventilation apparatus, the guide rib 612-1 may extend into the guide groove of the main body of the respiratory ventilation apparatus and continue to extend along the extending direction of the guide groove for further mounting. The match between the guide rib 612-1 and the guide groove may play a role in guiding and positioning. In some embodiments, there may be two guide ribs 612-1 disposed symmetrically on both sides of the reservoir lower housing 612. A structure of the guide rib 612-1 may be a strip, or may be an enclosed or a semi-enclosed structure, e.g., a semi-enclosed annular runway structure.

The gas inlet channel 630 may be configured to import gas into the reservoir cavity. As shown in FIG. 6C, the gas inlet channel 630 may include a reservoir first gas inlet 631 and reservoir first gas outlet 632. The reservoir first gas inlet 631 is provided on an outside of the reservoir upper housing 611 for supplying gas from outside of the reservoir 600 into the gas inlet channel 630. The reservoir first gas inlet 631 may be connected to a gas outlet of a main body of respiratory ventilation apparatus, and the gas output from the gas outlet of the main body of the respiratory ventilation apparatus may enter the gas inlet channel 630 through the reservoir first gas inlet 631. The reservoir first gas outlet 632 is provided inside the reservoir upper housing 611 for gas in the gas inlet channel 630 to enter into the reservoir cavity. When the gas in the gas inlet channel 630 enters the reservoir cavity, it mixes with steam in a space above the reservoir cavity to produce mixed gas. The gas inlet channel 630 may be a hollow pipe of various shapes. For example, the gas inlet channel 630 may be a round hollow pipe. Furthermore, for example, the gas inlet channel 630 may also be a rectangular hollow pipe.

The gas outlet channel 640 may be configured to export gas in the reservoir cavity. As shown in FIG. 6C, the gas outlet channel 640 may include a reservoir second gas inlet 641 and a reservoir second gas outlet 642. The reservoir second gas inlet 641 is provided inside the reservoir upper housing 611 for supplying gas inside the reservoir cavity into the gas outlet channel 640. The reservoir second gas outlet 642 is provided outside the reservoir upper housing 611 for exporting gas from the gas outlet channel 640. The reservoir second gas outlet 642 may be connected to an external gas hose, one end of the external gas hose may be connected to the reservoir second gas outlet 642, and the other end may be connected to a patient's user interface 7000 (e.g., a respiratory mask or a nasal cannula, etc.). Similar to the gas inlet channel 630, the gas outlet channel 640 may be a hollow pipe of various shapes.

The reflux prevention component is provided on the reservoir upper housing 611 and may be configured to prevent the liquid in the reservoir cavity from refluxing into the gas inlet channel 630 and/or the gas outlet channel 640. In some embodiments, the reflux prevention component may include one or both of a first bending portion 633 on the gas inlet channel 630 and a second bending portion 643 on the gas outlet channel 640 and/or a flow guide rib 650. Further descriptions regarding the first bending portion 633, the second bending portion 643, and the flow guide rib 650 may be found hereinafter.

In some embodiments of the present disclosure, by providing the reflux prevention component in the reservoir 600, the liquid in the reservoir 600 is prevented from flowing back into the gas inlet channel 630 and/or the gas outlet channel 640, which prevents the liquid from flowing through the gas inlet channel 630 into the main body of the respiratory ventilation apparatus to causing the respiratory ventilation apparatus to malfunction, and prevents the liquid from flowing to the patient through the gas outlet channel 640 causing the patient to choke. On the other hand, by forming the reservoir cavity through a separable reservoir upper housing 611 and a separable reservoir lower housing 612 that passes directly through the reservoir housing 610. An overall small size of the reservoir 600 may be ensured while maintaining a capacity of the reservoir 600 for the liquid, avoiding a waste of space caused by adding another built-in reservoir cavity.

In some embodiments, the gas inlet channel 630 and/or the gas outlet channel 640 may be integrally/non-integrally provided with an inner side of the reservoir upper housing 611. When the gas inlet channel 630 and/or the gas outlet channel 640 are integrally provided with the inner side of the reservoir upper housing 611, the gas inlet channel 630 and/or the gas outlet channel 640 is connected to the inner side of the reservoir upper housing 611, and a pipe structure that is sealed around and has openings on both sides is formed by the inner side of the reservoir upper housing 611 and other sidewalls of the gas inlet channel 630 and/or the gas outlet channel 640. As shown in FIG. 6C, one side of the gas inlet channel 630 and one side of the gas outlet channel 640 are directly connected to the inner side of the reservoir upper housing 611 so that the pipe structure that is sealed around and has openings on both sides is formed by the gas inlet channel 630 and the gas outlet channel 640. In some embodiments of the present disclosure, by integrally setting the gas inlet channel 630 and/or the gas outlet channel 640 with the inner side of the reservoir upper housing 611, one-piece molding production may be carried out by an integrally set structure, which reduces difficulty and cost of producing the reservoir. The cross-sections of the gas inlet channel 630 and the gas outlet channel 640 may be circular, rectangular, or other irregular shapes, which are determined based on the actual needs or the processing.

When the gas inlet channel 630 and/or the gas outlet channel 640 are non-integrally provided with the inner side of the reservoir upper housing 611, the gas inlet channel 630 and/or the gas outlet channel 640 may be separated from the inner side of the reservoir upper housing 611. Further, the gas inlet channel 630 and/or the gas outlet channel 640 may also be removably connected to the reservoir upper housing 611. Some embodiments of the present disclosure facilitate disassembly, cleaning, and replacement of the gas inlet channel 630 and/or the gas outlet channel 640 by setting the gas inlet channel 630 and/or the gas outlet channel 640 non-integrally with the inner side of the reservoir upper housing 611 of the reservoir, and making at least a portion of the structures of the gas inlet channel 630 and/or the gas outlet channel 640 be detachable relative to the reservoir upper housing 611, so as to ensure that the gas channel is clean to improve convenience when using.

In some embodiments, a distance between the reservoir first gas outlet 632 and the reservoir second gas inlet 641 may be greater than a threshold (e.g., 4 cm) so that the gas input through the reservoir first gas inlet 631 needs to enter the reservoir cavity firstly and mix with the steam inside the reservoir cavity to form the mixed gas, which then enters the gas outlet channel 640 from the reservoir second gas inlet 641. The distance between the reservoir first gas outlet 632 and the reservoir second gas inlet 641 may be a minimum distance between the reservoir first gas outlet 632 and the reservoir second gas inlet 641. As shown in FIG. 6C, the distance between the reservoir first gas outlet 632 and the reservoir second gas inlet 641 may be a distance between a lower-right corner of the reservoir first gas outlet 632 and an upper-left corner of the reservoir second gas inlet 641.

In some embodiments, the reservoir first gas outlet may be an outlet in a first opening direction, and a second reservoir outlet may be an outlet in a second opening direction. The first opening direction and the second opening direction are in opposite directions or perpendicular to each other. As shown in FIG. 6D, when the reservoir 600 is normally horizontally placed, a spatial coordinate system is constructed with a center of the reservoir cavity as an origin. A Z-axis may be a vertical direction of the reservoir 600, a +X-direction and a −X-direction may be respectively forward and backward, a +Y direction and a −Y direction may be respectively right and left, and a +Z direction and a −Z direction may be respectively upward and downward. The first opening direction corresponding to the reservoir first gas outlet 632 may be in the −Z direction, the second opening direction corresponding to the reservoir second gas inlet 641 may be in the +X direction, and the first opening direction and the second opening direction may be perpendicular to each other.

In some embodiments of the present disclosure, by restricting the distance or the opening direction between the reservoir first gas outlet 632 and the reservoir second gas inlet 641, a short circuit of a gas circuit between the reservoir first gas outlet 632 and the reservoir second gas inlet 641 may be avoided. Therefore, the gas input through the reservoir first gas outlet 632 does not mix with the steam inside the reservoir cavity but directly enters the reservoir second gas inlet 641, ensuring a humidification effect of the gas inside the reservoir 600.

In some embodiments, the spatial coordinate system of the reservoir 600 is constructed based on the foregoing manner, and a distance between the reservoir first gas inlet 631 and the reservoir first gas outlet 632 is greater than a preset threshold (e.g., 6 cm) on a Y-axis, and/or a distance between the reservoir second gas inlet 641 and the reservoir second gas outlet 642 on the Y-axis is greater than the preset threshold. Based on the foregoing setting, it is possible to make portion of the liquid not immediately flow out from the other side even if it flows back into the gas channel, which enhances a static reflux prevention effect of the reservoir 600 to avoid affecting using the respiratory ventilation apparatus and to avoid causing the patient to choke. For example, when the respiratory ventilation apparatus is placed at an inclined position, even though the liquid in the reservoir cavity submerges the reservoir second gas inlet 641, since the distance between the reservoir second gas inlet 641 and the reservoir second gas outlet 642 on the Y-axis is greater than the preset threshold, the inclined position of the respiratory ventilation apparatus causes a height difference between the reservoir second gas inlet 641 and the reservoir second gas outlet 642, and the liquid submerged into the reservoir second gas inlet 641 does not immediately overflow from the reservoir second gas outlet 642.

In some embodiments, the static reflux prevention effect of the reservoir 600 is correlated to a distance between the reservoir first gas inlet 631 and the reservoir first gas outlet 632 and a distance between the reservoir second gas inlet 641 and the reservoir second gas outlet 642. The spatial coordinate system of the reservoir 600 is constructed based on the foregoing manner, the further the distance between the reservoir first gas inlet 631 and the reservoir first gas outlet 632 on the Y-axis and the distance between the reservoir second gas inlet 641 and the reservoir second gas outlet 642 on the Y-axis, the better the static reflux prevention effect of the reservoir 600. When the liquid in the reservoir 600 is inclined in the Y-axis direction, the further the distance between the reservoir first gas inlet 631 and the reservoir first gas outlet 632 on the Y-axis and the distance between the reservoir second gas inlet 641 and the reservoir second gas outlet 642 on the Y-axis, the less likely the liquid flowing from the reservoir first gas outlet 632 back to the gas inlet channel 630 may flow out of the reservoir first gas inlet 631, and the less likely the liquid flowing from the reservoir second gas inlet 641 back into the gas outlet channel 640 may flow out of the reservoir second gas outlet 642, which improves the static reflux prevention effect of the reservoir 600. Similarly, the spatial coordinate system of the reservoir 600 is constructed based on the foregoing manner, a distance between the reservoir first gas inlet 631 and the reservoir first gas outlet 632 on the X-axis and a distance between the reservoir second gas inlet 641 and the reservoir second gas outlet 642 on the X-axis, the better the static reflux prevention effect of the reservoir 600. When the liquid in the reservoir 600 is inclined in the X-axis direction, the further the distance between the reservoir first gas inlet 631 and the reservoir first gas outlet 632 on the X-axis and the distance between the reservoir second gas inlet 641 and the reservoir second gas outlet 642 on the X-axis, the less likely the liquid flowing from the reservoir first gas outlet 632 back into the gas inlet channel 630 may flow out of the reservoir first gas inlet 631, and the less likely the liquid flowing from the reservoir second gas inlet 641 back into the gas outlet channel 640 may flow out of the reservoir second gas outlet 642, which improves the static reflux prevention effect of the reservoir 600.

In some embodiments, the reflux prevention component may include the first bending portion 633 and/or the second bending portion 643. As shown in FIGS. 6D-6G, the gas inlet channel 630 may include a first bending portion 633, and the gas outlet channel 640 may include the second bending portion 643. The first bending portion 633 and the second bending portion 643 are bent toward the center of the reservoir cavity, the reservoir first gas outlet 632 is provided on the first bending portion 633, and the reservoir second gas inlet 641 is provided on the second bending portion 643. The first bending portion 633 and the second bending portion 643 may be to be bent at a right angle or to be bent in a circle. As shown in FIG. 6D, the first bending portion 633 may be bent at a right angle, and the second bending portion 643 may be bent in the circle. When the first bending portion 633 and the second bending portion 643 are bent in the circle, a specific curvature of bending may be preset according to a requirement. In some embodiments, the first bending portion 633 and the second bending portion 643 are bent toward the center of the reservoir cavity, with the extension end exceeding the center of the reservoir cavity. The first bending portion 633 includes all structures of the gas inlet channel 630 after bending, and the second bending portion 643 includes all structures of the gas outlet channel 640 after bending. In some embodiments of the present disclosure, by providing the first bending portion 633 and the second bending portion 643, providing the reservoir first gas outlet 632 on the first bending portion 633, and providing the reservoir second gas inlet 641 on the second bending portion 643, which may make the reservoir first gas outlet 632 and the reservoir second gas inlet 641 far away from a sidewall of the reservoir upper housing 611. In such cases, a liquid is avoided to be splashed when the liquid collides with a sidewall inside the reservoir cavity and flows back into the reservoir first gas outlet 632 and/or the reservoir second gas inlet 641, improving the static reflux prevention effect and a dynamic reflux prevention effect of the reservoir 600, so as to ensure normal operation of the respiratory ventilation apparatus, avoid the patient to choke. Meanwhile, gas flowing into each gas inlet may pass through the first bending portion 633 and/or the second bending portion 643 to occur gasflow steering, so as to prolong a gas path, thus making the reservoir 600 have a good reflux prevention effect.

In some embodiments, the gas inlet channel 630 and the gas outlet channel 640 may be a one-piece molding structure. In some embodiments, the gas inlet channel 630 and the gas outlet channel 640 may be an assembled structure. At least one side of the gas inlet channel 630 and/or the gas outlet channel 640 is a detachable structure. The detachable structure may be fixed to the gas inlet channel 630 and/or the gas outlet channel 640 in a plurality of ways (e.g., screw fixing). As shown in FIG. 6H, the gas inlet channel 630 and the gas outlet channel 640 are close to a side of a liquid surface, i.e., a sealing plate 660 is the detachable structure, and the sealing plate 660 may be a shared component of the gas inlet channel 630 and the gas outlet channel 640. The sealing plate 660 may be provided with a snap 661, and the gas inlet channel 630 may be provided with a protruding portion 662 on one side edge. A user (e.g., a patient or a medical worker) may fix the sealing plate 660 to other side edges of the gas inlet channel 630 and the gas outlet channel 640 by snapping the snap 661 with the protruding portion 662 to form a sealed channel all around. The user may also disassemble the sealing plate 660 by separating the snap 661 from the protruding portion 662 to clean an interior of the gas inlet channel 630 and the gas outlet channel 640. By providing at least one side of the gas inlet channel 630 and/or the gas outlet channel 640 as the detachable structure in some embodiments of the present disclosure, it is possible to facilitate cleaning of the gas inlet channel 630 and/or the gas outlet channel 640 to ensure cleanliness and safety of the reservoir 600.

In some embodiments, as shown in FIG. 6G, the reflux prevention component may also include the flow guide rib 650. The flow guide rib 650 may be provided between the reservoir first gas outlet 632 and a sidewall of an upper housing adjacent thereto and/or between the reservoir second gas inlet 641 and a sidewall of an upper housing adjacent thereto, so as to avoid the liquid splashed by colliding with the sidewall of the reservoir cavity flows back to the reservoir first gas outlet 632 and/or the reservoir second gas inlet 641.

In some embodiments, the flow guide rib 650 may include a first flow guide rib 651 and a second flow guide rib 652. When the flow guide rib 650 is provided between the reservoir first gas outlet 632 and a sidewall of the reservoir upper housing adjacent thereto, a distance between the first flow guide rib 651 and the reservoir first gas outlet 632 may be greater than a distance between the second flow guide rib 652 and the reservoir first gas outlet 632. When the flow guide rib 650 is provided between the reservoir second gas inlet 641 and a sidewall of the reservoir upper housing adjacent thereto, a distance between the first flow guide rib 651 and the reservoir second gas inlet 641 may be greater than a distance between the second flow guide rib 652 and the reservoir second gas inlet 641. In some embodiments of the present disclosure, by providing the first flow guide rib 651 and the second flow guide rib 652, a splashing range of the liquid is limited, improving the dynamic reflux prevention effect of the reservoir 600 to prevent the liquid from flowing back to the reservoir first gas outlet 632, and/or the reservoir second gas inlet 641 when splashing on different surfaces.

As shown in FIG. 6G, a length of the first flow guide rib 651 in a vertical direction of the reservoir housing 610 may be less than a length of the second flow guide rib 652 in the vertical direction of the reservoir housing 610. When the length of the first flow guide rib 651 in the vertical direction of the reservoir housing 610 is less than the length of the second flow guide rib 652 in the vertical direction of the reservoir housing 610, the splashing range of the liquid when colliding with the sidewall may be limited, further preventing the liquid from splashing into the reservoir first gas outlet 632 and/or the reservoir second gas inlet 641.

In some embodiments, in a state in which the reservoir 600 is normally placed, when the flow guide rib 650 is provided between the reservoir first gas outlet 632 and the sidewall of the upper housing sidewall adjacent thereto, a minimum distance between the second flow guide rib 652 and the liquid surface of the reservoir cavity is less than a minimum distance between the reservoir first gas outlet 632 and the liquid surface of the reservoir cavity. When the flow guide rib 650 is provided between the reservoir second gas inlet 641 and the sidewall of the upper housing adjacent thereto, the minimum distance between the second flow guide rib 652 and the liquid surface of the reservoir cavity is less than a minimum distance between the reservoir second gas inlet 641 and the liquid surface of the reservoir cavity. Therefore, when the liquid flows to the liquid surface along the second flow guide rib 652, a flowing liquid is prevented from colliding with the liquid surface and bouncing to the reservoir first gas outlet 632 and/or the reservoir second gas inlet 641.

In some embodiments, the second flow guide rib 652 has a third bending portion 652-1 on an end away from an inner side of the reservoir upper housing 611. When the flow guide rib 650 is provided between the reservoir first gas outlet 632 and a sidewall of the reservoir upper housing adjacent thereto, the third bending portion 652-1 is bent toward the reservoir first gas outlet 632. When the flow guide rib 650 is provided between the reservoir second gas inlet 641 and a sidewall of the reservoir upper housing adjacent thereto, the third bending portion 652-1 is bent toward the first reservoir second gas inlet 641. As shown in FIG. 6G, the first flow guide rib 651 and the second flow guide rib 652 are provided between the reservoir second gas inlet 641 and a sidewall of the reservoir upper housing adjacent thereto, and the second flow guide rib 652 has the third bending portion 652-1 provided at an end of the second flow guide rib 652 away from the inner side of the reservoir upper housing, and the third bending portion 652-1 is bent toward the reservoir second gas inlet 641.

In some embodiments of the present disclosure, by providing the third bending portion 652-1, the reservoir first gas outlet 632 and/or the reservoir second gas inlet 641 of the reservoir may be partially covered effectively to enhance the dynamic reflux prevention effect of the reservoir 600, to prevent water on the liquid surface in the reservoir 600 from splashing making the water flow back to the reservoir first gas outlet 632 and/or the reservoir second gas inlet 641 when the respiratory ventilation apparatus is being transported or moved.

In some embodiments, the gas inlet channel 630 and the gas outlet channel 640 may form a gas channel module. The gas channel module and the reservoir 600 may be set as independent components. The gas channel module may be fixed on the reservoir upper housing 611 in the reservoir 600 by bonding or ultrasonic welding, or the gas channel module may be detachably and fixedly connected to the reservoir upper housing 611 in the reservoir 600.

In some embodiments, the gas inlet channel 630 may include at least two sections of pipe structure, and a gasflow transmission direction may change among the plurality of sections of the pipe structure, e.g., a first gas inlet section, a second gas inlet section, and a third gas inlet section connected in sequence. The first gasflow direction change may occur at the connection between the first gas inlet section and the second gas inlet section, and the second gasflow direction change may occur at the connection between the second gas inlet section and the third gas inlet section. The gas inlet channel with the multi-section pipe structure may extend a gas transmission path of the inlet gasflow in the gas inlet channel, and the multiple gasflow changes may improve the noise reduction effect of the reservoir, and at the same time improve the reflux prevention effect of the reservoir. In some embodiments, a gas inlet end of the gas inlet channel may be the side of the first gas inlet section away from the second gas inlet section, and a gas outlet end of the gas inlet channel 630 may be the side of the final pipe far away from the first gas inlet section. In some embodiments, a gas outlet direction of the gas outlet end of the gas inlet channel 630 may be opposite to a gas inlet direction of the gas inlet end of the gas outlet channel 640. In some embodiments, the gas outlet end of the gas inlet 630 and the gas inlet end of the gas outlet channel 640 may be set at different heights. When the reservoir 600 is in a normal use position, the gas outlet end of the gas inlet channel 630 may be set at a lower height than the gas inlet end of the gas outlet channel 640. In some embodiments, at least one section of the gas inlet channel 630 may be parallel to the gas outlet channel 640. In some embodiments, the gas outlet end of the gas inlet channel 630 may be provided with a flow guide structure to change the direction and angle of the gasflow output to the reservoir 600. In some embodiments, the gas inlet channel 630 may include at least two pipe sections with different sectional areas, so that a gasflow transmission area of the gasflow transmitted in the gas inlet channel 630 changes to achieve a good reservoir noise reduction effect, and the gas inlet channel 630 outputs a smooth gasflow.

In some embodiments, the gas inlet of the gas outlet channel 640 may be approximately located at a center of a whole structure of the reservoir 600, so as to effectively improve the reflux prevention effect.

The respiratory ventilation apparatus in the above embodiment, by providing each structure on the reservoir as described above, technical problems of the liquid in the reservoir returning into the gas inlet channel or the gas outlet channel, thereby damaging the respiratory ventilation apparatus or causing the patient to choke, ensuring the normal use of the respiratory ventilation apparatus and enhance a patient's experience.

In some embodiments, a liquid inlet for adding liquid may be disposed on the reservoir 600. When the liquid needs to be added to the reservoir, a lock switch may be manually opened to smoothly fill the liquid, and then the lock switch may be closed to prevent the liquid from flowing out when the water tank is tipped. In some embodiments, the lock switch may be a rotating shaft-bayonet structure. For example, the rotating shaft may rotate to enable liquid filling when the bayonet is in an initial position. After the rotating shaft rotates at a certain angle, the reservoir housing may be locked by the bayonet. The rotating shaft may be a hinge structure, for example, including a groove provided on the reservoir lower housing 612, and a rotatable hinge head provided on the reservoir upper housing 611 that is engaged in the groove. When the groove and the hinge head are snap-fitted, the hinge structure may be formed, allowing the reservoir 600 to switch between open and closed states. In addition, a limiting structure for limiting an opening angle of the reservoir 600 may be provided on the reservoir upper housing 611 and the reservoir lower housing 612 corresponding to the hinge structure.

FIG. 7A is a schematic diagram illustrating an exemplary appearance of a reservoir according to some other embodiments of the present disclosure; FIG. 7B is a schematic diagram illustrating an exemplary mating structure of a press button and a recessed structure according to some embodiments of the present disclosure; FIG. 7C is a schematic diagram illustrating an exemplary structure of a press button according to some embodiments of the present disclosure; FIG. 7D is a schematic diagram illustrating an exemplary structure of a press button from another perspective according to some embodiments of the present disclosure; FIG. 7E is a schematic diagram illustrating an exemplary structure of a recessed structure according to some embodiments of the present disclosure; and FIG. 7F is a schematic diagram illustrating another exemplary recessed structure according to some embodiments of the present disclosure. A structure of the reservoir 700 is described below in connection with FIGS. 7A-7G.

As shown in FIG. 7A, some embodiments of the present disclosure provide the reservoir 700 for a respiratory ventilation apparatus, which mainly includes a reservoir housing 710 and a pressing button 750 provided on an outer surface of the reservoir housing 710. As shown in FIG. 7A and FIG. 7B, the pressing button 750 is configured to be able to be pressed in a direction close to an interior of the reservoir housing 710 under an external force to enable the reservoir 700 to be assembled to or disassembled from a main body of respiratory ventilation apparatus. In some embodiments, the pressing button 750 may include a pressing main body including an elastic structure (not shown in the figures), and the elastic structure may be configured to produce an elastic deformation when the pressing button 750 is pressed in a direction close to the interior of the reservoir housing 710. When the external force is released, the elastic structure may also be configured to recover deformation to drive the pressing button 750 to rebound to reset in a direction away from the interior of the reservoir housing 710, so that the reservoir 700 is fixed to the main body of the respiratory ventilation apparatus or is reset after disassembly. As a result, the reservoir 700 may be maintained stable (e.g., a relative position of each portion of the reservoir housing 710 is maintained unchanged, etc.) during a process of assembly or release of the reservoir 700, which facilitates a connection of the reservoir 700 to the main body of the respiratory ventilation apparatus.

In some embodiments, the elastic structure may include a fixed end and a free end disposed opposite to each other. When the pressing button 750 is pressed in a direction close to the inside of the reservoir housing 710, the free end may move from a first position to a second position, and the elastic structure may have an elastic deformation. When the external force is released, the free end may move from the second position to the first position, and the elastic structure may restore its deformation and drive the pressing button 750 to spring back in a direction away from the inside of the reservoir housing 710, so that the reservoir 700 is connected to the main body or is reset after being removed from the main body. In this embodiment, due to the elastic deformation of the elastic structure, at least a portion of the structure of the pressing button 750 may have a moving space relative to the reservoir housing 710. By moving at least a portion of the structure of the pressing button 750, the connection and separation of the reservoir 700 and a corresponding docking position on the main body may be realized. In this embodiment, as the free end of the elastic structure moves between the first position and the second position, at least a portion of the structure of the pressing button 750 may have space for movement. In practical applications, the above-mentioned movable portion of the pressing button 750 may not have a great moving range, for example, within a range of 0.1 mm-1 mm. Of course, depending on a structure size of the actual application, the above-mentioned movable portion of the pressing button 750 may also move within a range of less than 0.1 mm or greater than 1 mm. For a stability of a pressing operation of the pressing button 750, the maximum pressing range may not exceed 15 mm.

In some embodiments, the pressing button 750 may be provided on an upper surface of the reservoir housing 710, and the pressing button 750 may be pressed downwardly under an action of the external force. After the external force is released, the pressing button 750 may be rebounded upwardly to reset under actuation of the elastic structure. In some embodiments, the pressing button 750 may also be provided on other surfaces of the reservoir housing 710, such as a bottom surface, a front surface, a side surface, and the like. In some embodiments, a count of the pressing button 750 may be one or more. For example, the count of the pressing button 750 may be one, which is provided on an upper surface or a front surface of the reservoir housing 710, etc. As another example, the count of pressing button 750 may be two, which are provided on different surfaces of the reservoir housing 710. Considering operability of the reservoir 700 when it is actually disassembled and assembled with the main body of the respiratory ventilation apparatus, when the count of the pressing button 750 is excessive, it may cause difficulty in an operation for an operator to operate with one hand, thus the count of the pressing button 750 may be one or two. And, when the count of the pressing button 750 is two, the two pressing buttons 750 are provided on two opposing surfaces on the reservoir housing 710, such as an upper surface and a bottom surface. For the convenience of description, the following illustrates a structure of the pressing button 750 and a structure of the reservoir housing 710 by taking an example of the pressing button 750 being provided on the upper surface of the reservoir housing 710. By way of example, the structure of the pressing button 750 provided on other surfaces of the reservoir housing 710 is the same as or similar to that of the pressing button 750 provided on the upper surface of the reservoir housing 710, and may not be described herein in the present disclosure.

In some embodiments, a length direction of the reservoir housing 710 may be defined as an X direction, a width direction of the reservoir housing 710 as a Y direction, and a height direction of the reservoir housing 710 as a Z direction. And, a right direction is the X direction, a left direction is an opposite direction of the X direction, and the reservoir housing 710 enters the main body of the respiratory ventilation apparatus in a left direction (i.e., along the opposite direction of the X direction) to realize a connection. A top direction is the Z direction and a bottom direction is an opposite direction of the Z direction. The pressing button 750 moves downward, i.e., the pressing button 750 moves in the opposite direction of the Z direction; the pressing button 750 moves upward i.e., the pressing button 750 moves in the Z direction. A front direction is the opposite direction of the Y direction, and a back direction is the Y direction.

In some embodiments, the pressing button 750 further includes a connection structure 752, the connection structure 752 is detachably connected to the main body of the respiratory ventilation apparatus. The pressing main body may include a pressing structure. The connection structure 752 may be disposed on an end of the pressing main body close to the main body, that is, the connection structure 752 may be disposed on an end of the pressing structure close to the main body. When the pressing button 750 is disposed on the upper surface of the reservoir housing 710, i.e., a top surface of the reservoir housing 710, and when the pressing button 750 is pressed in a direction close to the interior of the reservoir housing 710, a force in a direction close to the interior of the reservoir housing 710 may be applied on the upper surface of the pressing structure (i.e., the pressing surface 751). That is, the pressing structure may be pressed downward, then the connection structure 752 and the pressing structure may move toward the direction close to the interior of the reservoir housing 710 synchronously. That is, the connection structure 752 may move downward to leave the corresponding docking position on the main body, so that the connection structure 752 is separated from the main body of the respiratory ventilation apparatus, thereby making the reservoir 700 detached from the main body of the respiratory ventilation apparatus. When the pressing button 750 is reset, the connection structure 752 may be connected to the respiratory ventilation apparatus main body, thereby enabling the reservoir 700 to be fixed in connection with the main body of the respiratory ventilation apparatus.

As shown in FIG. 7B, in some embodiments, the connection structure 752 is provided at an end of the pressing button 750 near the main body of the respiratory ventilation apparatus, i.e., in the X direction, the connection structure 752 is provided at a left side of the pressing main body to facilitate the connection structure 752 to connect with the main body of the respiratory ventilation apparatus. In some embodiments, the connection structure 752 may include a connection component 752-1 and a buckle protrusion 752-2. The upper surface of the connection structure 752 (the direction away from the reservoir housing 710) may be disposed with the connection component 752-1 and the buckle protrusion 752-2 (or groove) used to be connected with the main body. The connection component 752-1 may be connected to an end of the pressing main body, and the buckle protrusion 752-2 may be disposed on an end of the connection component 752-1 away from the pressing main body. Correspondingly, an inner surface of the housing of the main body, specifically, in this embodiment, the inner surface of a top housing of the main body, may have the docking position corresponding to the connection structure 752, and a groove (or protrusion) docked with the buckle protrusion 752-2 may be disposed at the docking position. As the connection component 752-1 has a preset length, e.g., a length between 5 mm and 25 mm, the docking position of the main body, as well as the groove may be disposed at a position corresponding to the preset length of the connection component 752-1.. A movement freedom degree only along a movement direction of the pressing button 750 (e.g., the Z direction, i.e., the top direction and the bottom direction) exists between the buckle protrusion 752-2 and the groove, e.g., when the pressing button 750 is pressed downward, the connection structure 752 may move downward synchronously. At this time, the connection structure 752 may be below the docking position of the main body and have a certain distance (e.g., 0.05 mm or 0.3 mm) from the docking position. When installing the reservoir 700, when the pressing structure is pressed by an external force in a direction (e.g., downward) close to the interior of the reservoir housing 710, the buckle protrusion 752-2 moves in synchronization with the direction (e.g., downward) close to the interior of the reservoir housing 710. At this time, the reservoir 700 may be moved to the left (i.e., in the opposite direction along the X direction) into the main body of the respiratory ventilation apparatus until the buckle protrusion 752-2 is moved to the corresponding position below the groove. When the external force is released, the pressing structure may spring back in a direction away from the interior of the reservoir housing 710 (e.g., upward), and the buckle protrusion 752-2 may enter the groove to achieve snap fixation, thereby realizing locking of the connection of the reservoir 700 and the main body of the respiratory ventilation apparatus. During disassembly and assembly of the reservoir 700, when the pressing structure is pressed in the direction (e.g., downward) close to the interior of the reservoir housing 710 by an external force, the buckle protrusion moves in the direction (e.g., downward) close to the interior of the reservoir housing 710 and moves away from the groove to unlock the connection of the reservoir 700 with the main body of the respiratory ventilation apparatus. At this time, the reservoir 700 may be moved to the right (i.e., along the X-direction) out of the main body of the respiratory ventilation apparatus, thereby realizing separation of the reservoir 700 from the main body of the respiratory ventilation apparatus. After disassembling the reservoir 700, the external force may be released, and the pressing button 750 may be pressed to reset. In some embodiments, the buckle protrusion 752-2 used as the connection structure 752 may be a strip at a left end portion of the pressing button 750, or may be a plurality of buckle protrusions (e.g., two buckle protrusions used as the connection structure 752 as shown in FIG. 7C) spaced apart at the left end portion (along Y direction) of the pressing button 750. In some embodiments, the connection structure 752 may further be two claws extending from the left end of the pressing button 750. The main body of the respiratory ventilation apparatus may include two slots matching the two claws. The installation and removal modes of the claws may be the same as that of the above-mentioned buckle protrusions, which are not repeated here.

In some embodiments, the connection between the connection structure 752 and the main body of the respiratory ventilation apparatus may also include other connections capable of being locked or unlocked by movement. For example, the connection between the connection structure 752 and the main body of the respiratory ventilation apparatus may be a magnetic connection. When the pressing button 750 is moved in the direction away from the interior of the reservoir housing 710 (e.g., upward, i.e., in the Z-direction) to a point where the connection structure 752 contacts with the main body of the respiratory ventilation apparatus and in the magnetic connection, locking of the connection between the reservoir 700 and the main body of the respiratory ventilation apparatus is realized. When the pressing button 750 is pressed to move in the direction close to the interior of the reservoir housing 710 (e.g., downward, i.e., in the opposite direction of the Z-direction) to a point where the connection structure 752 is separated from the main body of the respiratory ventilation apparatus, unlocking of the connection between the reservoir 700 and the main body of the respiratory ventilation apparatus is achieved when the connection structure 752 does not automatically approach the main body of the respiratory ventilation apparatus under a magnetic force.

As shown in FIG. 7B, FIG. 7E, and FIG. 7F, in some embodiments, the upper surface of the reservoir housing 710 includes a recessed structure 720, and the pressing button 750 is accommodated in the recessed structure 720. The pressing button 750 may move in the recessed structure 720 in a direction (e.g., upwardly, or downwardly) close to or away from the interior of the reservoir housing 710, thereby enabling the connection between the reservoir 700 and the main body of the respiratory ventilation apparatus to be locked or unlocking. The elastic structure is provided between a side (i.e., the lower side) of the pressing button 750 near the interior of the reservoir housing 710 and a bottom wall of the recessed structure 720, and the elastic structure may drive the pressing button 750 to move to a direction away from the bottom wall of the recessed structure 720 (i.e., away from the interior of the reservoir housing 710, upward) when recovering deformation.

As shown in FIGS. 7B-7C, in some embodiments, the free end of the elastic structure may be configured to abut against the surface of the reservoir housing 710, and the fixed end may be fixedly connected to the pressing structure. The upper surface of the pressing structure may be a pressing surface 751. The pressing surface 751 may be provided on a surface (i.e., an upper surface of the pressing structure) of the pressing button 750 away from the interior of the reservoir housing 710 for providing a user with a region for pressing. Therefore, the elastic structure may be disposed on the lower surface of the pressing structure. The fixed end of the elastic structure may be connected to the lower surface of the pressing structure, and the free end of the elastic structure may abut against the top surface of the reservoir housing 710. In some embodiments, the connection structure 752 may be integrally molded with the pressing structure, or may be integrally molded with both the pressing structure and the elastic structure, so as to reduce operations and difficulty of processing and manufacturing the pressing button 750, and further make the connection structure 752 and the pressing structure, or the connection structure 752 and the pressing surface 751 move synchronously in the same direction. In some embodiments, the pressing structure may be disposed between the free end and the fixed end of the elastic structure. By applying force on the pressing structure, a relative position between the free end and the fixed end may change. For example, when applying force on the pressing structure, the free end may approach the fixed end, and an angle in the horizontal direction between the free end and the fixed end may gradually increase, i.e., the space between the free end and the fixed end in the horizontal direction may decrease.

As shown in FIG. 7C, in some embodiments, the pressing surface 751 may be provided with pressing stripes 751-1. A plurality of pressing stripes 751-1 may be provided at intervals along the X direction. The plurality of pressing stripes 751-1 may act as a tactile cue to remind a user of a pressing position of the pressing surface 751. At the same time, the plurality of pressing stripes 751-1 may also have functions of increasing friction and anti-slip, avoiding the hand slipping of the user on the pressing surface 751 when operating, and increasing operation safety of the reservoir 700.

As shown in FIGS. 7B, 7C, and 7D, in some embodiments, at least one of a limiting structure and a guiding structure is also provided between the pressing button 750 and an outer surface of the reservoir housing 710 (e.g., the recessed structure 720). The limiting structure may be configured to limit a range of the pressing button 750 to move in a direction away from the interior of the reservoir housing 710 (e.g., upward), and the pressing button 750 may be connected to the recessed structure 720 through the limiting structure. The guiding structure may be configured to guide the pressing button 750 to move in a preset path.

In some embodiments, a count of the limiting structure may be two or more. In some embodiments, the pressing button 750 may be provided with limiting structures on both sides (a front side and a back side) in the width direction (i.e., Y direction), and the count of the limiting structure on the both sides may be the same or different. Merely by way of example, the pressing button 750 may be provided with two limiting structures on both sides in the Y direction. Two limiting structures on either side are provided at intervals along the X direction to enhance strength of the connection between the pressing button 750 and the recessed structure 720. In some embodiments, a right side of the pressing button 750 in the X direction may also be provided with one or more limiting structures. In some embodiments, one or both or all three of a front side, a back side, and a right side of the pressing button 750 may be provided with limiting structures, and the count of limiting structures provided on each side may be the same or different.

In some embodiments, the limiting structure may include a limiting component 753 with a limiting groove 721. The limiting component 753 may be provided on one ofa side (i.e., a lower side) of the pressing button 750 close to the interior of the reservoir housing 710 or an outer surface (e.g., the recessed structure 720) of the reservoir housing 710. The limiting groove 721 may be provided on the other one of a side (i.e., the lower side) of the pressing button 750 close to the interior of the reservoir housing 710 or the outer surface (e.g., the recessed structure 720) of the reservoir housing 710. For example, the outer surface of the reservoir housing is disposed with the limiting component, and the side of the pressing button proximate to the interior of the reservoir housing is disposed with the limiting groove. As another example, the side of one end of the pressing button proximate to the interior of the reservoir housing is disposed with the limiting component, and the outer surface of the reservoir housing is disposed with the limiting groove. As shown in FIGS. 7E-7F, the limiting component 753 may be provided on the side of the pressing button 750 close to the interior of the reservoir housing 710 (i.e., on the lower side), and the limiting groove 721 may be provided in the recessed structure 720. The limiting groove 721 may cooperate with the limiting component 753 to limit the pressing button 750 during a resetting process of the pressing button 750 to limit the pressing button 750 in the recessed structure 720.

In some embodiments, the limiting component 753 is provided in the limiting groove 721, and the limiting component 753 may move in the limiting groove 721 in a direction (e.g., up and down) close to and/or away from the interior of the reservoir housing 710. A height of the limiting groove 721 refers to a range in which the pressing button 750 moves. In some embodiments, the limiting groove 721 is provided on an inner wall of the recessed structure 720 and corresponds to the limiting component 753. That is, a setting location and a setting count of the limiting groove 721 correspond one by one with a setting location and a setting count of the limiting component 753. Merely by way of example, four limiting structures are provided between the pressing button 750 and the recessed structure 720. Two of the four limiting structures are provided at intervals along the X direction on a front side of the pressing button 750 in the Y direction, and the other two limiting structures are provided at intervals in the X direction at the back side of the pressing button 750 in the Y direction, at this time, the pressing button 750 is provided with two limiting components 753 provided at intervals in the X direction at the front side and the back side of the pressing button 750 in the Y direction respectively. Correspondingly, the two inner sidewalls of the recessed structure 720 in the Y direction are also provided with two limiting grooves 721 provided at intervals in the X direction, respectively. In some embodiments, the limiting component 753 may include, but is not limited to, a structure such as a buckle hook, a hook, a block, or the like that is capable of cooperating with the limiting groove 721.

In some embodiments, the limiting groove 721 has a preset height in the Z-direction so that when the limiting component 753 is moved in the limiting groove 721 to contact with an inner wall of an end portion of the limiting groove 721 in the Z-direction, the limiting component 753 in the limiting groove 721 is snapped in the limiting groove 721 and is snapped and limited at an end edge of the limiting groove 721. At this time, the pressing surface 751 of the pressing button 750 may be flush with the outer surface (e.g., the upper surface) of the reservoir housing 710 as shown in FIG. 7B, to avoid the pressing surface 751 protruding or concave relative to the outer surface of the reservoir housing 710 at the recessed structure 720, and to avoid the pressing surface 751 interfering with the connection of the reservoir 700 to the main body of the respiratory ventilation apparatus.

In some embodiments, the guiding structure includes a guiding member 754 and a guiding groove 722. The guiding member 754 may be provided on one of a side of the pressing button 750 close to the interior of the reservoir housing 710 (i.e., the lower side) or the outer surface of the reservoir housing 710 (e.g., the recessed structure 720), and the guiding groove 722 may be provided on the other of a side of the pressing button 750 close to the interior of the reservoir housing 710 (i.e., the lower side) or the outer surface of the reservoir housing 710 (e.g., the recessed structure 720). For example, the outer surface of the reservoir housing is disposed with the guiding member, and one end of the pressing button proximate the side of the interior of the reservoir housing is disposed with the guiding groove. As another example, the side of the pressing button proximate to the interior of the reservoir housing is disposed with the guiding member, and the outer surface of the reservoir housing is disposed with the guiding groove. As shown in FIGS. 7B, 7C, & 7D, the guiding member 754 may be provided on a side (e.g., the lower side) of the pressing surface 751 close to the interior of the reservoir housing 710, and the guiding groove 722 may be provided in the recessed structure 720. The guiding member 754 may move upwardly in the guiding groove 722 in a direction (e.g., an up and down direction) close to and/or away from the interior of the reservoir housing 710. The guiding member 754 may cooperate with the guiding groove 722. The guiding groove 722 may serve as a preset path for guiding a movement direction of the pressing button 750 during a process of pressing, rebounding, and resetting, so that the guiding member 754 moves in the guiding groove 722 when the pressing button 750 is moving, avoiding the pressing button 750 from tilting in the X direction and/or the Y direction when moving. Therefore, the pressing surface 751 may maintain stability in the X direction and/or the Y direction, which enhances stability of the pressing button 750.

In some embodiments, the guiding structure may be provided in a region of a predicted load point of the pressing surface 751 (e.g., near a middle of a length direction). With the above setup, a distance between a force position of the pressing surface 751 and the guiding structure in the X-direction may be minimized. Therefore, when a user presses on the pressing surface 751, a force direction of the pressing button 750 may substantially coincide with an extension direction (i.e., the Z direction or the opposite direction of the Z direction) of the guiding structure (e.g., the guiding member 754 and/or the guiding groove 722), thereby reducing friction during a movement of the pressing button 750 and reducing pressing difficulty. Therefore, to effectively and smoothly drive the connection structure 752 to move, a pressing region on the pressing button 750 that is subjected to an external force may be preferably located near a middle region of the pressing button 750. Here, a position near the middle region of the pressing button 750 in the length direction refers to a position near the middle region of the pressing button 750 in the length direction apart from the connection structure 752, that is, near the middle region of the pressing main body in the length direction. In some embodiments, the above-mentioned pressing region may be preferably located near the middle region of the length direction of the pressing structure. In addition, generally, a force center of the elastic structure may also correspond to the position near the middle region of the pressing structure, that is, a vertical line of the force center of the elastic structure may basically coincide with a vertical line of a middle portion of the pressing structure.

As shown in FIG. 7D, in some embodiments, a side of the guiding member 754 away from an inner wall of the recessed structure 720 may also be provided with a reinforced protrusion 754-1, and the reinforced protrusion 754-1 may be provided by extending along the Z direction. The reinforced protrusion 754-1 may strengthen the guiding member 754 and reduce possibility of fracture or deformation of the guiding member 754 under a force. In some embodiments, the guiding member 754 may be provided with a plurality of reinforced protrusions 754-1 provided at intervals in the X direction.

As shown in FIG. 7D, in some embodiments, a guiding structure may be provided between the pressing button 750 and the recessed structure 720 on both sides (a front side and a back side) of the pressing button 750 in the Y direction. The two guiding structures work together to guide the movement of the pressing button 750, enhancing the stability of the pressing button 750. In some embodiments, in the X direction, the guiding structure (e.g., the guiding member 754 and/or the guiding groove 722) may be provided between the two limiting structures (e.g., the limiting component 753 and/or the limiting groove 721), as shown in FIG. 7D.

As shown in FIGS. 7E and 7F, in some embodiments, a setting position of the guiding groove 722, as well as a count of settings, may be one-to-one corresponding to a setting position of the guiding member 754, as well as a count of settings. Exemplarily, the two guiding structures are provided between the pressing button 750 and the recessed structure 720, one of the guiding structures is provided on the front side of the pressing button 750 in the Y direction, and the other guiding structure is provided on the back side of the pressing button 750 in the Y direction. At this time, the pressing button 750 may be provided with a guiding member 754 on both sides (the front side and the back side) in the Y direction, and the guiding member 754 on each side is provided in the region of the predicted load point of the pressing surface 751 (e.g., near the middle of the length direction). Correspondingly, two inner sidewalls of the recessed structure 720 may be provided with a corresponding guiding groove 722 in the Y direction, and the guiding groove 722 on each side is provided at a position corresponding to a middle of the pressing surface 751.

As shown in FIG. 7B to FIGS. 7D, in some embodiments, the elastic structure may include at least one of an elastic arm (e.g., a first elastic arm 755 and/or a second elastic arm 725) with an elastic component 760. The elastic arm includes a free end and a fixed end, the elastic arm is configured such that when the pressing button 750 is pressed in a direction close to the interior of the reservoir housing 710 (e.g., downward), that is, when the pressing structure and the connection structure 752 are pressed synchronously toward the direction close to the interior of the reservoir housing 710 (e.g., downward), the free end may move in a direction away from the fixed end, and the elastic arm may be elastically deformed, that is, a fixed end of the elastic arm fixedly connected to the lower surface of the pressing structure may move synchronously with the pressing structure in the direction close to the interior of the reservoir housing 710. The elastic arm may undergo the elastic deformation, and an angle between the lower surface of the pressing structure connected to the fixed end of the elastic arm and the elastic arm may decrease, so a distance component in the horizontal direction between the free end and the fixed end of the elastic arm may increase. When an external force is released, the free end may move in the direction close to the fixed end, and the elastic arm may recover from the deformation. That is, the distance component in the horizontal direction between the free end and the fixed end of the elastic arm may decrease. In some embodiments, a position of the fixed end of the elastic arm may be fixed, and the free end of the elastic arm may abut against other parts (e.g., the reservoir housing 710, or the pressing structure, etc.). When the free end of the elastic arm moves in the direction close to or away from the fixed end, the free end may slide with the surface of the parts it is abut against, that is, the free end may move from the first position to the second position. The moving spatial range of the first position and the second position is as mentioned above and is not repeated here. When sliding, the free end of the elastic arm may slide toward or away from the fixed end. For details, please refer to the descriptions of the elastic arm later. The elastic component 760 is configured such that when the pressing button 750 is pressed in a direction close to the interior of the reservoir housing 710 (e.g., downwardly), a height of the elastic component 760 decreases and the elastic component 760 is elastically deformed. When the external force is released, the height of the elastic component 760 increases and the elastic arm deforms. In some embodiments, during the pressing process of the pressing button 750, the height decrease of the elastic component 760 may be expressed as a lower position of the upper end of the elastic component 760 (e.g., the upper end is the end where the elastic component 760 is connected to the pressing button 750). At this time, a height of the position of the lower end of the elastic component 760 (such as the end of the elastic component 760 connecting to the reservoir housing 710) may remain unchanged. In some embodiments, the decrease in the height of the elastic component 760 may also be expressed by a decrease in a vertical distance between the upper end and the lower end of the elastic component 760 in the height direction. Correspondingly, the increase in the height of the elastic component 760 may be expressed as an increase in the upper end position of the elastic member (the height of the lower end position may remain unchanged) and an increase in the vertical distance between the upper end and the lower end of the elastic component 760 in the height direction.

In some embodiments, the fixed end of the elastic arm is fixedly connected to the one of a side of the pressing button 750 close to the interior of the reservoir housing 710 (e.g., the lower side) or the bottom wall of the recessed structure 720. And the free end of the elastic arm is resisted against the other of a side of the pressing button 750 close to the interior (e.g., lower side) of the reservoir housing 710 or the bottom wall of the recessed structure 720. When the pressing button 750 is pressed downward by the external force or when the external force is released, relative sliding is produced between the free end and the other of the side of the pressing button 750 close to the interior of the reservoir housing 710 (e.g., the lower side) or the bottom wall of the recessed structure 720. When the pressing button 750 is pressed by the external force in a direction (e.g., downward) close to the interior of the reservoir housing 710, the free end slides away from the fixed end, and the elastic arm is elastically deformed and accumulates elastic potential energy. When the external force is released, the free end slides in a direction close to the fixed end, the elastic arm is elastically deformed, and the pressing button 750 is driven to rebound and reset in the direction away from the interior of the reservoir housing 710 (e.g., upward). It should be noted that, in the present disclosure, to be resisted against an object is to be in contact but not under force, or in contact and subject to pre-pressure. Merely by way of example, when the pressing button 750 is in a normal state (i.e., not pressed by the external force), the free end is resisted against the bottom wall of the recessed structure 720, which indicates that the free end is just resisted against the bottom wall of the recessed structure 720 and is in an unstressed state, and the elastic arm is not elastically deformed. Or, the free end is resisted against the bottom wall of the recessed structure 720 and the first free end 755-2 is in a stressed state, the elastic arm is subjected to a force and is elastically deformed, and the elastic arm accumulates the elastic potential energy. In the embodiment where the elastic structure is in the form of the elastic arm, as the elastic arm includes the fixed end and the free end, and there is a certain length of arm body connecting the fixed end and the free end, the elastic arm may have a certain length, and the elastic arm may have a certain length component in the horizontal direction. The elastic arm with a certain length may enable the pressing button 750, especially the pressing structure connected to the elastic arm, to have a greater pressing force area. Therefore, an effective force may be applied to the elastic arm within the range of the length component of the elastic arm in the horizontal direction, thus increasing the pressing force area of the pressing button 750, facilitating the effective use of the pressing button 750 and increasing the stability and effectiveness of the pressing button 750. In some embodiments, when the angle between the lower surface of the pressing structure and the elastic arm (mainly referring to the arm body of the elastic arm) is constant, the longer the arm body, the greater the movable movement space of the pressing button 750 in the vertical direction. That is, the pressing button 750 may have a significant movement space in the vertical direction (such as the up and down direction), so the sensitivity, stability, and effectiveness of the pressing button 750 are improved, and the normal disassembly or connection between the reservoir and the main body is ensured.

In some embodiments, when the middle region of the pressing structure is subject to a force, a force center of the elastic arm may also correspond to a position near the middle position of the pressing structure, that is, a vertical line of the force center of the elastic arm basically coincides with a vertical line of the middle region of the pressing structure.

In some embodiments, the elastic arm may include at least one first elastic arm 755 and/or at least one second elastic arm 725. The first elastic arm 755 and/or the second elastic arm 725 may drive the pressing button 750 to rebound and reset in the direction away from the interior of the reservoir housing 710 (e.g., upward) after the pressing button 750 is pressed in the direction close to the interior of the reservoir housing 710 (e.g., downward).

In some embodiments, the first elastic arm 755 is provided on the side (e.g., the lower side) of the pressing surface 751 close to the interior of the reservoir housing 710. As shown in FIG. 7D, the first elastic arm 755 includes a first fixed end 755-1 and the first free end 755-2. As shown in FIG. 7B, the first fixed end 755-1 of the first elastic arm 755 is fixed to a side of the pressing button 750 that is backward to the pressing surface 751 (i.e., a side of the pressing surface 751 that is close to the interior of the reservoir housing 710, such as the lower side), and the first free end 755-2 of the first elastic arm 755 is resisted against the bottom wall of the recessed structure 720. In some embodiments, when the pressing button 750 is pressed in the direction (e.g., downward) close to the interior of the reservoir housing 710 by the external force, the first free end 755-2 of the first elastic arm 755 may slide relative to the bottom wall of the recessed structure 720, and the first elastic arm 755 is deformed to accumulate the elastic potential energy. In some embodiments, the first elastic arm 755 is provided in the X direction. When the pressing button 750 is pressed in the direction close to the interior of the reservoir housing 710 (e.g., downward) by the external force, the first free end 755-2 is away from the first fixed end 755-1 in the X direction. Correspondingly, the first free end 755-2 of the first elastic arm 755 is close to the first fixed end 755-1 in the Z direction. When the external force is released, the first elastic arm 755 releases the elastic potential energy, the first free end 755-2 of the first elastic arm 755 slides on the bottom wall of the recessed structure 720, the first free end 755-2 is close to the first fixed end 755-1 in the X direction, and the first free end 755-2 is away from the first fixed end 755-1 in the Z direction. Therefore, the pressing button 750 is driven in the Z direction away from the direction (i.e., upward) of the interior of the reservoir housing 710, causing the pressing button 750 to return to a pre-press position.

In some embodiments, when the pressing button 750 is in the normal state (i.e., not being pressed by the external force), the first free end 755-2 remains to be resisted against the bottom wall of the recessed structure 720 to allow the pressing button 750 to be pressed under stress in the direction (e.g., downward) close to the reservoir housing 710, and the external force is allowed to be released, in such case, the first elastic arm 755 may press the pressing button 750 to return to the pre-press position.

As shown in FIGS. 7C, 7D, in some embodiments, the first free end 755-2 may be a rounded corner setting. The rounded corner setting may reduce friction between the first free end 755-2 and the bottom wall of the recessed structure 720, and reduce difficulty of the first free end 755-2 sliding relative to the bottom wall of the recessed structure 720.

In some embodiments, a count of the first elastic arm 755 may be two or more. As shown in FIG. 7D, in some embodiments, the count of the first elastic arm 755 may be two, and the two first elastic arms 755 are provided at intervals and parallel along the Y direction. Therefore, the two first elastic arms 755 may be ensured to be consistent when pressing and resetting the pressing button 750, and the stability and effectiveness of the pressing button 750 is improved.

As shown in FIG. 7F, in some embodiments, the second elastic arm 725 has a similar or identical structure and function to the structure of the first elastic arm 755, e.g., the second free end 725-2 of the second elastic arm 725 may also be rounded to provide a rounded corner, etc. In some embodiments, the second fixed end 725-1 of the second elastic arm 725 is fixed to the bottom wall of the recessed structure 720, and the second free end 725-2 of the second elastic arm 725 is resisted against a side of the pressing button 750 away from the pressing surface 751 (i.e., the side of the pressing button 750 close to the interior of the reservoir housing 710, i.e., the lower side). When the pressing button 750 is pressed in a direction close to the interior of the reservoir housing 710 (e.g., downward) by the external force, the second free end 725-2 of the second elastic arm 725 slides relative to the pressing button 750, and the second elastic arm 725 is deformed to accumulate the elastic potential energy. When the external force is released, the second free end 725-2 of the second elastic arm 725 slides relative to the lower side of the pressing button 750, and the second elastic arm 725 recovers from deformation and releases the elastic potential energy to actuate the pressing button 750 to rebound and reset in the direction away from the interior of the reservoir housing 710 (e.g., upward). A working process and a principle of the second elastic arm 725 of pressing and resetting the pressing button 750 may be referred to in the relevant contents of the first elastic arm 755, and may not be repeated here.

In some embodiments, a setting direction of the second elastic arm 725 may be parallel to a setting direction of the first elastic arm 755. In the X direction, a position of the first fixed end 755-1 of the first elastic arm 755 corresponds to a position of the second free end 725-2 of the second elastic arm 725, a position of the first free end 755-2 of the first elastic arm 755 corresponds to a position of the second fixed end 725-1 of the second elastic arm 725. With the above setup, it is possible to make forces exerted by the second elastic arm 725 and the first elastic arm 755 on the pressing button 750 cancel each other in the X direction, thereby enhancing the stability of the pressing button 750.

In some embodiments, a count of the second elastic arm 725 may be two or more. As shown in FIG. 7F, in some embodiments, the count of the second elastic arm 725 may be two, and the two second elastic arm 725 are provided at intervals and parallel along the Y direction to ensure deformation of the two first elastic arms 755 may be consistent when pressing and resetting the pressing button 750, improving the stability of the pressing button 750.

In some embodiments, in the Y direction, the two second elastic arms 725 may be provided between the two first elastic arms 755. In some embodiments, in the Y direction, the two first elastic arms 755 may be provided between the two second elastic arms 725. In some embodiments, in the Y direction, the two first elastic arms 755, as well as the two second elastic arms 725, may be provided staggered from one another.

In the above embodiments, the plurality of elastic arms may be preferably symmetrically distributed. The plurality of elastic arms may support the vertical movement of the pressing structure at different positions during the movement of the pressing structure of the pressing button 750, thereby improving the stability and the smoothness of the use of the pressing button 750. The plurality of elastic arms disposed at different positions may enlarge the pressing force area of the elastic structure, thus increasing the pressing force area of the pressing structure of the pressing button 750, so that the pressing structure may be driven to move vertically (e.g., downward) by different regions of the pressing surface 751, thereby increasing the stability, smoothness, effectiveness, and usability of the pressing button 750. Moreover, by disposing the plurality of elastic arms, when one or more of the elastic arms are damaged, the elastic structure may still work, so that the pressing button 750 may work normally, thereby ensuring the normal disassembly or connection between the reservoir and the main body.

As shown in FIG. 7B, in some embodiments, a connection between one end of the elastic component 760 and the lower side of the pressing surface 751 or the one of the bottom walls of the recessed structure 720 may be a fixed connection, i.e., one end of the elastic component 760 is fixed, and the other end may be respectively resisted against the side of the pressing button 750 that is backward to the pressing surface 751 (i.e., the lower side of the pressing surface 751) or the other one of the bottom walls of the recessed structure 720. That is, the one end of the elastic component 760 may be fixed to the lower side of the pressing surface 751 and the other end may be resisted against the bottom wall of the recessed structure 720. Or, the one end of the elastic component 760 may be fixed to the bottom wall of the recessed structure 720, and the other end may be resisted against the lower side of the pressing surface 751. When the pressing button 750 is pressed in the direction close to the interior of the reservoir housing 710 (e.g., downward) by the external force, the height of the elastic component 760 decreases, and the elastic component 760 is elastically deformed to accumulate the elastic potential energy. When the external force is released, the elastic component 760 begins to recover from deformation and actuates the pressing button 750 in the direction away from the interior of the reservoir housing 710 (e.g., upward) to return to the pre-press position.

In some embodiments, a connection between one end of the elastic component 760 and the lower side of the pressing surface 751 or the one of the bottom wall of the recessed structure 720 may also be a plug-and-play connection. Specifically, as shown in FIG. 7B and FIG. 7D, a connection projection 756 may be provided on the side of the pressing button 750 that is backward to the pressing surface 751 (i.e., the lower side of the pressing button 750) or the bottom wall of the recessed structure 720, and the connection projection 756 may be connected to the elastic component 760. When the connection projection 756 is inserted into the elastic component 760, the elastic component 760 is fixedly provided through the connection projection 756. When the connection projection 756 is pulled out from the elastic component 760, the elastic component 760 may be realized to be disassembled and replaced.

In some embodiments, the elastic component 760 may be provided at an intermediate position (as shown in FIG. 7B and FIG. 7D) between the recessed structure 720 and the pressing button 750 in the X direction, on a basis that the pressing region of the pressing button 750 subjects to an external force is located at the middle region of the length direction of the pressing main body (the pressing structure). When the middle region of the pressing structure of the pressing main body subjects to a force, the force center of the elastic component 760 may correspond to the position near the middle region of the pressing structure. That is, the vertical line at the force center of the elastic component may be basically coincided with the vertical line at the middle region of the pressing structure, so as to avoid the pressing button 750 to be tilted in the X direction, enhancing the stability of the pressing button 750. As shown in FIG. 7B and FIG. 7D, at this time, in the X direction, the elastic component 760 may be provided between the connection structure 752 and the first free end 755-2 of the first elastic arm 755. In some embodiments, the elastic component 760 may be provided at an intermediate position between the recessed structure 720 and the pressing button 750 in the Y direction (as shown in FIG. 7D). The two first elastic arms 755 may be provided on both sides of the elastic component 760, and the two second elastic arms 725 may also be provided on the both sides of the elastic component 760 to enhance the stability of the pressing button 750.

In some embodiments, the elastic component 760 may include an elastic material (e.g., silicone, etc.) or an elastic structure (e.g., a spring, etc.). In some embodiments, the elastic component 760 may be a spring, which has a long service life and is easy to replace.

In some embodiments, either the elastic component 760 or the second elastic arm 725 may be optionally provided in the recessed structure 720. In some embodiments, both the elastic component 760 and the second elastic arm 725 may be provided in the recessed structure 720.

FIG. 7G is a schematic diagram illustrating another exemplary structure of a press button according to some embodiments of the present disclosure. Referring to FIG. 7G, in some embodiments, the pressing button 750 may also include a fixed structure 757, and the elastic structure may include an elastic piece mechanism 758. The fixed structure 757 may be disposed on a peripheral side of the pressing button 750 except the side where the connection structure 752 is disposed, and the pressing button 750 may be connected to the reservoir housing 710 through the fixed structure 757. In the X direction, the fixed end of the elastic piece mechanism 758 may be connected to the fixed structure 757, and the free end of the elastic piece mechanism 758 may be disposed with the connection structure 752.

In the Y direction, there may be a gap between each of the two sides of the elastic piece mechanism 758 and the fixed structure 757, so that the free end of the elastic piece mechanism 758 may move relative to the fixed end of the elastic piece mechanism 758.

In some embodiments, the pressing surface 751 may be disposed on a surface of the elastic piece mechanism 758 away from the interior of the reservoir housing 710 (i.e., the upper surface).

In some embodiments, when the pressing button 750 is pressed in a direction close to the interior of the reservoir housing 710 (e.g., downward), the free end of the elastic piece mechanism 758 may move in a direction close to the interior of the reservoir housing 710, and the elastic piece mechanism 758 may undergo an elastic deformation. When the external force is released, the free end of the elastic piece mechanism 758 may move in a direction away from the interior of the reservoir housing 710, and the elastic piece mechanism 758 may return to its original shape, and drive the pressing button 750 to spring back in the direction away from the interior of the reservoir housing 710.

In some embodiments, at least one of the elastic component 760 and the elastic arm (e.g., the first elastic arm 755 and/or the second elastic arm 725) may be disposed between the elastic piece mechanism 758 and the reservoir housing 710. The elastic arm and/or the elastic component may be disposed on a side of the elastic piece mechanism 758 facing the interior of the reservoir housing 710 (e.g., the lower surface of the elastic piece mechanism 758), or may be disposed on the surface of the recessed structure 720 of the reservoir housing 710 corresponding to the pressing button 750, the present disclosure makes no limitations on this.

Some embodiments of the present disclosure further provides a ventilation treatment device, including the above reservoir 700. In some embodiments, the ventilation treatment device may further include a ventilator device and a high flow respiratory humidification therapy device, etc.

In the respiratory ventilation apparatus of the above embodiment, setting the pressing button realizes a detachable connection between the reservoir and the main body of the respiratory ventilation apparatus, reducing operation difficulty of disassembling the reservoir, and making the connection between the reservoir and the main body of the respiratory ventilation apparatus flexible to be set. Setting the elastic structure on the pressing button makes it possible for the pressing button to be automatically reset after being pressed, with no additional operation required, and the operations are simple. Through a movable setup of the pressing button relative to the reservoir housing, the reservoir may be kept stable during disassembly and is not easy to shake relative to the main body of the respiratory ventilation apparatus. In such cases, not only the connection between the reservoir and the main body of the respiratory ventilation apparatus is improved, but also the difficulty of the reservoir to enter and exit the main body of the respiratory ventilation apparatus is reduced, ensuring a simpler disassembly and installation of the reservoir.

Currently, when using a respiratory ventilation apparatus, FIG. 8A is a schematic diagram illustrating an exemplary main body and humidifier according to some embodiments of the present disclosure.

The respiratory ventilation apparatus may improve a user's respiratory function. The respiratory ventilation apparatus may include a plurality of types, such as a home respiratory ventilation apparatus, etc. The respiratory ventilation apparatus may include components such as a main body, a humidifier, etc. The main body of the respiratory ventilation apparatus may draw gas into the humidifier. The humidifier may be configured to warm and/or humidify the gas, increasing a user's comfort level with the respiratory ventilation apparatus. The humidifier may include components such as a reservoir, a heating device, etc. The reservoir may be configured to store water needed for humidification of the humidifier. The heating device may heat a liquid (e.g., water, etc.) in the reservoir.

As shown in FIG. 8A, the reservoir of the humidifier 820 is connected to the main body of the respiratory ventilation apparatus 810, and gas flows from the main body of the respiratory ventilation apparatus 810 through a gas inlet channel into the reservoir of the humidifier 820. The gas is heated and/or humidified in the reservoir before flowing out through the gas outlet channel, which in turn is delivered to a user for use. FIG. 1B is an exemplary schematic diagram illustrating a respiratory ventilation apparatus according to some embodiments of the present disclosure. As shown in FIG. 1B, the respiratory ventilation apparatus 1000 includes a main body 1100 and a reservoir 1200 of a humidifier. In some embodiments, the main body 1100 may include a display 110. The display 110 may be configured to interact with a user. For example, a master control chip may indicate to the user how much liquid is in the reservoir through the display 110. More detailed descriptions illustrating the master control chip may be found in FIG. 8B and related description thereof.

In some embodiments, FIG. 1B is only exemplary, and a structure and a connection manner, etc. between the main body 1100 of the respiratory ventilation apparatus 1000 and the reservoir 1200 of the humidifier may be set up according to actual needs. For example, a chassis base plate of the main body of the respiratory ventilation apparatus may be provided with a metal heating plate, and the reservoir of the humidifier may be placed on the metal heating plate. The liquid in the reservoir may be heated by the metal heating plate. A bottom of the reservoir may be provided with a metal heat-conducting plate, etc., in contact with the metal heating plate. In some embodiments, a positioning fixing structure may be provided at a connection between a front end of the reservoir 1200 and a corresponding position of the main body 1100 to ensure that when the reservoir 1200 is connected to the main body 1100, the front end of the reservoir 1200 may be in a better contact with the metal heating plate of the main body 1100 to improve a heat transfer. For example, the front end of the reservoir 1200 may be disposed with a pair of protrusions extending forward. Docking components may be provided at the corresponding positions of the main body 1100 for exerting a pressing force on the upper surface of the protrusions. When the reservoir 1200 is connected to the main body 1100, the docking components may contact the protrusions and exert a force on the protrusion in a direction close to the metal heating plate.

FIG. 8B is a schematic diagram illustrating an exemplary liquid level detection device according to some embodiments of the present disclosure. FIG. 8C is a schematic diagram illustrating an exemplary level detection device according to some other embodiments of the present disclosure. FIG. 8B and FIG. 8C are enlarged schematic illustrations of a portion of structure where the reservoir of humidifier 820 and a main body of respiratory ventilation apparatus 810 are connected.

In some embodiments, the level detection device may be configured to detect a liquid level of a humidifier of the respiratory ventilation apparatus. In some embodiments, the level detection device may include at least one sensor that may be configured to detect the liquid level of the humidifier. The sensor may be provided on a sidewall of the main body of the respiratory ventilation apparatus. The sensor may be provided on a side of a sidewall of facing the reservoir or on a side of the sidewall away from the reservoir. A side of the main body may be adjacent to the sidewall of the reservoir.

In some embodiments, the level detection device may be configured to detect a liquid level of a reservoir of a humidifier.

In some embodiments, the level detection device may include at least one sensor. The sensor may be configured to detect the liquid level in the reservoir of the humidifier. As shown in FIG. 8B, the sensor may include one or two. For example, a first sensor 8211 and/or a second sensor 8212. A count of the sensor may be set according to actual needs. Different sensors may independently detect liquid levels at different locations in the reservoir of the humidifier. As shown in FIG. 8B, the first sensor 8211 is close to a bottom plate of the humidifier 821-1, and the first sensor 8211 may be configured to detect whether the liquid in the reservoir is depleted. The second sensor 8212 is at a distance from a bottom of the reservoir, and the second sensor 8212 may be configured to detect whether the liquid in the reservoir is sufficient.

The sensor may include a plurality of types, such as a capacitive sensor, etc.

In some embodiments, the sensor may be provided on the sidewall of the main body 811 of the respiratory ventilation apparatus. The sidewall of the main body 811 of the respiratory ventilation apparatus may be a sidewall adjacent to the sidewall of the humidifier 821. The sidewall of the main body 811 of the respiratory ventilation apparatus may include a side away from the sidewall of the humidifier 811-1 or a side facing the sidewall of the humidifier 811-2. As shown in FIG. 8B, the first sensor 8211 and/or the second sensor 8212 may be provided on the side away from the sidewall of the humidifier 811-1. As shown in FIG. 8C, the first sensor 8211 and/or the second sensor 8212 may be provided on a side facing the sidewall of the humidifier 811-2.

In some embodiments, the sensor may be provided on the sidewall of the main body 811 of the respiratory ventilation apparatus in a plurality of ways. For example, the sensor may be affixed directly to the sidewall of the main body, such as by gluing. As another example, the sensor may be set in a space with a certain gap from the sidewall of the main body by fixing (e.g., snapping, etc.), removing, etc.

A distance range of the gap may be set according to actual needs. Merely by way of example, a maximum gap distance of the distance range of the gap is a maximum distance of the gap that may realize detection of the liquid level in the reservoir.

In some embodiments of the present disclosure, the liquid level of the reservoir may be detected and determined in real time by the sensor of the liquid level detection device, which may improve accuracy and timeliness of determining the liquid level, reduce a probability of danger, and improve a usage effect and comfort of a user.

In some embodiments, a material of the sensor may include a metallic material. The metallic material may include copper, aluminum, gold, silver, stainless steel, etc. The metallic material may be selected according to an actual situation.

In some embodiments, the sensor may be an independent metal sheet or a metal sheet attached to a carrier, etc.

In some embodiments, the sensor may include at least one layer of metal sheet. For example, the sensor may include a single layer of metal sheet, a double layer of metal sheet, etc. The double layer of metal sheet may be obtained by covering a second layer of metal sheet after adding an insulating layer to one layer of metal sheet. By means of the double layer of metal sheet and in combination with a software algorithm, the sensor may be made to shield interference from electrical instruments other than the humidifier (e.g., an electrical instrument in the main body of the respiratory ventilation apparatus, etc.), and to increase the sensing speed and accuracy of the sensor.

Based on the actual situation, choosing different materials for the sensor may make the sensor more suitable for the actual needs. By attaching a metal sheet to a carrier, it is possible to make the metal sheet easier to process and easier to set on the sidewall of the main body of the respiratory ventilation apparatus.

In some embodiments, the carrier to which the metal sheet attached may include at least one of a PCB board, a flexible circuit board, etc.

A shape of the sensor may be set according to the actual needs. In some embodiments, the shape of the sensor may be a sheet. A size of the sensor in the shape of the sheet may be expressed in terms of length*width*thickness. In some embodiments, a length of the sensor may be within a range of 10 mm-100 mm, a range of 30 mm-60 mm, a range of 40 mm-50 mm, etc. When the sensor is placed horizontally, it indicates that a length direction is set along a horizontal direction, and the horizontal direction refers to a direction parallel to the ground. A width of the sensor may be within a range of 1 mm-7 mm, a range of 2 mm-5 mm, a range of 3 mm-4 mm, etc. A direction of the width of the sensor when it is placed horizontally is a direction perpendicular to the horizontal direction. A thickness of the sensor may be within a range of 0.03 mm-0.6 mm, a range of 0.05 mm-0.5 mm, a range of 0.08 mm-0.4 mm, etc. A size and a placement direction of the sensor may be set according to the actual needs. For example, a direction of the sensor when it is set on the sidewall of the main body of the respiratory ventilation apparatus may be set in the horizontal direction according to the length direction of the sensor, or it may be set in a direction perpendicular to the ground according to the length direction of the sensor.

In some embodiments of the present disclosure, by controlling the size of the sensor within a certain range, the sensor may be made more rationally designed and more sensitive. For example, setting a thickness range of the sensor to be within a range of 0.05 mm-0.5 mm may avoid a problem of the sensor being too thick, which causes a stronger overall interference with the detection to cause poorer detection accuracy due to the sensor being too thick.

In some embodiments, a height difference between a first positional height of the sensor and a second positional height of the bottom plate of the humidifier may be within a range of 1 mm-60 mm, a range of 2 mm-50 mm, a range of 3 mm-40 mm, etc. The height difference between the first positional height and the second positional height of the bottom plate of the humidifier may be set according to the actual needs.

The first positional height refers to data that may represent the height of the sensor in the direction perpendicular to the horizontal. For example, the first positional height may be a height of a center point of the sensor from the ground, the height of the center point of the sensor from a bottom plate of the main body of the respiratory ventilation apparatus, etc.

The second positional height refers to data that may represent the height of the bottom plate of the humidifier in the direction perpendicular to the horizontal. For example, the second positional height may be the height of the bottom plate of the humidifier from the ground, the height of the bottom plate of the humidifier from the bottom plate of the main body of the respiratory ventilation apparatus, etc.

Corresponding references are the same when indicating the height of the bottom plate of the sensor and the humidifier in the direction perpendicular to the horizontal. For example, the references are both the ground or the bottom plate of the main body of the respiratory ventilation apparatus, etc.

A height difference between the first positional height and the second positional height may reflect a height difference between the center point of the sensor and the bottom plate of the humidifier.

FIG. 8D is a schematic diagram illustrating an exemplary height difference according to some embodiments of the present disclosure. As shown in FIG. 8D, a height difference H1-H2 between a first positional height H1 (e.g., a height of a center point of the sensor from the ground) of the first sensor 8211 and a second positional height H2 (e.g., a height of a bottom plate of a humidifier from the ground) of the bottom plate of the humidifier.

In some embodiments of the present disclosure, a real-time detection of a liquid level at different heights may be achieved by setting a height difference between the sensor and the bottom plate of the humidifier. For example, if the height difference H1-H2 between the first positional height H1 of the sensor and the second positional height H2 of the bottom plate of the humidifier is 2 mm, the sensor may sense in real time if the liquid level of a reservoir is lower (near the bottom plate of the humidifier). When the sensor senses that the liquid level of the reservoir is lower than 2 mm, it may determine that the liquid in the reservoir is about to run out, and then it may control a heating device to stop heating, which may avoid the liquid in the reservoir being burned to dry and be in dangerous situations, thereby improving a usage effect and comfort of a user. As another example, when the height difference between the first positional height of the sensor and the second positional height of the bottom plate of the humidifier is 50 mm, the sensor may sense in real time whether the liquid level of the reservoir is lower than 50 mm. When the sensor senses that the liquid level of the reservoir is lower than 50 millimeters, it may determine a preset time when the liquid in the reservoir is close to being depleted, which in turn may prompt the user on a display screen of the respiratory ventilation apparatus to indicate that at this time, a time of the liquid may be expected to be consumed, whether liquid replenishment is required, etc. The preset time when the liquid in the reservoir is close to being depleted may be determined by sensors with different height differences, which may then prompt the user at different stages as to whether or not the liquid replenishment is required, etc., thereby enhancing a user's experience.

In some embodiments, the sensor may include a plurality of sensors, each of which may be separately provided at a preset positional height in a vertical direction. The vertical direction refers to a direction perpendicular to a horizontal direction.

A height of each sensor from the bottom plate of the humidifier varies. A count of the sensor and a preset positional height of the sensor set in the vertical direction may be set according to the actual needs. Sensors with different preset positional heights may detect a liquid level of the reservoir corresponding to a preset height.

In some embodiments of the present disclosure, by means of a plurality of sensors, and by setting the sensors at different positional heights in the vertical direction, it is possible to realize the real-time detection of whether or not the liquid level is lower than a specific height, and it is possible to further improve accuracy and rapidity of detection.

In some embodiments, when the sensor is provided on a side of a sidewall of the main body away from a sidewall of the humidifier, a distance between the sensor and an inner wall of the sidewall of the humidifier in the horizontal direction is within a range of 1 mm-8 mm.

The distance between the sensor and the inner wall of the sidewall of the humidifier in the horizontal direction may include a sum of a thickness of the sidewall of the main body, a thickness of the sidewall of the humidifier, and a width of a gap between the sidewall of the main body and the sidewall of the humidifier.

In some embodiments of the present disclosure, accuracy and sensitivity of the liquid level detection device may be further improved by setting the distance between the sensor and the inner wall of the sidewall of the humidifier in the horizontal direction within a certain range. For example, setting the distance between the sensor and the inner wall of the sidewall of the humidifier in the horizontal direction to be within a range of 1 mm-8 mm may avoid that, due to the distance between the sensor and the sidewall of the humidifier being too large, interference on a detection (e.g., a capacitance value, etc.) is strong, leading to a problem of poor detection accuracy of the sensor.

In some embodiments, the sensor may include a capacitive sensor, and the liquid level detection device may include a master control chip. The master control chip may be connected to a circuit of the sensor, and the master control chip may be configured to receive a capacitance value captured by the sensor and to judge, based on the capacitance value, whether the liquid level is lower than the positional heigh of the sensor in the vertical direction.

The master control chip may be connected to the circuit of the sensor by a wire, etc. Whether there is a liquid in the reservoir with the same height as a position of the sensor in the vertical direction, makes the capacitance value corresponding to the sensor varies greatly. For example, when the liquid level of the reservoir exceeds an upper edge of the sensor in the vertical direction, the capacitance value is a. When the liquid level of the reservoir is lower than a lower edge of the sensor in the vertical direction, the capacitance value is b. A difference between the capacitance value of a and the capacitance value of b is large. The master control chip may collect the capacitance value of the capacitance sensor in real time through the wire, and then determine the liquid level of the reservoir. For example, whether the liquid level of the reservoir is lower than the height of the position of the sensor in the vertical direction.

In some embodiments, the above wire may be an ordinary single-core wire, an RF signal wire, etc. Through the RF signal wire, and in combination with a software algorithm, the RF signal wire may be shielded from interference from a surrounding appliance.

In some embodiments, the master control chip may, based on a correspondence between the height of the position of the sensor in the vertical direction set in advance and the liquid level of the reservoir, determine whether the liquid in the reservoir is depleted or whether there is a residual liquid, and then control whether the heating device stops heating or to prompt the user to replenish the liquid. For example, the master control chip may determine, based on a change in the capacitance value of the first sensor 8211, whether the liquid level of the reservoir is lower than the lower edge of the first sensor 8211 in the vertical direction, and thereby determine whether the liquid in the reservoir is depleted, and thus control whether the heating device stops heating. As another example, the master control chip may determine whether the respiratory ventilation apparatus is turned on to a humidification gear. If the humidification gear is turned on, the master control chip can collect the capacitance value of the second sensor 8212 in real-time, and based on the change in the capacitance value of the second sensor 8212, determine whether the liquid level of the reservoir is lower than the lower edge of the second sensor 8212 in the vertical direction. By judging whether the liquid level of the reservoir is lower than the lower edge of the second sensor 8212 in the vertical direction, the master control chip may prompt the user on the display of the respiratory ventilation apparatus that an amount of liquid at this time is insufficient for a preset period of time (e.g., 8 hours) of consumption, and whether it needs to be replenished, etc.

In some embodiments of the present disclosure, the master control chip is connected to the circuit of the capacitance sensor, and based on the capacitance value, whether the liquid level is lower than the height of the position of the sensor in the vertical direction is determined. Capacitance sensing of the liquid level detection device is a kind of remote sensing effect, in which direct contact is not required between the sensor and a measured object (such as water, etc.). With simple structures, the sidewall of the main body and the sidewall of the reservoir do not need to be punched and do not limit a style of the reservoir, making circuit detection simpler, detection more accurate, an anti-interference ability higher, and a cost lower.

FIG. 9A is a schematic diagram illustrating a structure of a noise reduction device disposed with a flow detection device according to some embodiments of the present disclosure; FIG. 9B is a schematic diagram illustrating an exemplary structure of a flow detection device according to some embodiments of the present disclosure; and FIG. 9C is a diagram illustrating an exemplary structure of a flow detection device according to some embodiments of the present disclosure.

In some embodiments, as shown in FIG. 9A, a noise reduction device 900 in a respiratory ventilation apparatus may include a gas inlet pipe 910, a noise reduction housing 920, a blower cavity 930, a gas channel 940, and a flow detection device 950. The flow detection device 950 is configured to detect a gasflow rate output from the respiratory ventilation apparatus. The flow detection device 950 is provided in the noise reduction housing 920, so that a gasflow creates a pressure difference between two sides of the flow detection device 950 along a direction of the gasflow, thus a flow rate detection may be realized by using a principle of pressure difference.

In some embodiments, as shown in FIG. 9A and FIG. 9B, the flow detection device 950 is provided with a low pressure detection point 951 and a high pressure detection point 952. The low pressure detection point 951 and the high pressure detection point 952 may be through holes, lower ends of which are connected to the gas channel 940 and near a cavity gas outlet of the blower cavity 930, respectively, and upper ends of which are connected to two flow sensors 953, respectively. The two flow sensors 953 may be in a form of a column, snapped in the low pressure detection point 951 and the high pressure detection point 952 for detecting different gas pressures when the gasflow flows through the low pressure detection point 951 and the high pressure detection point 952. Then a pressure difference between the two points is obtained by calculating through the processor, so that the principle of pressure difference may be used to realize the flow rate detection. The low pressure detection point 951 is located at a beginning end of the gas channel 940, and the high pressure detection point 952 is near the cavity gas outlet of the blower cavity 930.

In some embodiments, as shown in FIG. 9C, a back side of the flow detection device (i.e., a side facing the gas channel and the blower cavity when provided), the high pressure detection point 952 may be a through hole that is vertical in shape, and the low pressure detection point 951 may be a turning hole opened in the flow detection device 950. A gas hole 9511 at a lower end of the low pressure detection point 951 connected to the gas channel 940 is close to the cavity gas inlet 931 (the cavity gas inlet 931 may be a first gas inlet 231 in the embodiment described above), where a gas pressure is closest to the atmospheric pressure outside the respiratory ventilation apparatus. Thus, the pressure difference detected by the flow detection device 950 may be larger and more stable, which may improve comfort of using the respiratory ventilation apparatus.

FIG. 10A is a schematic diagram illustrating an exemplary structure of a pipe structure of a respiratory ventilation apparatus according to some embodiments of the present disclosure.

In some embodiments, as shown in FIG. 1A and FIG. 10A, the respiratory ventilation apparatus 1000 further includes a pipeline interface 120 communicated with the user interface 7000, such as a respiratory mask. In some embodiments, a humidifier shown hereinabove may include components such as a reservoir, a heating device, etc. The reservoir may be configured to store water needed to humidify the humidifier. The heating device may heat a liquid in the reservoir (e.g., the water, etc.). External gas flows from the main body 1100 of the respiratory ventilation apparatus 1000 into the reservoir of the humidifier through a gas inlet channel, and the gas is heated and/or humidified in the reservoir and then flows out through a gas outlet channel. The gas is then delivered to the user through the pipeline interface 120 and the external device (e.g., the respiration pipe 6000, the user interface 7000) connected thereto. In order to better detect whether a temperature of the gas flowing out of the pipeline interface 120 is suitable for the user, a temperature detection device 121 may be provided in the pipeline interface 120 to objectively detect a temperature of the gas at the pipeline interface 120. A heating temperature of the heating device in the humidifier is further adjusted based on the temperature detected by the temperature detection device 121. In some embodiments, a display screen on the main body 1100 may also display in real time the temperature of the gas detected by the temperature detection device 121.

In some embodiments, the respiratory ventilation apparatus 1000 further includes a power supply interface 130 for connecting to an external power plug. The external power plug may be integrated in the external gasflow pipe, and when the external gasflow pipe is connected to the pipeline interface 120, the external power pipe may be electrically connected to the power supply interface 130 to heat the gas in the external gasflow pipe. The power supply interface 130 is elastically electrically connected to an external power plug. As shown in FIG. 10B, the power supply interface 130 is provided with a metal elastic sheet 131 inside. When an external power plug is inserted into the power supply interface 130, the metal elastic sheet 131 springs upward under an action of an external force and its own elasticity, causing a line region contact electrical connection to be formed between the external power plug and the metal elastic sheet 131. A contact region between the external power plug and the metal elastic sheet 131 is small, which reduces friction and allows for quick insertion and connection of the external power plug, as well as improving a service life of the power supply interface 130.

FIG. 11A is a schematic diagram illustrating an exemplary exploded structure of a main body button according to some embodiments of the present disclosure; and FIG. 11B is a schematic diagram illustrating an exemplary structure of a main body case according to some embodiments of the present disclosure.

In some embodiments, as shown in FIG. 1B, FIG. 11A, and FIG. 11B, the respiratory ventilation apparatus 1000 further includes a main body button 140 provided on a main body housing. The main body button 140 includes a plurality of functional buttons for a user to operate the respiratory ventilation apparatus. The main body button 140 includes a silicone layer 141, a metal bracket 142, and a button panel 143 provided sequentially on the main body housing from bottom to top. As shown in FIG. 11A, a plurality of hot melt holes 1421 are provided in the metal bracket 142. The main body housing of the respiratory ventilation apparatus 1000 has a plurality of plastic posts (not shown in the figures) extending upwardly from a main body upper housing, a size of the plurality of plastic posts matches a size of the plurality of hot melt holes 1421 provided in the metal bracket 142. When assembling the main body button 140 to the main body housing, the silicone layer 141 is first provided on the main body housing through positioning holes 1411, and the plurality of hot melt holes 1421 are provided over the plurality of plastic posts of the main body housing to enable the metal bracket 142 press the silicone layer 141 to seal and fit on the main body housing. Then the plurality of plastic posts is melted through hot melting to make the metal bracket 142 fixed on the main body housing to ensure a sealing effect of the silicone layer 141, and to achieve a waterproof effect of the main body button 140.

In some embodiments, the button panel 143 is snap-attached to the metal bracket 142. A plurality of buckle hooks 1431 are provided on a lower end face of the button panel 143, and a protruding end 1422 is provided on the metal bracket 142 that mates with the plurality of buckle hooks 1431. When the button panel 143 needs to be mounted, the button panel 143 is placed on top of the metal bracket 142, and the plurality of buckle hooks 1431 may be hooked to the protruding end 1422 under an action of an external force to realize installation of the button panel 143 in coordination with the metal bracket 142. As shown in FIG. 111B, buckle hooks 1431 may be four, and corresponding protruding ends 1422 are also four.

In some embodiments, a plurality of elastic buttons 1412 are provided on the silicone layer 141, and a lower end of each of the plurality of elastic button 1412 has a button patch 1413 that may be electrically connected to a circuit board in the main body. A lower end of the button panel 143 is provided with downwardly a button protrusion (not shown in the figures) that matches a position of an elastic button 1412. The button panel 143 may dispose a plurality of buttons or button regions, and the elastic button 1412 corresponding to each button or button regions may be disposed respectively on the silicone layer 141. When there is a need to operate the respiratory ventilation apparatus 1000, the user may press one of the buttons of the button panel 143, and then the button protrusion moves downwardly to make the button patch 1413 at a lower end of the elastic button 1412 connect with the circuit board in the main body, which triggers a corresponding function (e.g., startup, heating, etc.) of the button.

The embodiments of the present disclosure also provide a button. As shown in FIG. 1B, a schematic diagram illustrating a structure of a respiratory ventilation apparatus including the main body 1100 and the reservoir 1200 of the humidifier provided on the main body 1100 to be able to supply humidified gas through a respiration pipe (not shown) to a patient. The main body 1100 is connected to the button 150, and the main body 1100 may be operated by pressing the button 150, so as to realize operations such as power on and off control and parameter setting. The present disclosure provides the button 150 that may be used in the respiratory ventilation apparatus, and in combination with a plurality of different embodiments shown in FIGS. 12A-12R, the button 150 is integrated with at least two button function portions, such as a first button function portion 1511 and a second button function portion 1512. By selectively pressing at least one of the first button function portion 1511 and the second button function portion 1512, manipulation of a working state of the main body 1100, such as switching on and off, gas flow rate adjustment, and gas supply humidity adjustment, etc., may be realized. It may be understood that although the button 150 provided in the present disclosure is described in detail herein using the respiratory ventilation apparatus as an example, application scenarios of the button 150 are not limited, and the button 150 may also be used in other powered devices.

The button 150 provided in the present disclosure includes a button body 151 and a button connection portion 152. The button body 151 includes the first button function portion 1511 and the second button function portion 1512, which may be used as different function regions to be respectively triggered to make the main body 1100 perform different functions (including the on and off control). In other embodiments, the button body 151 may, for example, also include the first button function portion, the second button function portion, a third button function portion, etc. In the embodiment, end portions of the first button function portion 1511 and the second button function portion 1512 are opposite to each other. A narrow slot 1513 is formed between the opposite end portions, which opens at least at a pressing side 151a of the button body 151, i.e., through the pressing side 151a of the button body 151. The button connection portion 152 has a stiffness that is less than a stiffness of the first button function portion 1511 and a stiffness of the second button function portion 1512, and is connected to the first button function portion 1511 and the second button function portion 1512 by spanning the narrow slot 1513.

By integrally providing the first button function portion 1511 and the second button function portion 1512 connected by the button connection portion 152. The button 150 of the embodiment of the present disclosure, as compared to a separate button, may effectively reduce a production cost, design complexity, and space occupation. When a user presses one of the first button function portion 1511 and the second button function portion 1512, the narrow slot 1513 formed therein effectively reduces transmission of a force to the other. Because the button connection portion 152 has a relatively small stiffness, it is not necessary for the second button function portion 1512 to be connected to the other, thus ensuring high reliability and operability. In order to make the button connection portion 152 have a relatively small stiffness relative to the first button function portion 1511 and the second button function portion 1512, the methods as described in the following embodiments may be obtained by selecting material hardness, or by forming the button connection portion 152 to have a relatively thin connection diaphragm.

Although the button body 151 shown in a plurality of embodiments includes only the first button function portion 1511 and the second button function portion 1512, in other embodiments, it may further include more other button function portions. Two adjacent button function portions have end portions opposite to each other and form narrow slot 1513 between the portions, and are connected to each other by the button connection portion 152.

The first button function portion 1511 and the second button function portion 1512 may be set to be parallel to each other at one end opposite to each other and have an appropriate gap to reduce a risk of dust accumulation and water ingress under a premise of allowing the two to be able to have a certain degree of relative motion when passing through the narrow slot 1513. And at ends of the first button function portion 1511 and the second button function portion 1512 opposite to each other, curved transition corners may be formed respectively on each end, thereby providing better aesthetics when attached to the main body 1100. Additionally, the first pressing surface 1511a of the first button function portion 1511 and the second pressing surface 1512a of the second button function portion 1512 may be provided in a same plane, which may facilitate ensuring a surface of a device is flat and beautiful.

FIGS. 12A-12F illustrate a button according to some embodiments of the present disclosure. As previously described, the button includes the button body 151 and the button connection portion 152. The button body 151 has the first button function portion 1511 and the second button function portion 1512 with ends opposite to each other, and the narrow slot 1513 is provided between the first button function portion 1511 and the second button function portion 1512. The button connection portion 152 spans the narrow slot 1513 and is connected to the first button function portion 1511 and the second button function portion 1512, respectively.

The first button function portion 1511 and the second button function portion 1512 may be made of a relatively rigid material such as PC, ABS, etc. The button connection portion 152 may be made of a relatively soft material such as TPU, TPE, etc. As a result, the button connection portion 152 may be formed to have a stiffness that is less than that of the first button function portion 1511 and the second button function portion 1512.

The button connection portion 152 is provided on a side of the button body 151 that is back from the pressing side 151a (i.e., the first pressing surface 151 la and the second pressing surface 1512a) and is connected across the narrow slot 1513 to the first button function portion 1511 and the second button function portion 1512, respectively. In a manufacturing process, the button connection portion 152 may be connected to the first button function portion 1511 and the second button function portion 1512 by a two-color injection molding manner, which has advantages of simple process, high safety, and reliability.

To ensure that there is a high connection strength, the first button function portion 1511 and the second button function portion 1512 may be formed with a protrusion 1514 protruding toward the button connection portion 152. Accordingly, the button connection portion 152 is formed with a connection hole 1522 corresponding to the protrusion 1514. In other embodiments, positions of the protrusion 1514 and the connection hole 1522 may be wholly or partially interchanged. For example, if a connection hole (e.g., a blind hole) is formed on the first button function portion 1511 and the second button function portion 1512, a protrusion corresponding to the connection hole is formed in the button connection portion 152, which may likewise enhance a connection strength.

In the embodiment, as shown in FIG. 12E and FIG. 12F, at a position corresponding to the narrow slot 1513 on the button body 151, the button connecting portion 152 may be formed with an arc extension 1521 that protrudes towards the pressing side 151a away from the button body 151. Therefore, when one of the first button function portion 1511 and the second button function portion 1512 is operated, a force transfer and deformation occurring on the button connection portion 152 are increased by the arc extension 1521. Thus, a trigger linkage of other one of the first button function portion 1511 and the second button function portion 1512 may be effectively avoided, thereby ensuring reliability of the button.

Alternatively, at the ends opposite to each other, two sides of the first button function portion 1511 and the second button function portion 1512 are connected to each other so that the narrow slot 1513 is open on a side departing the pressing side 151a of the button body 151, i.e. the narrow slot 1513 runs through a side of departing the pressing side 151a of the button body 151. By using a hard material of the button body 151 to connect the first button function portion 1511 and the second button function portion 1512 and retaining a connecting region only on both sides, which may avoid deformation and warping of one side of the two button function portions that would occur if the two key function portions rely only on the button connection portion 152 of a soft material for connecting.

FIGS. 12G-12L illustrate a button illustrated in some other embodiments of the present disclosure. Similar to the foregoing embodiments, the button includes the button body 151 and the button connection portion 152. The button body 151 has the first button function portion 1511 and the second button function portion 1512, and the first button function portion 1511 and the second button function portion 1512 are provided with the narrow slot 1513 between them. The button connection portion 152 spans the narrow slot 1513 and is connected to the first button function portion 1511 and the second button function portion 1512, respectively.

The first button function portion 1511 and the second button function portion 1512 may be made of a material such as PC, ABS, etc. The button connection portion 152 may be a connection diaphragm made of a material such as PC, PET, etc. Thus, a stiffness of the button connection portion 152 that is less than that of the first button function portion 1511 and the second button function portion 1512 may be formed.

The button connection portion 152 is provided on a side of the button body 151 away from the pressing side 151a and is connected across the narrow slot 1513 to the first button function portion 1511 and the second button function portion 1512, respectively. In a manufacturing process, the button connection portion 152 may be connected to the first button function portion 1511 and the second button function portion 1512 as a single unit through in-mold molding (IML), which has advantages of simple process, high safety, and reliability.

In the embodiment, as shown in FIG. 12K and FIG. 12L, at a position corresponding to the narrow slot 1513 of the button body 151, the button connection portion 152 may be formed with the arc extension 1521 that protrudes towards the pressing side 151a away from the button body 151. Therefore, when one of the first button function portion 1511 and the second button function portion 1512 is operated, the force transfer and deformation occurring on the button connection portion 152 are increased by the arc extension 1521. Thus, a trigger linkage of the other one of the first button function portion 1511 and the second button function portion 1512 may be effectively avoided, thereby ensuring reliability of the button.

The first pressing surface 151 la and the second pressing surface 1512a of the first button function portion 1511 and the second button function portion 1512 may also be provided with an in-mold insert injection molding diaphragm to form an indication of a covered pattern or text on each button function portion, thereby keeping a color sharp for a long period of time without being easily faded or scratched.

Unlike the previous embodiment, the embodiment does not connect the first button function portion 1511 and the second button function portion 1512 at opposite ends to each other, instead, the narrow slot 1513 is left completely open on the side departing the pressing side 151a of the button body 151, and the first button function portion 1511 and the second button function portion 1512 are connected as a single unit only by the button connection portion 152.

In alternative embodiments, at ends opposite to each other, each side of the first button function portion 1511 and the second button function portion 1512 may also be separately attached to each other so that the narrow slot 1513 is open on the side departing the pressing side 151a of the button body 151. By using a hard material of the button body 151 to connect the first button function portion 1511 and the second button function portion 1512 and retaining a connecting region only on both sides, which may avoid deformation and warping of one side of the two button function portions that would occur if the two key function portions rely only on the button connection portion 152 of a soft material for connecting.

FIGS. 12M-12R illustrate a button according to another embodiment of the present disclosure. Similar to the two preceding embodiments, the button includes a button body 151 and a button connection portion 152. The button body 151 has a first button function portion 1511 and a second button function portion 1512 with end portions provided opposite to each other, and a narrow slot 1513 is provided between the first button function portion 1511 and the second button function portion 1512. The button connection portion 152 spans the narrow slot 1513 and is connected to the first button function portion 1511 and the second button function portion 1512, respectively.

The first button function portion 1511 and the second button function portion 1512 may be made of a material such as PC, ABS, etc. And the button connection portion 152 may be a connection diaphragm made of a material such as PC, PET, etc. Thus, a stiffness of the button connection portion 152 that is less than that of the first button function portion 1511 and the second button function portion 1512 may be formed.

Unlike the foregoing embodiment, the embodiment forms the button connection portion 152 to enclose the pressing side 15 la and a periphery of the button body 151, thereby connecting across the narrow slot 1513 to the first button function portion 1511 and the second button function portion 1512. In a manufacturing process, the button connection portion 152 may be connected to the first button function portion 1511 and the second button function portion 1512 by in-mold molding (IML), which has advantages of simple process, high safety, and reliability.

In the embodiment, as shown in FIG. 12P and FIG. 12R, at a position corresponding to the narrow slot 1513 on the button body 151, the button connection portion 152 may be formed with an arc extension 1521 that protrudes towards a pressing side 151a away from the button body 151, and the arc extension 1521 inserts into the narrow slot 1513. As a result, when one of the first button function portion 1511 and the second button function portion 1512 is operated, force transfer and deformation occurring at the button connection portion 152 are increased by the arc extension 1521. Thus, a trigger linkage of the other one of the first button function portion 1511 and the second button function portion 1512 may be effectively avoided, thereby ensuring reliability of the button.

Alternatively, at the end portions opposite to each other, both sides of the first button function portion 1511 and the second button function portion 1512 are connected to each other so that the narrow slot 1513 is open on the side of the button body 151 away from the pressing side 151a, i.e., the narrow slot 1513 runs through the side of the button body 151 away from the pressing side 151a. By using a hard material of the button body 151 to connect the first button function portion 1511 and the second button function portion 1512 and retaining a connecting region only on both sides, which may avoid deformation and warping of one side of the two button function portions that would occur if the two key function portions rely only on the button connection portion 152 of a soft material for connecting.

The button 150 shown in the various embodiments described above may be used in a respiratory ventilation apparatus including a main body 1100 and a reservoir 1200 of a humidifier provided on the main body 1100. The button 150 is connected to the main body 1100, and the main body 1100 is capable of being manipulated by the first button function portion 1511 and the second button function portion 1512 of the button 150 to perform different functions.

In the embodiments of the present disclosure, the respiratory ventilation apparatus may include a household ventilator, a medical ventilator, or a high-flow respiratory humidification device, etc.

It should be noted that beneficial effects that may be produced by different embodiments are different, and the beneficial effects that may be produced in different embodiments may be any one or a combination of any of the foregoing, or any other beneficial effect that may be obtained.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. In addition, some features, structures, or characteristics of one or more embodiments in the present disclosure may be properly combined.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A noise reduction device comprising: a noise reduction housing, a gas inlet structure, a blower cavity, and a gas channel;
wherein a gas enters the gas channel through a gas outlet of the gas inlet structure; the gas channel is disposed between an inner wall of the noise reduction housing and an outer wall of the blower cavity; the gas channel is disposed along the outer wall of the blower cavity; the blower cavity is disposed with a cavity gas inlet; and the gas in the gas channel enters the blower cavity through the cavity gas inlet; and
wherein the gas inlet structure is located at a beginning of an extension direction of the gas channel, and the cavity gas inlet is located at an end of the extension direction of the gas channel; an angle between a first connecting line connecting a gas outlet of the gas inlet structure and a center of the gas channel and a second connecting line connecting the cavity gas inlet and the center of the gas channel is greater than 180°.

2. The noise reduction device of claim 1, wherein the gas inlet structure includes a gas inlet pipe with a bending structure, so that the gas flows into the gas channel after turning in the gas inlet pipe.

3. The noise reduction device of claim 2, wherein a first gas flow direction of a gas inlet of the gas inlet pipe is different from a second gas flow direction of a gas outlet of the gas inlet pipe.

4. The noise reduction device of claim 3, wherein the bending structure makes a gas inlet of the gas inlet pipe and a gas outlet of the gas inlet pipe located at different heights.

5. The noise reduction device of claim 4, wherein the bending structure includes upward bending so that the gas outlet of the gas inlet pipe is higher than the gas inlet of the gas inlet pipe; the first gasflow direction is horizontal; and the second gasflow direction is vertical.

6. The noise reduction device of claim 5, wherein the gas inlet pipe includes a horizontal direction section and a vertical direction section, the vertical direction section having an overall height of 2 to 2.8 times a diameter of the horizontal direction section.

7. The noise reduction device of claim 1, wherein the cavity gas inlet is disposed vertically at a height close to a motor portion of the blower in the blower cavity, so that a gas flow cools the blower in the blower cavity after entering the blower cavity through the cavity gas inlet.

8. The noise reduction device of claim 1, wherein
the noise reduction housing further includes a water collection cavity; and
the blower cavity further includes a cavity gas outlet, the cavity gas outlet being communicated with the water collection cavity.

9. The noise reduction device of claim 8, wherein the noise reduction housing is disposed with a main outlet, the gas flows out from the cavity gas outlet into the water collection cavity, and flows out of the main outlet after being lifted.

10. The noise reduction device of claim 9, wherein the cavity gas outlet is located at a height higher than a bottom surface of the water collection cavity in a vertical direction.

11. The noise reduction device of claim 1, wherein
a porous sound-absorbing plate and/or a sound-absorbing cotton is placed in the gas channel; and
the porous sound-absorbing plate is disposed with a plurality of sound-absorbing holes in a thickness direction.

12. The noise reduction device of claim 11, wherein the porous sound-absorbing plate includes a plurality of regions, and apertures of the plurality of sound-absorbing holes on each of the plurality of regions is disposed according to a preset rule.

13. The noise reduction device of claim 12, wherein the porous sound-absorbing plate includes a porous top plate, a porous side plate and a porous bottom plate; the porous sound-absorbing plate is disposed with the plurality of sound-absorbing holes in the thickness direction; and the apertures of the plurality of sound-absorbing holes on the porous top plate, the porous side plate and/or the porous bottom plate are set according to the preset rule.

14. The noise reduction device of claim 11, wherein the sound-absorbing cotton is a non-porous sound-absorbing cotton.

15. A respiratory ventilation apparatus comprising the noise reduction device of claim 1.
